(19) United States
(12) Patent Application Publication
(10) Pub. No.: US 2007/0240243 A9
(43) Pub. Date: Oct. 11, 2007

(54) Plant transcriptional regulators of drought stress

(75) Inventors: Jacqueline E. Heard, Stonington, CT (US); Jose Luis Riechmann, Pasadena, CA (US); Robert A. Creelman, Castro Valley, CA (US); Oliver Ratcliffe, Oakland, CA (US); Roger D. Canales, San Francisco, CA (US); Peter P. Repetti, Emeryville, CA (US); Roderick W. Kumimoto, San Bruno, CA (US); Neal I. Gutterson, Oakland, CA (US); T. Lynne Reuber, San Mateo, CA (US); Omaira Pineda, Vero Beach, FL (US); Bradley K. Sherman, Berkeley, CA (US)

Correspondence Address: MENDEL BIOTECHNOLOGY C/O MOFO SF, 425 MARKET STREET, SAN FRANCISCO, CA, 94105, US

(73) Assignee: Mendel Biotechnology, Inc., Hayward, CA (US)

(21) Appl. No.: 10/714,887

(22) Filed: Nov. 13, 2003

Related U.S. Application Data
(63) Continuation-in-part of application No. 10/295,403, filed on Nov. 15, 2002, now abandoned.
Application No. 10/295,403 is a division of application No. 09/394,519, filed on Sep. 13, 1999, now abandoned.
(63) Continuation-in-part of application No. 10/302,267, filed on Nov. 22, 2002, now Pat. No. 7,223,904.
Application No. 10/302,267 is a division of application No. 09/506,720, filed on Feb. 17, 2000, now abandoned.
(63) Continuation-in-part of application No. 10/286,264, filed on Nov. 1, 2002.
Application No. 10/286,264 is a division of application No. 09/533,030, filed on Mar. 22, 2000, now abandoned.
(63) Continuation-in-part of application No. 10/278,173, filed on Oct. 21, 2002, now abandoned.
Application No. 10/278,173 is a division of application No. 09/533,392, filed on Mar. 22, 2000, now abandoned.
(63) Continuation-in-part of application No. 09/533,029, filed on Mar. 22, 2000, now Pat. No. 6,664,446.
(63) Continuation-in-part of application No. 10/278,536, filed on Oct. 22, 2002, now abandoned.
Application No. 10/278,536 is a division of application No. 09/532,591, filed on Mar. 22, 2000, now abandoned.
(63) Continuation-in-part of application No. 09/713,994, filed on Nov. 16, 2000, now abandoned.
(63) Continuation-in-part of application No. 10/675,852, filed on Sep. 30, 2003.
(63) Continuation-in-part of application No. 09/934,455, filed on Aug. 22, 2001, now abandoned.
Application No. 09/934,455 is a continuation-in-part of application No. 09/713,994, filed on Nov. 16, 2000, now abandoned.
Application No. 09/934,455 is a continuation-in-part of application No. 09/837,944, filed on Apr. 18, 2001, now abandoned.
(63) Continuation-in-part of application No. 10/225,068, filed on Aug. 9, 2002, now Pat. No. 7,193,129.
(63) Continuation-in-part of application No. 09/837,944, filed on Apr. 18, 2001, now abandoned.
(63) Continuation-in-part of application No. 10/171,468, filed on Jun. 14, 2002, now abandoned.
(63) Continuation-in-part of application No. 10/225,066, filed on Aug. 9, 2002, now Pat. No. 7,238,860.
(63) Continuation-in-part of application No. 09/837,944, filed on Apr. 18, 2001, now abandoned.
(63) Continuation-in-part of application No. 10/171,468, filed on Jun. 14, 2002, now abandoned.
(63) Continuation-in-part of application No. 10/225,067, filed on Aug. 9, 2002, now Pat. No. 7,135,616.
(63) Continuation-in-part of application No. 09/837,944, filed on Apr. 18, 2001, now abandoned.
(63) Continuation-in-part of application No. 10/171,468, filed on Jun. 14, 2002, now abandoned.
(63) Continuation-in-part of application No. 09/823,676, filed on Mar. 30, 2001, now Pat. No. 6,717,034.
(63) Continuation-in-part of application No. 10/669,824, filed on Sep. 23, 2003.
(63) Continuation-in-part of application No. 10/112,887, filed on Mar. 18, 2002, now abandoned.
(63) Continuation-in-part of application No. 10/412,699, filed on Apr. 10, 2003.
Application No. 10/412,699 is a continuation-in-part of application No. 09/489,376, filed on Jan. 21, 2000, now abandoned.
Application No. 10/412,699 is a continuation-in-part of application No. 09/819,142, filed on Mar. 27, 2001, now abandoned.
Application No. 10/412,699 is a continuation-in-part of application No. 10/374,780, filed on Feb. 25, 2003.
(63) Continuation-in-part of application No. 10/374,780, filed on Feb. 25, 2003.
(63) Continuation-in-part of application No. 10/666,642, filed on Sep. 18, 2003, now Pat. No. 7,196,245.
(60) Provisional application No. 60/101,349, filed on Sep. 22, 1998.
(60) Provisional application No. 60/103,312, filed on Oct. 6, 1998.
(60) Provisional application No. 60/108,734, filed on Nov. 17, 1998.
(60) Provisional application No. 60/113,409, filed on Dec. 22, 1998.
(60) Provisional application No. 60/120,880, filed on Feb. 18, 1999.
(60) Provisional application No. 60/121,037, filed on Feb. 22, 1999.
(60) Provisional application No. 60/124,278, filed on Mar. 11, 1999.
(60) Provisional application No. 60/129,450, filed on Apr. 15, 1999.
(60) Provisional application No. 60/135,134, filed on May 20, 1999.
(60) Provisional application No. 60/144,153, filed on Jul. 15, 1999.
(60) Provisional application No. 60/161,143, filed on Oct. 22, 1999.
(60) Provisional application No. 60/162,656, filed on Nov. 1, 1999.
(60) Provisional application No. 60/125,814, filed on Mar. 23, 1999.
(60) Provisional application No. 60/125,814, filed on Mar. 23, 1999.
(60) Provisional application No. 60/125,814, filed on Mar. 23, 1999.
(60) Provisional application No. 60/125,814, filed on Mar. 23, 1999.
(60) Provisional application No. 60/166,228, filed on Nov. 17, 1999.
(60) Provisional application No. 60/197,899, filed on Apr. 17, 2000.
(60) Provisional application No. 60/227,439, filed on Aug. 22, 2000.
(60) Provisional application No. 60/227,439, filed on Aug. 22, 2000.
(60) Provisional application No. 60/310,847, filed on Aug. 9, 2001.
(60) Provisional application No. 60/336,049, filed on Nov. 19, 2001.
(60) Provisional application No. 60/338,692, filed on Dec. 11, 2001.
(60) Provisional application No. 60/310,847, filed on Aug. 9, 2001.
(60) Provisional application No. 60/336,049, filed on Nov. 19, 2001.
(60) Provisional application No. 60/338,692, filed on Dec. 11, 2001.
(60) Provisional application No. 60/310,847, filed on Aug. 9, 2001.
(60) Provisional application No. 60/336,049, filed on Nov. 19, 2001.
(60) Provisional application No. 60/338,692, filed on Dec. 11, 2001.
(60) Provisional application No. 60/116,841, filed on Jan. 22, 1999.
(60) Provisional application No. 60/411,837, filed on Sep. 18, 2002.
(60) Provisional application No. 60/434,166, filed on Dec. 17, 2002.
(60) Provisional application No. 60/465,809, filed on Apr. 24, 2003.

(51) Int. Cl. A01H 1/00; C12Q 1/68; C12N 15/82; C12N 5/04; C07K 14/415

(52) U.S. Cl. 800/289; 435/6; 435/419; 435/468; 530/370

(57) ABSTRACT

The invention relates to plant transcription factor polypeptides, polynucleotides that encode them, homologs from a variety of plant species, and methods of using the polynucleotides and polypeptides to produce transgenic plants having improved abiotic stress tolerance, such as drought stress tolerance, as compared to wild-type or reference plants. Sequence information related to these polynucleotides and polypeptides can also be used in bioinformatic search methods to identify related sequences and is also disclosed.

### RELATIONSHIP TO COPENDING APPLICATIONS

This application claims priority from: (a) U.S. application Ser. No. 10/412,699, filed Apr. 10, 2003, which in turn claims priority from U.S. Non-provisional application Ser. No. 09/506,720, filed Feb. 17, 2000, which in turn claims priority from U.S. Provisional Application No. 60/135,134, filed May 20, 1999; U.S. Non-provisional application Ser. No. 09/394,519, filed Sep. 13, 1999; U.S. Non-provisional application Ser. No. 09/533,392, filed Mar. 22, 2000; U.S. Non-provisional application Ser. No. 09/533,029, filed Mar. 22, 2000; U.S. Non-provisional application Ser. No. 09/532,591, filed Mar. 22, 2000; U.S. Non-provisional application Ser. No. 09/533,030, filed Mar. 22, 2000, which in turn claims priority from U.S. Provisional Application No. 60/125,814, filed Mar. 23, 1999; U.S. Non-provisional application Ser. 09/713,994, filed Nov. 16, 2000, which in turn claims priority from U.S. Provisional Application No. 60/166,228, filed Nov. 17, 1999, U.S. Provisional Application No. 60/197,899, filed Apr. 17, 2000, and U.S. Provisional Application No. 60/227,439, filed Aug. 22, 2000; (b) U.S. Non-provisional application Ser. No. 10/456,882, filed Jun. 6, 2003; (c) U.S. patent application Ser. No. 09/810,836, filed Mar. 16, 2001; (d) U.S. Non-provisional application Ser. No. 10/421,138, filed Apr. 23, 2003; (e) U.S. Non-provisional application Ser. No. 09/823,676, filed Mar. 30, 2001; (f) U.S. Non-provisional application Ser. No. 09/996,140, filed Nov. 26, 2001; (g) U.S. Non-provisional application Ser. No. 09/934,455, filed Aug. 22, 2001; (h) U.S. Non-provisional application Ser. No. 10/112,887, filed Mar. 18, 2002; (i) U.S. Non-provisional application Ser. No. 10/286,264, filed Nov. 1, 2002; (.) U.S. Non-provisional application Ser. No. 10/225,066, filed Aug. 9, 2002; (k) U.S. Non-provisional application Ser. No. 10/225,067, filed Aug. 9, 2002; (1) U.S. Non-provisional application Ser. No. 10/225,068, filed Aug. 9, 2002; which claims priority from U.S. Provisional Application No. 60/310,847, filed Aug. 9, 2001, U.S. Provisional Application No. 60/338,692, filed Dec. 11, 2001, and from U.S. Provisional Application No.60/336,049, filed Nov. 19, 2001; (m) U.S. Non-provisional application Ser. No. 10/374,780, filed Feb. 25, 2003, which claims priority from U.S. Non-provisional application Ser. No. 09/837,944, filed Apr. 18, 2001, and U.S. Non-provisional application Ser. No. 10/171,468, filed Jun. 14, 2002; and (n) U.S. Non-provisional application Ser. No. 10/666,642, filed Sep. 18, 2003, which claims priority from U.S. Provisional Application No. 60/434,166, filed Dec. 17, 2002, U.S. Provisional Application No. 60/411,837, filed Sep. 18, 2002, and U.S. Provisional Application No. 60/465,809, filed Apr. 24, 2003. The entire contents of all of these applications are hereby incorporated by reference.

### FIELD OF THE INVENTION

The present invention relates to compositions and methods for modifying a plant phenotypically, said plant having increased tolerance to drought stress.

### BACKGROUND OF THE INVENTION

Control of Cellular Processes by Transcription Factors.

Phylogenetic relationships among organisms have been demonstrated many times, and studies from a diversity of prokaryotic and eukaryotic organisms suggest a more or less gradual evolution of biochemical and physiological mechanisms and metabolic pathways. Despite different evolutionary pressures, proteins that regulate the cell cycle in yeast, plant, nematode, fly, rat, and man have common chemical or structural features and modulate the same general cellular activity. Comparisons of Arabidopsis gene sequences with those from other organisms where the structure and/or function may be known allow researchers to draw analogies and to develop model systems for testing hypotheses. These model systems are of great importance in developing and testing plant varieties with novel traits that may have an impact upon agronomy. These traits, such as a plant's biochemical, developmental, or phenotypic characteristics, may be controlled through a number of cellular processes. One important way to manipulate that control is through transcription factors, proteins that influence the expression of a particular gene or sets of genes. Transformed and transgenic plants that comprise cells having altered levels of at least one selected transcription factor, for example, possess advantageous or desirable traits. Strategies for manipulating traits by altering a plant cell's transcription factor content can therefore result in plants and crops with new and/or improved commercially valuable properties. Transcription factors can modulate gene expression, either increasing or decreasing (inducing or repressing) the rate of transcription. This modulation results in differential levels of gene expression at various developmental stages, in different tissues and cell types, and in response to different exogenous (e.g., environmental) and endogenous stimuli throughout the life cycle of the organism.

Because transcription factors are key controlling elements of biological pathways, altering the expression levels of one or more transcription factors can change entire biological pathways in an organism. For example, manipulation of the levels of selected transcription factors may result in increased expression of economically useful proteins or biomolecules in plants or improvement in other agriculturally relevant characteristics. Conversely, blocked or reduced expression of a transcription factor may reduce biosynthesis of unwanted compounds or remove an undesirable trait. Therefore, manipulating transcription factor levels in a plant offers tremendous potential in agricultural biotechnology for modifying a plant's traits, including traits that improve a plant's survival and yield during periods of drought and other abiotic stresses, as noted below.

Problems Associated with Water Deprivation.

In the natural environment, plants often grow under unfavorable conditions, such as drought (low water availability), high salinity, chilling, freezing, high temperature, flooding, or strong light. Any of these abiotic stresses can delay growth and development, reduce productivity, and in extreme cases, cause the plant to die. In general, tolerance to abiotic stress is associated with a host of morphological and physiological traits; these include root structure, shoot architecture, variation in leaf cuticle thickness, stomatal regulation, osmotic adjustment, antioxidant capacity, hormonal regulation, desiccation tolerance (membrane and protein stability), maintenance of photosynthesis, and the timing of events during reproduction (Bohnert et al. (1995) Plant Cell 7: 1099-1111; Shinozaki and Yamaguchi-Shinozaki (1996) Curr. Opin. Biotechnol. 7: 161-167; Bray (1997) Trends Plant Sci. 2: 48-54; Nguyen et al. (1997) Crop Sci. 37: 1426-1434).

Of these stresses, low water availability, which in a severe form is referred to as a drought, is a major factor in crop yield reduction worldwide. A drought is a period of dry weather that persists long enough to produce a hydrologic imbalance, which can result, for example, in wilting, senescence, and general crop damage. Short periods of dry weather can lead to hydrologic imbalances of economic important, but while most weather changes are brief and short-lived, drought can be a more gradual phenomenon, slowly taking hold of an area and tightening its grip with time. In severe cases, drought can last for many years and can have devastating effects on agriculture and water supplies. For maize, loss to drought in the tropics alone is thought to exceed 20 million tons of grain per year. With burgeoning population and chronic shortage of available fresh water, drought is not only the number one weather related problem in agriculture, it also ranks as one of the major natural disasters, causing not only economic damage, but also loss of human lives. For example, losses from the US drought of 1988 exceeded $40 billion, exceeding the losses caused by Hurricane Andrew in 1992, the Mississippi River floods of 1993, and the San Francisco earthquake in 1989. In some areas of the world, the effects of drought can be far more severe. In severely affected regions (such as southern Africa in 1991-92), this can correspond to a loss of up to 60% of the potential yield (Edmeades et al. (1992) “47th Annual Corn & Sorghum Research Conference”; in D. Wilkinson, ed., Washington, D.C.: American Seed Trade Association—ASTA, pp. 93-111). In the Horn of Africa the 1984-1985 drought led to a famine that killed 750,000 people.

Problems for plants caused by low water availability include mechanical stresses caused by the withdrawal of cellular water. Drought also causes plants to become more susceptible to various diseases (Simpson (1981), ed., “The Value of Physiological Knowledge of Water Stress in Plants”, In Water Stress on Plants, Praeger, N.Y., pp. 235-265).

In addition to the many land regions of the world that are too arid for most if not all crop plants, overuse and over-utilization of available water is resulting in an increasing loss of agriculturally-usable land, a process which, in the extreme, results in desertification. The problem is further compounded by increasing salt accumulation in soils, which adds to the loss of available water in soils.

Water deficit is a common component of many plant stresses. Water deficit occurs in plant cells when the whole plant transpiration rate exceeds the water uptake. In addition to drought, other stresses, such as salinity and low temperature, produce cellular dehydration (McCue and Hanson (1990) Trends Biotechnol. 8: 358-362

Salt and drought stress signal transduction include ionic and osmotic homeostasis signaling pathways. The ionic aspect of salt stress is signaled via the SOS pathway where a calcium-responsive SOS3-SOS2 protein kinase complex controls the expression and activity of ion transporters such as SOS1. The pathway regulating ion homeostasis in response to salt stress has been described recently by Xiong and Zhu (2002) Plant Cell Environ. 25: 131-139 and Ohta et al. (2003) Proc Natl Acad Sci USA 100: 11771-11776.

The osmotic component of salt stress involves complex plant reactions that overlap with drought and/or low temperature stress responses.

Common aspects of drought, cold and salt stress response have been reviewed recently by Xiong and Zhu (2002) supra). Those include:
(a) transient changes in the cytoplasmic calcium levels very early in the signaling event (Knight, (2000) Int. Rev. Cytol. 195: 269-324; Sanders et al. (1999) Plant Cell 11: 691-706);
(b) signal transduction via mitogen-activated and/or calcium dependent protein kinases (CDPKs; see Xiong et al., 2002) and protein phosphatases (Merlot et al. (2001) Plant J. 25: 295-303; Tähtiharju and Palva (2001) Plant J. 26: 461-470);
(c) increases in abscisic acid levels in response to stress triggering a subset of responses (Xiong et al. (2002) supra, and references therein);
(d) inositol phosphates as signal molecules (at least for a subset of the stress responsive transcriptional changes (Xiong et al. (2001) Genes Dev. 15: 1971-1984);
(e) activation of phospholipases which in turn generate a diverse array of second messenger molecules, some of which might regulate the activity of stress responsive kinases (phospholipase D functions in an ABA independent pathway, Frank et al. (2000) Plant Cell 12: 111-124);
(f) induction of late embryogenesis abundant (LEA) type genes including the CRT/DRE responsive CORIRD genes (Xiong and Zhu (2002) supra);
(g) increased levels of antioxidants and compatible osmolytes such as proline and soluble sugars (Hasegawa et al. (2000) Annu. Rev. Plant Mol. Plant Physiol. 51: 463-499); and
(h) accumulation of reactive oxygen species such as superoxide, hydrogen peroxide, and hydroxyl radicals (Hasegawa et al. (2000) supra).

Abscisic acid biosynthesis is regulated by osmotic stress at multiple points. Both ABA-dependent and -independent osmotic stress signaling first modify constitutively expressed transcription factors, leading to the expression of early response transcriptional activators, which then activate downstream transcriptional activators and stress tolerance effector genes.

Based on the commonality of many aspects of low-temperature, drought and salt stress responses, it can be concluded that genes that increase tolerance to low temperature or salt stress can also improve drought stress protection. In fact, this has already been demonstrated for transcription factors, as in the case of AtCBF/DREB1, and for other genes such as OsCDPK7 (Saijo et al. (2000) Plant J. 23: 319-327) or AVP1 (a vacuolar pyrophosphatase-proton-pump, Gaxiola et al. (2001) Proc. Natl. Acad. Sci. USA 98: 11444-11449).

The present invention relates to methods and compositions for producing transgenic plants with improved tolerance to drought and other abiotic stresses. This provides significant value in that the plants may thrive in hostile environments where low water availability limits or prevents growth of non-transgenic plants. We have identified polynucleotides encoding transcription factors, including G2133, G1274, G922, G2999, G3086, G354, G1792, G2053, G975, G1069, G916, G1820, G2701, G47, G2854, G2789, G634, G175, G2839, G1452, G3083, G489, G303, G2992, G682, functionally related sequences listed in the Sequence Listing, and structurally and functionally similar sequences, developed numerous transgenic plants using these polynucleotides, and analyzed the plants for their tolerance to drought stress. In so doing, we have identified important polynucleotide and polypeptide sequences for producing commercially valuable plants and crops as well as the methods for making them and using them. Other aspects and embodiments of the invention are described below and can be derived from the teachings of this disclosure as a whole.

### SUMMARY OF THE INVENTION

The present method is directed to recombinant polynucleotides that confer abiotic stress tolerance in plants when the expression of any of these recombinant polynucleotides is altered (e.g., by overexpression). Related sequences that are also encompassed by the invention include nucleotide sequences that hybridize to the complement of the sequences of the invention under stringent conditions. One example of a stringent condition that defines the invention, includes a hybridization procedure that incorporates two wash steps of 6×SSC and 65° C., each step being 10-30 minutes in duration. For example, G2133 (polynucleotide SEQ ID NO: 11 and polypeptide SEQ ID NO: 12) confer tolerance to a number of abiotic stresses, including drought, cold conditions during germination, cold conditions with respect to more mature plants (chilling), and low nitrogen conditions, when this polypeptide is overexpressed in plants. The invention thus includes the G2133 polynucleotide and polypeptide, as well as nucleotide sequences that are structurally similar in that they or their complement hybridize to SEQ ID NO: 11 under stringent hybridization conditions.

The invention also pertains to a transgenic plant that comprises a recombinant polynucleotide that encodes a polypeptide that regulates transcription. For example, a sizeable number of polypeptides that contain the AP2 domain have been shown to possess gene-regulating activity. In this aspect of the invention, the polypeptide has the property of a polypeptide of the Sequence Listing of regulating abiotic stress tolerance in a plant when the polypeptide is overexpressed in a plant. An example of a recombinant polynucleotide that is comprised by the transgenic plant is G2133, and in this case the polypeptide that is overexpressed is the G2133 polypeptide. In this aspect of the invention, the AP2 domain is sufficiently homologous to the AP2 domain of the G2133 polypeptide that the polypeptide binds to a transcription-regulating region. This binding confers increased abiotic stress tolerance in the transgenic plant when the plant is compared to a non-transformed plant that does not overexpress the polypeptide.

The invention also includes a transgenic plant that overexpresses a recombinant polynucleotide comprising a nucleotide sequence that hybridizes to the complement of any polynucleotide of the invention under stringent conditions. This transgenic plant has increased abiotic stress tolerance as compared to a non-transformed plant that does not overexpress a polypeptide encoded by the recombinant polynucleotide. One example of a polynucleotide of the invention that functions in this regard is the G2133 polynucleotide (SEQ ID NO 11).

The invention also encompasses a method for producing a transgenic plant having increased tolerance to abiotic stress. These method steps include first providing an expression vector that contains a nucleotide sequence that hybridizes to the complement of the a polynucleotide of the invention (e.g., the G2133 polynucleotide, SEQ ID NO 11) under stringent hybridization conditions. The expression vector is then introduced into a plant cell, the plant cell is cultured, from which a plant is generated. Due to the presence of the expression vector in the plant, the polypeptide encoded by the nucleotide sequence is overexpressed. This polypeptide has the property of regulating abiotic stress tolerance in a plant, compared to a non-transformed plant that does not overexpress the polypeptide. After the abiotic stress-tolerant transgenic plant is produced, it may be identified by comparing it with one or more non-transformed plants that do not overexpress the polypeptide. These method steps may further include selfing or crossing the abiotic stress-tolerant plant with itself or another plant, respectively, to produce seed; (“selfing” refers to self-pollinating, or using pollen from one plant to fertilize the same plant or another plant in the same line, whereas “crossing” generally refers to cross pollination with plant from a different line, such as a non-transformed or wild-type plant, or another transformed plant from a different transgenic line of plants). Crossing provides the advantage of being able to produce new varieties. The resulting seed may then be used to grow a progeny plant that is transgenic and has increased tolerance to abiotic stress.

The invention is also directed to a method for increasing a plant's tolerance to abiotic stress. This method includes first providing a vector that comprises (i) regulatory elements effective in controlling expression of a polynucleotide sequence in a target plant, where the regulatory elements flank the polynucleotide sequence; and (ii) the polynucleotide sequence itself, which encodes a polypeptide that has the ability to regulate abiotic stress tolerance in a plant, as compared to a non-transformed plant that does not overexpress the polypeptide. The plant is transformed with the vector in order to generate a transformed plant with increased tolerance to abiotic stress. An example of a polynucleotide sequence that may be used to transform the target plant includes G2133; in this case, the polypeptide that is overexpressed is the G2133 polypeptide.

### BRIEF DESCRIPTION OF THE SEQUENCE LISTING AND FIGURES

The file of this patent contains at least one drawing executed in color. Copies of this patent with color drawing(s) will be provided by the Patent and Trademark Office upon request and payment of the necessary fee.

The Sequence Listing provides exemplary polynucleotide and polypeptide sequences of the invention. The traits associated with the use of the sequences are included in the Examples.

CD-ROM1 is a read-only memory computer-readable compact disc and contains a copy of the Sequence Listing in ASCII text format. The Sequence Listing is named “MBI0058CIP.ST25.txt” and is 740 kilobytes in size. The copies of the Sequence Listing on the CD-ROM disc are hereby incorporated by reference in their entirety.

FIG. 1 shows a conservative estimate of phylogenetic relationships among the orders of flowering plants (modified from Angiosperm Phylogeny Group (1998) Ann. Missouri Bot. Gard. 84: 1-49). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al. (2001) Plant Physiol. 127: 1328-1333.

FIG. 2 shows a phylogenic dendogram depicting phylogenetic relationships of higher plant taxa, including clades containing tomato and Arabidopsis; adapted from Ku et al. (2000) Proc. Natl. Acad. Sci. 97: 9121-9126; and Chase et al. (1993) Ann. Missouri Bot. Gard. 80: 528-580.

FIGS. 3A-3M present a multiple amino acid sequence alignment of G47 and G47 orthologs and paralogs. Clade orthologs and paralogs are indicated by the black bar on the left side of the figure. Conserved regions of identity and similarity are boxed.

FIG. 4 illustrates the relationship of G47 and related sequences in this phylogenetic tree of the G47 clade and similar sequences. The tree building method used was “Neighbor Joining” with “Systematic Tie-Breaking” and Bootstrapping with 1000 replicates (Uncorrected (“p”), with gaps distributed proportionally). Full-length polypeptides were used to build the phylogeny as defined in FIG. 4. The members of the clade shown within the box are predicted to contain functional homologs of G47. Abbreviations: At Arabidopsis thaliana; Os (jap) Oryza sativa (japonica cultivar group); Zm Zea mays; Gm Glycine max; Mt Medicago truncatula; Br Brassica rapa; Bo Brassica oleracea; Ze: Zinnia elegans.

FIG. 5 Alignment of portion of AP2 domain for G47 clade. The three residues indicated by the arrows define the G47 clade. All clade members have a valine, valine and histidine residue at these positions, respectively.

FIG. 6A, which shows the results of an experiment conducted with G47-overexpressing lines, illustrates an example of an osmotic stress assay. The medium used in this root growth assay contained polyethylene glycol (PEG). After germination, the seedlings of a 35S::G47 overexpressing line (the eight seedlings on left labeled “OE.G47-22”) appeared larger and had more root growth than the four wild-type seedlings on the right. As would be predicted by the osmotic stress assay, G47 plants showed enhanced survival and drought tolerance in a soil-based drought assay, as did G2133, a paralog of G47 (see FIGS. 7A and 7B). FIG. 6B also demonstrates an interesting effect of G47 overexpression; the 35S::G47 plants on the left and in the center of this photograph had short, thick, fleshy inflorescences with reduced apical dominance compared with the wild-type plant on the right.

FIGS. 7A and 7B compare the recovery from a drought treatment of wild-type controls and two lines of Arabidopsis plants overexpressing G2133, a paralog of G47. FIG. 7A shows plants of 35S::G2133 line 5 (left) and control plants (right). FIG. 7B shows plants of 35S::G2133 line 3 (left) and control plants (right). Each pot contained several plants grown under 24 hours light. All were deprived of water for eight days, and are shown after re-watering. All of the plants of the G2133 overexpressor lines recovered, and all of the control plants were either dead or severely and adversely affected by the drought treatment.

FIGS. 8A-8S present a multiple amino acid sequence alignment of G2999 and G2999 orthologs and paralogs. Consensus residues that are identical between sequences appear in boldface, and similar residues appear within the boxes.

FIGS. 9A-9C compare a number of homeodomains from the zinc-finger-homeodomain-type (ZF-HD) proteins related to G2999. Homeodomains from the ZF-HD type proteins are distinct from classical types of homeodomains and lie on the distinct branch of the tree shown in FIG. 10. The relationships established from this type of alignment of homeodomains were used to generate the phylogenetic tree shown in FIG. 10.

FIG. 10A illustrates the relationship of G2999 and related sequences in this phylogenetic tree of the G2999 clade and similar sequences comprising ZF-HD-type proteins. The tree building method used was “Neighbor Joining” with “Systematic Tie-Breaking” and Bootstrapping with 1000 replicates (Uncorrected (“p”), with gaps distributed proportionally. All of the sequences shown are members of the clade and are predicted to be functional homologs of G2999. Abbreviations: At Arabidopsis thaliana; Os (jap) Oryza sativa (japonica cultivar group); Os (ind) Oryza sativa (indica cultivar group); Zm Zea mays; Lj Lotus corniculatus var. japonicus; Bn Brassica napus; Fb Flaveria bidentis.

FIG. 10B is a phylogenetic tree (neighbor-joining, 1000 bootstraps) highlighting the relational differences between the ZF-HD type proteins and the “classical” homeodomain (HD) proteins. The homeodomains from ZF-HD type proteins lie on a distinct branch of the tree compared to classical types of homeodomains (arrow).

FIG. 11A illustrates the results of root growth assays with G2999-overexpressing seedlings and controls in a high sodium chloride medium. The eight 35S::G2999 Arabidopsis seedlings on the left were larger, greener, and had more root growth than the four control seedlings on the right. Another member of the G2999 clade, G2998, also showed a salt tolerance phenotype and performed similarly in the plate-based salt stress assay seen FIG. 11B. In the latter assay 35S::G2998 seedlings appeared large and green, whereas wild-type seedlings in the control assay plate shown in FIG. 11C were small and had not yet expanded their cotyledons. As is noted below, high sodium chloride growth assays often are used to indicate osmotic stress tolerance such as drought tolerance, which was subsequently confirmed with soil-based assays conducted with G2999-overexpressing plants.

FIGS. 12A-12L represent a multiple amino acid sequence alignment of G1792 orthologs and paralogs. Clade orthologs and paralogs are indicated by the black bar on the left side of the figure. Conserved regions of identity are boxed and bolded while conserved sequences of similarity are boxed with no bolding. The AP2 conserved domains span alignment coordinates 196-254. The S conserved domain spans alignment coordinates of 301-304. The EDLL conserved domain spans the alignment coordinates of 391-406 (see FIG. 13). Abbreviations: At Arabidopsis thaliana; Os Oryza sativa; Zm Zea mays; Ta Triticum aestivum; Gm Glycine max; Mt Medicago truncatula.

FIG. 13 shows a novel conserved domain for the G1792 clade, herein referred to as the “EDLL domain”. All clade members contain a glutamic acid residue at position 3, an aspartic acid residue at position 8, and a leucine residue at positions 12 and 16.

FIG. 14 illustrates the relationship of G1792 and related sequences in this phylogenetic tree of the G1792 clade. The tree building method used was “Neighbor Joining” with “Systematic Tie-Breaking” and Bootstrapping with 1000 replicates. Only conserved domains were used to build the phylogeny as defined in FIG. 12. The members of the clade are shown within the box.

FIGS. 15A and 15B compare soil-based drought assays for G1792 overexpressors and wild-type control plants. 35S::G1792 lines had a much healthier appearance after a period of water deprivation (FIG. 15A) than control plants (FIG. 15B).

FIG. 16A-16U show a multiple amino acid sequence alignment of G3086 and its orthologs and paralogs. The G3086 clade is indicated by the black bar on the left side of the figure.

FIG. 17 is a phylogenetic tree of the G3086 lade, including G3086 and its paralogs and orthologs. Full length, predicted protein sequences were used to construct a pairwise comparison, bootstrapped (1000 replicates) neighbor-joining tree, consensus view. Sequences within the G3086 clade are located within the box. Abbreviations: At Arabidopsis thaliana; Os Oryza sativa; Zm Zea mays; Gm Glycine max; Pt Pinus taeda.

FIG. 18A shows the effects of a heat assay on Arabidopsis wild-type and G3086-overexpressing plants. Generally, the overexpressors on the left were larger, paler, and bolted earlier than the wild type plants seen on the right in this plate. The same G3086 overexpressing lines, as exemplified by the eight seedlings on the left of FIG. 18B, were also found to be larger, greener, and had more root growth in a high salt root growth assay than control plants, including the four on the right in FIG. 18B.

FIGS. 19A-19R show a multiple amino acid sequence alignment of G922 orthologs and paralogs. Clade orthologs and paralogs are indicated by black bar on the left side of the figure. Residues that appear in boldface represent an acidic, ser/pro-rich domain that is unique to the G922 clade. Abbreviations: At Arabidopsis thaliana; Os Oryza sativa; Zm Zea mays; Ta Triticum aestivum; Gm Glycine max; Le Lycopersicon esculentum; Ps Pisum sativum.

FIG. 20 is a phylogenetic tree of the G922 paralogs and orthologs. Full length, predicted protein sequences were used to construct a pairwise comparison, bootstrapped (1000 replicates) neighbor-joining tree, consensus view. Sequences within the G922 clade are located within the box.

As seen in FIG. 21A, which shows a root growth assay on media containing high concentrations (150 mM) of salt, G922 overexpressors exhibited greener seedlings with longer roots than wild-type seedlings seen in FIG. 21B. FIG. 21C shows seedlings of several G922 overexpressing lines on media containing a high sucrose concentration (9.4%). A number of these seedlings have greener cotyledons and longer roots than the wild-type seedlings on the same media in FIG. 21D.

FIGS. 22A-22R show a multiple sequence alignment of predicted protein sequences from G1274 paralogs and orthologs. The sequences within the G1274 clade are indicated by the black bar on the margin. Amino acid identities and similarities are outlined and shown in bold.

FIG. 23 represents a phylogenetic tree for the G1274 paralogs and orthologs. Full length, predicted protein sequences were used to construct a bootstrapped (1000 replicates) neighbor-joining tree. Gaps and missing data were handled using pairwise deletion and the distance method used was p-distance. Sequences within the G1274 clade appear within the box.

FIG. 24 depicts a multiple sequence alignment of a portion of the conserved WRKY domain from G1274 paralogs and potential orthologs. The sequences within the G1274 clade are indicated by the black bar in the margin. Conserved identities and similarities are outlined and bolded. Amino acid residues within this domain that distinguish the G1274 clade sequences, and are putatively responsible for conserved functionality, are indicated with an asterisk.

FIG. 25A is a photograph of Arabidopsis 35S::G1274 seedlings grown on low nitrogen media supplemented with sucrose plus glutamine. Seedlings of two overexpressing lines are present on this plate (not distinguished), and both lines contained less anthocyanin than the wild-type seedlings seen in FIG. 25B. The lack of anthocyanin production indicated that these lines were less stressed than control seedlings under the same conditions. G1274 overexpressors in FIG. 25C and wild-type in FIG. 25D were also compared in a cold germination assay, in which the overexpressors were found to be generally larger and greener than the controls.

FIGS. 26A-26D compare soil-based drought assays for G1274 overexpressors and wild-type control plants, which confirms the results predicted after the performance of G1274 overexpressors in plate-based osmotic stress assays. 35S::G1274 lines fared much better after a period of water deprivation (FIG. 26A) than control plants (FIG. 26B). This distinction was particularly evident in the overexpressor plants when the drought period was followed by rewatering; the overexpressor plants recovered to a healthy and vigorous state (FIG. 26C). Conversely, none of the wild-type plants recovered after rewatering (FIG. 26D).

FIGS. 27A-27BB show a multiple sequence alignment of predicted protein sequences from G2053, and its paralogs and orthologs. The sequences within the G2053 clade are indicated by the black bar to the left of the alignment. The amino acid residues in boldface are consensus residues, and those within the boxes represent conserved, similar residues.

FIG. 28 is a phylogenetic tree for the G2053 paralogs and orthologs. Full length, predicted protein sequences were used to construct a bootstrapped (1000 replicates) neighbor-joining tree. Gaps and missing data were handled using pairwise deletion and the distance method used was p-distance. Sequences within the G2053 clade appear within the box.

FIG. 29 shows the results of a G2053-overexpressor root growth assay on media containing high concentrations of PEG. The eight G2053 overexpressor seedlings to the left of the plate showed more root growth, and were generally larger, than the four wild-type controls on the right.

### DESCRIPTION OF THE INVENTION

In an important aspect, the present invention relates to polynucleotides and polypeptides, for example, for modifying phenotypes of plants, particularly those associated with drought stress tolerance. Throughout this disclosure, various information sources are referred to and/or are specifically incorporated. The information sources include scientific journal articles, patent documents, textbooks, and World Wide Web browser-inactive page addresses, for example. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a,” “an,” and “the” include plural reference unless the context clearly dictates otherwise. Thus, for example, a reference to “a plant” includes a plurality of such plants, and a reference to “a stress” is a reference to one or more stresses and equivalents thereof known to those skilled in the art, and so forth.

Definitions

“Nucleic acid molecule” refers to a oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides, optionally at least about 30 consecutive nucleotides, at least about 50 consecutive nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as splicing and folding to obtain a functional protein or polypeptide. A gene may be isolated, partially isolated, or be found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and which may be used to determine the limits of the genetically active unit (Rieger et al. (1976) Glossary of Genetics and Cytogenetics: Classical and Molecular, 4th ed., Springer Verlag. Berlin). A gene generally includes regions preceding (“leaders”; upstream) and following (“trailers”; downstream) of the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence. Additionally, the terms “homology” and “homologous sequence(s)” may refer to one or more polypeptide sequences that are modified by chemical or enzymatic means. The homologous sequence may be a sequence modified by lipids, sugars, peptides, organic or inorganic compounds, by the use of modified amino acids or the like. Protein modification techniques are illustrated in Ausubel et al. (eds) Current Protocols in Molecular Biology, John Wiley & Sons (1998).

“Hybridization complex” refers to a complex between two nucleic acid molecules by virtue of the formation of hydrogen bonds between purines and pyrimidines.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

With regard to polypeptides, the terms “substantial identity” or “substantially identical” may refer to sequences of sufficient similarity and structure to the transcription factors in the Sequence Listing to produce similar function when expressed or overexpressed in a plant; in the present invention, this function is increased tolerance to drought. Sequences that are at least about 50% identical, and preferably at least 82% identical, to the instant polypeptide sequences are considered to have “substantial identity” with the latter. Sequences having lesser degrees of identity but comparable biological activity are considered to be equivalents. The structure required to maintain proper functionality is related to the tertiary structure of the polypeptide. There are discreet domains and motifs within a transcription factor that must be present within the polypeptide to confer function and specificity. These specific structures are required so that interactive sequences will be properly oriented to retain the desired activity. “Substantial identity” may thus also be used with regard to subsequences, for example, motifs, that are of sufficient structure and similarity, being at least about 50% identical, and preferably at least 82% identical, to similar motifs in other related sequences so that each confers or is required for increased tolerance to drought.

The term “amino acid consensus motif” refers to the portion or subsequence of a polypeptide sequence that is substantially conserved among the polypeptide transcription factors listed in the Sequence Listing.

“Alignment” refers to a number of nucleotide or amino acid residue sequences aligned by lengthwise comparison so that components in common (i.e., nucleotide bases or amino acid residues) may be visually and readily identified. The fraction or percentage of components in common is related to the homology or identity between the sequences. Alignments such as those found the Figures may be used to identify conserved domains and relatedness within these domains. An alignment may suitably be determined by means of computer programs known in the art, such as MacVector (1999) (Accelrys, Inc., San Diego, Calif.).

A “conserved domain” or “conserved region” as used herein refers to a region in heterologous polynucleotide or polypeptide sequences where there is a relatively high degree of sequence identity between the distinct sequences. AP2 domains are examples of conserved domains.

With respect to polynucleotides encoding presently disclosed transcription factors, a conserved domain is preferably at least 10 base pairs (bp) in length.

A “conserved domain”, with respect to presently disclosed polypeptides refers to a domain within a transcription factor family that-exhibits a higher degree of sequence homology, such as at least 70% sequence similarity, including conservative substitutions, and more preferably at least 79% sequence identity, and even more preferably at least 81%, or at least about 86%, or at least about 87%, or at least about 89%, or at least about 91%, or at least about 95%, or at least about 98% amino acid residue sequence identity of a polypeptide of consecutive amino acid residues. A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (see, for example, Riechmann et al. (2000) supra). Thus, by using alignment methods well known in the art, the conserved domains (i.e., the AP2 domains) of the AP2 plant transcription factors (Riechmann and Meyerowitz (1998) Biol. Chem. 379:633-646) may be determined.

The conserved domains for a number of the sequences of the Sequence Listing are found in Table 1. A comparison of the regions of the polypeptides in Table 1 allows one of skill in the art to identify conserved domains for any of the polypeptides listed or referred to in this disclosure.

“Complementary” refers to the natural hydrogen bonding by base pairing between purines and pyrimidines. For example, the sequence A-C-G-T (5′→3′) forms hydrogen bonds with its complements A-C-G-T (5′→3′) or A-C-G-U (5′→3′). Two single-stranded molecules may be considered partially complementary, if only some of the nucleotides bond, or “completely complementary” if all of the nucleotides bond. The degree of complementarity between nucleic acid strands affects the efficiency and strength of the hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y. (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure (for a more detailed description of establishing and determining stringency, see below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, transcription factors having 60% identity, or more preferably greater than about 70% identity, most preferably 72% or greater identity with disclosed transcription factors.

Regarding the terms “paralog” and “ortholog”, homologous polynucleotide sequences and homologous polypeptide sequences may be paralogs or orthologs of the claimed polynucleotide or polypeptide sequence. Orthologs and paralogs are evolutionarily related genes that have similar sequence and similar functions. Orthologs are structurally related genes in different species that are derived by a speciation event. Paralogs are structurally related genes within a single species that are derived by a duplication event. Sequences that are sufficiently similar to one another will be appreciated by those of skill in the art and may be based upon percentage identity of the complete sequences, percentage identity of a conserved domain or sequence within the complete sequence, percentage similarity to the complete sequence, percentage similarity to a conserved domain or sequence within the complete sequence, and/or an arrangement of contiguous nucleotides or peptides particular to a conserved domain or complete sequence. Sequences that are sufficiently similar to one another will also bind in a similar manner to the same DNA binding sites of transcriptional regulatory elements using methods well known to those of skill in the art.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) world wide web (www) website, “tigr.org” under the heading “Terms associated with TIGRFAMs”.

The term “variant”, as used herein, may refer to polynucleotides or polypeptides, that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences o may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine (for more detail on conservative substitutions, see Table 3). More rarely, a variant may have “non-conservative” changes, for example, replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the term refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. This, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

“Ligand” refers to any molecule, agent, or compound that will bind specifically to a complementary site on a nucleic acid molecule or protein. Such ligands stabilize or modulate the activity of nucleic acid molecules or proteins of the invention and may be composed of at least one of the following: inorganic and organic substances including nucleic acids, proteins, carbohydrates, fats, and lipids.

“Modulates” refers to a change in activity (biological, chemical, or immunological) or lifespan resulting from specific binding between a molecule and either a nucleic acid molecule or a protein.

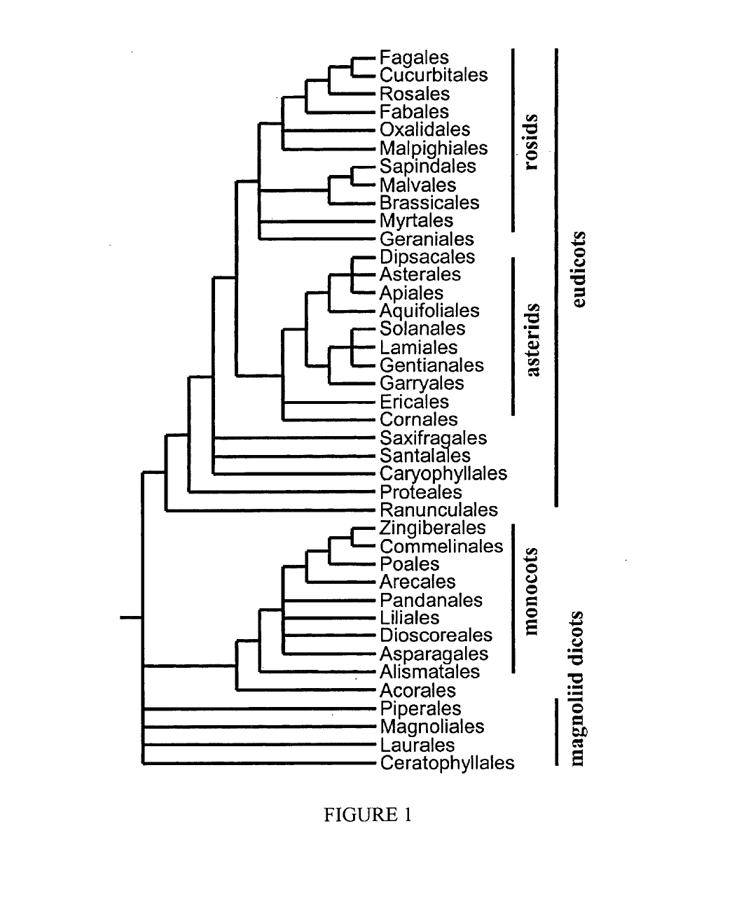

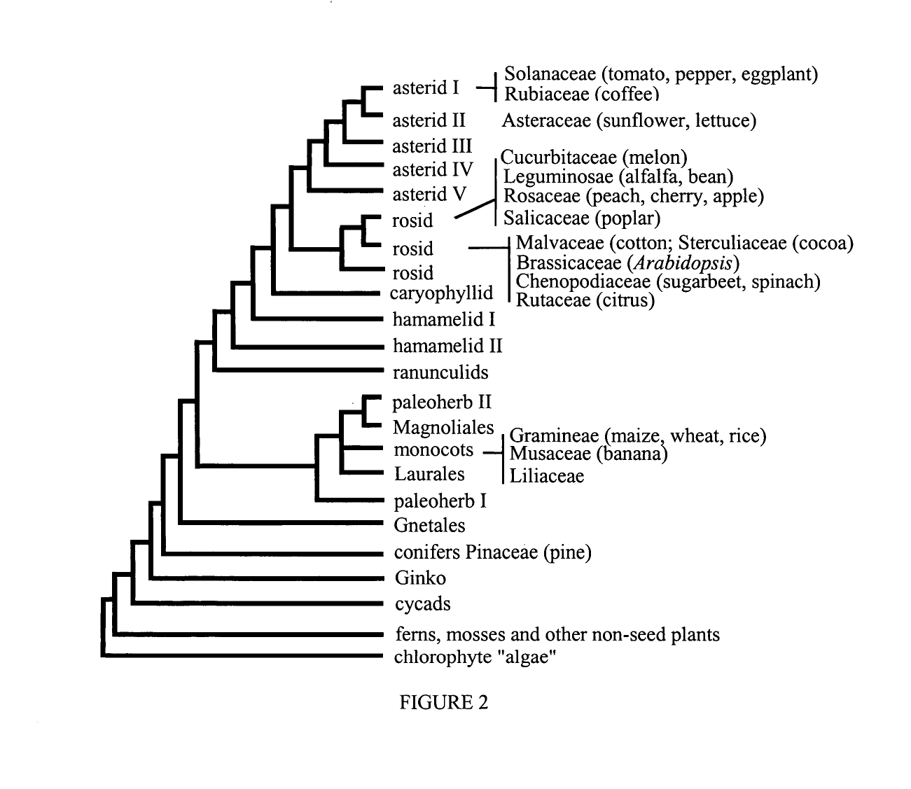

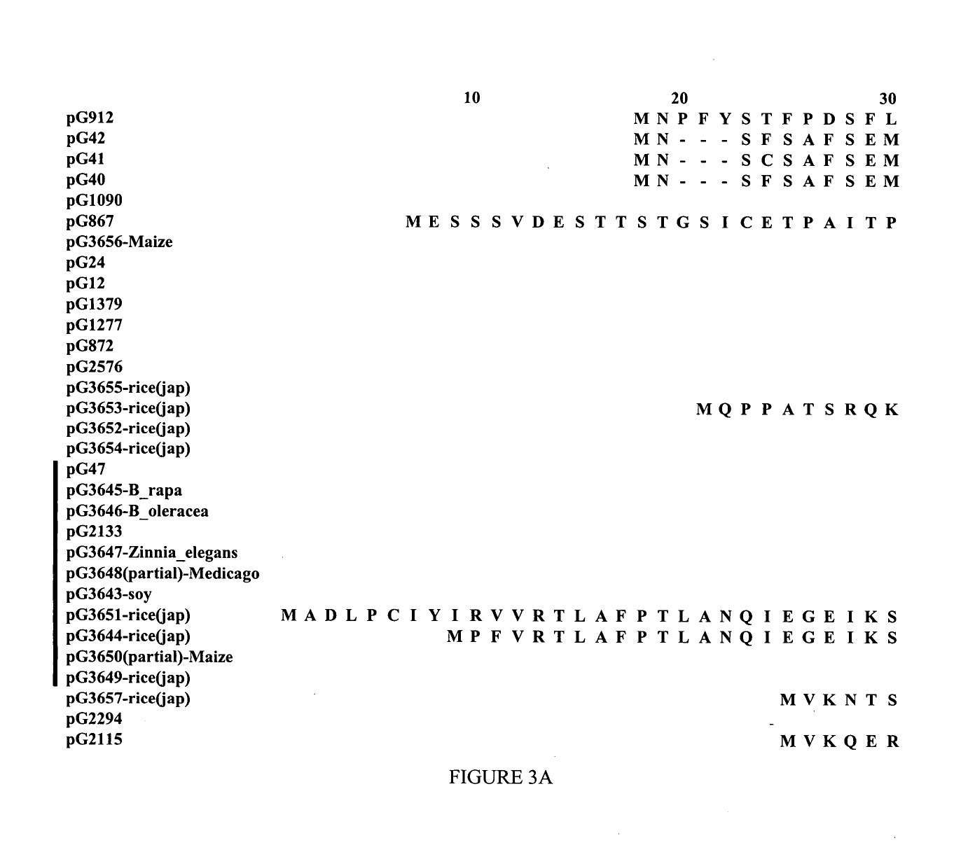

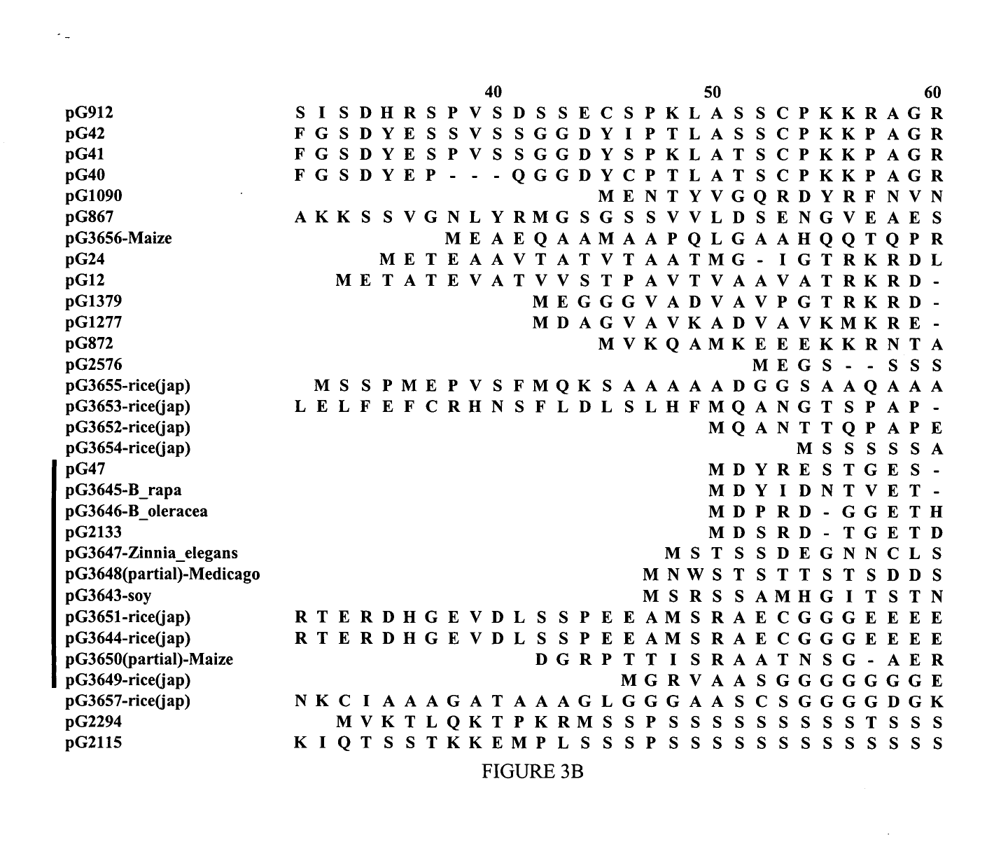

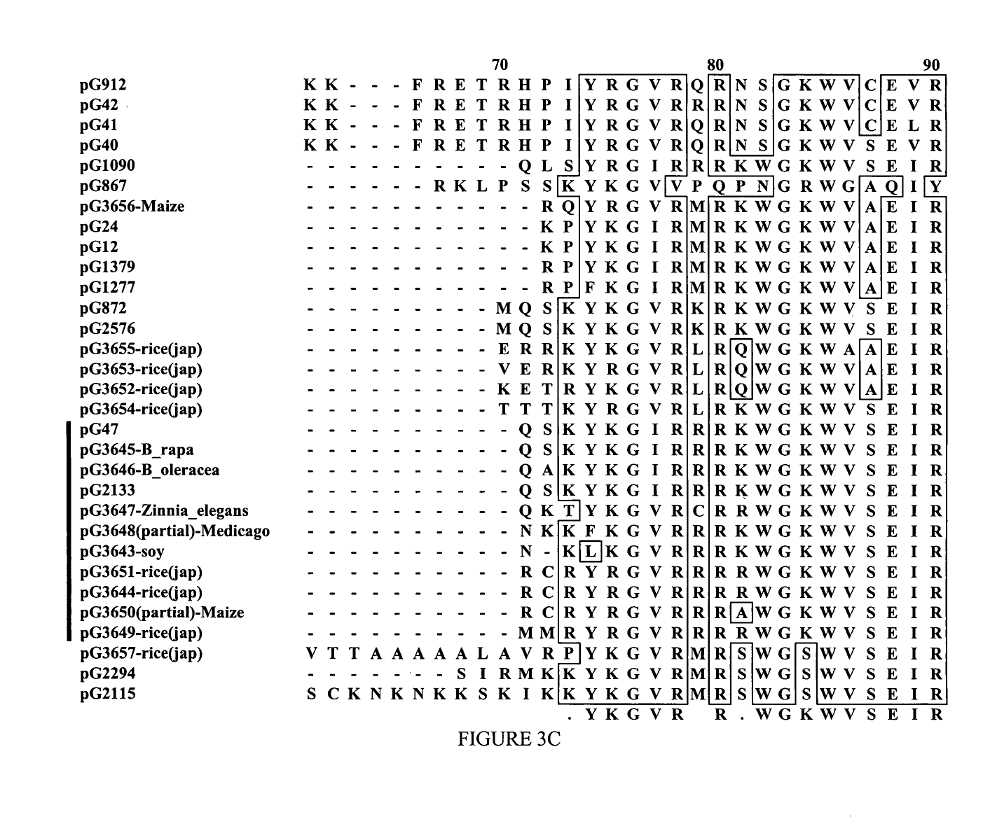

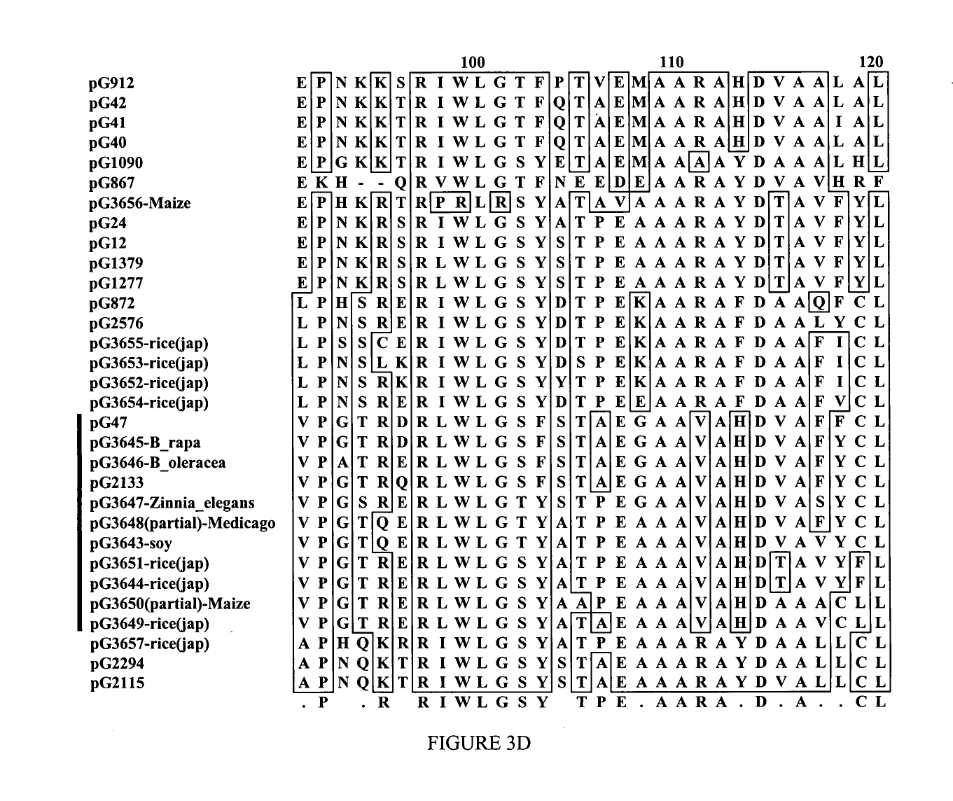

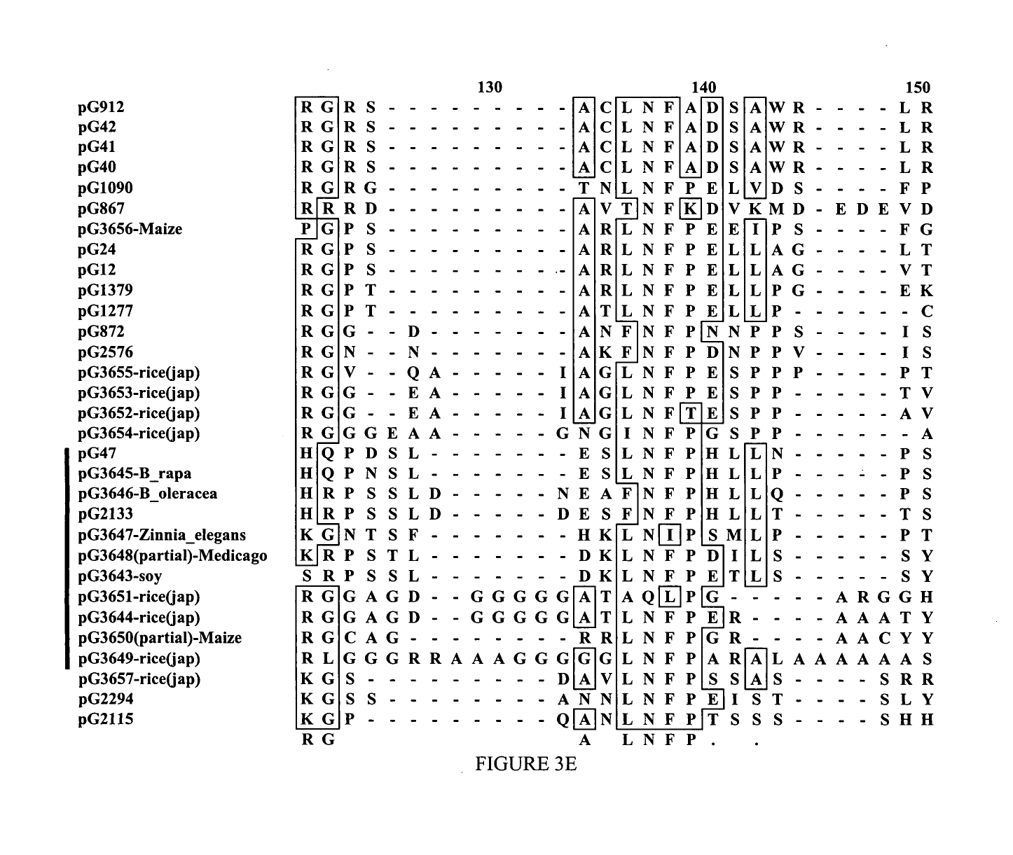

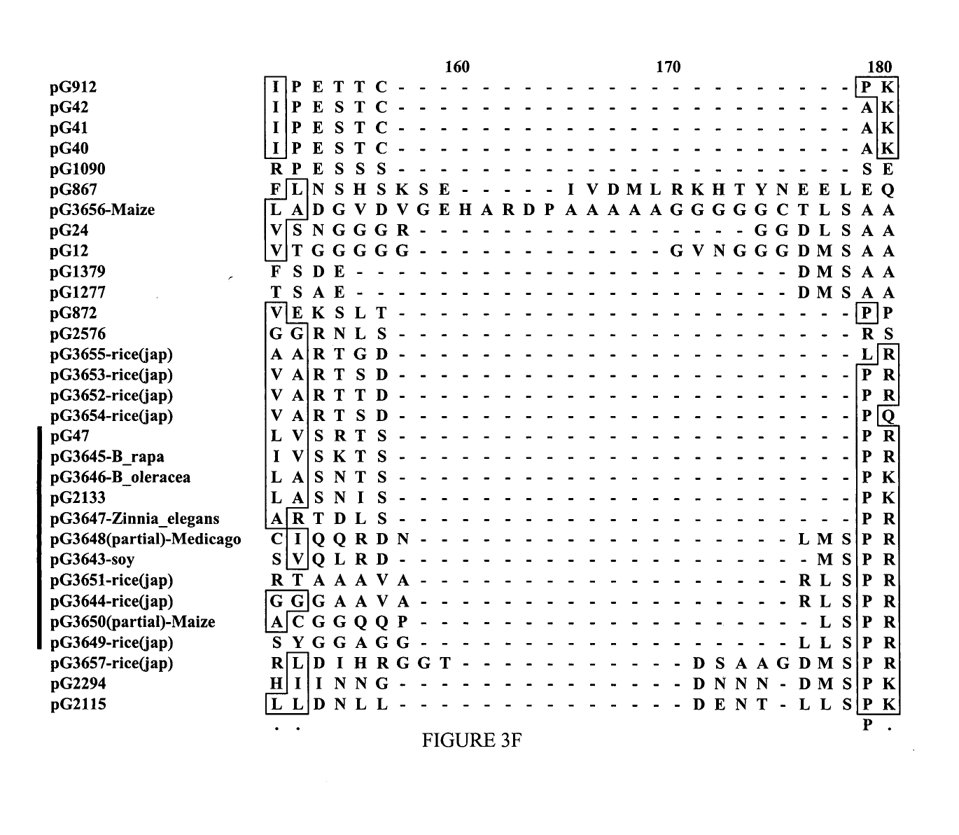

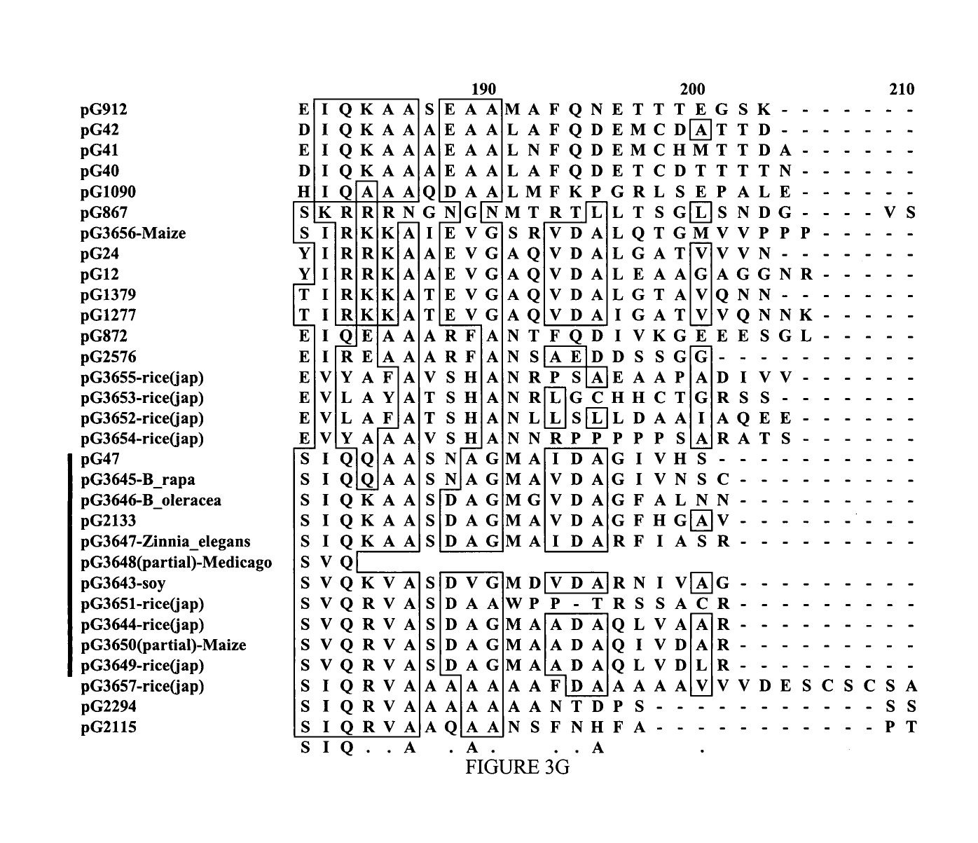

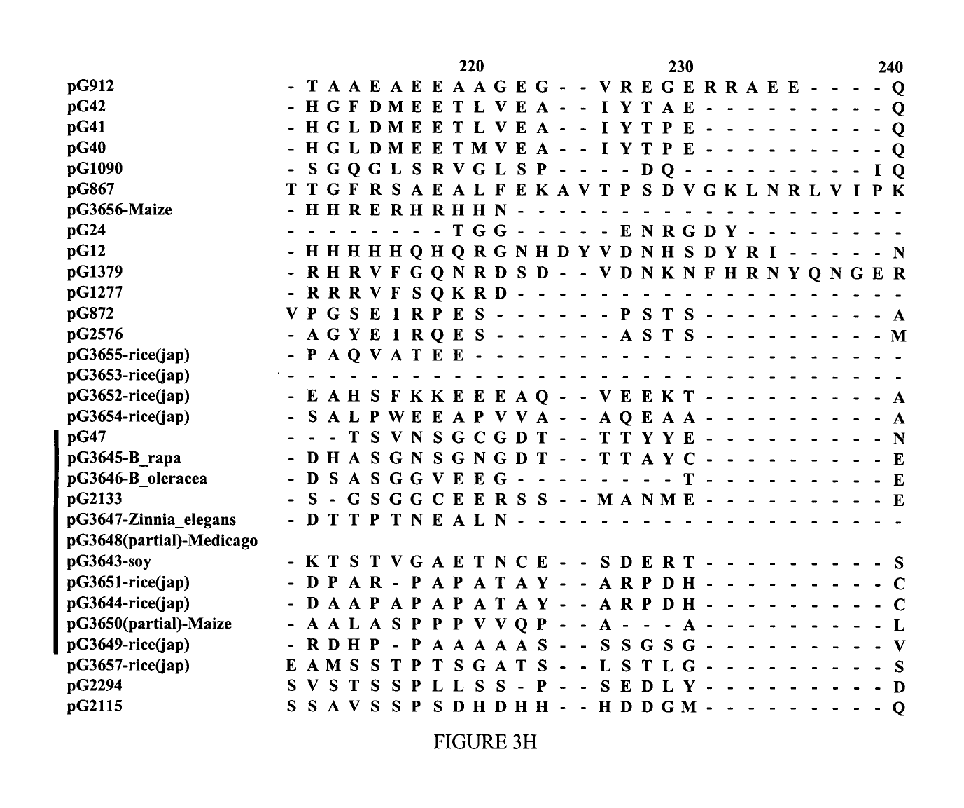

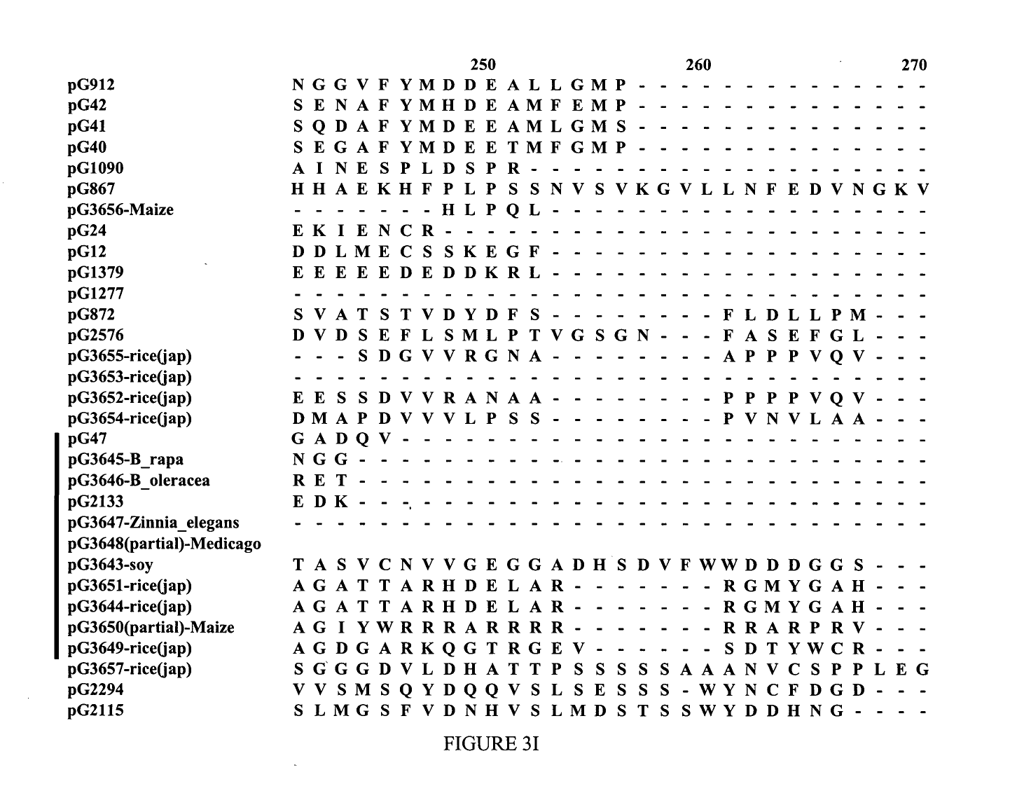

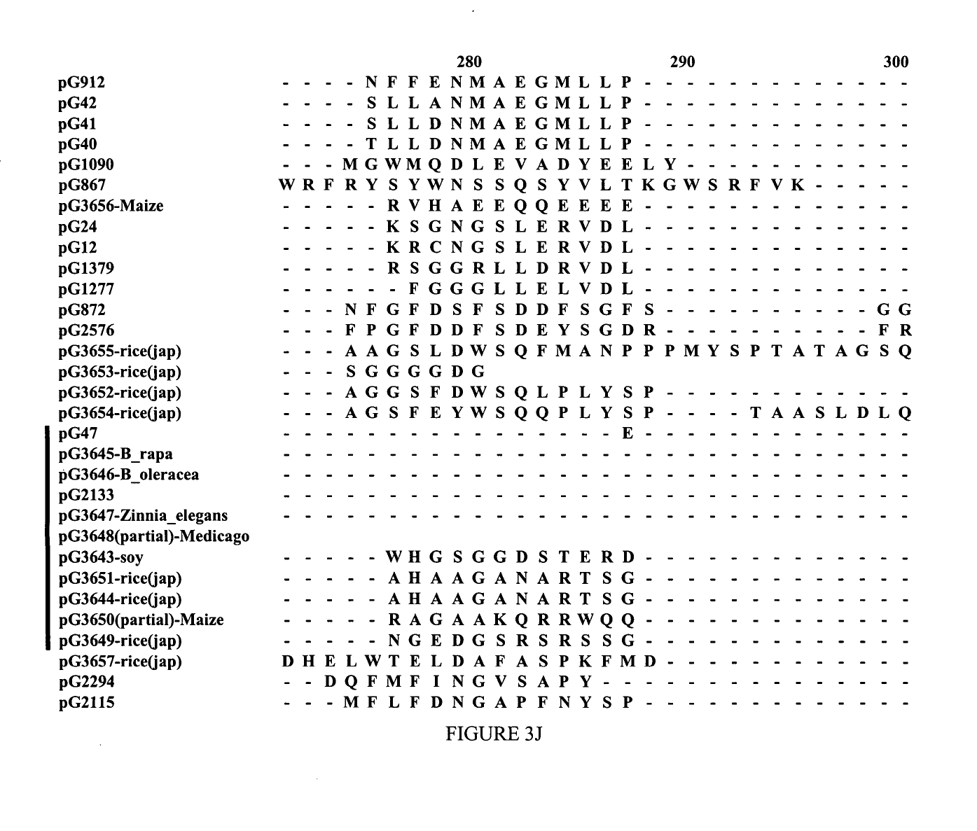

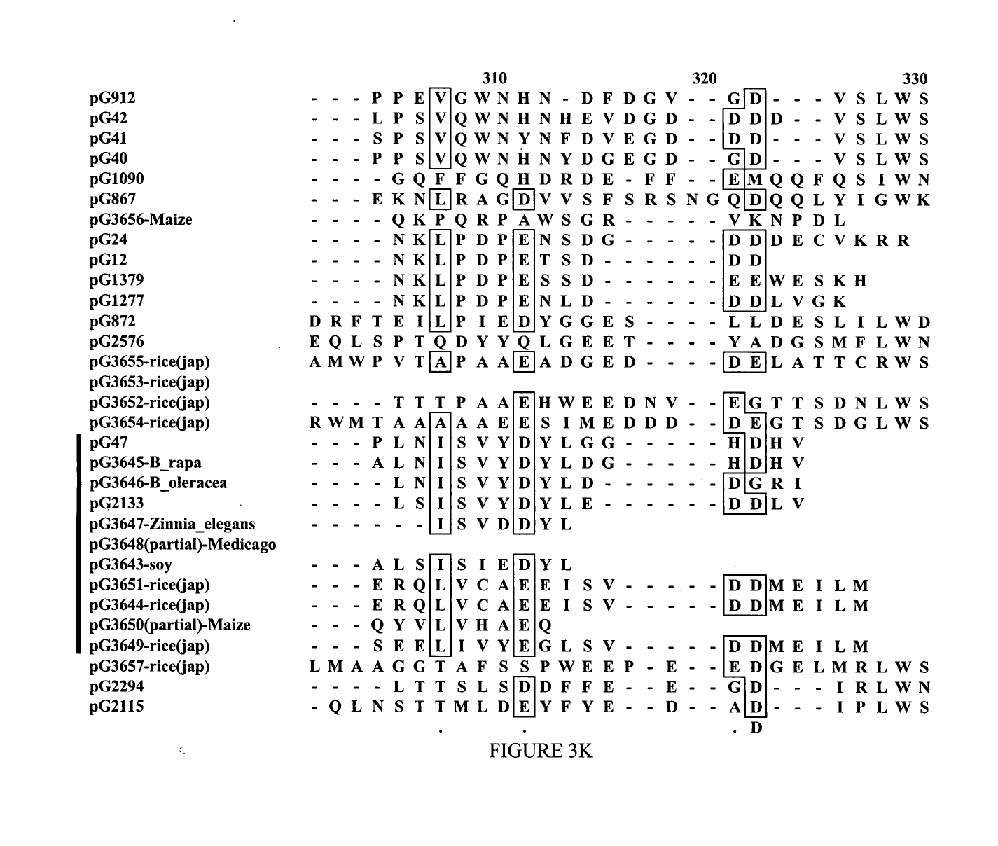

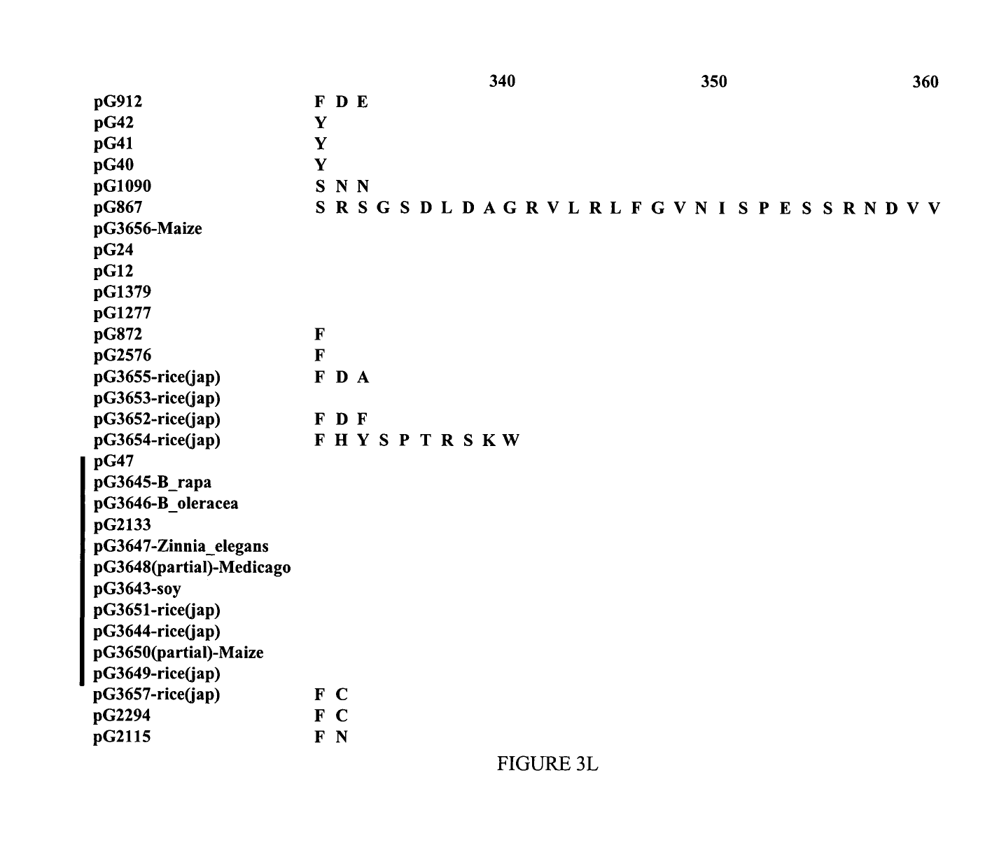

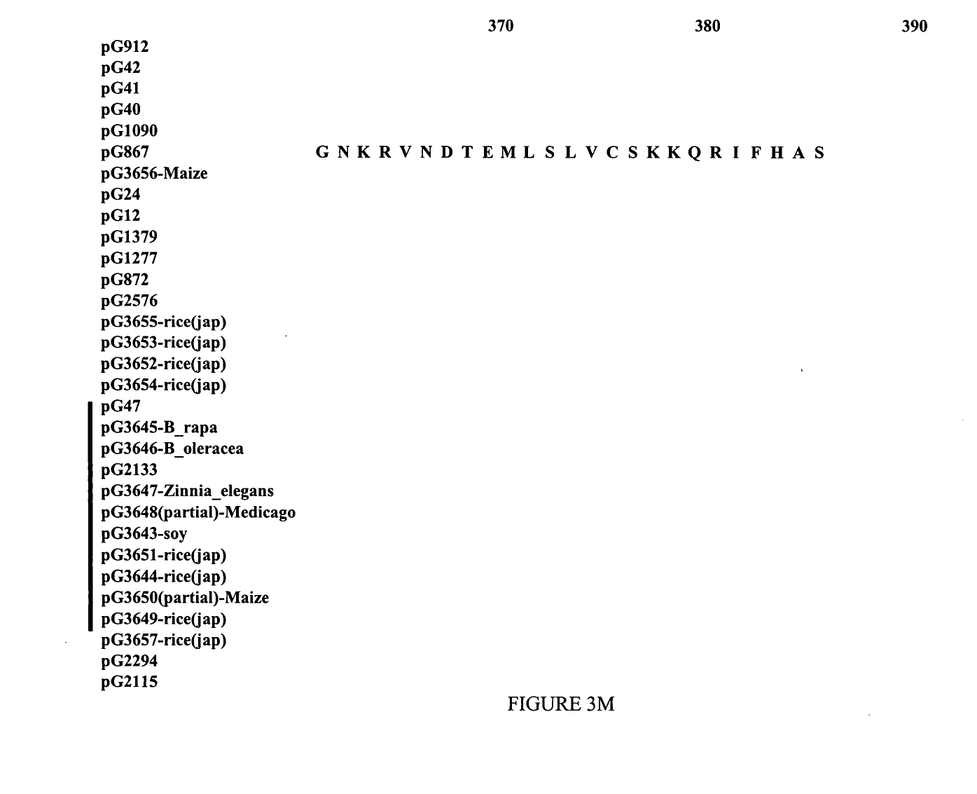

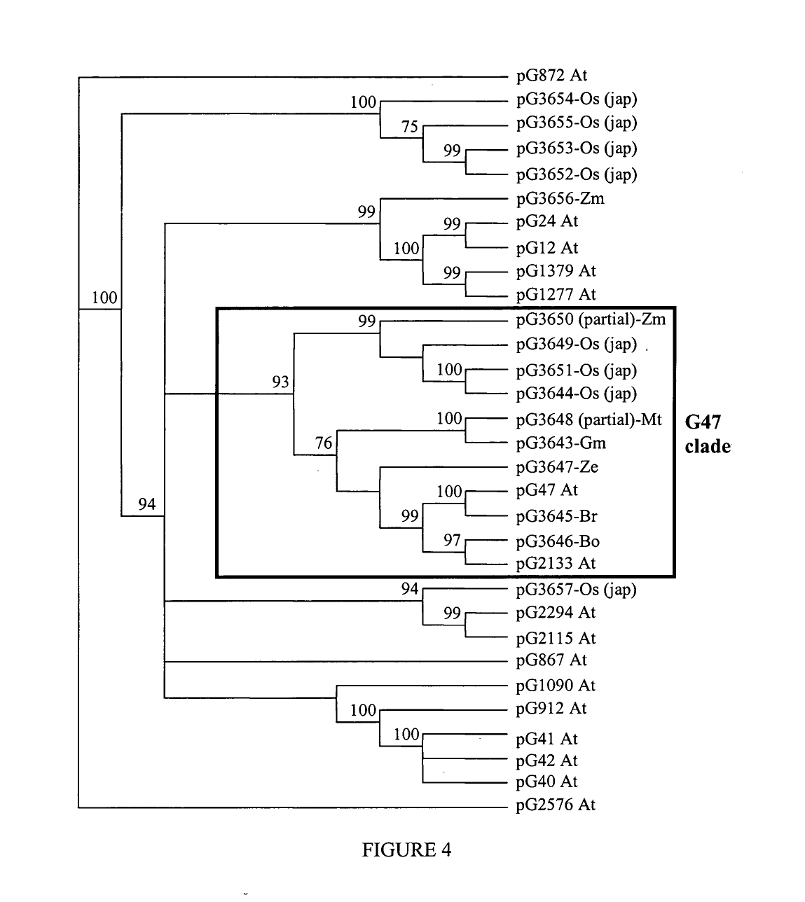

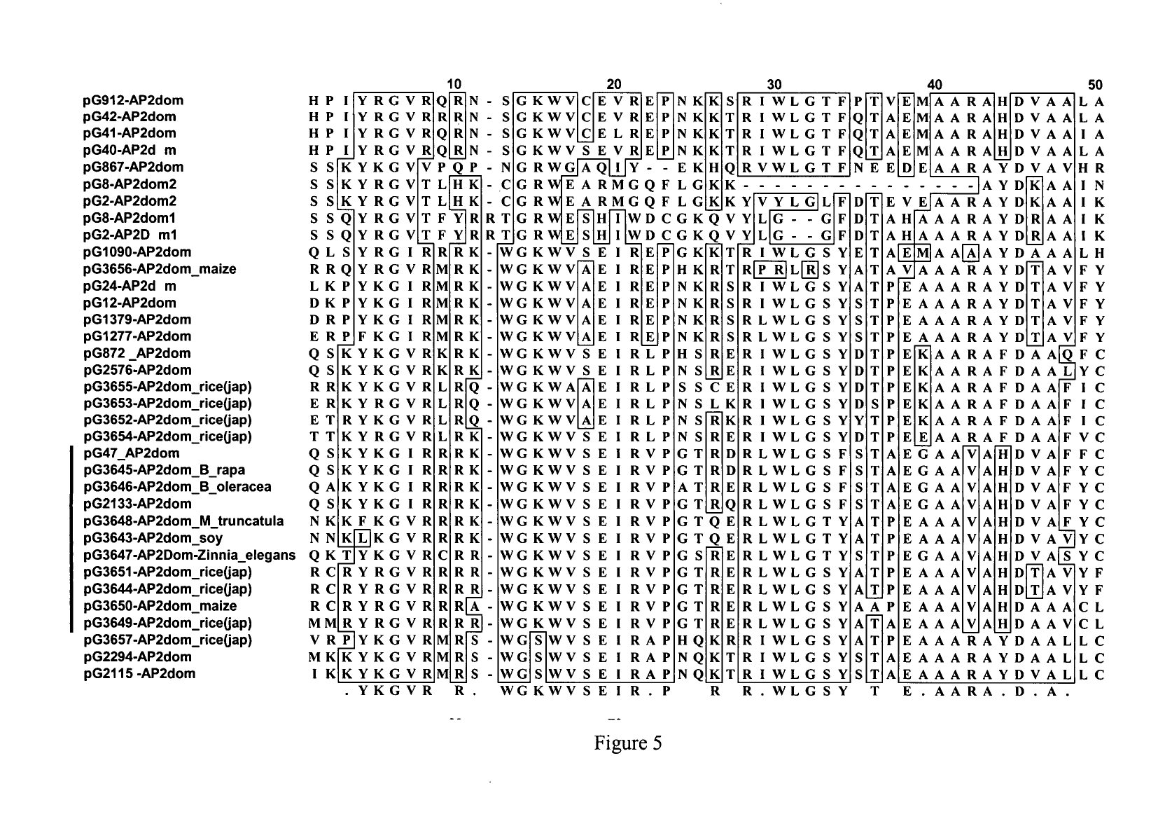

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. (See for example, FIG. 1, adapted from Daly et al. (2001) Plant Physiol. 127: 1328-1333; FIG. 2, adapted from Ku et al. (2000) Proc. Natl. Acad. Sci. 97: 9121-9126; and see also Tudge in The Variety of Life, Oxford University Press, New York, N.Y. (2000) pp. 547-606).

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, for example, a plant explant, as well as to progeny thereof, and to in vitro systems that mimic biochemical or cellular components or processes in a cell.

“Control plant” refers to a plant that serves as a standard of comparison for testing the results of a treatment or genetic alteration, or the degree of altered expression of a gene or gene product. Examples of control plants include plants that are untreated, or genetically unaltered (i.e., wild type).

“Wild type”, as used herein, refers to a cell, tissue or plant that has not been genetically modified to knock out or overexpress one or more of the presently disclosed transcription factors. Wild-type cells, tissue or plants may be used as controls to compare levels of expression and the extent and nature of trait modification with cells, tissue or plants in which transcription factor expression is altered or ectopically expressed, e.g., in that it has been knocked out or overexpressed.

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the transcription factor polynucleotides listed in the Sequence Listing. Exemplary fragments also include fragments that comprise a region that encodes an AP2 domain of a transcription factor.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length. Exemplary polypeptide fragments are the first twenty consecutive amino acids of a mammalian protein encoded by are the first twenty consecutive amino acids of the transcription factor polypeptides listed in the Sequence Listing. Exemplary fragments also include fragments that comprise an AP2 domain of a transcription factor, for example, amino acid residues 10-77 of G2133 (SEQ ID NO: 12), as noted in Table 1.

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, for example, by employing Northern analysis, RT-PCR, microarray gene expression assays, or reporter gene expression systems, or by agricultural observations such as drought stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference compared with a wild-type plant. It is known that there can be a natural variation in the modified trait. Therefore, the trait modification observed entails a change of the normal distribution of the trait in the plants compared with the distribution observed in wild-type plants.

The term “transcript profile” refers to the expression levels of a set of genes in a cell in a particular state, particularly by comparison with the expression levels of that same set of genes in a cell of the same type in a reference state. For example, the transcript profile of a particular transcription factor in a suspension cell is the expression levels of a set of genes in a cell repressing or overexpressing that transcription factor compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may also be evaluated and calculated using statistical and clustering methods.

“Ectopic expression or altered expression” in reference to a polynucleotide indicates that the pattern of expression in, for example, a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more transcription factors are under the control of a strong expression signal, such as one of the promoters described herein (for example, the cauliflower mosaic virus 35S transcription initiation region). Overexpression may occur throughout a plant or in specific tissues of the plant, depending on the promoter used, as described below.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level.

Overexpression thus results in a greater than normal production, or “overproduction” of the transcription factor in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors of the present invention may possess, for example, an AP2 domain, in which case the AP2 domain of the transcription factor binds to a transcription regulating region, such as AtERF1, which binds to the motif AGCCGCC (the “GCC box”) that are present in promoters of genes such as PDF1.2. The transcription factors of the invention also comprise an amino acid subsequence that forms a transcription activation domain that regulates expression of one or more abiotic stress tolerance genes in a plant when the transcription factor binds to the regulating region.

The term “phase change” refers to a plant's progression from embryo to adult, and, by some definitions, the transition wherein flowering plants gain reproductive competency. It is believed that phase change occurs either after a certain number of cell divisions in the shoot apex of a developing plant, or when the shoot apex achieves a particular distance from the roots. Thus, altering the timing of phase changes may affect a plant's size, which, in turn, may affect yield and biomass.

A “sample” with respect to a material containing nucleic acid molecules may comprise a bodily fluid; an extract from a cell, chromosome, organelle, or membrane isolated from a cell; genomic DNA, RNA, or cDNA in solution or bound to a substrate; a cell; a tissue; a tissue print; a forensic sample; and the like. In this context “substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores. A substrate may also refer to a reactant in a chemical or biological reaction, or a substance acted upon (for example, by an enzyme).

“Substantially purified” refers to nucleic acid molecules or proteins that are removed from their natural environment and are isolated or separated, and are at least about 60% free, preferably about 75% free, and most preferably about 90% free, from other components with which they are naturally associated.

### DETAILED DESCRIPTION

Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (see, for example, Riechmann et al. (2000) Science 290: 2105-2110). The plant transcription factors may belong to the AP2 protein transcription factor family (Riechmann and Meyerowitz (1998) supra).

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to drought stress. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, for example, mutation reactions, PCR reactions, or the like; as substrates for cloning for example, including digestion or ligation reactions; and for identifying exogenous or endogenous modulators of the transcription factors. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, for example, genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) Genes Development 11: 3194-3205, and Peng et al. (1999) Nature, 400: 256-261). In addition, many others have demonstrated that an Arabidopsis transcription factor expressed in an exogenous plant species elicits the same or very similar phenotypic response (see, for example, Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500).

In another example, Mandel et al. (1992) Cell 71-133-143), and Suzuki et al. (2001) Plant J. 28: 409-418 teach that a transcription factor expressed in another plant species elicits the same or very similar phenotypic response of the endogenous sequence, as often predicted in earlier studies of Arabidopsis transcription factors in Arabidopsis (see Mandel et al. (1992) supra; Suzuki et al. (2001) supra).

Other examples include Müller et al. (2001) Plant J. 28: 169-179); Kim et al. (2001) Plant J. 25: 247-259); Kyozuka and Shimamoto (2002) Plant Cell Physiol. 43: 130-135); Boss and Thomas (2002) Nature, 416: 847-850); He et al. (2000) Transgenic Res. 9: 223-227); and Robson et al. (2001) Plant J. 28: 619-631).

In yet another example, Gilmour et al. (1998) Plant J. 16: 433442, teach an Arabidopsis AP2 transcription factor, CBF1 (SEQ ID NO: 422), which, when overexpressed in transgenic plants, increases plant freezing tolerance. Jaglo et al. (2001) Plant Physiol. 127: 910-917, further identified sequences in Brassica napus which encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, PKK/RPAGRxKFxETRHP and DSAWR, that bracket the AP2/EREBP DNA binding domains of the proteins and distinguish them from other members of the AP2/EREBP protein family (Jaglo et al. (2001) supra).

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (for example, by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene (and other genes in the MYB family) have been shown to control anthocyanin biosynthesis through regulation of the expression of genes known to be involved in the anthocyanin biosynthetic pathway (Bruce et al. (2000) Plant Cell, 12: 65-79; Borevitz et al. (2000) Plant Cell 12: 2383-93). Further, global transcript profiles have been used successfully as diagnostic tools for specific cellular states (for example, cancerous vs. non-cancerous; Bhattacharjee et al. (2001) Proc Natl. Acad. Sci., USA, 98: 13790-13795; Xu et al. (2001) Proc. Natl. Acad. Sci., USA, 98: 15089-15094). Consequently, it is evident to one skilled in the art that similarity of transcript profile upon overexpression of different transcription factors would indicate similarity of transcription factor function.

Polypeptides and Polynucleotides of the Invention

The present invention provides, among other things, transcription factors (TFs), and transcription factor homolog polypeptides, and isolated or recombinant polynucleotides encoding the polypeptides, or novel sequence variant polypeptides or polynucleotides encoding novel variants of transcription factors derived from the specific sequences provided here.

The polynucleotides of the invention can be or were ectopically expressed in overexpressor plant cells and the changes in the expression levels of a number of genes, polynucleotides, and/or proteins of the plant cells observed. Therefore, the polynucleotides and polypeptides can be employed to change expression levels of a genes, polynucleotides, and/or proteins of plants. These polypeptides and polynucleotides may be employed to modify a plant's characteristics, particularly drought tolerance. The polynucleotides of the invention can be or were ectopically expressed in overexpressor or knockout plants and the changes in the characteristic(s) or trait(s) of the plants observed. Therefore, the polynucleotides and polypeptides can be employed to improve the characteristics of plants. The polypeptide sequences of the sequence listing, including Arabidopsis sequences G2133, G1274, G922, G2999, G3086, G354, G1792, G2053, G975, G1069, G916, G1820, G2701, G47, G2854, G2789, G634, G175, G2839, G1452, G3083, G489, G303, G2992, and G682, (SEQ ID NOs: 12, 6, 4, 14, 16, 228, 8, 10, 238, 240, 236, 244, 246, 2, 252, 248, 232, 224, 250, 242, 254, 230, 226, 50 and 234, respectively) have been shown to confer increased drought tolerance when these polypeptides are overexpressed in Arabidopsis plants. These polynucleotides have been shown to have a strong association with drought stress tolerance, in that plants that overexpress these sequences are more tolerant to drought. The invention also encompasses a complement of the polynucleotides. The polynucleotides are also useful for screening libraries of molecules or compounds for specific binding and for creating transgenic plants having increased osmotic stress tolerance. Altering the expression levels of equivalogs of these sequences, including paralogs and orthologs in the Sequence Listing, and other orthologs that are structurally and sequentially similar to the former orthologs, has been shown and is expected to confer similar phenotypes, including drought tolerance, in plants in some cases, exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences were subsequently recovered by the rapid amplification of cDNA ends (RACE) procedure, using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

The polynucleotides are particularly useful when they are hybridizable array elements in a microarray. Such a microarray can be employed to monitor the expression of genes that are differentially expressed in response to drought or other osmotic stresses. The microarray can be used in large scale genetic or gene expression analysis of a large number of polynucleotides; or in the diagnosis of drought stress before phenotypic symptoms are evident. Furthermore, the microarray can be employed to investigate cellular responses, such as cell proliferation, transformation, and the like.

When the polynucleotides of the invention may also be used as hybridizable array elements in a microarray, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular stress, pathology, or treatment.

The invention also entails an agronomic composition comprising a polynucleotide of the invention in conjunction with a suitable carrier and a method for altering a plant's trait using the composition.

Examples of specific polynucleotide and polypeptides of the invention, and equivalog sequences, along with descriptions of the gene families that comprise these polynucleotides and polypeptides, are provided below.

The AP2 family, including the G47/G2133 and G1792 clades. AP2 (APETALA2) and EREBPs (Ethylene-Responsive Element Binding Proteins) are the prototypic members of a family of transcription factors unique to plants, whose distinguishing characteristic is that they contain the so-called AP2 DNA-binding domain (for a review, see Riechmann and Meyerowitz (1998) Biol. Chem. 379: 633-646). The AP2 domain was first recognized as a repeated motif within the Arabidopsis thaliana AP2 protein (Jofuku et al. (1994) Plant Cell 6: 1211-1225). Shortly afterwards, four DNA-binding proteins from tobacco were identified that interact with a sequence that is essential for the responsiveness of some promoters to the plant hormone ethylene, and were designated as ethylene-responsive element binding proteins (EREBPs; Ohme-Takagi et al. (1995) Plant Cell 7: 173-182). The DNA-binding domain of EREBP-2 was mapped to a region that was common to all four proteins (Ohme-Takagi et al (1995) supra), and that was found to be closely related to the AP2 domain (Weigel (1995) Plant Cell 7: 388-389) but that did not bear sequence similarity to previously known DNA-binding motifs.

AP2/EREBP genes form a large family, with many members known in several plant species (Okamuro et al. (1997) Proc. Natl. Acad. Sci. USA 94: 7076-7081; Riechmann and Meyerowitz (1998) supra). The number of AP2/EREBP genes in the Arabidopsis thaliana genome is approximately 145 (Riechmann et al. (2000) Science 290: 2105-2110). The APETALA2 class is characterized by the presence of two AP2 DNA binding domains, and contains 14 genes. The AP2/ERF is the largest subfamily, and includes 125 genes which are involved in abiotic (DREB subgroup) and biotic (ERF subgroup) stress responses and the RAV subgroup includes 6 genes which all have a B3 DNA binding domain in addition to the AP2 DNA binding domain (Kagaya et al. (1999) Nucleic Acids Res. 27: 470-478).

Arabidopsis AP2 is involved in the specification of sepal and petal identity through its activity as a homeotic gene that forms part of the combinatorial genetic mechanism of floral organ identity determination and it is also required for normal ovule and seed development (Bowman et al. (1991) Development 112: 1-20; Jofuku et al. (1994) supra). Arabidopsis ANT is required for ovule development and it also plays a role in floral organ growth (Elliott et al. (1996) Plant Cell 8: 155-168; Klucher et al. (1996) Plant Cell 8: 137-153). Finally, maize G115 regulates leaf epidermal cell identity (Moose et al. (1996) Genes Dev. 10: 3018-3027).

The attack of a plant by a pathogen may induce defense responses that lead to resistance to the invasion, and these responses are associated with transcriptional activation of defense-related genes, among them those encoding pathogenesis-related (PR) proteins. The involvement of EREBP-like genes in controlling the plant defense response is based on the observation that many PR gene promoters contain a short cis-acting element that mediates their responsiveness to ethylene (ethylene appears to be one of several signal molecules controlling the activation of defense responses). Tobacco EREBP-1, -2, -3, and -4, and tomato Pti4, Pti5 and Pti6 proteins have been shown to recognize such cis-acting elements (Ohme-Takagi (1995) supra; Zhou et al. (1997) EMBO J. 16: 3207-3218). In addition, Pti4, Pti5, and Pti6 proteins have been shown to directly interact with Pto, a protein kinase that confers resistance against Pseudomonas syringae pv tomato (Zhou et al, (1997) supra). Plants are also challenged by adverse environmental conditions like cold or drought, and EREBP-like proteins appear to be involved in the responses to these abiotic stresses as well. COR (for cold-regulated) gene expression is induced during cold acclimation, the process by which plants increase their resistance to freezing in response to low unfreezing temperatures. The Arabidopsis EREBP-like gene CBF1 (Stockinger et al. (1997) Proc. Natl. Acad. Sci. USA 94: 1035-1040) is a regulator of the cold acclimation response, because ectopic expression of CBF1 in Arabidopsis transgenic plants induced COR gene expression in the absence of a cold stimulus, and the plant freezing tolerance was increased (Jaglo-Ottosen et al. (1998) Science 280: 104-106). Finally, another Arabidopsis EREBP-like gene, ABI4, is involved in abscisic acid (ABA) signal transduction, because abi4 mutants are insensitive to ABA (ABA is a plant hormone that regulates many agronomically important aspects of plant development; Finkelstein et al. (1998) Plant Cell 10: 1043-1054).

The SCR family, including the G922 clade. The SCARECROW gene, which regulates an asymmetric cell division essential for proper radial organization of root cell layers, was isolated from Arabidopsis thaliana by screening a genomic library with sequences flanking a T-DNA insertion causing a “scarecrow” mutation (Di Laurenzio et al. (1996) Cell 86, 423433). The gene product was tentatively described as a transcription factor based on the presence of homopolymeric stretches of several amino acids, the presence of a basic domain similar to that of the basic-leucine zipper family of transcription factors, and the presence of leucine heptad repeats. The presence of several Arabidopsis ESTs with gene products homologous to the SCARECROW gene were noted. The ability of the SCARECROW gene to complement the scarecrow mutation was also demonstrated (Malamy et al. (1997) Plant J. 12, 957-963).

More recently, the SCARECROW homologue RGA, which encodes a negative regulator of the gibberellin signal transduction pathway, was isolated from Arabidopsis by genomic subtraction (Silverstone et al. (1998) Plant Cell 10, 155-169). The RGA gene was shown to be expressed in many different tissues and the RGA protein was shown to be localized to the nucleus. The same gene was isolated by Truong (Truong et al. (1997) FEBS Lett. 410: 213-218) by identifying cDNA clones which complement a yeast nitrogen metabolism mutant, suggesting that RGA may be involved in regulating diverse metabolic processes. Another SCARECROW homologue designated GAI, which also is involved in gibberellin signaling processes, has been isolated by Peng (Peng et al. (1997) Genes Dev. 11, 3194-3205). Interestingly, GAI is the gene that initiated the Green Revolution. Peng et al. (Peng et al. (1999) Nature 6741, 256-261) have recently shown that maize GAI orthologs, when mutated, result in plants that are shorter, have increased seed yield, and are more resistant to damage by rain and wind than wild type plants. Based on the inclusion of the GAI, RGA and SCR genes in this family, it has also been referred to as the GRAS family (Pysh et al. (1999) Plant J 18, 111-19).

The scarecrow gene family has 32 members in the Arabidopsis genome.

The WRKY family, including the G1274 clade. The WRKY family of transcription factors is thus far only found in plants. It is primarily characterized by a 60 amino acid conserved DNA binding domain and a zinc finger domain. The family is divided into groups based on whether the protein has two or only one WRKY domain (Groups I and II, respectively), and further subdivided based on a unique variation of the zing finger motif (Group III) as described by Eulgem (Eulgem et al. (2000) Trends Plant Science 5:199-206). G1274 (polynucleotide SEQ ID NO: 5 and polypeptide SEQ ID NO: 6) belongs to the so-called Group II class of WRKY proteins, which can be further subdivided into 5 groups (a-e) based on conserved structural features outside of the WRKY domain. G1274 is a member of the IIc subgroup.

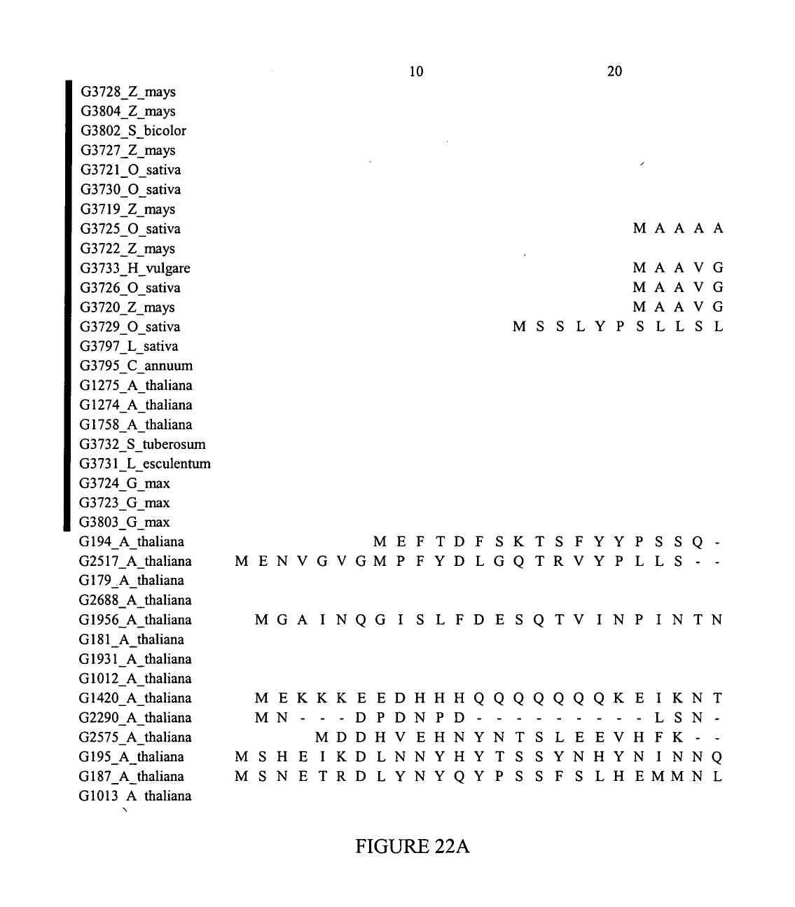

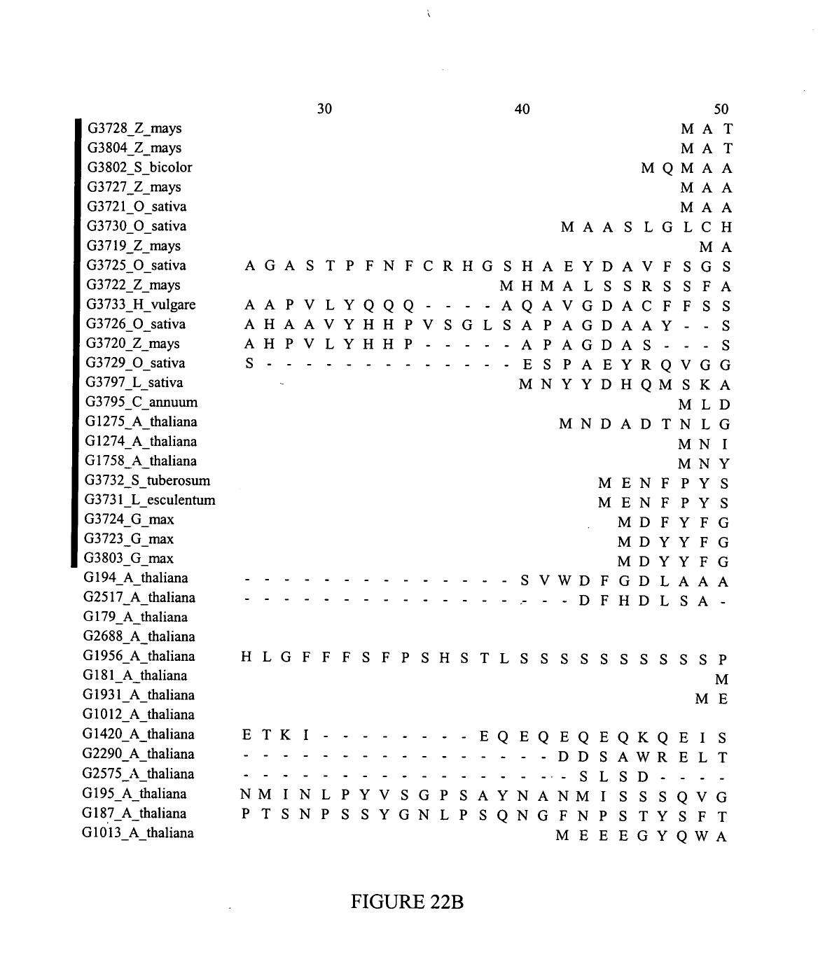

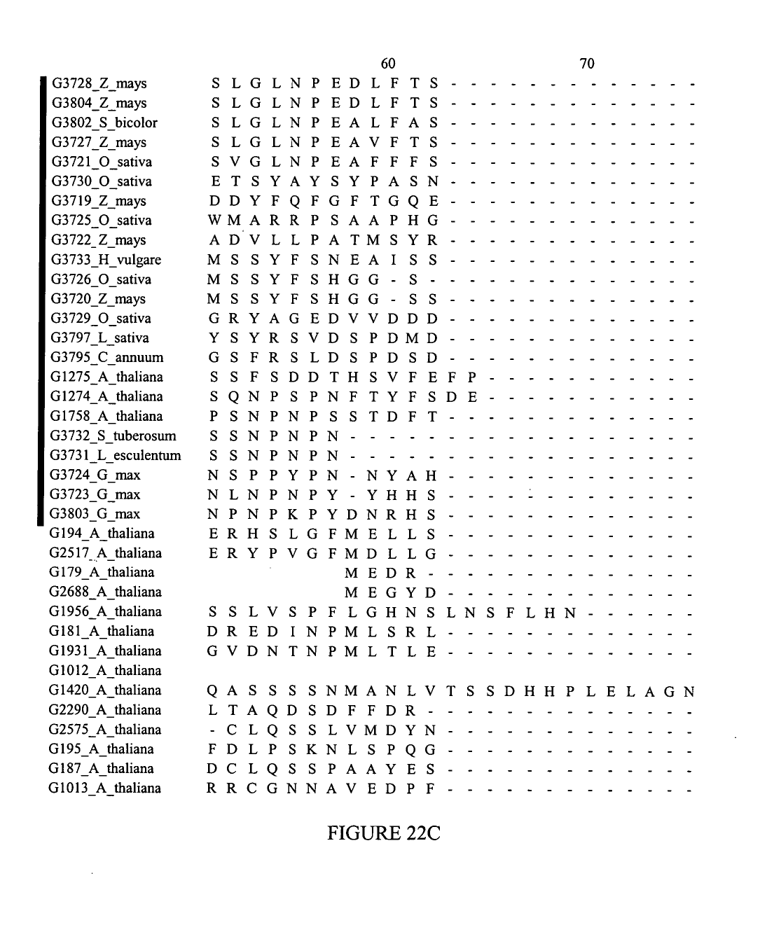

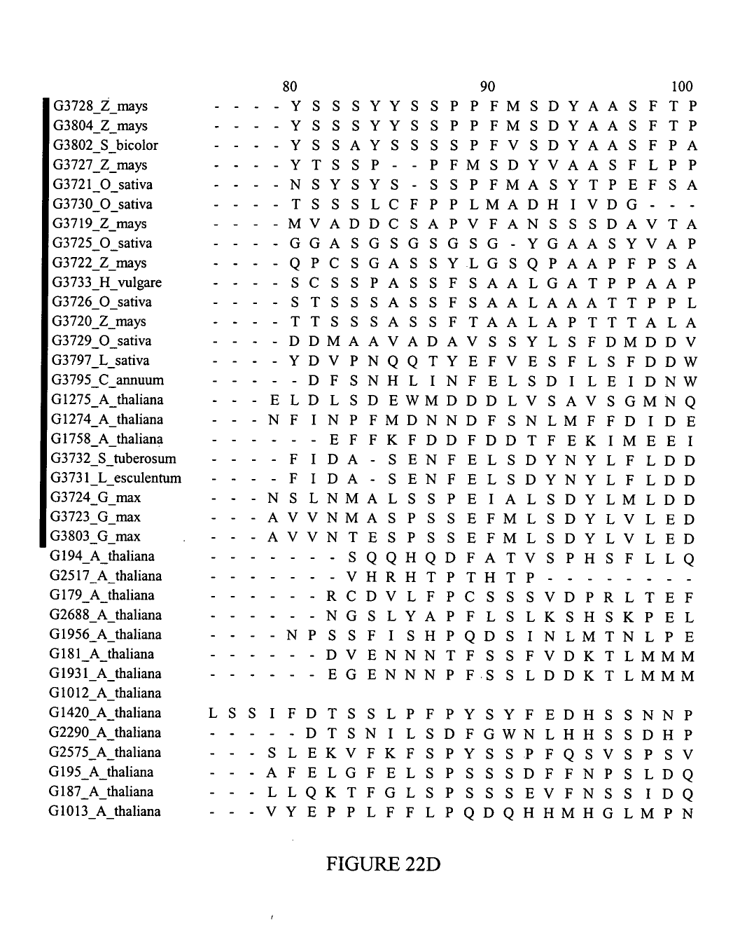

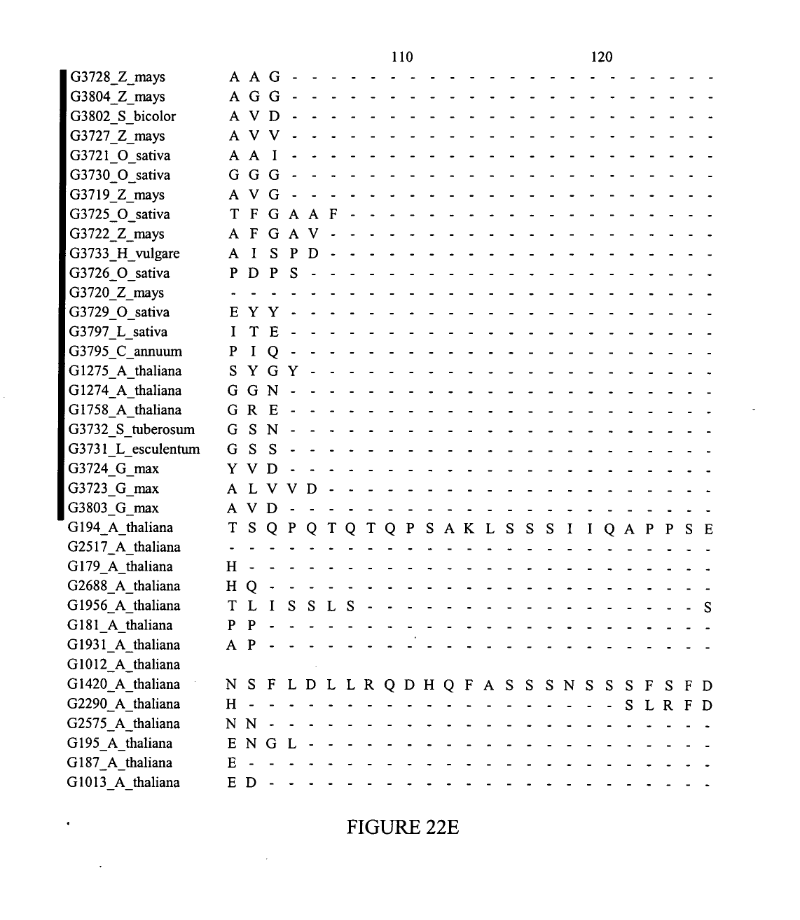

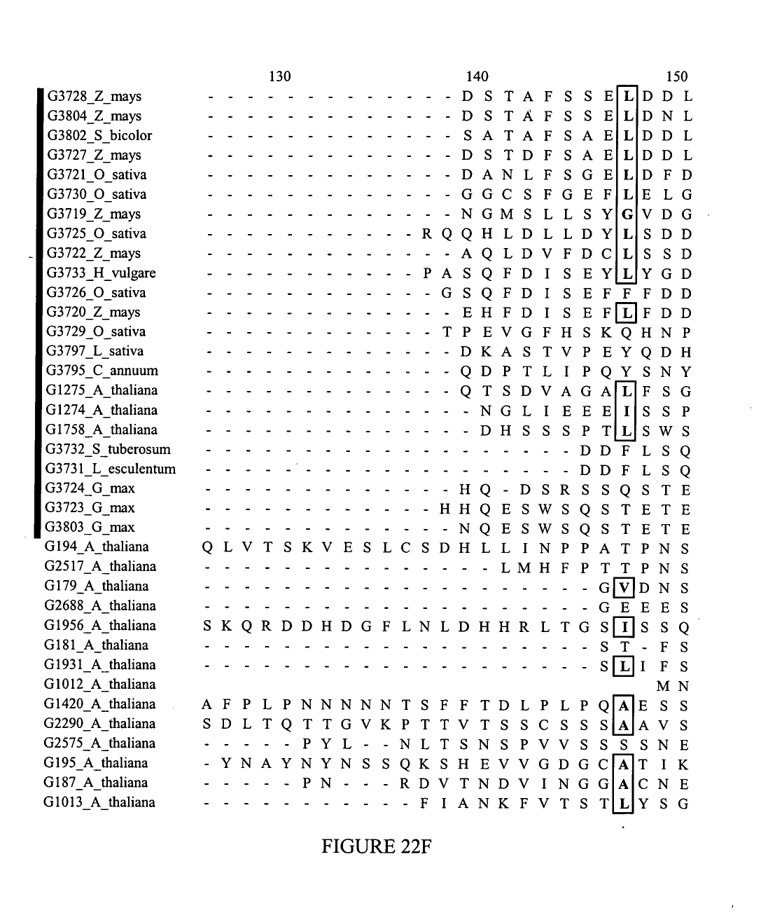

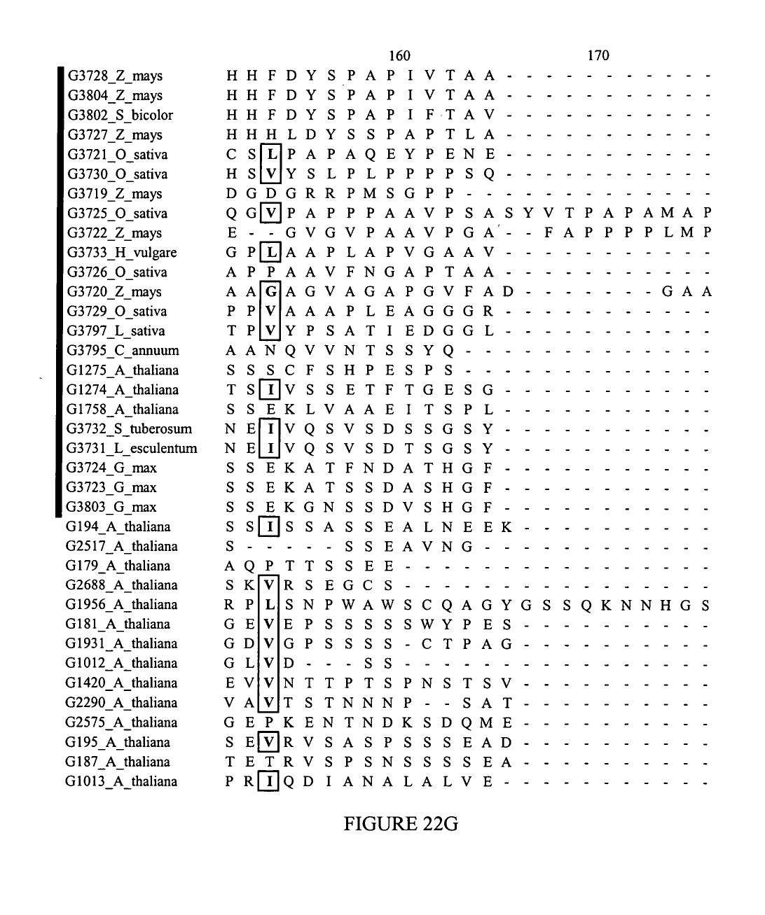

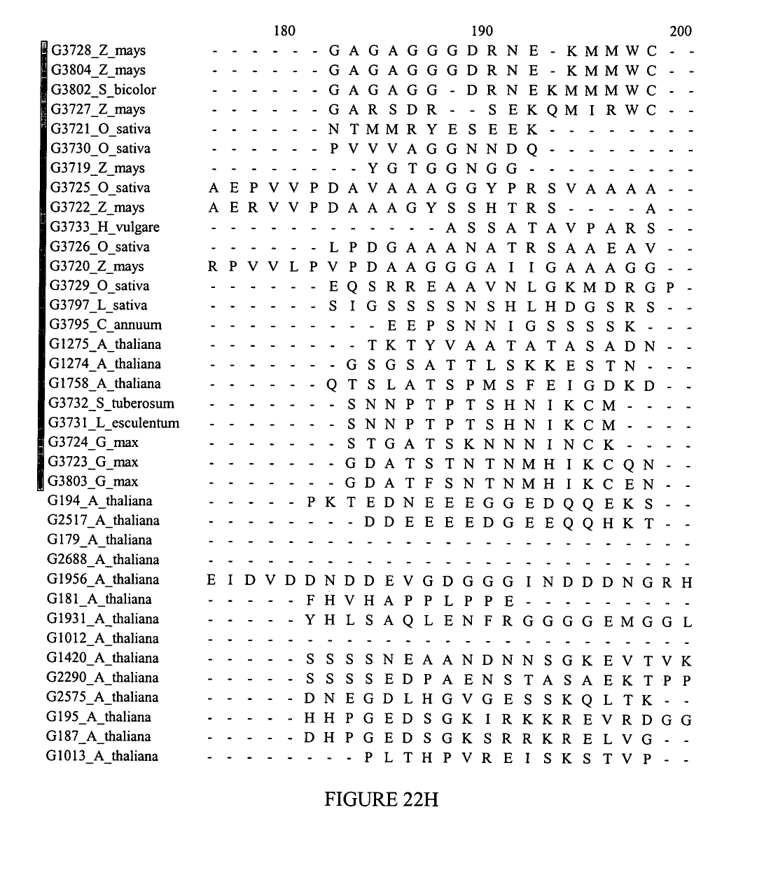

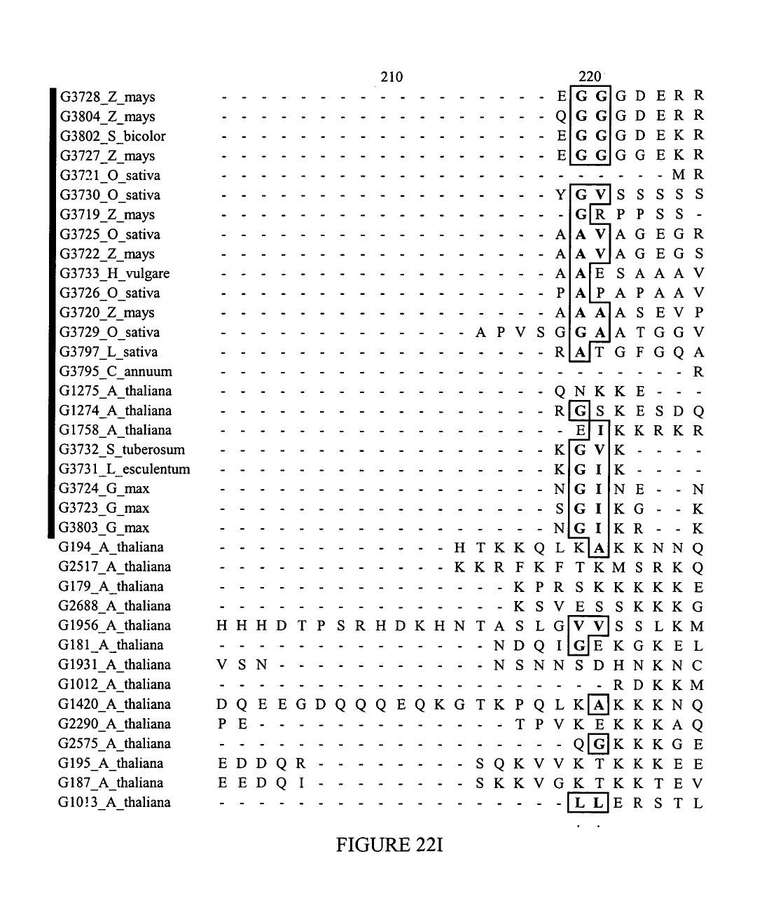

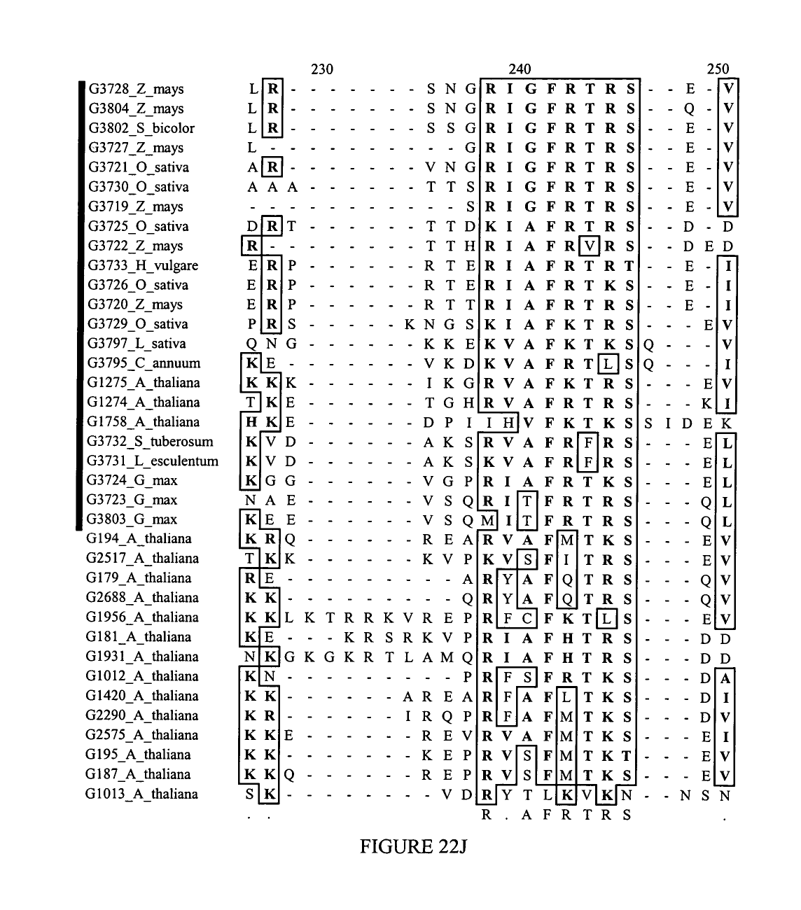

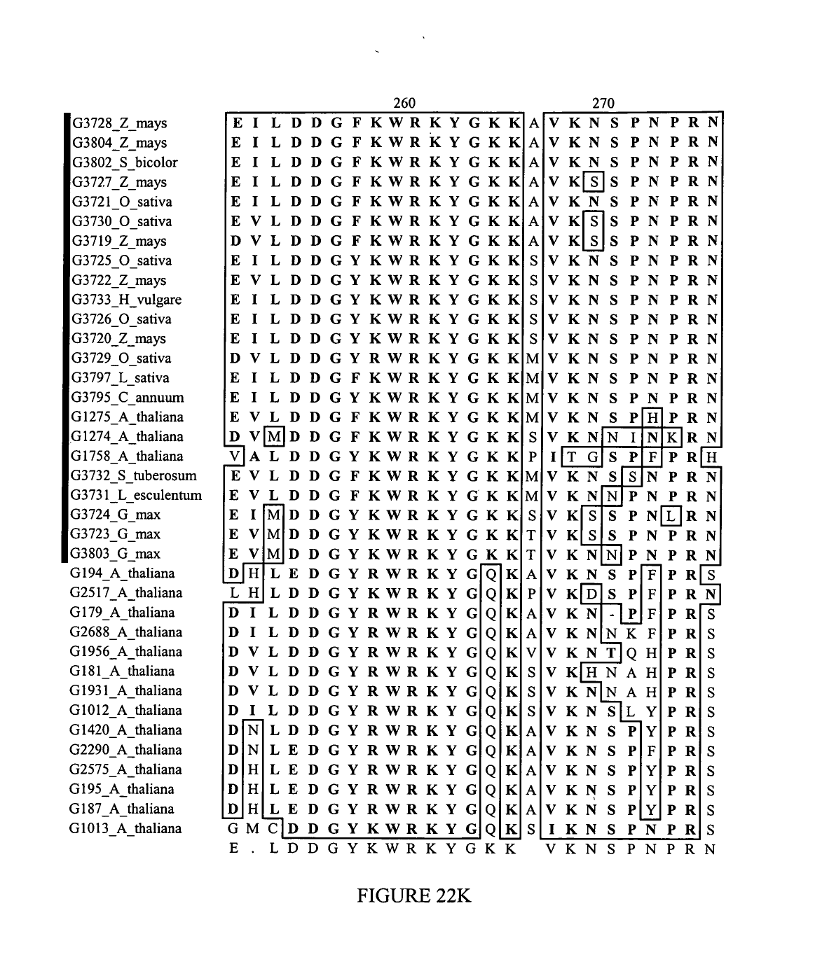

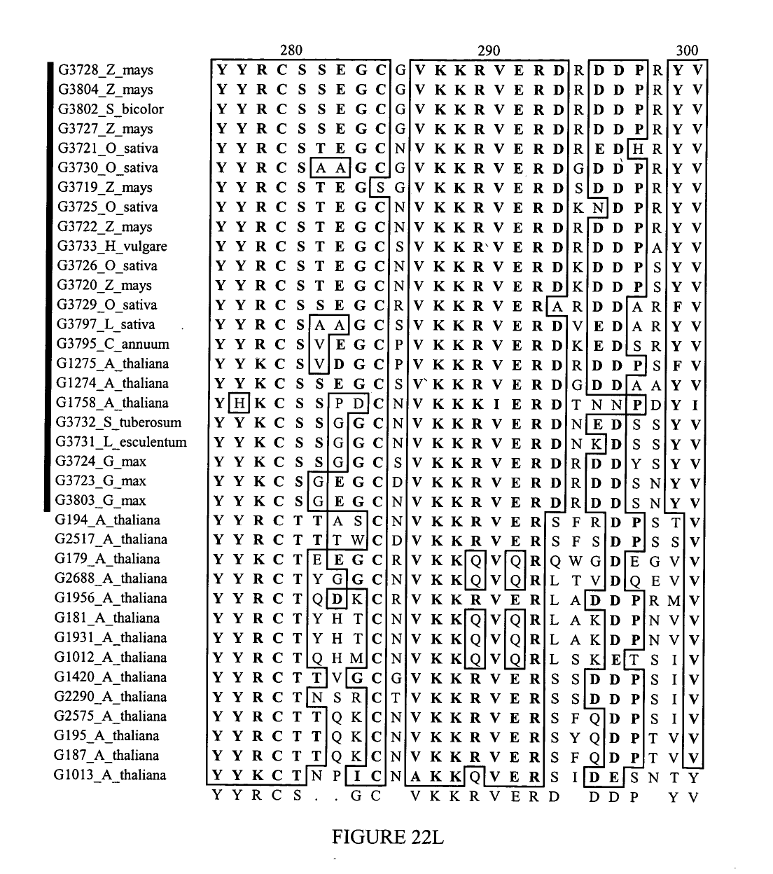

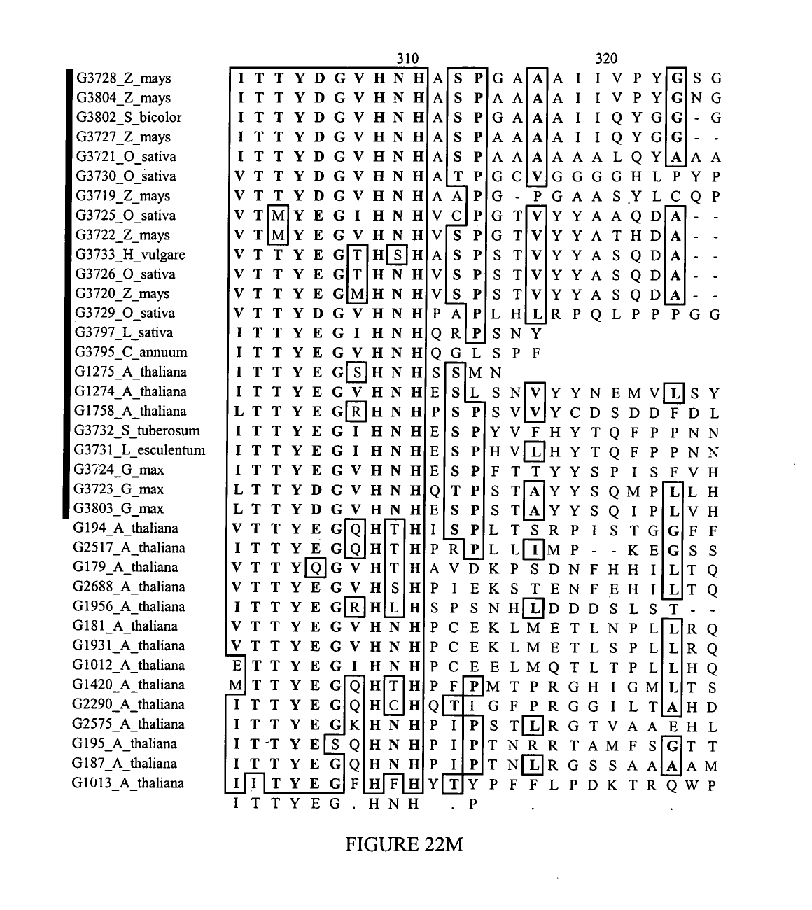

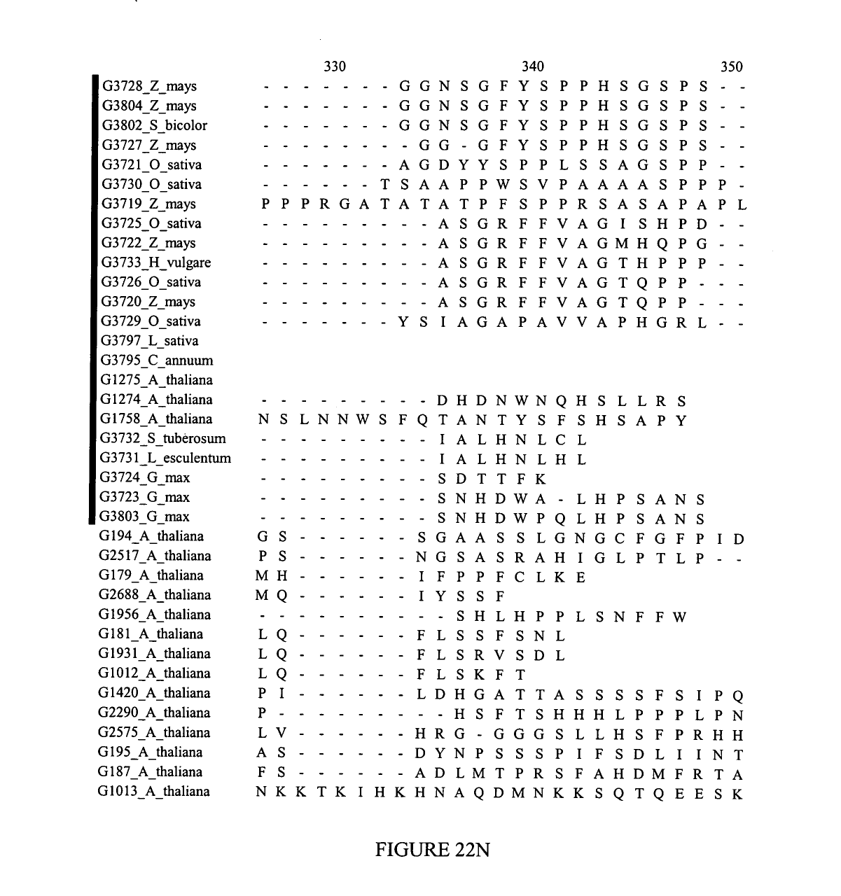

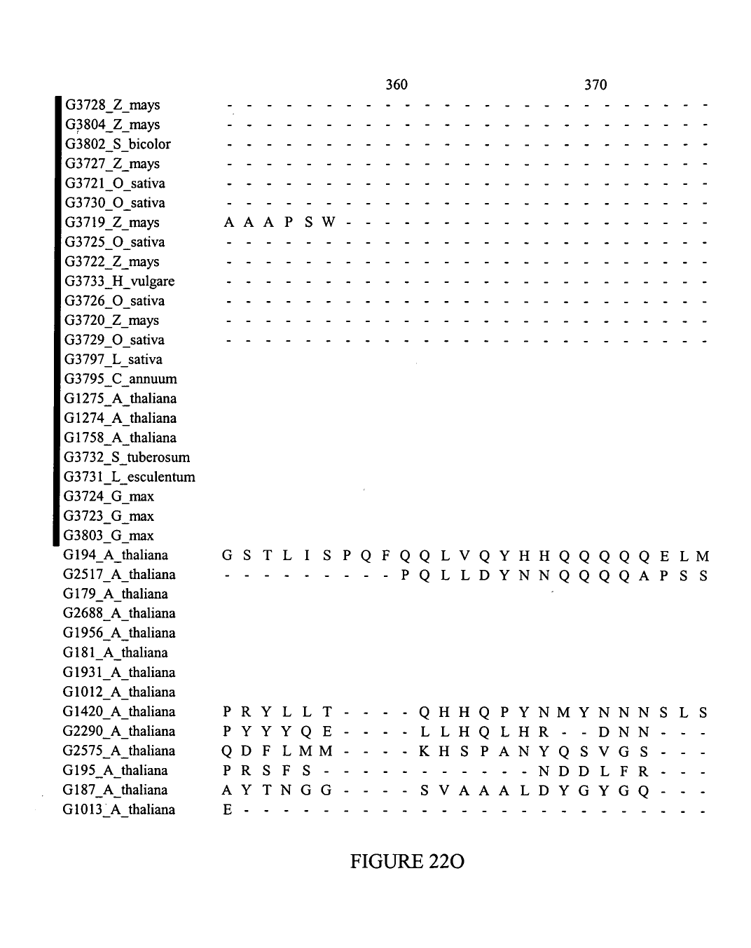

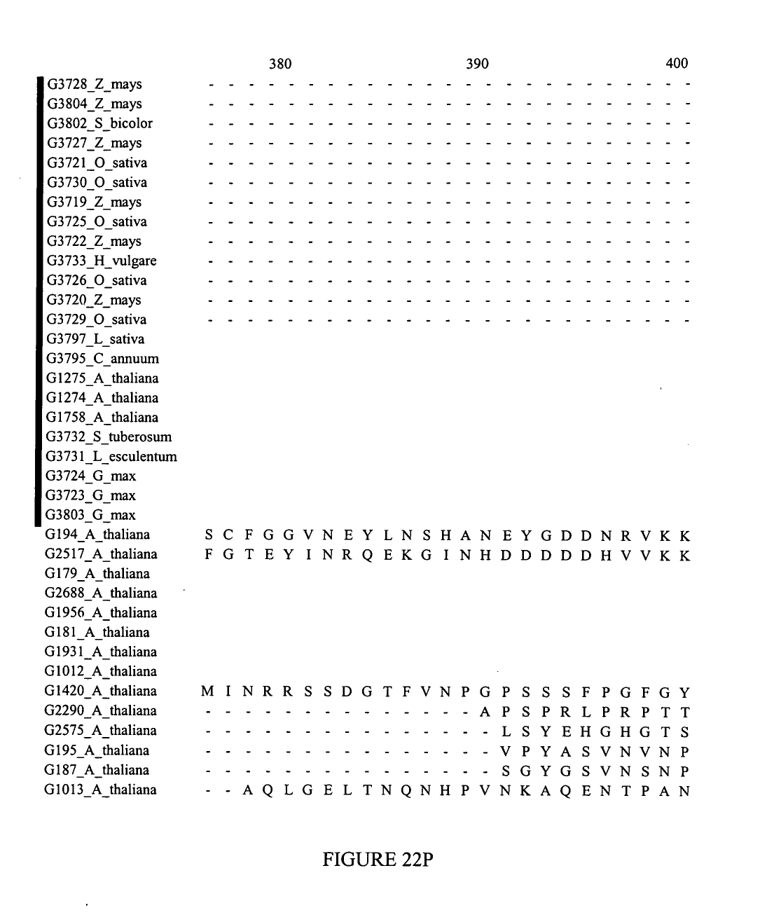

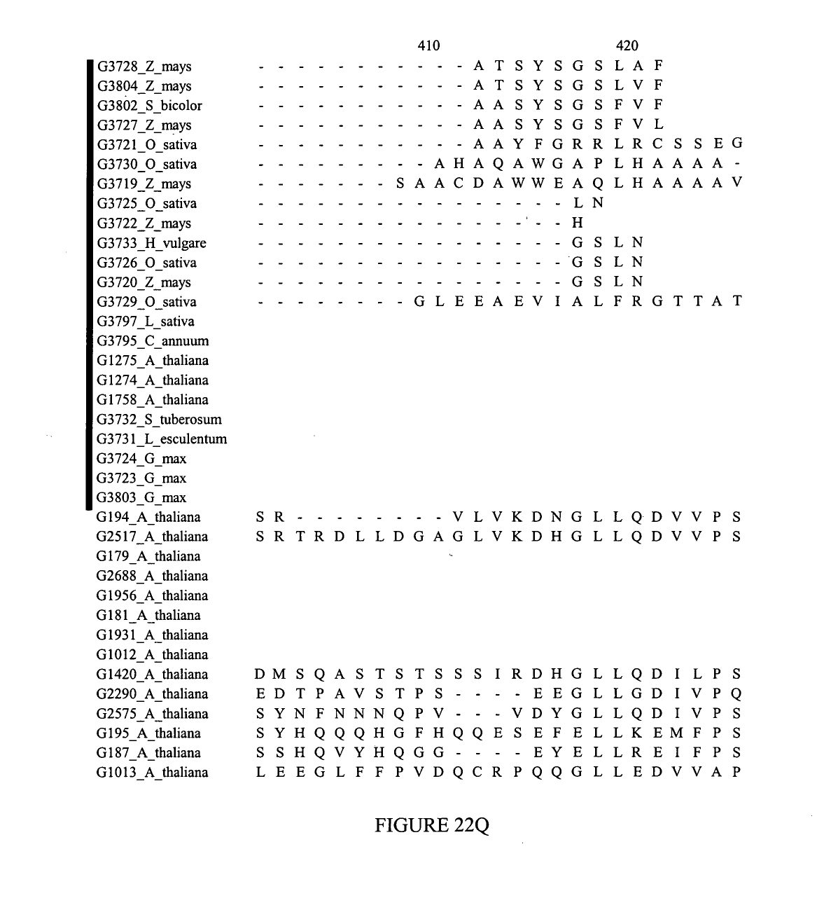

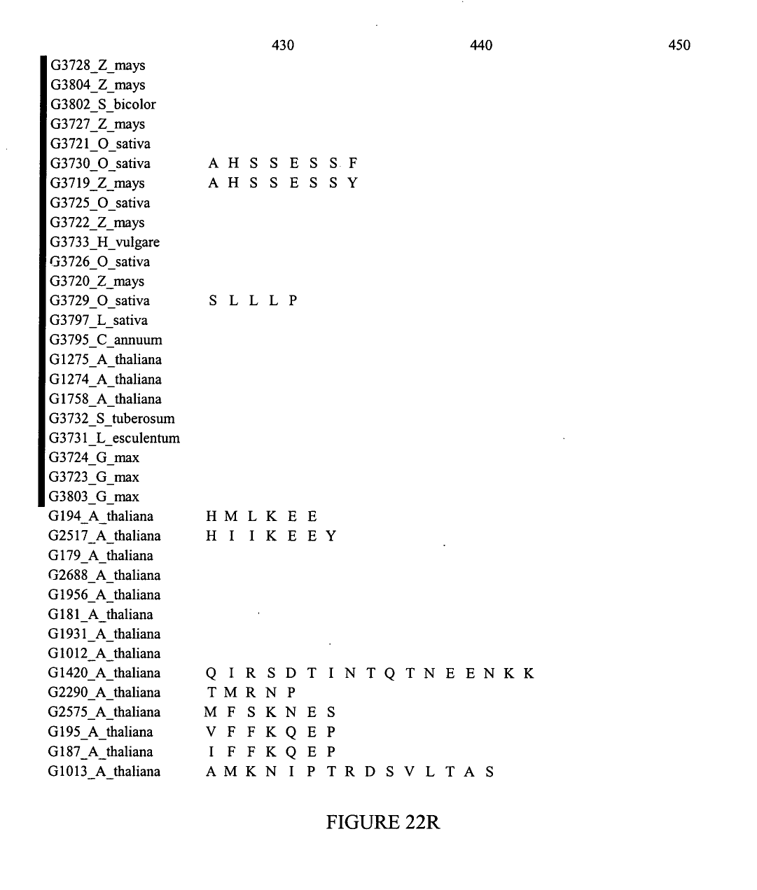

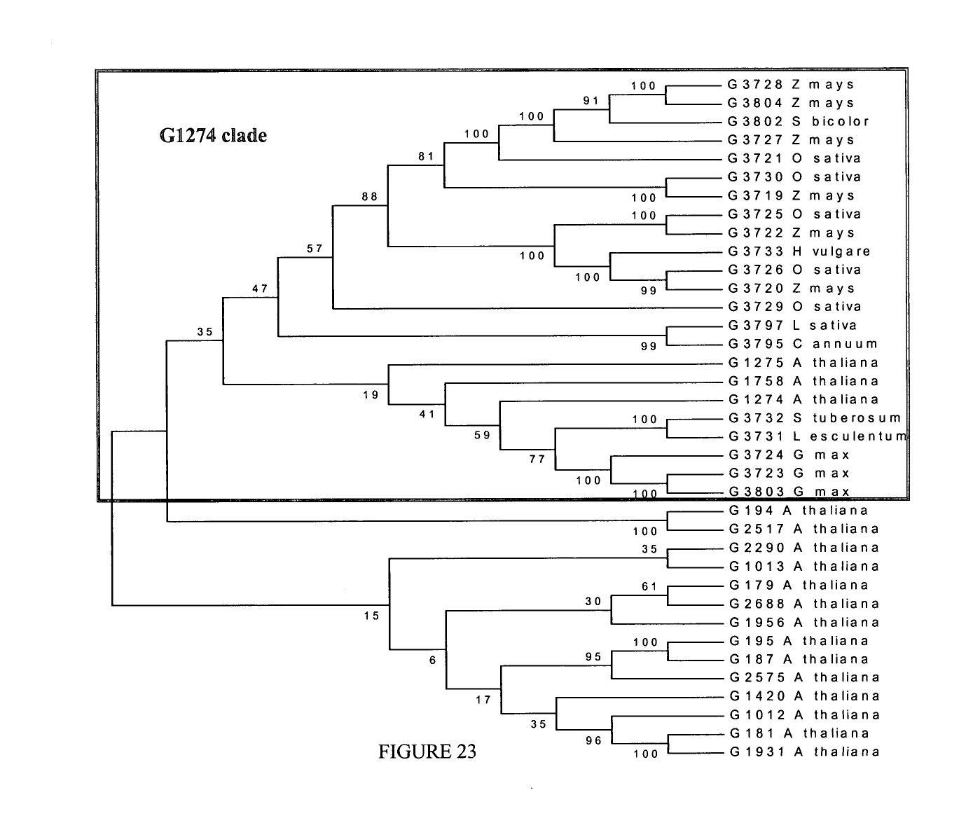

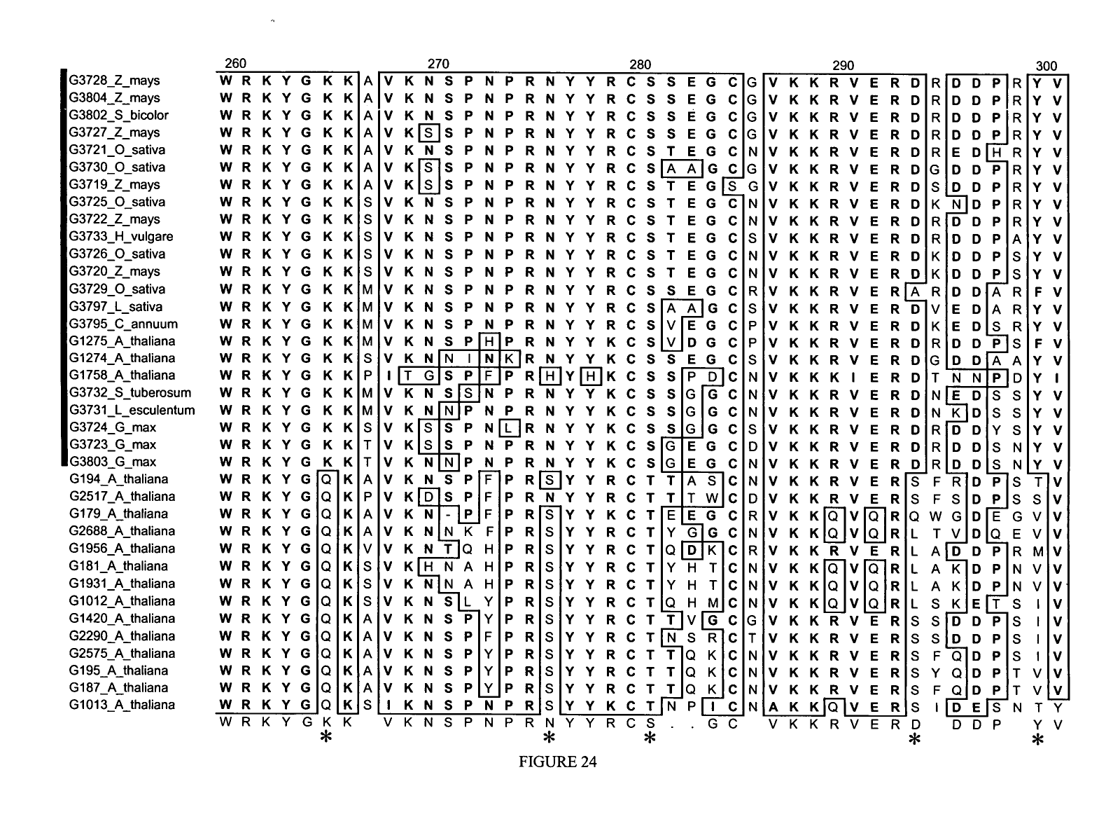

The phylogenetic tree in FIG. 23 uses other closely related members of the WRKY Group IIc family as a natural out-group to the G1274 clade. Using either the full protein, or WRKY domain, the potentially orthologous sequences shown on the tree appear most closely related to the G1274 paralog clade. FIG. 22 shows the aligned sequences of the full-length proteins, and FIG. 24 indicates amino acids within the WRKY domain that differentiate the G1274 clade from the out-group. Most notable in FIG. 24 are the conserved K at position 264, the N at position 275, the S at position 280, the D at 293 and the F/Y at position 299 (indicated by asterisks). These residues are potentially responsible for the conserved structure/function of this clade with regard to drought tolerance. Based on full-length protein sequence, G1758 appears firmly in the G1274 clade. FIG. 24 shows that, within the WRKY domain, G1758 is intermediate between the out-group and the claimed sequences. These amino acid differences may represent specific changes that retain drought tolerance function, or possibly more finely delineate the key residues required for function.

The NAC family, including the G2053 clade. The NAC family is a group of transcription factors that share a highly conserved N-terminal domain of about 150 amino acids, designated the NAC domain (NAC stands for Petunia, NAM, and Arabidopsis, ATAF1, ATAF2 and CUC2). This is believed to be a novel domain that is present in both monocot and dicot plants but is absent from yeast and animal proteins. One hundred and twelve members of the NAC family have been identified in the Arabidopsis genome. The NAC class of proteins can be divided into at least two sub-families on the basis of amino acid sequence similarities within the NAC domain. One sub-family is built around the NAM and CUC2 (cup-shaped cotyledon) proteins whilst the other sub-family contains factors with a NAC domain similar to those of ATAF1 and ATAF2.

Thus far, little is known about the function of different NAC family members. This is surprising given that there are 113 members in Arabidopsis. However, NAM, CUC1 and CUC2 are thought to have vital roles in the regulation of embryo and flower development. In Petunia, nam mutant embryos fail to develop a shoot apical meristem (SAM) and have fused cotyledons. These mutants sometimes generate escape shoots that produce defective flowers with extra petals and fused organs. In Arabidopsis, the cuc1 and cuc2 mutations have somewhat similar effects, causing defects in SAM formation and the separation of cotyledons, sepals and stamens.

Although nam and cuc mutants exhibit comparable defects during embryogenesis, the penetrance of these phenotypes is much lower in cuc mutants. Functional redundancy of the CUC genes in Arabidopsis may explain this observation. In terms of the flower phenotype there are notable differences between nam and cuc mutants. Flowers of cuc mutants do not contain additional organs and the formation of sepals and stamens is most strongly affected. In nam mutants, by contrast, the flowers do carry additional organs and petal formation is more markedly affected than that of other floral organs. These apparent differences might be explained in two ways: the NAM and CUC proteins have been recruited into different roles in development of Arabidopsis and Petunia flowers. Alternatively, the proteins could share a common function between the two species, with the different mutant floral phenotypes arising from variations in the way other genes (that participate in the same developmental processes) are affected by defects in NAM or CUC.

A further gene from this family, NAP (NAC-like activated by AP3/PI) is also involved in flower development and is thought to influence the transition between cell division and cell expansion in stamens and petals. Overall, then, the NAC proteins mainly appear to regulate developmental processes.

The ZF-HD family, including the G2999 clade. Since their discovery in 1983, the homeobox genes (the name of which derives from the homeotic mutations that affect Drosophila development) have been found in all eukaryotes examined, including yeast, plants, and animals (McGinnis et al. (1984) Nature 308: 428-433; McGinnis et al. (1984) Cell 37: 403-408; Scott et al. (1984) Proc. Natl. Acad. Sci. U.S.A. 81: 4115-4119; Scott et al. (1989) Biochim. Biophys. Acta. 989, 25-48; Shepherd et al. (1984) Nature 310: 70-71; Gehring et al. (1987) Science 236: 1245-1252; Vollbrecht et al. (1991) Nature 350: 241-243; Ruberti et al. (1991) EMBO J. 10: 1787-1791; and Schena and Davis (1992) Genes. Dev. 7, 367-379. The homeobox (HB) is a conserved DNA stretch that encodes an approximate 61 amino acid region termed the homeodomain (HD). It is well demonstrated that homeodomain proteins are transcription factors, and that the homeodomain is responsible for sequence specific recognition and binding of DNA (Affolter et al. (1990) Curr Opin Cell Biol. 2: 485-495; Hayashi and Scott (1990) Cell 63: 883-894, and references therein). Genetic and structural analysis indicate that the homeodomain operates by fitting the most conserved of three alpha helices, helix 3, directly into the major groove of the DNA (Hanes and Brent (1989) Cell 57: 1275-1283; Hanes and Brent (1991) Science 251: 426-430; Kissinger et al. (1990) Cell 63: 579-590; and Wolberger et al. (1991) Cell 67: 517-528). For a general review on the homeobox genes, see Duboule, D. (1994). Guidebook to the Homeobox Genes. Oxford, Oxford University Press.

Homeobox genes play many important roles in the developmental processes of multicellular animals. In Drosophila, for example, a variety of these genes have functions in embryo development. Initially, they act maternally to establish anterior-posterior polarity. Later, homeobox genes are known to regulate the segmentation process, dorso-ventral differentiation, and control cell fate determination in the eye and nervous system (Scott et al. (1989) supra).

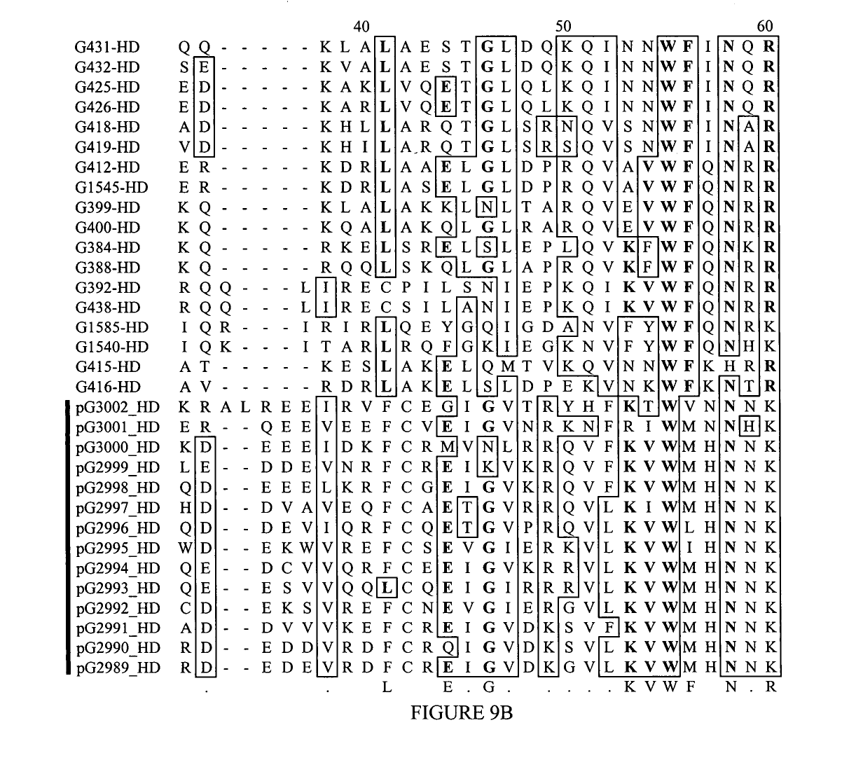

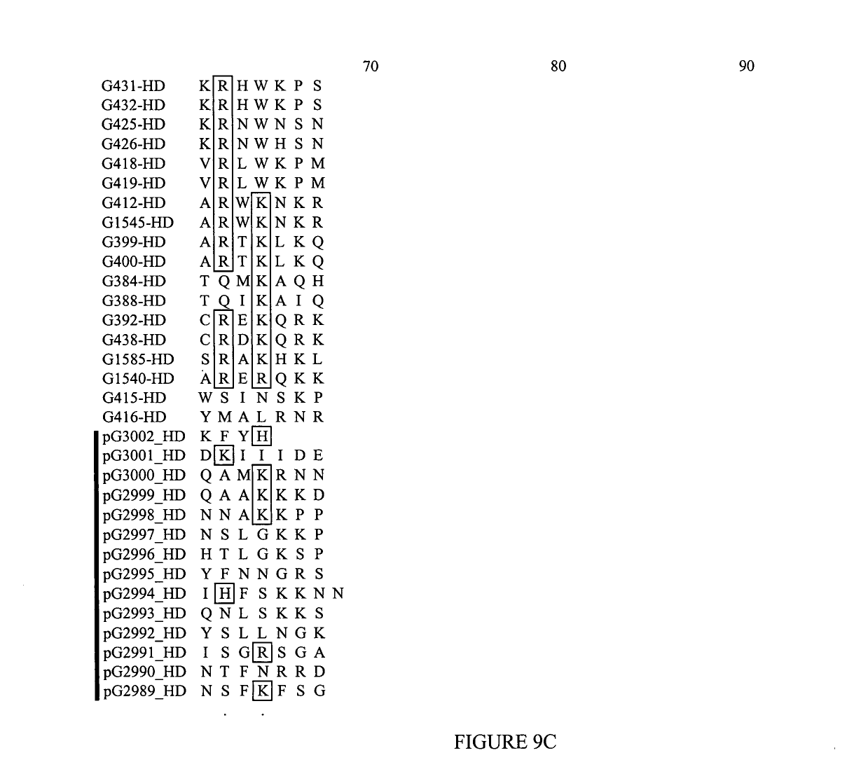

A large number of homeodomain proteins have now been identified in a range of higher plants (Burglin (1997) Nucleic Acids Res. 25: 4173-4180; Burglin (1998) Dev. Genes Evol. 208: 113-116), which are herein defined as the containing the ‘classical’ type of homeodomain (FIG. 9). These exhibit many differences to animal homeodomain proteins outside the conserved domain, but all contain the signature WFXNX[RK] (X=any amino acid, [RK] indicates either an R or K residue at this position) within the third helix. Data from the Genome Initiative indicate that there are around 90 Arabidopsis classical homeobox genes. These are now being implicated in the control of a wide range of different processes. In many cases, plant homeodomains are found in proteins in combination with additional regulatory motifs such as leucine zippers. Classical plant homeodomain proteins can be broadly categorized into the following different classes based on homologies within the family, and the presence of other types of domain: KNOX class I, KNOX class II, HD-BEL1, HD-ZIP class I, HD-ZIP class II, HD-ZIP class III, HD-ZIP class IV (GL2 like), PHD finger type, and WUSCHEL-like (Freeling and Hake (1985); Genetics 111: 617-634 Vollbrecht et al. (1991) supra; Schindler et al. (1993) Plant J. 4:137-150; Sessa et al. (1994)). In: Puigdomenech P, Coruzzi G, (eds) Molecular genetic analysis of plant development and metabolism, pp. 411-426. Springer Verlag, Berlin; Kerstetter et al. (1994) Plant Cell 6: 1877-1887; Kerstetter et al. (1997) Development 124: 3045-3054; Burglin (1997) supra; Burglin (1998) supra; Schoof et al. (2000) Cell 100: 635-644).

Recently a novel class of proteins was discovered that contain a domain similar to the classical homeodomain, in combination with N-terminal zinc finger motifs, by Windhovel (Windhovel et al. (2001) Plant Mol. Biol. 45: 201-214), while studying the regulatory mechanisms responsible for the mesophyll specific expression of the C4 phosphoenolpyruvate gene of Flavaria trinervia. Using a yeast one-hybrid screen, these workers recovered five cDNA clones, which encoded proteins that were capable of specifically binding the promoter of the Flavaria C4 phosphoenolpyruvate gene, but not the promoter of a Flavaria C3 phosphoenolpyruvate gene. One-hybrid experiments and in vitro DNA binding studies were then used to confirm that these proteins specifically interact with the proximal region of the C4 phosphoenolpyruvate gene. Four of five clones [FtHB1 (GenBank accession Y18577), FbHB2 (GenBank accession Y18579), FbHB3 (GenBank accession Y18580), and FbHB4 (GenBank accession Y18581), (the fifth clone encoded a histone)] all encoded a novel type of protein that contained two types of highly conserved domains. At the C-termini, a region was apparent that had many of the features of a homeodomain, whereas at the N-termini, two putative zinc finger motifs were present. Yeast two-hybrid experiments were used to show that the zinc finger motifs are sufficient to confer homo and hetero-dimerization between the proteins, and mutagenesis experiments demonstrated that conserved cysteine residues within the motifs are essential for such dimerization. Given the presence of the potential homeodomain and zinc fingers, Windhovel (Windhovel et al. (2001) supra) named this new class of proteins as the ZF-HD group.

That four proteins of this type were identified in the above studies suggested that the family might have a specific role in establishing expression of the C4 phosphoenolpyruvate gene within mesophyll cells. However, database searches revealed that proteins of this class are also present in C3 species, indicating that they likely have additional roles outside of C4 photosynthesis (Windhovel et al. (2001) supra). In particular, the Arabidopsis genome encodes fourteen proteins of this type, but the functional analysis of these proteins has yet to be publicly reported.

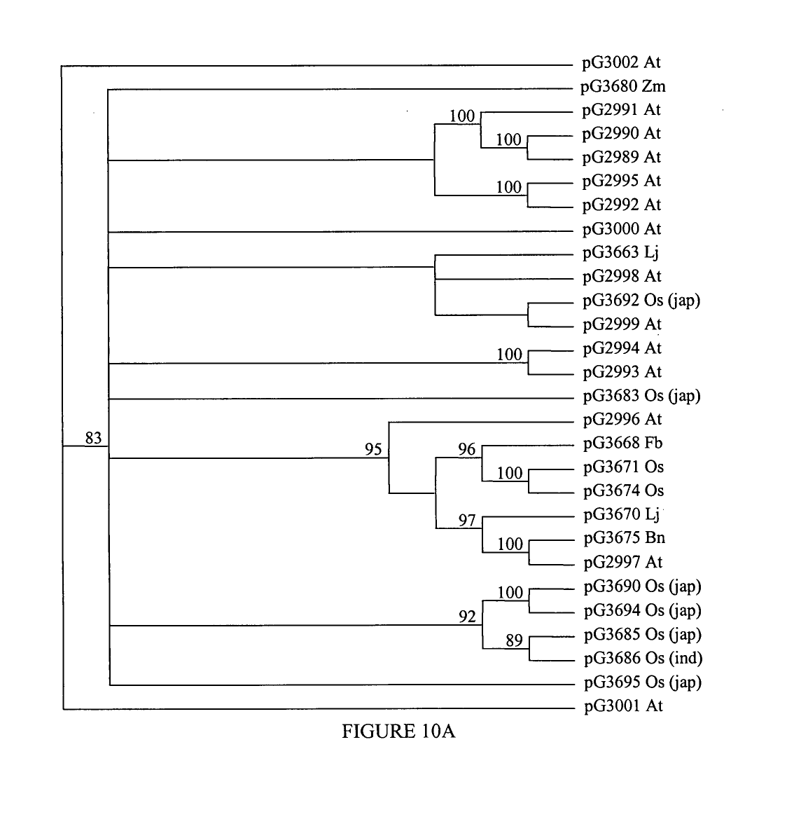

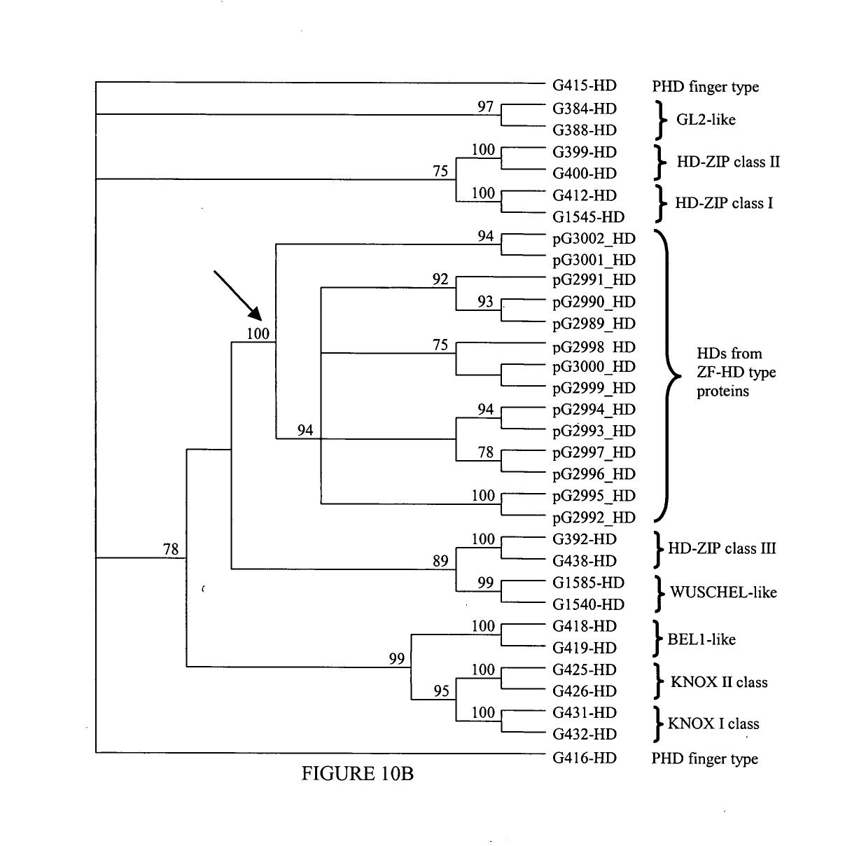

Secondary structure analyses performed by Windhovel (Windhovel et al. (2001) supra) indicated that the putative homeodomains of the ZF-HD proteins contain three alpha helices similar to those recognized in the classes of homeodomain already found in plants (Duboule (1994) supra). Interestingly, though, if full-length proteins of the ZF-HD group are blasted against databases, they do not preferentially align with the known classes of plant homeodomain proteins. Furthermore, a phylogenetic tree based on comparing the classical versus ZF-HD type homeodomains reveal that the latter occupy a distinct node of the tree (FIG. 10).

A careful examination of the ZF-HD proteins reveals a particular striking difference to the classical plant homeodomain. All of the 90 or so previously recognized plant homeodomain proteins contain the signature WFXNX[RK] (X=any amino acid) within the third helix. However, the ZF-HD proteins all lack the invariant F residue in this motif and generally contain an M in its place. This structural distinction, combined with the presence of ZF motifs in other regions of the protein, could confer functional properties on ZF-HD proteins that are different to those found in other HD containing proteins.

The HLH/MYC family, including the G3086 clade. The bHLH protein family is a group of transcription factors found in mammals and plants. The typical feature of this family of transcription factors is that they share a highly conserved approximately 50 amino acid DNA-binding domain. This domain consists of a basic region of 14 amino acids followed by a first helix, a loop region of seven amino acids and a second helix (Littlewood et al. (1994) Prot. Profile 1: 639-709). In plants, members of this family also share, besides the bHLH domain, a highly conserved 200 amino acid N-terminal domain. Functional analysis revealed that small deletions in the N-terminal domain inactivate the B protein, a member of bHLH protein family, in Z. mays (Goff et al. (1992) Genes Dev. 6: 864-875). It has also been shown that the N-terminal domain can interact with one of other transcription factors (Myb proteins) to regulate anthocyanin biosynthesis in Z. mays (Goff et al. (1992) supra).

In mammalian systems, members of this family have been shown to control development and differentiation of a variety of cell types. The bHLH proteins play essential roles in neurogenesis or neural development, and myogenesis (Littlewood et al. (1994) supra).

Plant bHLH proteins have been shown to play an important role in the regulation of anthocyanin biosynthesis, in the control of trichome development, in phytochrome signaling transduction pathway, and in the regulation of dehydration- and ABA-inducible gene expression. It has suggested that the R locus of maize is responsible for determining the temporal and spatial pattern of anthocyanin pigmentation in the plant. The R gene family consists of B, S, and Lc genes, which encode a transcription factor of the basic helix-loop-helix class (Goff et al. (1992) supra, Ludwig (1990) Cell 62: 849-851). A gene encoding a basic helix-loop-helix protein has been cloned as a phytochrome-interacting factor in a genetic screen for T-DNA-tagged Arabidopsis mutants as well as in a yeast two-hybrid screen. The protein functions as a positively-acting signaling intermediate (Halliday et al. (1999) Proc. Natl. Acad. Sci. USA. 96:5832-5837, Ni et al. (1998) Cell 95: 657-667). A new mutant, hfrl (long hypocotyl in far-red) has been isolated from Quail's lab. The hfrl mutant exhibits a reduction in seedling responsiveness specifically to continuous far-red light (FRc), thereby suggesting a locus likely to be involved in phytochrome A (phyA) signal transduction. HFR1 encodes a nuclear protein with strong similarity to the bHLH family of DNA-binding proteins but with an atypical basic region. In contrast to PIF3, a related bHLH protein previously shown to bind phyB, HFR1 did not bind either phyA or B. However, HFR1 did bind PIF3, suggesting heterodimerization, and both the HFR1/PIF3 complex and PIF3 homodimer bound preferentially to the Pfr form of both phytochromes. Thus, HFR1 may function to modulate phyA signaling via heterodimerization with PIF3. HFR1 mRNA is 30-fold more abundant in FRc than in continuous red light, suggesting a potential mechanistic basis for the specificity of HFR1 to phyA signaling.

The rd22BP1 protein of Arabidopsis has a typical DNA-binding domain of a basic region helix-loop-helix motif. It has been shown that transcription of the rd22BP1 gene is induced by dehydration stress and phytohormone ABA treatment, and its induction precedes that of rd22, a dehydration-responsive gene (Abe et al. (1997) Plant Cell 9: 1859-1868).

Plant bHLH proteins may also play a crucial role in the process of nitrogen fixation, probably not acting as a transcription factor. A protein with a helix-loop-helix motif was identified as a symbiotic ammonium transport protein by functional complementation of the yeast NH4+ transport mutant with a soybean nodule cDNA (Kaiser et al. (1998) Science 1998 281: 1202-1206). Using similar complementation approach of the yeast fet3fet4 mutant strain, an iron transport protein was isolated from an iron-deficient maize root cDNA expression library. The protein had 44% identity with an Arabidopsis bHLH-like protein RAP1 that binds the G-box sequence via a basic region helix-loop-helix (Loulergue (1998) Gene 225:47-57).

Another bHLH gene has been recently identified as indl by M. Yanofsky's group in UC San Diego. They found that fruit from a knockout mutant do not show dehiscence zone differentiation. In addition, their results suggest that ind1 may mediate cell differentiation during Arabidopsis fruit development. A cytokinin-repressed gene CRR12 with a basic region/helix-loop-helix motif was identified from a cucumber cotyledon cDNA library. It was found that the level of CRR12 transcripts decreased in response to either cytokinins or light in etiolated cotyledons. The mRNA was low in cotyledons and leaves of light-grown plants, but it increased during dark incubation.

Table 1 shows the polypeptides identified by polypeptide SEQ ID NO and Identifier (e.g., Mendel Gene ID (GID) No., accession number or other name), presented in order of similarity to the first Arabidopsis sequence listed for each set, and includes the conserved domains of the polypeptide in amino acid coordinates, the respective domain sequences, and the extent of identity in percentage terms to the first Arabidopsis sequence listed for each set.

TABLE 1

Gene families and binding domains for exemplary sequences
of the invention, including paralogs and orthologs

| | | | | | | |
|---|---|---|---|---|---|---|
| | | | Conserved | | | |
| | | | Domains in | Conserved | | |
| | | | Polypeptide | Domains in | Conserved | % ID in |
| SEQ ID | | | Amino Acid | Polynucleotide | Domain | conserved |
| NO: | GID | Species | Coordinates | Base Coordinates | Sequence | domain |

| | | | | | | |
|---|---|---|---|---|---|---|
| | | | | | | % ID to |
| | | | | | | G2133 |
| 12 | G2133 | Arabidopsis | AP2: 10-77 | AP2: 53-256 | DQSKYKGIRRRKWGK | 100% |
| | | thaliana | | | WVSEIRVPGTRQRLWL | |
| | | | | | GSFSTAEGAAVAHDVA | |
| | | | | | FYCLHRPSSLDDESFNF | |
| | | | | | PHLL | |
| 94 | G3646 | Brassica | AP2: 10-77 | AP2: 203-406 | HQAKYKGIRRRKWGK | 91% |
| | | oleracea | | | WVSEIRVPATRERLWL | |
| | | | | | GSFSTAEGAAVAHDVA | |
| | | | | | FYCLHRPSSLDNEAFNF | |
| | | | | | PHLL | |
| 92 | G3645 | Brassica rapa | AP2: 10-75 | AP2: 40-237 | TQSKYKGIRRRKWGK | 89% |
| | | subsp. | | | WVSEIRVPGTRDRLWL | |
| | | Pekinensis | | | GSFSTAEGAAVAHDVA | |
| | | | | | FYCLHQPNSLESLNFPH | |
| | | | | | LL | |
| 2 | G47 | Arabidopsis | AP2: 10-75 | AP2: 65-262 | SQSKYKGIRRRKWGK | 88% |
| | | thaliana | | | WVSEIRVPGTRDRLWL | |
| | | | | | GSFSTAEGAAVAHDVA | |
| | | | | | FFCLHQPDSLESLNFPH | |
| | | | | | LL | |
| 88 | G3643 | Glycine max | AP2: 13-78 | AP2: 101-298 | TNNKLKGVRRRKWGK | 69% |
| | | | | | WVSEIRVPGTQERLWL | |
| | | | | | GTYATPEAAAVAHDV | |
| | | | | | AVYCLSRPSSLDKLNFP | |
| | | | | | ETL | |
| 96 | G3647 | Zinnia elegans | AP2: 13-78 | AP2: 53-250 | SQKTYKGVRCRRWGK | 63% |
| | | | | | WVSEIRVPGSRERLWL | |
| | | | | | GTYSTPEGAAVAHDVA | |
| | | | | | SYCLKGNTSFHKLNIPS | |
| | | | | | ML | |
| 90 | G3644 | Oryza sativa | AP2: 52-122 | AP2: 154-366 | ERCRYRGVRRRRWGK | 54% |
| | | (japonica | | | WVSEIRVPGTRERLWL | |
| | | cultivar- | | | GSYATPEAAAVAHDTA | |
| | | group) | | | VYFLRGGAGDGGGGG | |
| | | | | | ATLNFPERA | |
| 98 | G3649 | Oryza sativa | AP2: 15-87 | AP2: 43-26 | EMMRYRGVRRRRRWGK | 53% |
| | | (japonica | | | WVSEIRVPGTRERLWL | |
| | | cultivar- | | | GSYATAEAAAVAHDA | |
| | | group) | | | AVCLLRLGGGRRAAA | |
| | | | | | GGGGGLNFPARA | |
| 100 | G3651 | Oryza sativa | AP2: 60-130 | AP2: 178-390 | ERCRYRGVRRRRW0K | 52% |
| | | (japonica | | | WVSEIRVPGTRERLWL | |
| | | cultivar- | | | GSYATPEAAAVAHDTA | |
| | | group) | | | VYFLRGGAGDGGGGG | |
| | | | | | ATAQLPGAR | |
| | | | | | | % ID to |
| | | | | | | G922 |
| 4 | G922 | Arabidopsis | 1st SCR: 134-199 | 1st SCR: 400-597 | RRLFFEMFPILKVSYLL | 100% |
| | | thaliana | | | TNRAILEAMEGEKMVH | |
| | | | | | VIDLDASEPAQWLALL | |
| | | | | | QAFNSRPEGPPHLRITG | |
| 4 | G922 | Arabidopsis | 2nd SCR: 332- | 2nd SCR: 994- | FLNAIWGLSPKVMVVT | 100% |
| | | thaliana | 401 | 1203 | EQDSDHNGSTLMERLL | |
| | | | | | ESLYTYAALFDCLETK | |
| | | | | | VPRTSQDRIKVEKMLF | |
| | | | | | GEEIKN | |
| 4 | G922 | Arabidopsis | 3rd SCR: 405-478 | 3rd SCR: 1213- | CEGFERRERHEKLEKW | 100% |
| | | thaliana | | 1434 | SQRIDLAGFGNVPLSYY | |
| | | | | | AMLQARRLLQGCGFD | |
| | | | | | GYRIKEESGCAVICWQ | |
| | | | | | DRPLYSVSAW | |
| 220 | G3824 | Lycopersicon | 1st SCR: 42-107 | 1st SCR: 134-331 | RKMFFEIFPFLKVAFVV | 69% |
| | | esculentum | | | TNQAIIEAMEGEKMVH | |
| | | | | | IVDLNAAEPLQWRALL | |
| | | | | | QDLSARPEGPPHLRITG | |
| 220 | G3824 | Lycopersicon | 2nd SCR: 235- | 2nd SCR: 713- | FLNALWGLSPKVMVV | 78% |
| | | esculentum | 304 | 922 | TEQDANHNGTTLMERL | |
| | | | | | SESLHFYAALFDCLEST | |
| | | | | | LPRTSLERLKVEKMLL | |
| | | | | | GEEIRN | |
| 220 | G3824 | Lycopersicon | 3rd SCR: 308-381 | 3rd SCR: 932- | CEGIERKERHEKLEKW | 77% |
| | | esculentum | | 1153 | FQRFDTSGFGNVIPLSY | |
| | | | | | YAMLQARRLLQSYSCE | |
| | | | | | GYKIKEDNGCVVICWQ | |
| | | | | | DRPLFSVSSW | |
| 212 | G3810 | Glycine max | 1st SCR: 106-171 | 1st SCR: 316-513 | QKLFFELFPFLKVAFVL | 68% |
| | | | | | TNQAIIEAMEGEKVIHII | |
| | | | | | DLNAAEAAQWIALLRV | |
| | | | | | LSAHPEGPPHLRITG | |
| 212 | G3810 | Glycine max | 2nd SCR: 305- | 2nd SCR: 913- | FLNALWGLSPKVMVV | 80% |
| | | | 374 | 1122 | TEQDCNHNGPTLMDRL | |
| | | | | | LEALYSYAALFDCLEST | |
| | | | | | VSRTSLERLRVEKMLF | |
| | | | | | GEEIKN | |
| 212 | G3810 | Glycine max | 3rd SCR: 378-451 | 3rd SCR: 1132- | CEGSERKERHEKLEKW | 71% |
| | | | | 1353 | FQRFDLAGFGNVPLSY | |
| | | | | | FGMVQARRFLQSYGCE | |
| | | | | | GYRMRDENGCVLICW | |
| | | | | | EDRPMYSISAW | |
| 214 | G3811 | Glycine max | 1st SCR: 103-168 | 1st SCR: 361-558 | QKLFFELLPFLKFSYILT | 68% |
| | | | | | NQAIVEAMEGEKMVHI | |
| | | | | | VDLYGAGPAQWISLLQ | |
| | | | | | VLSARPEGPPHLRITG | |
| 214 | G3811 | Glycine max | 2nd SCR: 296- | 2nd SCR: 940- | FLNALWGLSPKVMVV | 74% |
| | | | 365 | 1149 | TEQDFNHNCLTMMERL | |
| | | | | | AEALFSYAAYFDCLES | |
| | | | | | TVSRASMDRLKLEKML | |
| | | | | | FGEEIKN | |
| 214 | G3811 | Glycine max | 3rd SCR: 369-442 | 3rd SCR: 1159- | CEGCERKERHEKMDR | 60% |
| | | | | 1380 | WIQRLDLSGFANVPISY | |
| | | | | | YGMLQGRRFLQTYGCE | |
| | | | | | GYKMREECGRVMICW | |
| | | | | | QERSLFSITAW | |
| 218 | G3814 | Oryza sativa | 1st SCR: 123-190 | 1st SCR: 367-570 | RRHMFDVLPFLKLAYL | 60% |
| | | (japonica | | | TTNHAILEAMEGERFV | |
| | | cultivar- | | | HVVDFSGPAANPVQWI | |
| | | group) | | | ALFHAFRGRREGPPHL | |
| | | | | | RITA | |
| 218 | G3814 | Oryza sativa | 2nd SCR: 332- | 2nd SCR: 994- | FLSAVRSLSPKIMVMTE | 48% |
| | | (japonica | 400 | 1200 | QEANHNGGAFQERFDE | |
| | | cultivar- | | | ALNYYASLFDCLQRSA | |
| | | group) | | | AAAAERARVERVLLGE | |
| | | | | | EIRG | |
| 218 | G3814 | Oryza sativa | 3rd SCR: 404-480 | 3rd SCR: 1210- | CEGAERVERHERARQ | 46% |
| | | (japonica | | 1440 | WAARMEAAGMERVGL | |
| | | cultivar- | | | SYSGAMEARKLLQSCG | |
| | | group) | | | WAGPYEVRIHDAGGHG | |
| | | | | | FFFCWHKRPLYAVTAW | |
| 216 | G3813 | Oryza sativa | 1st SCR: 129-194 | 1st SCR: 385-582 | RRHFLDLCPFLRLAGA | 53% |
| | | (japonica | | | AANQSILEAMESEKIVH | |
| | | cultivar- | | | VIDLGGADATQWLELL | |
| | | group) | | | HLLAARPEGPPHLRLTS | |
| 216 | G3813 | Oryza sativa | 2nd SCR: 290- | 2nd SCR: 868- | FLGALWGLSPKVMVV | 61% |
| | | (japonica | 359 | 1077 | AEQEASHNAAGLTERF | |
| | | cultivar- | | | VEALNYYAALFDCLEV | |
| | | group) | | | GAARGSVERARVERW | |
| | | | | | LLGEEIKN | |
| 216 | G3813 | Oryza sativa | 3rd SCR: 363-436 | 3rd SCR: 1087- | CDGGERRERHERLERW | 64% |
| | | (japonica | | 1308 | ARRLEGAGFGRVPLSY | |
| | | cultivar- | | | YALLQARRVAQGLGC | |
| | | group) | | | DGFKVREEKGNFFLCW | |
| | | | | | QDRALFSVSAW | |
| 222 | G3827 | Oryza sativa | 2nd SCR: 226- | 2nd SCR: 676- | DVESLRGLSLKVMVVT | 55% |
| | | (japonica | 295 | 885 | EQEVSHNAAGLTERFV | |
| | | cultivar- | | | EALNYYAALFDCLEVG | |
| | | group) | | | GARGSVERTRVERWLL | |
| | | | | | GEEIKN | |
| 222 | G3827 | Oryza sativa | 3rd SCR: 299-365 | 3rd SCR: 895- | CDGGERRIERHERLEGA | 60% |
| | | (japonica | | 1095 | GFGRVPLSYYALLQAR | |
| | | cultivar- | | | RVAQGLGCDGFKVREE | |
| | | group) | | | KGNFFLCWQDRALFSV | |
| | | | | | SAW | |
| | | | | | | % ID to |
| | | | | | | G1274 |
| 6 | G1274 | Arabidopsis | WRKY: 110-166 | WRKY: 328-498 | DDGFKWRKYGKKSVK | 100% |
| | | thaliana | | | NNINKRNYYKCSSEGC | |
| | | | | | SVKIKRVERDGDDAAY | |
| | | | | | VITTYEGVHNH | |
| 140 | G3724 | Glycine max | WRKY: 107-163 | WRKY: 390-560 | DDGYKWRKYGKKSVK | 84% |
| | | | | | SSPNLRNYYKCSSGGC | |
| | | | | | SVKKRVERDRDDYSYV | |
| | | | | | ITTYEGVHNH | |
| 148 | G3728 | Zea mays | WRKY: 108-164 | WRKY: 1075- | DDGFKWRKYGKKAVK | 82% |
| | | | | 1245 | NSPNPRNYYRCSSEGC | |
| | | | | | GVKKRVERDRDDPRY | |
| | | | | | VITTYDGVHNH | |
| 206 | G3802 | Sorghum | WRKY: 110-166 | WRKY: 386-556 | DDGFKWRKYGKKAVK | 82% |
| | | bicolor | | | NSPNPRNYYRCSSEGC | |
| | | | | | GVKKRVERDRDDPRY | |
| | | | | | VITTYDGVHNH | |
| 210 | G3804 | Zea mays | WRKY: 108-164 | WRKY: 438-608 | DDGFKWRXYGKKAVK | 82% |
| | | | | | NSPNPRNYYRCSSEGC | |
| | | | | | GVKKRVERDRDDPRY | |
| | | | | | VITTYDGVHNH | |
| 146 | G3727 | Zea mays | WRKY: 102-158 | WRKY: 391-561 | DDGFKWRKYGKKAVK | 80% |
| | | | | | SSPNPRNYYRCSSEGCG | |
| | | | | | VKKRVERDRDDPRYVI | |
| | | | | | TTYDGVHNH | |
| 154 | G3731 | Lycopersicon | WRKY: 95-151 | WRKY: 297-467 | DDGFKCRKYGKKMVK | 80% |
| | | esculentum | | | NNPNPRNYYKCSSGGC | |
| | | | | | NVKKRVERDNKDSSY | |
| | | | | | VITTYEGIHNH | |
| 156 | G3732 | Solanum | WRKY: 95-151 | WRKY: 309-479 | DDGFKWRKYGKKMV | 80% |
| | | tuberosum | | | KNSSNPRNYYKCSSGG | |
| | | | | | CNVKKRVERDNEDSSY | |
| | | | | | VITTYEGIHNH | |
| 158 | G3733 | Hordeum | WRKY: 131-187 | WRKY: 641-811 | DDGYKWRKYGKKSVK | 80% |
| | | vulgare | | | NSPNPRNYYRCSTEGC | |
| | | | | | SVKKRVERDRDDPAYV | |
| | | | | | VTTYEGTHSH | |
| 204 | G3797 | Lactuca sativa | WRKY: 118-174 | WRKY: 363-533 | DDGFKWRKYGKKMV | 80% |
| | | | | | KNSPNPRNYYRCSAAG | |
| | | | | | CSVKKRVERDVEDARY | |
| | | | | | VITTYEGIHNH | |
| 208 | G3803 | Glycine max | WRKY: 111-167 | WRKY: 367-537 | DDGYKWRKYGKKTVK | 80% |
| | | | | | NNPNPRNYYKCSGEGC | |
| | | | | | NVKKRVERDRDDSNY | |
| | | | | | VLTTYDGVHNH | |
| 132 | G3720 | Zea mays | WRKY: 135-191 | WRKY: 403-573 | DDGYKWRKYGKKSVK | 78% |
| | | | | | NSPNPRNYYRCSTEGC | |
| | | | | | NVKKRVERDKDDPSY | |
| | | | | | VVTTYEGMHNH | |
| 134 | G3721 | Oryza sativa | WRKY: 96-152 | WRKY: 342-512 | DDGFKWRKYGKKAVK | 78% |
| | | (japonica | | | NSPNPRNYYRCSTEGC | |
| | | cultivar- | | | NVKKRVERDREDHRY | |
| | | group) | | | VITTYDGVHNH | |
| 136 | G3722 | Zea mays | WRKY: 129-185 | WRKY: 430-600 | DDGYKWRKYGKKSVK | 78% |
| | | | | | NSPNPRNYYRCSTEGC | |
| | | | | | NVKKRVERDRDDPRY | |
| | | | | | VVTMYEGVHNH | |
| 144 | G3726 | Oryza sativa | WRKY: 135-191 | WRKY: 459-629 | DDGYKWRKYGKKSVK | 78% |
| | | (japonica | | | NSPNPRNYYRCSTEGC | |
| | | cultivar- | | | NVKKRVERDKDDPSY | |
| | | group) | | | VVTTYEGTHNH | |
| 202 | G3795 | Capsicum | WRKY: 95-151 | WRKY: 302-472 | DDGYKWRKYGKKMV | 78% |
| | | annuum | | | KNSPNPRNYYRCSVEG | |
| | | | | | CPVKKRVERDKEDSRY | |
| | | | | | VITTYEGVHNH | |
| 30 | G1275 | Arabidopsis | WRKY: 113-169 | WRKY: 394-564 | DDGFKWRKYGKKMV | 77% |
| | | thaliana | | | KNSPHPRNYYKCSVDG | |
| | | | | | CPVKKRVERDRDDPSF | |
| | | | | | VITTYEGSHNH | |
| 138 | G3723 | Glycine max | WRKY: 113-169 | WRKY: 715-885 | DDGYKWRKYGKKTVK | 77% |
| | | | | | SSPNPRNYYKCSGEGC | |
| | | | | | DVKKRVERDRDDSNY | |
| | | | | | VLTTYDGVHNH | |
| 152 | G3730 | Oryza sativa | WRKY: 107-163 | WRKY: 385-555 | DDGFKWRKYGKKAVK | 77% |
| | | (japonica | | | SSPNPRNYYRCSAAGC | |
| | | cultivar- | | | GVKIKRVERDGDDPRY | |
| | | group) | | | VVTTYDGVHNH | |
| 130 | G3719 | Zea mays | WRKY: 91-147 | WRKY: 428-598 | DDGFKWRKYGKKAVK | 75% |
| | | | | | SSPNPRNYYRCSTEGSG | |
| | | | | | VKKRVERDSDDPRYVV | |
| | | | | | TTYDGVHNH | |
| 142 | G3725 | Oryza sativa | WRKY: 158-214 | WRKY: 688-858 | DDGYKWRKYGKKSVK | 75% |
| | | (japonica | | | NSPNPRNYYRCSTEGC | |
| | | cultivar- | | | NVKKRVERDKNDPRY | |
| | | group) | | | VVTMYEGIHNH | |
| 150 | G3729 | Oryza sativa | WRKY: 137-193 | WRKY: 452-622 | DDGYRWRKYGKKMV | 75% |
| | | (japonica | | | KNSPNPRNYYRCSSEG | |
| | | cultivar- | | | CRVKKRVERARDDARF | |
| | | group) | | | VVTTYDGVHNH | |
| 32 | G1758 | Arabidopsis | WRKY: 109-165 | WRKY: 393-563 | DDGYKWRKYGKKIPIT | 57% |
| | | thaliana | | | GSPFPRHYHKCSSPDCN | |
| | | | | | VKKKIERDTNNPDYILT | |
| | | | | | TYEGRHNH | |
| | | | | | | % ID to |
| | | | | | | G1792 |
| 8 | G1792 | Arabidopsis | AP2: 16-80 | AP2: 122-316 | KQARIFRGVRiRRPWGK | 100% |
| | | thaliana | | | FAAEIRDPSRNGARLW | |
| | | | | | LGTFETAEEAARAYDR | |
| | | | | | AAFNLRGHLAILNFPNEY | |
| 86 | G3520 | Glycine max | AP2: 14-78 | AP2: 50-244 | EEPRYRGVRRRPWGKF | 80% |
| | | | | | AAEIRDPARHGARVWL | |
| | | | | | GTFLTAEEAARAYDRA | |
| | | | | | AYEMRGALAVLNFPNEY | |
| 82 | G3518 | Glycine max | AP2: 13-77 | AP2: 134-328 | VEVRYRGIRRRPWGKF | 76% |
| | | | | | AAEIRDPTRKGTRIWLG | |
| | | | | | TFDTAEQAARAYDAA | |
| | | | | | AFHFRGHRAILNFPNEY | |
| 84 | G3519 | Glycine max | AP2: 13-77 | AP2: 93-287 | CEVRYRGIRRRPWGKF | 76% |
| | | | | | AAEIRDPTRKGTRIWLG | |
| | | | | | TFDTAEQAARAYDAA | |
| | | | | | AFHFRGHRAILNFPNEY | |
| 160 | G3735 | Medicago | AP2: 23-87 | AP2: 148-342 | DQIKYRGIRRRPWGKF | 76% |
| | | truncatula | | | AAEIRDPTRKGTRIWLG | |
| | | | | | TFDTAEQAARAYDAA | |
| | | | | | AFHFRGHRAILNFPNEY | |
| 34 | G1791 | Arabidopsis | AP2: 10-74 | AP2: 63-257 | NEMKYRGVRKRPWGK | 72% |
| | | thaliana | | | YAAEIRDSARHGARVW | |
| | | | | | LGTFNTAEDAARAYDR | |
| | | | | | AAFGMRGQRAILNFPHEY | |
| 70 | G3380 | Oryza sativa | AP2: 18-82 | AP2: 138-332 | ETTKYRGVRRRPSGKF | 72% |
| | | (japonica | | | AAEIRDSSRQSVRVWL | |
| | | cultivar- | | | GTFDTAEEAARAYDRA | |
| | | group) | | | AYAMRGHLAVLNFPAEA | |
| 74 | G3383 | Oryza sativa | AP2: 9-73 | AP2: 25-219 | TATKYRGVRRRPWGK | 72% |
| | | (japonica | | | FAAEIRDPERGGARVW | |
| | | cultivar- | | | LGTFDTAEEAARAYDR | |
| | | group) | | | AAYAQRGAAAVLNFPAAA | |
| 18 | G30 | Arabidopsis | AP2: 16-80 | AP2: 86-280 | EQGKYRGVRRRPWGK | 70% |
| | | thaliana | | | YAAEIRDSRKHGERVW | |
| | | | | | LGTFDTAEDAARAYDR | |
| | | | | | AAYSMRGKAAILNFPHEY | |
| 72 | G3381 | Oryza sativa | AP2: 14-78 | AP2: 122-316 | LVAKYRGVRRIRPWGK | 70% |
| | | (japonica | | | FAAEIRDSSRHGVRVW | |
| | | cultivar- | | | LGTFDTAEEAARAYDR | |
| | | group) | | | SAYSMRGANAVLNFPADA | |
| 76 | G3515 | Oryza sativa | AP2: 11-75 | AP2: 53-247 | SSSSYRGVRKRPWGKF | 70% |
| | | (japonica | | | AAEIRDPERGGARVWL | |
| | | cultivar- | | | GTFDTAEEAARAYDRA | |
| | | group) | | | AFAMKGATAMLNFPGDH | |
| 78 | G3516 | Zea mays | AP2: 6-70 | AP2: 16-210 | KEGKYRGVRKRPWGK | 70% |
| | | | | | FAAEIRDPERGGSRVW | |
| | | | | | LGTFDTAEEAARAYDR | |
| | | | | | AAFAMKGATAVLNFPASG | |
| 164 | G3737 | Oryza sativa | AP2: 8-72 | AP2: 233-427 | AASKYRGVRRRLPWGK | 70% |
| | | (japonica | | | FAAEIRDPERGGSRVW | |
| | | cultivar- | | | LGTFDTAEEAARAYDR | |
| | | group) | | | AAFAMKGAMAVLNFPGRT | |
| 36 | G1795 | Arabidopsis | AP2: 11-75 | AP2: 57-251 | EHGKYRGVRRRPWGK | 69% |
| | | thaliana | | | YAAEIRDSRKHGERVW | |
| | | | | | LGTFDTAEEAARAYDQ | |
| | | | | | AAYSMRGQAAILNFPHEY | |
| 200 | G3794 | Zea mays | AP2: 6-70 | AP2: 135-329 | EPTKYRGVRRRPSGKF | 69% |
| | | | | | AAEIRDSSRQSVRMWL | |
| | | | | | GTFDTAEEAARAYDRA | |
| | | | | | AYAMRGQIAVLNFPAEA | |
| 80 | G3517 | Zea mays | AP2: 13-77 | AP2: 76-270 | EPTKYRGVRRRPWGK | 67% |
| | | | | | YAAEIRDSSRHGVRIW | |
| | | | | | LGTFDTAEEAARAYDR | |
| | | | | | SANSMRGANAVLNFPEDA | |
| 162 | G3736 | Triticum | AP2: 12-76 | AP2: 163-357 | EPTKYRGVRRRPWGKF | 67% |
| | | aestivum | | | AAEIRDSSRHGVRMWL | |
| | | | | | GTFDTAEEAAAAYDRS | |
| | | | | | AYSMRGRNAVLNFPDRA | |
| 166 | G3739 | Zea mays | AP2: 13-77 | AP2: 211-405 | EPTKYRGVRRRPWGK | 67% |
| | | | | | YAAEIRDSSRIIGVRIW | |
| | | | | | LGTFDTAEEAARAYDR | |
| | | | | | SAYSMRGANAVLNFPEDA | |
| | | | | | | % ID to |
| | | | | | | G2053 |
| 10 | G2053 | Arabidopsis | NAC: 6-152 | NAC: 16-456 | GLRFRPTDKEIVVDYLR | 100% |
| | | thaliana | | | PKNSDRDTSHVDRVIST | |
| | | | | | VTIRSFDPWELPGQSRI | |
| | | | | | KLKDESWGFFSPKENK | |
| | | | | | YGRGDQQIRKTKSGY | |
| | | | | | WKITGKLPKPILRNRQEI | |
| | | | | | GEKKVLMFYMSKELG | |
| | | | | | GSKSDWVMHEYHAFS | |
| | | | | | PTQMMMTYTICKVMFKGD | |
| 20 | G515 | Arabidopsis | NAC: 6-149 | NAC: 93-524 | GLRFGPTDEEIVVDYL | 78% |
| | | thaliana | | | WPKNSDRDTSHVDRFI | |
| | | | | | NTVPVGRLDPWELPGQ | |
| | | | | | SRIKLKDVAWGFFRPK | |
| | | | | | ENKYGRGDQQMRKTK | |
| | | | | | SGFWKSTGRPKPIMRN | |
| | | | | | RQQIGEKKLLMFYTSKE | |
| | | | | | SKSDWVIHEYHGFSHN | |
| | | | | | QMMMTYTLCKVMFNGG | |
| 24 | G517 | Arabidopsis | NAC: 6-153 | NAC: 16-459 | GFRFRLPNDEEIVDHYLR | 62% |
| | | thaliana | | | PKNLDSDTSHVDEVIST | |
| | | | | | VDICSFEPWDLPSKSMI | |
| | | | | | KSRDGVWYFFSVKEM | |
| | | | | | KYNRGDQQRRRTNSGF | |
| | | | | | WKKTGKTMTVMRKRG | |
| | | | | | NREKIGEKRVLVFKNR | |
| | | | | | DGSKTDWVMHEYHAT | |
| | | | | | SLFPNQMMTYTVCKVE | |
| | | | | | FKGE | |
| 22 | G516 | Arabidopsis | NAC: 6-141 | NAC: 16-423 | GFRFRPTDGEIVDIYLR | 55% |
| | | thaliana | | | PKNLESNTSHVDEVIST | |
| | | | | | VDICSFDPWDLPSHSR | |
| | | | | | MKTRDQVWYFFGRKE | |
| | | | | | NKYGKGDRQIRKTKSG | |
| | | | | | FWKKTGVTMDIMRKT | |
| | | | | | GDREKIGEKRVLVFKN | |
| | | | | | HGGSKSDWAMHEYHA | |
| | | | | | TFSSPNQGE | |
| | | | | | | % ID to |
| | | | | | | G2999 |
| 14 | G2999 | Arabidopsis | ZF: 80-133 | ZF: 280-441 | ARYRECQKNHAASSGG | 100% |
| | | thaliana | | | HVVDGCGEFMSSGEEG | |
| | | | | | TVESLLCAACDCHRSF | |
| | | | | | HRKEID | |
| 14 | G2999 | Arabidopsis | HB: 198-261 | HB: 634-825 | KKRFRTKFNEEQKEKM | 100% |
| | | thaliana | | | MEFAEKIGWRMTKLED | |
| | | | | | DEVNRIFCREIKVKRQV | |
| | | | | | FKVWMHNNKQAAKKKD | |
| 62 | G2998 | Arabidopsis | ZF: 74-127 | ZF: 220-381 | VRYREGLKNHAASVG | 79% |
| | | thaliana | | | GSVHDGCGEFMPSGEE | |
| | | | | | GTIEALRCAACDCHRN | |
| | | | | | FHRKEMD | |
| 62 | G2998 | Arabidopsis | HB: 240-303 | HB: 718-909 | KKRFRTKFTTDQKERM | 78% |
| | | thaliana | | | MDFAEKLGWRMNKQD | |
| | | | | | EEELKRFCGEIGVKRQ | |
| | | | | | VFKVWMHNNKNNAKKPP | |
| 64 | G3000 | Arabidopsis | ZF: 58-111 | ZF: 318-479 | AKYRECQKNHAASTG | 77% |
| | | thaliana | | | GHVVDGCCEFMAGGE | |
| | | | | | EGTLGALKCAACNCHR | |
| | | | | | SFHRKEVY | |
| 64 | G3000 | Arabidopsis | HB: 181-244 | HB: 687-878 | KKRVRTKIINEEQKEKM | 65% |
| | | thaliana | | | KEFAERLGWRMQKKD | |
| | | | | | EEEIDKFCRMVNLRRQ | |
| | | | | | VFKVWMHNNKQAMKRNN | |
| 106 | G3670 | Lotus | ZF: 62-115 | ZF: 184-345 | VRYRECQKNHAVSFGG | 74% |
| | | corniculatus | | | HAVDGCCEFMAAGDE | |
| | | var. japonicus | | | GTLEAVICAACNCHRN | |
| | | | | | FHRKEID | |
| 106 | G3670 | Lotus | HB: 207-270 | HB: 619-810 | KKRYRTKFTPEQKEKM | 57% |
| | | corniculatus | | | LAFAEELGWRIQKHQE | |
| | | var. japonicus | | | AAVEQFCAETCVRRNV | |
| | | | | | LKVWMHNNKNTLGKKP | |
| 110 | G3674 | Oryza sativa | ZF: 61-114 | ZF: 274-435 | ARYRECLKNHAVGIGG | 72% |
| | | (indica | | | HAVDGCGEFMASGEE | |
| | | cultivar- | | | GSIDALRCAACGCHRN | |
| | | group) | | | FHRKESE | |
| 110 | G3674 | Oryza sativa | HB: 226-289 | HB: 769-960 | KKRFRTKFTQEQKDKM | 59% |
| | | (indica | | | LAFAERLGWRIQKHDE | |
| | | cultivar- | | | AAVQQFCEEVCVKRH | |
| | | group) | | | VLKVWMHNNKHTLGKKA | |
| 102 | G3663 | Lotus | ZF: 88-141 | ZF: 262-423 | IRYRECLRNHAARLGS | 70% |
| | | corniculatus | | | HVTDGCGEFMPNGEQ | |
| | | var. japonicus | | | GTPESLICAACECHRNF | |
| | | | | | HRKEAQ | |
| 102 | G3663 | Lotus | HB: 219-282 | HB: 655-846 | KKRFRTKFTQQQKDR | 64% |
| | | corniculatus | | | MMEFAEKLGWKIQKQ | |
| | | var. japonicus | | | DEEEVKQFCSHVGVKR | |
| | | | | | QAFKVWMHNSKQAMKKKQ | |
| 108 | G3671 | Oryza sativa | ZF: 40-93 | ZF: 233-394 | GRYRECLKNHAVGIGG | 70% |
| | | (japonica | | | HAVDGCGEFMAAGEE | |
| | | cultivar- | | | GTIDALRCAACNCHRN | |
| | | group) | | | FHRKESE | |
| 108 | G3671 | Oryza sativa | HB: 200-263 | HB: 713-904 | KKRFRTKFTQEQKDKM | 59% |
| | | (japonica | | | LAFAERVGWRIQKLHDE | |
| | | cultivar- | | | AAVQQFCDEVGVKRH | |
| | | group) | | | VLKVWMHNNKHTLGKKL | |
| 60 | G2997 | Arabidopsis | ZF: 47-100 | ZF: 263-424 | IRYRECLKNHAVNIGG | 68% |
| | | thaliana | | | HAVDGCCEFMPSGEDG | |
| | | | | | TLDALKCAACGCHRNF | |
| | | | | | HRKETE | |
| 60 | G2997 | Arabidopsis | HB: 157-220 | HB: 593-784 | TKRERTKFTAEQKEKM | 59% |
| | | thaliana | | | LAFAERLGWRIQKHDD | |
| | | | | | VAVEQFCAETGVRRQV | |
| | | | | | LKIWMHNNKNSLGKKP | |
| 116 | G3683 | Oryza sativa | ZF: 72-125 | ZF: 214-375 | ARYRECLKLNHAAAIGG | 68% |
| | | (japonica | | | SATDGCGEFMPGGEEG | |
| | | cultivar- | | | SLDALRCSACGCHRNF | |
| | | group) | | | HRKELD | |
| 116 | G3683 | Oryza sativa | HB: 193-256 | HB: 577-768 | RKRFRTKFTAEQKARM | 59% |
| | | (japonica | | | LGFAEEVGWRLQKLED | |
| | | cultivar- | | | AVVQRFCQEVGVKRR | |
| | | group) | | | VLKVWMHNNKHTLARRH | |
| 112 | G3675 | Brassica | ZF: 49-102 | ZF: 201-362 | VRYRECLKNHAVNIGG | 66% |
| | | napus | | | HAVDGCCEFMIPSGEDG | |
| | | | | | SLDALKCAACGCHRNF | |
| | | | | | HRKETE | |
| 112 | G3675 | Brassica | HB: 162-225 | HB: 540-731 | AKRFRTKFTAEQKDKM | 56% |
| | | napus | | | LAFAERLGWRIQKHDD | |
| | | | | | AAVEQFCAETGVRRQV | |
| | | | | | LKIWMHNNKNSLGRKP | |
| 122 | G3690 | Oryza sativa | ZF: 161-213 | ZF:481-639 | WRYRECLKNHAARMG | 66% |
| | | (japonica | | | AHVLDGCGEFMSSPGD | |
| | | cultivar- | | | GAAALACAACGCHRSF | |
| | | group) | | | HRREPA | |
| 122 | G3690 | Oryza sativa | HB: 318-381 | HB: 952-1143 | KKRFRTKFTAEQKERM | 56% |
| | | (japonica | | | REFAHRVGWRIHKPDA | |
| | | cultivar- | | | AAVDAFCAQVGVSRR | |
| | | group) | | | VLKVWMHNNKHLAKTPP | |
| 104 | G3668 | Flaveria | ZF: 42-95 | ZF: 410-571 | YRYKECLKNHAVGIGG | 64% |
| | | bidentis | | | QAVDGCGEFMAAGDE | |
| | | | | | GTLDALKCAACNCHR | |
| | | | | | NFHRKEVE | |
| 104 | G3668 | Flaveria | HB: 174-237 | HB: 806-997 | KKRFRTKFTQDQKDR | 54% |
| | | bidentis | | | MLAFSEALGWRIQKHD | |
| | | | | | EAAVQQFCNETGVKRH | |
| | | | | | VLKVWMHNNKHTIGKKP | |
| 58 | G2996 | Arabidopsis | ZF: 73-126 | ZF: 241-402 | FRFRECLKNQAVNIGG | 64% |
| | | thaliana | | | HAVDGCGEFMPAGIEG | |
| | | | | | TIDALKCAACGCHRNF | |
| | | | | | HRKELP | |
| 58 | G2996 | Arabidopsis | HB: 191-254 | HB: 595-786 | RKRHRTKFTAEQKERM | 53% |
| | | thaliana | | | LALAERIGWRIQRQDD | |
| | | | | | EVIQRFCQETGVPRQVL | |
| | | | | | KVWLHNNKHTLGKSP | |
| 54 | G2994 | Arabidopsis | ZF: 88-141 | ZF: 329-490 | IKYKECLKNHAAAMG | 62% |
| | | thaliana | | | GNATDGCGEFMPSGED | |
| | | | | | GSIEALTCSACNCHRNF | |
| | | | | | HRKEVE | |
| 54 | G2994 | Arabidopsis | HB: 218-281 | HB: 719-910 | KKRFRTKFTPEQKEKM | 65% |
| | | thaliana | | | LSFAEKVGWKIQRQED | |
| | | | | | CVVQRFCEEIGVKRRV | |
| | | | | | LKVWMHNNKIHFSKKN | |
| 120 | G3686 | Oryza sativa | ZF: 38-88 | ZF: 112-264 | CRYHECLRNHAAASGG | 62% |
| | | (indica | | | HVVDGCGEFMPASTEE | |
| | | cultivar- | | | PLACAACGCHRSFHRR | |
| | | group) | | | DPS | |
| 120 | G3686 | Oryza sativa | HB: 159-222 | HB: 475-666 | RRRSRTTFTREQKEQM | 50% |
| | | (indica | | | LAFAERVGWRIQRQEE | |
| | | cultivar- | | | ATVEHFCAQVGVRRQ | |
| | | group) | | | ALKVWMHNNKHSFKQKQ | |
| 52 | G2993 | Arabidopsis | ZF: 85-138 | ZF: 442-603 | IKYKECLKNHAATMGG | 61% |
| | | thaliana | | | NAIDGCGEFMPSGEEG | |
| | | | | | SIEALTCSVCNCHRNFH | |
| | | | | | RRETE | |
| 52 | G2993 | Arabidopsis | HB: 222-285 | HB: 853-1044 | KKRFRTKFTQEQKEKM | 57% |
| | | thaliana | | | ISFAERVGWKIQRQEES | |
| | | | | | VVQQLCQEIGIRRRVLK | |
| | | | | | VWMHNNKQNLSKKS | |
| 48 | G2991 | Arabidopsis | ZF: 54-109 | ZF: 218-385 | ATYKECLKNHAAGIGG | 60% |
| | | thaliana | | | HALDGCGEFMPSPSFN | |
| | | | | | SNDPASLTCAACGCHR | |
| | | | | | NFHRREED | |
| 48 | G2991 | Arabidopsis | HB: 179-242 | HB: 593-784 | RKRFRTKFSQYQKEKM | 59% |
| | | thaliana | | | FEFSERVGWRMPKADD | |
| | | | | | VVVKEFCREIGVDKSV | |
| | | | | | FKVWMHNNKISGRSGA | |
| 114 | G3680 | Zea mays | ZF: 34-89 | ZF: 223-390 | PLYRECLKNHAASLGG | 60% |
| | | | | | HAVDGCGEFMPSPGAN | |
| | | | | | PADPTSLKCAACGCHR | |
| | | | | | NFHRRTLE | |
| 114 | G3680 | Zea mays | HB: 222-285 | HB: 787-978 | RKRFRTKLFTAEQKQRM | 50% |
| | | | | | QELSERLGWRLQKRDE | |
| | | | | | AIVDEWCRDIGVGKGV | |
| | | | | | FKVWMHNNKHNFLGGH | |
| 118 | G3685 | Oryza sativa | ZF: 43-95 | ZF: 216-374 | VRYHECLRNHAAAMG | 59% |
| | | (japonica | | | GHVVDGCREFMPMPG | |
| | | cultivar- | | | DAADALKCAACGCHR | |
| | | group) | | | SFHRKDDG | |
| 118 | G3685 | Oryza sativa | HB: 172-235 | HB: 603-794 | RKRFRTKFTPEQKEQM | 56% |
| | | (japonica | | | LAFAERVGWRMQKQD | |
| | | cultivar- | | | EALVEQFCAQVGVRRQ | |
| | | group) | | | VFKVWMHNNKSSIGSSS | |
| 44 | G2989 | Arabidopsis | ZF: 50-105 | ZF: 208-375 | VTYKECLKNHAAAIGG | 58% |
| | | thaliana | | | HALDGCGEFMPSPSSTP | |
| | | | | | SDPTSLKCAACGCHRN | |
| | | | | | FHRRETD | |
| 44 | G2989 | Arabidopsis | HB: 192-255 | HB: 634-825 | RKRFRTKFSSNQKEKM | 59% |
| | | thaliana | | | HEFADRIGWKIQKRDE | |
| | | | | | DEVRDFCREIGVDKGV | |
| | | | | | LKVWMHNNKNSFKFSG | |
| 46 | G2990 | Arabidopsis | ZF: 54-109 | ZF: 206-373 | FTYKECLKNHAAALGG | 57% |
| | | thaliana | | | HALDGCGEFMPSPSSIS | |
| | | | | | SDPTSLKCAACGCHRN | |
| | | | | | FHRRDPD | |
| 46 | G2990 | Arabidopsis | HB: 200-263 | HB: 644-835 | RKRFRTKFSQFQKEKM | 57% |
| | | thaliana | | | HEFAERVGWKMQKRD | |
| | | | | | EDDVRDFCRQIGVDKS | |
| | | | | | VLKVWMHNNKNTFNRRD | |
| 66 | G3001 | Arabidopsis | ZF: 62-113 | ZF: 222-377 | PHYYECRKNHAADIGT | 57% |
| | | thaliana | | | TAYDGCGEFVSSTGEE | |
| | | | | | DSLNCAACGCHRNFHR | |
| | | | | | EELI | |
| 66 | G3001 | Arabidopsis | HB: 179-242 | HB: 573-764 | VKRLKTKFTAEQTEKM | 42% |
| | | thaliana | | | RDYAEKLRWKVRPER | |
| | | | | | QEEVEEFCVEIGVNRK | |
| | | | | | NFRIWMNNHKDKIIIDE | |
| 50 | G2992 | Arabidopsis | ZF: 29-84 | ZF: 85-252 | VCYKECLKNHAANLG | 55% |
| | | thaliana | | | GHALDGCGEFMPSPTA | |
| | | | | | TSTDPSSLRCAACGCH | |
| | | | | | RNFHRRDPS | |
| 50 | G2992 | Arabidopsis | HB: 156-219 | HB: 466-657 | RKRTRTKFTPEQKIKM | 48% |
| | | thaliana | | | RAFAEKAGWKINGCDE | |
| | | | | | KSVREFCNEVGIERGVL | |
| | | | | | KVWMHNNKYSLLNGK | |
| 128 | G3695 | Oryza sativa | ZF: 22-71 | ZF: 64-213 | GKYKECMRNHAAAMG | 51% |
| | | (japonica | | | GQAFDGCGEYMPASPD | |
| | | cultivar- | | | SLKCAACGCHRSFHRR | |
| | | group) | | | AAA | |
| 128 | G3695 | Oryza sativa | HB: 164-227 | HB: 490-681 | RKRFRTKFTPEQKERM | 57% |
| | | (japonica | | | REFAEKQGWRINRNDD | |
| | | cultivar- | | | GALDRFCVEIGVKRHV | |
| | | group) | | | LKVWMHNHKNQLASSP | |
| 56 | G2995 | Arabidopsis | ZF: 3-58 | ZF: 143-310 | VLYNECLKNHAVSLGG | 50% |
| | | thaliana | | | HALDGCGEFTPKSTTIL | |
| | | | | | TDPPSLRCDACGCHRN | |
| | | | | | FHRRSPS | |
| 56 | G2995 | Arabidopsis | HB: 115-178 | HB: 479-670 | KKHKRTKFTAEQKVK | 45% |
| | | thaliana | | | MRGFAERAGWKINGW | |
| | | | | | DEKWVREFCSEVGIER | |
| | | | | | KVLKVWIHNNKYFNN | |
| | | | | | GRS | |
| 124 | G3692 | Oryza sativa | ZF: 10-61 | ZF: 28-183 | EVYRECMRNHAAKLG | 48% |
| | | (japonica | | | TYANDGCCEYTPDDGH | |
| | | cultivar- | | | PAGLLCAACGCHRNFH | |
| | | group) | | | RKDFL | |
| 124 | G3692 | Oryza sativa | HB: 119-188 | HB: 355-564 | RRRTRTKFTEEQKARM | 58% |
| | | (japonica | | | LRFAERLGWRMPKREP | |
| | | cultivar- | | | GRAPGDDEVARFCREI | |
| | | group) | | | GVNRQVFKVWMHNH | |
| | | | | | KAGGGGGG | |
| 126 | G3694 | Oryza sativa | ZF: 1-40 | ZF: 1-120 | MGAHVLDGCGEFMSSP | 48% |
| | | (japonica | | | GDGAAALACAACGCH | |
| | | cultivar- | | | RSFHRREPA | |
| | | group) | | | | |
| 126 | G3694 | Oryza sativa | HB: 145-208 | HB: 433-624 | KKRFRTKFTAEQKERM | 56% |
| | | (japonica | | | REFAHRVGWRIHKPDA | |
| | | cultivar- | | | AAVDAFCAQVGVSRR | |
| | | group) | | | VLKVWMHNNKLLAKTPP | |
| 68 | G3002 | Arabidopsis | ZF: 5-53 | ZF: 81-227 | CVYRECMRNHAAKLG | 42% |
| | | thaliana | | | SYAIDGCREYSQPSTGD | |
| | | | | | LCVACGCHRSYHRRIDV | |
| 68 | G3002 | Arabidopsis | HB: 106-168 | HB: 384-572 | QRRRKSKFTAEQREAM | 35% |
| | | thaliana | | | KDYAAKLGWTLKDKR | |
| | | | | | ALREEIRVFCEGIGVTR | |
| | | | | | YHFKTWVNNNKXFYH | |
| | | | | | | % ID to |
| | | | | | | G3086 |
| 16 | G3086 | Arabidopsis | HLH/MYC: 307- | HLH/MYC: | KRGCATHPRSIAERVR | 100% |
| | | thaliana | 365 | 1059-1235 | RTKISERMRKLQDLVP | |
| | | | | | NMDTQTNTADMLDLA | |
| | | | | | VQYIKDLQEQVK | |
| 188 | G3767 | Glycine max | HLH/MYC: 146- | HLH/MYC: 436- | KRGCATHPRSIAERVR | 93% |
| | | | 204 | 612 | RTKISERMRKLQDLVP | |
| | | | | | NMDKQTNTADMLDLA | |
| | | | | | VDYIKDLQKQVQ | |
| 190 | G3768 | Glycine max | HLH/MYC: 190- | HLH/MYC: 568- | KRGCATHPRSIAERVR | 93% |
| | | | 248 | 744 | RTKISERMRKLQDLVP | |
| | | | | | NMDKQTNTADMLDLA | |
| | | | | | VDYIKDLQKQVQ | |
| 192 | G3769 | Glycine max | HLH/MYC: 240- | HLH/MYC: 718- | KRGCATHPRSIAERVR | 93% |
| | | | 298 | 894 | RTKISERMRKLQDLVP | |
| | | | | | NMDKQTNTADMLDLA | |
| | | | | | VEYIKDLQNQVQ | |
| 174 | G3744 | Oryza sativa | HLH/MYC: 71- | HLH/MYC: 211- | KRGCATHPRSIAERVR | 89% |
| | | (japonica | 129 | 387 | RTRISERIRKLQELVPN | |
| | | cultivar- | | | MDKQTNTADMLDLAV | |
| | | group) | | | DYIKDLQKQVK | |
| 178 | G3755 | Zea mays | HLH/MYC: 97- | HLH/MYC: 289- | KRGCATHPRSIAERVR | 89% |
| | | | 155 | 465 | RTKISERJRKLQELVPN | |
| | | | | | MDKQTNTSDMLDLAV | |
| | | | | | DYIKDLQKQVK | |
| 26 | G592 | Arabidopsis | HLH/MYC: 282- | HLH/MYC: 964- | KRGCATHPRSIAERVR | 88% |
| | | thaliana | 340 | 1140 | RTRISERMRKLQELVPN | |
| | | | | | MDKQTNTSDMLDLAV | |
| | | | | | DYIKDLQRQYK | |
| 186 | G3766 | Glycine max | HLH/MYC: 35- | HLH/MYC: 103- | KRGCATHPRSIAERVR | 88% |
| | | | 93 | 279 | RTRLISERMRKLQELVPH | |
| | | | | | MDKQTNTADMLDLAV | |
| | | | | | EYIKDLQKQFK | |
| 172 | G3742 | Oryza sativa | HLH/MYC: 199- | HLH/MYC: 595- | KRGCATHPRSLAERVR | 86% |
| | | (japonica | 257 | 771 | RTRISERIRKLQELVPN | |
| | | cultivar- | | | MEKQTNTADMLDLAV | |
| | | group) | | | DYIKELQKQVK | |
| 198 | G3782 | Pinus taeda | HLH/MYC: 471- | HLH/MYC: | KRGCATHPRSIAERVR | 80% |
| | | | 530 | 1411-1590 | RTRISERMRKLQELVPN | |
| | | | | | SDKQTVNIADMLDEAV | |
| | | | | | EYVKSLQKQVQ | |
| 176 | G3746 | Oryza sativa | HLH/MYC: 312- | HLH/MYC: 934- | KRGCATHPRSIAERERR | 79% |
| | | (japonica | 370 | 1110 | TRISKRILKKLQDLVPN | |
| | | cultivar- | | | MDKQTNTSDMLDIAVT | |
| | | group) | | | YIKELQGQVE | |
| 184 | G3765 | Glycine max | HLH/MYC: 147- | HLH/MYC: 439- | KRGFATHPRSIAERVRR | 79% |
| | | | 205 | 615 | TRISERIRKLQELVPTM | |
| | | | | | DKQTSTAEMLDLALDY | |
| | | | | | IKDLQKQFK | |
| 194 | G3771 | Glycine max | HLH/MYC: 84- | HLH/MYC: 250- | KRGCATHPRSIAERVR | 79% |
| | | | 142 | 426 | RTRISDRIRKLQELVPN | |
| | | | | | MDKQTNTADMLDEAV | |
| | | | | | AYVKFLQKQIE | |
| 28 | G1134 | Arabidopsis | HLH/MYC: 187- | HLH/MYC: 619- | KRGCATHPRSIAERVR | 77% |
| | | thaliana | 245 | 795 | RTRISDRIRKLQELVPN | |
| | | | | | MDKQTNTADMLEEAV | |
| | | | | | EYVKVLQRQIQ | |
| 168 | G3740 | Oryza sativa | HLH/MYC: 141- | HLH/MYC: 421- | KRGCATHPRSIAERERR | 77% |
| | | (japonica | 199 | 597 | TRISEKLRKLQELVPNM | |
| | | cultivar- | | | DKQTSTADMLDLAVE | |
| | | group) | | | HIKGLQSQLQ | |
| 180 | G3763 | Glycine max | HLH/MYC: 161- | HLH/MYC: 481- | KRGFATHPRSIAERERR | 77% |
| | | | 219 | 657 | TRISARIKKLQDLFPKS | |
| | | | | | DKQTSTADMLDLAVE | |
| | | | | | YIKDLQKQVK | |
| 182 | G3764 | Glycine max | HLH/MYC: 370- | HLH/MYC: | KRGFATHPRSIAERVRR | 77% |
| | | | 428 | 1108-1284 | TRISERIKKLQDLFPKSE | |
| | | | | | KQTSTADMLDLAVEYI | |
| | | | | | KDLQQKVK | |
| 196 | G3772 | Glycine max | HLH/MYC: 211- | HLH/MYC: 631- | KRGCATHPRSIAERERR | 77% |
| | | | 269 | 807 | TRISGKLKKLQDLVPN | |
| | | | | | MDKQTSYADMLDLAV | |
| | | | | | QHIKGLQTQVQ | |
| 40 | G2555 | Arabidopsis | HLH/MYC: 184- | HLH/MYC: 726- | KRGCATHPRSIAERVR | 76% |
| | | thaliana | 242 | 902 | RTRISDRIRRLQELVPN | |
| | | | | | MDKQTNTADMLEEAV | |
| | | | | | EYVKALQSQIQ | |
| 170 | G3741 | Oryza sativa | HLH/MYC: 288- | HLH/MYC: 862- | KRGCATHPRSIAERERR | 76% |
| | | (japonica | 346 | 1038 | TRISEKLRKLQALVPN | |
| | | cultivar- | | | MDKQTSTSDMLDLAV | |
| | | group) | | | DHIKGLQSQLQ | |
| 38 | G2149 | Arabidopsis | HLH/MYC: 286- | HLH/MYC: 927- | KRGCATHPRSIAERERR | 74% |
| | | thaliana | 344 | 1103 | TRISGKLKKLQDLVPN | |
| | | | | | MDKQTSYSDMLDLAV | |
| | | | | | QHIKGLQHQLQ | |
| 42 | G2766 | Arabidopsis | HLH/MYC: 234- | HLH/MYC: 778- | KRGFATHPRSIAERERR | 72% |
| | | thaliana | 292 | 954 | TRISGKLKKLQELVPN | |
| | | | | | MDKQTSYADMLDLAV | |
| | | | | | EHIKGLQHQVE | |

Table 2 shows a number of polypeptides of the invention not listed in Table 1, identified by SEQ ID NO; Identifier (e.g., Gene ID (GID) No); the transcription factor family to which the polypeptide belongs, and conserved domains of the polypeptide. The first column shows the polypeptide SEQ ID NO; the third column shows the transcription factor family to which the polynucleotide belongs; and the fourth column shows the amino acid residue positions of the conserved domain in amino acid (AA) coordinates.

TABLE 2
Gene families and conserved domains

| Polypeptide | | | Conserved Domains in |
|---|---|---|---|
| SEQ ID NO: | Identifier | Family | Amino Acid Coordinates |
| 224 | G175 | WRKY | 178-234, 372-428 |
| 226 | G303 | HLH/MYC | 92-161 |
| 228 | G354 | Z-C2H2 | 42-62, 88-109 |
| 230 | G489 | CAAT | 57-156 |
| 232 | G634 | TH | 62-147, 189-245 |
| 234 | G682 | MYB-related | 27-63 |
| 236 | G916 | WRKY | 293-349 |
| 238 | G975 | AP2 | 4-71 |
| 240 | G1069 | AT-hook | 67-74 |
| 242 | G1452 | NAC | 55-196 |
| 244 | G1820 | CAAT | 70-133 |
| 246 | G2701 | MYB-related | 33-81, 129-183 |
| 248 | G2789 | AT-hook | 53-73, 121-165 |
| 250 | G2839 | Z-C2H2 | 34-60, 85-113 |
| 252 | G2854 | ACBF-like | 110-250 |
| 254 | G3083 | bZIP-ZW2 | 75-105, 188-215 |
| 256 | G184 | WRKY | 295-352 |
| 258 | G186 | WRKY | 312-369 |
| 260 | G353 | Z-C2H2 | 41-61, 84-104 |
| 262 | G512 | NAC | 24-166 |
| 264 | G596 | AT-hook | 89-96 |
| 266 | G714 | CAAT | 58-148 |
| 268 | G877 | WRKY | 272-328, 487-603 |
| 270 | G1357 | NAC | 17-158 |
| 272 | G1387 | AP2 | 4-71 |
| 274 | G1634 | MYB-related | 129-180 |
| 276 | G1889 | Z-C2H2 | 80-100 |
| 278 | G1940 | ACBF-like | 156-228 |
| 280 | G1974 | Z-C2H2 | 32-60, 72-116 |
| 282 | G2153 | AT-hook | 75-94, 162-206 |
| 284 | G2583 | AP2 | 4-71 |
| 288 | G226 | MYB-related | 28-78 |
| 290 | G481 | CAAT | 20-109 |
| 292 | G482 | CAAT | 25-116 |
| 294 | G485 | CAAT | 21-116 |
| 296 | G486 | CAAT | 5-66 |
| 298 | G1067 | AT-hook | 86-92, 94-247 |
| 300 | G1070 | AT-hook | 98-120 |
| 302 | G1073 | AT-hook | 34-40, 42-187 |
| 304 | G1075 | AT-hook | 78-85 |
| 306 | G1076 | AT-hook | 82-89 |
| 308 | G1248 | CAAT | 46-155 |
| 310 | G1364 | CAAT | 29-118 |
| 312 | G1781 | CAAT | 35-130 |
| 314 | G1816 | MYB-related | 31-81 |
| 316 | G1945 | AT-hook | 49-71 |
| 318 | G2155 | AT-hook | 18-38 |
| 320 | G2156 | AT-hook | 72-78, 80-232 |
| 322 | G2345 | CAAT | 26-152 |
| 324 | G2657 | AT-hook | 116-129 |
| 326 | G2718 | MYB-related | 21-76 |
| 328 | G3392 | MYB-related | 21-72 |
| 330 | G3393 | MYB-related | 20-71 |
| 332 | G3394 | CAAT | 37-126 |
| 334 | G3395 | CAAT | 19-108 |
| 336 | G3396 | CAAT | 21-110 |
| 338 | G3397 | CAAT | 23-112 |
| 340 | G3398 | CAAT | 21-110 |
| 342 | G3399 | AT-hook | 99-105, 107-253 |
| 344 | G3400 | AT-hook | 83-89, 91-237 |
| 346 | G3401 | AT-hook | 35-41, 43-186 |
| 348 | G3403 | AT-hook | 58-64, 66-207 |
| 350 | G3404 | AT-hook | 111-117, 119-263 |
| 352 | G3405 | AT-hook | 97-103, 105-248 |
| 354 | G3406 | AT-hook | 82-88, 90-232 |
| 356 | G3407 | AT-hook | 63-69, 71-220 |
| 358 | G3408 | AT-hook | 83-89, 91-247 |
| 360 | G3429 | CAAT | 35-124 |
| 362 | G3431 | MYB-related | 20-71 |
| 364 | G3434 | CAAT | 18-107 |
| 366 | G3435 | CAAT | 22-111 |
| 368 | G3436 | CAAT | 20-109 |
| 370 | G3437 | CAAT | 54-143 |
| 372 | G3444 | MYB-related | 20-71 |
| 374 | G3445 | MYB-related | 15-65 |
| 376 | G3446 | MYB-related | 16-66 |
| 378 | G3447 | MYB-related | 16-66 |
| 380 | G3448 | MYB-related | 15-66 |
| 382 | G3449 | MYB-related | 15-66 |
| 384 | G3450 | MYB-related | 9-60 |
| 386 | G3456 | AT-hook | 44-50, 52-195 |
| 388 | G3458 | AT-hook | 56-62, 64-207 |
| 390 | G3459 | AT-hook | 77-83, 85-228 |
| 392 | G3460 | AT-hook | 74-80, 82-225 |
| 394 | G3462 | AT-hook | 82-88, 90-237 |
| 396 | G3470 | CAAT | 27-116 |
| 398 | G3471 | CAAT | 26-115 |
| 400 | G3472 | CAAT | 25-114 |
| 402 | G3473 | CAAT | 23-113 |
| 404 | G3474 | CAAT | 25-114 |
| 406 | G3475 | CAAT | 23-112 |
| 408 | G3476 | CAAT | 26-115 |
| 410 | G3477 | CAAT | 27-116 |
| 412 | G3478 | CAAT | 23-112 |
| 414 | G3556 | AT-hook | 45-51, 53-196 |
| 416 | G3835 | CAAT | 4-92 |
| 418 | G3836 | CAAT | 34-122 |
| 420 | G3837 | CAAT | 35-123 |

Producing Polypeptides

The polynucleotides of the invention include sequences that encode transcription factors and transcription factor homolog polypeptides and sequences complementary thereto, as well as unique fragments of coding sequence, or sequence complementary thereto. Such polynucleotides can be, for example. DNA or RNA, the latter including mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (e.g., a purification tag, a localization signal, as a fusion-protein, as a pre-protein, or the like), in combination with non-coding sequences (for example, introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art, and are described in, for example, Berger and Kimmel, Guide to Molecular Cloning Techniques, Methods in Enzymology, vol. 152 Academic Press, Inc., San Diego, Calif. (“Berger”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, Ausubel et al. eds., Current Protocols, a joint venture between Greene Publishing Associates, Inc. and John Wiley & Sons, Inc., (supplemented through 2000) (“Ausubel”).

Alternatively, polynucleotides of the invention, can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qbeta-replicase amplification and other RNA polymerase mediated techniques (for example, NASBA), e.g., for the production of the homologous nucleic acids of the invention are found in Berger (supra), Sambrook (supra), and Ausubel (supra), as well as Mullis et al. (1987) PCR Protocols A Guide to Methods and Applications (Innis et al. eds) Academic Press Inc. San Diego, Calif. (1990) (Innis). Improved methods for cloning in vitro amplified nucleic acids are described in Wallace et al. U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, e.g., a polynucleotide encoding all or part of a transcription factor. For example, chemical synthesis using the phosphoramidite method is described, e.g., by Beaucage et al. (1981) Tetrahedron Letters 22: 1859-1869; and Matthes et al. (1984) EMBO J. 3: 801-805. According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

Homologous Sequences

Sequences homologous, i.e., that share significant sequence identity or similarity, to those provided in the Sequence Listing, derived from Arabidopsis thaliana or from other plants of choice, are also an aspect of the invention. Homologous sequences can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam, and sweet potato; and beans. The homologous sequences may also be derived from woody species, such pine, poplar and eucalyptus, or mint or other labiates. In addition, homologous sequences may be derived from plants that are evolutionarily related to crop plants, but which may not have yet been used as crop plants. Examples include deadly nightshade (Atropa belladona), related to tomato; jimson weed (Datura strommium), related to peyote; and teosinte (Zea species), related to corn (maize).

Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360). For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) Plant J. 16: 433442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001), in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Transcription factor gene sequences are conserved across diverse eukaryotic species lines (Goodrich et al. (1993) Cell 75: 519-530; Lin et al. (1991) Nature 353: 569-571; Sadowski et al. (1988) Nature 335: 563-564). Plants are no exception to this observation; diverse plant species possess transcription factors that have similar sequences and functions.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related paralogs is the CBF family, with four well-defined members in Arabidopsis (SEQ ID NOs: 422, 424, 426, and GenBank accession number AB015478) and at least one ortholog in Brassica napus, (SEQ ID NO: 428), all of which control pathways involved in both freezing and drought stress (Gilmour et al. (1998) Plant J. 16: 433-442; Jaglo et al. (1998) Plant Physiol. 127: 910-917).

The following references represent a small sampling of the many studies that demonstrate that conserved transcription factor genes from diverse species are likely to function similarly (i.e., regulate similar target sequences and control the same traits), and that transcription factors may be transformed into diverse species to confer or improve traits.

(1) The Arabidopsis NPR1 gene regulates systemic acquired resistance (SAR) (Cao et al. (1997) Cell 88: 57-63); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. Oryzae, the transgenic plants displayed enhanced resistance (Chem et al. (2001) Plant J. 27: 101-113). NPR1 acts through activation of expression of transcription factor genes, such as TGA2 (Fan and Dong (2002) Plant Cell 14: 1377-1389).

(2) E2F genes are involved in transcription of plant genes for proliferating cell nuclear antigen (PCNA). Plant E2Fs share a high degree of similarity in amino acid sequence between monocots and dicots, and are even similar to the conserved domains of the animal E2Fs. Such conservation indicates a functional similarity between plant and animal E2Fs. E2F transcription factors that regulate meristem development act through common cis-elements, and regulate related (PCNA) genes (Kosugi and Ohashi, (2002) Plant J. 29: 45-59).

(3) The ABI5 gene (ABA insensitive 5) encodes a basic leucine zipper factor required for ABA response in the seed and vegetative tissues. Co-transformation experiments with ABI5 cDNA constructs in rice protoplasts resulted in specific transactivation of the ABA-inducible wheat, Arabidopsis, bean, and barley promoters. These results demonstrate that sequentially similar ABI5 transcription factors are key targets of a conserved ABA signaling pathway in diverse plants. (Gampala et al. (2001) J. Biol. Chem. 277: 1689-1694).

(4) Sequences of three Arabidopsis GAMYB-like genes were obtained on the basis of sequence similarity to GAMYB genes from barley, rice, and L. temulentum. These three Arabadopsis genes were determined to encode transcription factors (AtMYB33, AtMYB65, and AtMYB101) and could substitute for a barley GAMYB and control alpha-amylase expression (Gocal et al. (2001) Plant Physiol. 127: 1682-1693).

(5) The floral control gene LEAFY from Arabidopsis can dramatically accelerate flowering in numerous dictoyledonous plants. Constitutive expression of Arabidopsis LEAFY also caused early flowering in transgenic rice (a monocot), with a heading date that was 26-34 days earlier than that of wild-type plants. These observations indicate that floral regulatory genes from Arabidopsis are useful tools for heading date improvement in cereal crops (He et al. (2000) Transgenic Res. 9: 223-227).

(6) Bioactive gibberellins (GAs) are essential endogenous regulators of plant growth. GA signaling tends to be conserved across the plant kingdom. GA signaling is, mediated via GAI, a nuclear member of the GRAS family of plant transcription factors. Arabidopsis GAI has been shown to function in rice to inhibit gibberellin response pathways (Fu et al. (2001) Plant Cell 13: 1791-1802).

(7) The Arabidopsis gene SUPERMAN (SUP), encodes a putative transcription factor that maintains the boundary between stamens and carpels. By over-expressing Arabidopsis SUP in rice, the effect of the gene's presence on whorl boundaries was shown to be conserved. This demonstrated that SUP is a conserved regulator of floral whorl boundaries and affects cell proliferation (Nandi et al. (2000) Curr. Biol. 10: 215-218).

(8) Maize, petunia and Arabidopsis myb transcription factors that regulate flavonoid biosynthesis are very genetically similar and affect the same trait in their native species, therefore sequence and function of these myb transcription factors correlate with each other in these diverse species (Borevitz et al. (2000) Plant Cell 12: 2383-2394).

(9) Wheat reduced height-1 (Rht-B1/Rht-D1) and maize dwarf-8 (d8) genes are orthologs of the Arabidopsis gibberellin insensitive (GAM) gene. Both of these genes have been used to produce dwarf grain varieties that have improved grain yield. These genes encode proteins that resemble nuclear transcription factors and contain an SH2-like domain, indicating that phosphotyrosine may participate in gibberellin signaling. Transgenic rice plants containing a mutant GAI allele from Arabidopsis have been shown to produce reduced responses to gibberellin and are dwarfed, indicating that mutant GAI orthologs could be used to increase yield in a wide range of crop species (Peng et al. (1999) Nature 400: 256-261).

Transcription factors that are homologous to the listed sequences will typically share at least about 70% amino acid sequence identity in the AP2 domain. More closely related transcription factors can share at least about 79% or about 90% or about 95% or about 98% or more sequence identity with the listed sequences, or with the listed sequences but excluding or outside a known consensus sequence or consensus DNA-binding site, or with the listed sequences excluding one or all conserved domains. Factors that are most closely related to the listed sequences share, e.g., at least about 85%, about 90% or about 95% or more % sequence identity to the listed sequences, or to the listed sequences but excluding or outside a known consensus sequence or consensus DNA-binding site or outside one or all conserved domain. At the nucleotide level, the sequences will typically share at least about 40% nucleotide sequence identity, preferably at least about 50%, about 60%, about 70% or about 80% sequence identity, and more preferably about 85%, about 90%, about 95% or about 97% or more sequence identity to one or more of the listed sequences, or to a listed sequence but excluding or outside a known consensus sequence or consensus DNA-binding site, or outside one or all conserved domain. The degeneracy of the genetic code enables major variations in the nucleotide sequence of a polynucleotide while maintaining the amino acid sequence of the encoded protein. AP2 domains within the AP2 transcription factor family may exhibit a higher degree of sequence homology, such as at least 70% amino acid sequence identity including conservative substitutions, and preferably at least 80% sequence identity, and more preferably at least 85%, or at least about 86%, or at least about 87%, or at least about 88%, or at least about 90%, or at least about 95%, or at least about 98% sequence identity. Transcription factors that are homologous to the listed sequences should share at least 30%, or at least about 60%, or at least about 75%, or at least about 80%, or at least about 90%, or at least about 95% amino acid sequence identity over the entire length of the polypeptide or the homolog.

Percent identity. can be determined electronically, e.g., by using the MEGALIGN program (DNASTAR, Inc. Madison, Wis.). The MEGALIGN program can create alignments between two or more sequences according to different methods, for example, the clustal method. (See, for example, Higgins and Sharp (1988) Gene 73: 237-244.) The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Other techniques for alignment are described in Methods in Enzymology, vol. 266, Computer Methods for Macromolecular Sequence Analysis (1996), ed. Doolittle, Academic Press, Inc., San Diego, Calif., USA. Preferably, an alignment program that permits gaps in the sequence is utilized to align the sequences. The Smith-Waterman is one type of algorithm that permits gaps in sequence alignments (see Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method. (See, e.g., Hein (1990) Methods Enzymol. 183: 626-645.) Identity between sequences can also be determined by other methods known in the art, e.g., by varying hybridization conditions (see U.S. patent application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997) Nucleic Acids Res. 25: 217-221), PFAM, and other databases which contain previously identified and annotated motifs, sequences and gene functions. Methods that search for primary sequence patterns with secondary structure gap penalties (Smith et al. (1992) Protein Engineering 5: 35-51) as well as algorithms such as Basic Local Alignment Search Tool (BLAST; Altschul (1993) J. Mol. Evol. 36: 290-300; Altschul et al. (1990) supra), BLOCKS (Henikoff and Henikoff (1991) Nucleic Acids Res. 19: 6565-6572), Hidden Markov Models (HMM; Eddy (1996) Curr. Opin. Str. Biol. 6: 361-365; Sonnhammer et al. (1997) Proteins 28: 405-420), and the like, can be used to manipulate and analyze polynucleotide and polypeptide sequences encoded by polynucleotides. These databases, algorithms and other methods are well known in the art and are described in Ausubel et al. (1997; Short Protocols in Molecular Biology, John Wiley & Sons, New York, N.Y., unit 7.7) and in Meyers (1995; Molecular Biology and Biotechnology, Wiley VCH, New York, N.Y., p 856-853).

A further method for identifying or confirming that specific homologous sequences control the same function is by comparison of the transcript profile(s) obtained upon overexpression or knockout of two or more related transcription factors. Since transcript profiles are diagnostic for specific cellular states, one skilled in the art will appreciate that genes that have a highly similar transcript profile (e.g., with greater than 50% regulated transcripts in common, more preferably with greater than 70% regulated transcripts in common, most preferably with greater than 90% regulated transcripts in common) will have highly similar functions. Fowler et al. (2002, Plant Cell, 14: 1675-79) have shown that three paralogous AP2 family genes (CBF1, CBF2 and CBF3), each of which is induced upon cold treatment, and each of which can condition improved freezing tolerance, have highly similar transcript profiles. Once a transcription factor has been shown to provide a specific function, its transcript profile becomes a diagnostic tool to determine whether putative paralogs or orthologs have the same function.

Furthermore, methods using manual alignment of sequences similar or homologous to one or more polynucleotide sequences or one or more polypeptides encoded by the polynucleotide sequences may be used to identify regions of similarity and AP2 binding domains. Such manual methods are well-known of those of skill in the art and can include, for example, comparisons of tertiary structure between a polypeptide sequence encoded by a polynucleotide which comprises a known function with a polypeptide sequence encoded by a polynucleotide sequence which has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs can be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated using, for example, PCR, using primers designed from a presently disclosed transcription factor gene sequence, or by probing with a partial or complete cDNA or with one or more sets of degenerate probes based on the disclosed sequences. The cDNA library may be used to transform plant cells. Expression of the cDNAs of interest is detected using, for example, methods disclosed herein such as microarrays, Northern blots, quantitative PCR, or any other technique for monitoring changes in expression. Genomic clones may be isolated using similar techniques to those.

Identifying Polynucleotides or Nucleic Acids by Hybridization

Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:
Tₘ(° C.)=81.5+16.6(log [Na+])+0.41(% G+C)−0.62(% formamide)−500/L
(II) DNA-RNA:
Tₘ(° C.)=79.8+18.5(log [Na+])+0.58(% G+C)+0.12(% G+C)²−0.35(% formamide)−820/L
(III) RNA-RNA:
Tₘ(° C.)=79.8+18.5(log [Na+])+0.58(% G+C)+0.12(% G+C)²−0.35(% formamide)−820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 min, or about 0.1×SSC, 0.1% SDS at 65° C. and washing twice for 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C. −68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, U.S. patent application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, for example, to those shown in SEQ ID NO: 1, 11, 87, 89, 91, 93, 95, 97, and 99, and fragments thereof under various conditions of stringency. (See, e.g., Wahl and Berger (1987) Methods Enzymol. 152: 399-407; Kimmel (1987) Methods Enzymol. 152: 507-511). Estimates of homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

Identifying Polynucleotides or Nucleic Acids with Expression Libraries

In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (e.g., E. coli) and used to raise antibodies (monoclonal or polyclonal) specific for the polypeptide(s) in question. Antibodies can also be raised against synthetic peptides derived from transcription factor, or transcription factor homolog, amino acid sequences. Methods of raising antibodies are well known in the art and are described in Harlow and Lane (1988), Antibodies: A Laboratory Manual, Cold Spring Harbor Laboratory, New York. Such antibodies can then be used to screen an expression library produced from the plant from which it is desired to clone additional transcription factor homologs, using the methods described above. The selected cDNAs can be confirmed by sequencing and enzymatic activity.

Sequence Variations

It will readily be appreciated by those of skill in the art, that any of a variety of polynucleotide sequences are capable of encoding the transcription factors and transcription factor homolog polypeptides of the invention. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the Sequence Listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that, for example, G2133, SEQ ID NO: 12, represents a single transcription factor; allelic variation and alternative splicing may be expected to occur. Allelic variants of SEQ ID NO: 11 can be cloned by probing cDNA or genomic libraries from different individual organisms according to standard procedures. Allelic variants of the DNA sequence shown in SEQ ID NO: 11, including those containing silent mutations and those in which mutations result in amino acid sequence changes, are within the scope of the present invention, as are proteins which are allelic variants of SEQ ID NO: 12. cDNAs generated from alternatively spliced mRNAs, which retain the properties of the transcription factor are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (see U.S. Pat. No. 6,388,064).

Thus, in addition to the sequences set forth in the Sequence Listing, the invention also encompasses related nucleic acid molecules that include allelic or splice variants of the sequences of the invention, for example, SEQ ID NO: 1, 11, 87, 89, 91, 93, 95, 97, and 99, and include sequences which are complementary to any of the above nucleotide sequences. Related nucleic acid molecules also include nucleotide sequences encoding a polypeptide comprising or consisting essentially of a substitution, modification, addition and/or deletion of one or more amino acid residues compared to the polypeptide sequences of the invention, for example, SEQ ID NO: 2, 12, 88, 90, 92, 94, 96, 98, 100, and equivalogs. Such related polypeptides may comprise, for example, additions and/or deletions of one or more N-linked or O-linked glycosylation sites, or an addition and/or a deletion of one or more cysteine residues.

For example, Table 3 illustrates, e.g., that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 3

| | |
|---|---|
| Amino acid | Possible Codons |

| | | | |
|---|---|---|---|
| Alanine | Ala | A | GCA GGC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC AGT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, e.g., site-directed mutagenesis, available in the art. Accordingly, any and all such variations of a sequence selected from the above table are a feature of the invention.

In addition to silent variations, other conservative variations that alter one, or a few amino acid residues in the encoded polypeptide, can be made without altering the function of the polypeptide, these conservative variants are, likewise, a feature of the invention.

For example, substitutions, deletions and insertions introduced into the sequences provided in the Sequence Listing, are also envisioned by the invention. Such sequence modifications can be engineered into a sequence by site-directed mutagenesis (Wu (ed.) Methods Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, e.g., a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 4 when it is desired to maintain the activity of the protein. Table 4 shows amino acids which can be substituted for an amino acid in a protein and which are typically regarded as conservative substitutions.

TABLE 4

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp;Phe |
| Val | Ile; Leu |

Similar substitutions are those in which at least one residue in the amino acid sequence has been removed and a different residue inserted in its place. Such substitutions generally are made in accordance with the Table 5 when it is desired to maintain the activity of the protein. Table 5 shows amino acids which can be substituted for an amino acid in a protein and which are typically regarded as structural and functional substitutions. For example, a residue in column 1 of Table 5 may be substituted with a residue in column 2; in addition, a residue in column 2 of Table 5 may be substituted with the residue of column 1.

TABLE 5

| | |
|---|---|
| Residue | Similar Substitutions |

| | |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 5 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Further Modifying Sequences of the Invention—Mutation/Forced Evolution

In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, e.g., according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel, supra, provides additional details on mutagenesis methods. Artificial forced evolution methods are described, for example, by Stemmer (1994) Nature 370: 389-391, Stemmer (1994) Proc. Natl. Acad. Sci. 91: 10747-10751, and U.S. Pat. Nos. 5,811,238, 5,837,500, and 6,242,568. Methods for engineering synthetic transcription factors and other polypeptides are described, for example, by Zhang et al. (2000) J Biol. Chem. 275: 33850-33860, Liu et al. (2001) J. Biol. Chem. 276: 11323-11334, and Isalan et al. (2001) Nature Biotechnol. 19: 656-660. Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel, supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches.

For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for Saccharomyces cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations are optionally introduced using techniques which are well known in the art, e.g., site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998) Proc. Natl. Acad. Sci. 95: 376-381; Aoyama et al. (1995) Plant Cell 7: 1773-1785), peptides derived from bacterial sequences (Ma and Ptashne (1987) Cell 51: 113-119) and synthetic peptides (Giniger and Ptashne (1987) Nature 330: 670-672).

Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homolog.

The transgenic plants of the present invention comprising recombinant polynucleotide sequences are generally derived from parental plants, which may themselves be non-transformed (or non-transgenic) plants. These transgenic plants may either have a transcription factor gene “knocked out” (for example, with a genomic insertion by homologous recombination, an antisense or ribozyme construct) or expressed to a normal or wild-type extent. However, overexpressing transgenic “progeny” plants will exhibit greater mRNA levels, wherein the mRNA encodes a transcription factor, that is, a DNA-binding protein that is capable of binding to a DNA regulatory sequence and inducing transcription, and preferably, expression of a plant trait gene. Preferably, the mRNA expression level will be at least three-fold greater than that of the parental plant, or more preferably at least ten-fold greater mRNA levels compared to said parental plant, and most preferably at least fifty-fold greater compared to said parental plant.

Vectors, Promoters, and Expression Systems

The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger, Sambrook, supra and Ausubel, supra. Any of the identified sequences can be incorporated into a cassette or vector, e.g., for expression in plants. A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucleic Acids Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol. 102: 1077-1084; Vasil (1993) Bio/Technology 10: 667-674; Wan and Lemeaux (1994) Plant Physiol. 104: 37-48, and for Agrobacterium-mediated DNA transfer (Ishida et al. (1996) Nature Biotechnol. 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally-or developmentally-regulated, or cell- or tissue-specific expression), a transcription initiation start site, an RNA processing signal (such as intron splice sites), a transcription termination site, and/or a polyadenylation signal.

A potential utility for the transcription factor polynucleotides disclosed herein is the isolation of promoter elements from these genes that can be used to program expression in plants of any genes. Each transcription factor gene disclosed herein is expressed in a unique fashion, as determined by promoter elements located upstream of the start of translation, and additionally within an intron of the transcription factor gene or downstream of the termination codon of the gene. As is well known in the art, for a significant portion of genes, the promoter sequences are located entirely in the region directly upstream of the start of translation. In such cases, typically the promoter sequences are located within 2.0 kb of the start of translation, or within 1.5 kb of the start of translation, frequently within 1.0 kb of the start of translation, and sometimes within 0.5 kb of the start of translation.

The promoter sequences can be isolated according to methods known to one skilled in the art.

Examples of constitutive plant promoters which can be useful for expressing the TF sequence include: the cauliflower mosaic virus (CaMV) 35S promoter, which confers constitutive, high-level expression in most plant tissues (see, e.g., Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

The transcription factors of the invention may be operably linked with a specific promoter that causes the transcription factor to be expressed in response to environmental, tissue-specific or temporal signals. A variety of plant gene promoters that regulate gene expression in response to environmental, hormonal, chemical, developmental signals, and in a tissue-active manner can be used for expression of a TF sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al., (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Molec. Biol. 38: 817-825) and the like. Additional promoters are those that elicit expression in response to heat (Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffner and Sheen (1991) Plant Cell 3: 997-1012); wounding (e.g., wunI, Siebertz et al. (1989) Plant Cell 1: 961-968); pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz (1997) Annu. Rev. Plant Physiol. Plant Mol. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458).

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Additional Expression Elements

Specific initiation signals can aid in efficient translation of coding sequences. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

Expression Hosts

The present invention also relates to host cells which are transduced with vectors of the invention, and the production of polypeptides of the invention (including fragments thereof) by recombinant techniques. Host cells are genetically engineered (i.e., nucleic acids are introduced, e.g., transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook, supra and Ausubel, supra.

The host cell can be a eukaryotic cell, such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985) Proc. Natl. Acad. Sci. 82: 5824-5828, infection by viral vectors such as cauliflower mosaic virus (CaMV) (Hohn et al. (1982) Molecular Biology of Plant Tumors Academic Press, New York, N.Y., pp. 549-560; U.S. Pat. No. 4,407,956), high velocity ballistic penetration by small particles with the nucleic acid either within the matrix of small beads or particles, or on the surface (Klein et al. (1987) Nature 327: 70-73), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. 80: 4803-4807).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, e.g., produced by transduction of a vector of the invention, are an additional feature of the invention.

For long-term, high-yield production of recombinant proteins, stable expression can be used. Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature proteins of the invention can be designed with signal sequences which direct secretion of the mature polypeptides through a prokaryotic or eukaryotic cell membrane.

Modified Amino Acid Residues

Polypeptides of the invention may contain one or more modified amino acid residues. The presence of modified amino acids may be advantageous in, for example, increasing polypeptide half-life, reducing polypeptide antigenicity or toxicity, increasing polypeptide storage stability, or the like. Amino acid residue(s) are modified, for example, co-translationally or post-translationally during recombinant production or modified by synthetic or chemical means.

Non-limiting examples of a modified amino acid residue include incorporation or other use of acetylated amino acids, glycosylated amino acids, sulfated amino acids, prenylated (e.g., farnesylated, geranylgeranylated) amino acids, PEG modified (e.g., “PEGylated”) amino acids, biotinylated amino acids, carboxylated amino acids, phosphorylated amino acids, etc. References adequate to guide one of skill in the modification of amino acid residues are replete throughout the literature.

The modified amino acid residues may prevent or increase affinity of the polypeptide for another molecule, including, but not limited to, polynucleotide, proteins, carbohydrates, lipids and lipid derivatives, and other organic or synthetic compounds.

Identification of Additional Protein Factors

A transcription factor provided by the present invention can also be used to identify additional endogenous or exogenous molecules that can affect a phentoype or trait of interest. Such molecules include endogenous molecules that are acted upon either at a transcriptional level by a transcription factor of the invention to modify a phenotype as desired. For example, the transcription factors can be employed to identify one or more downstream genes that are subject to a regulatory effect of the transcription factor. In one approach, a transcription factor or transcription factor homolog of the invention is expressed in a host cell, e.g., a transgenic plant cell, tissue or explant, and expression products, either RNA or protein, of likely or random targets are monitored, e.g., by hybridization to a microarray of nucleic acid probes corresponding to genes expressed in a tissue or cell type of interest, by two-dimensional gel electrophoresis of protein products, or by any other method known in the art for assessing expression of gene products at the level of RNA or protein. Alternatively, a transcription factor of the invention can be used to identify promoter sequences (such as binding sites on DNA sequences) involved in the regulation of a downstream target. After identifying a promoter sequence, interactions between the transcription factor and the promoter sequence can be modified by changing specific nucleotides in the promoter sequence or specific amino acids in the transcription factor that interact with the promoter sequence to alter a plant trait. Typically, transcription factor DNA-binding sites are identified by gel shift assays. After identifying the promoter regions, the promoter region sequences can be employed in double-stranded DNA arrays to identify molecules that affect the interactions of the transcription factors with their promoters (Bulyk et al. (1999) Nature Biotechnol. 17: 573-577).

The identified transcription factors are also useful to identify proteins that modify the activity of the transcription factor. Such modification can occur by covalent modification, such as by phosphorylation, or by protein-protein (homo or-heteropolymer) interactions. Any method suitable for detecting protein-protein interactions can be employed. Among the methods that can be employed are co-immunoprecipitation, cross-linking and co-purification through gradients or chromatographic columns, and the two-hybrid yeast system.

The two-hybrid system detects protein interactions in vivo and is described in Chien et al. ((1991) Proc. Natl. Acad. Sci. 88: 9578-9582) and is commercially available from Clontech (Palo Alto, Calif.). In such a system, plasmids are constructed that encode two hybrid proteins: one consists of the DNA-binding domain of a transcription activator protein fused to the TF polypeptide and the other consists of the transcription activator protein's activation domain fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae that contains a reporter gene (e.g., lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. Then, the library plasmids responsible for reporter gene expression are isolated and sequenced to identify the proteins encoded by the library plasmids. After identifying proteins that interact with the transcription factors, assays for compounds that interfere with the TF protein-protein interactions can be preformed.

Subsequences

Also contemplated are uses of polynucleotides, also referred to herein as oligonucleotides, typically having at least 12 bases, preferably at least 15, more preferably at least 20, 30, or 50 bases, which hybridize under at least highly stringent (or ultra-high stringent or ultra-ultra-high stringent conditions) conditions to a polynucleotide sequence described above. The polynucleotides may be used as probes, primers, sense and antisense agents, and the like, according to methods as noted supra.

Subsequences of the polynucleotides of the invention, including polynucleotide fragments and oligonucleotides are useful as nucleic acid probes and primers. An oligonucleotide suitable for use as a probe or primer is at least about 15 nucleotides in length, more often at least about 18 nucleotides, often at least about 21 nucleotides, frequently at least about 30 nucleotides, or about 40 nucleotides, or more in length. A nucleic acid probe is useful in hybridization protocols, e.g., to identify additional polypeptide homologs of the invention, including protocols for microarray experiments. Primers can be annealed to a complementary target DNA strand by nucleic acid hybridization to form a hybrid between the primer and the target DNA strand, and then extended along the target DNA strand by a DNA poiymerase enzyme. Primer pairs can be used for amplification of a nucleic acid sequence, e.g., by the polymerase chain reaction (PCR) or other nucleic-acid amplification methods. See Sambrook, supra, and Ausubel, supra.

In addition, the invention includes an isolated or recombinant polypeptide including a subsequence of at least about 15 contiguous amino acids encoded by the recombinant or isolated polynucleotides of the invention. For example, such polypeptides, or domains or fragments thereof, can be used as immunogens, e.g., to produce antibodies specific for the polypeptide sequence, or as probes for detecting a sequence of interest. A subsequence can range in size from about 15 amino acids in length up to and including the full length of the polypeptide.

To be encompassed by the present invention, an expressed polypeptide which comprises such a polypeptide subsequence performs at least one biological function of the intact polypeptide in substantially the same manner, or to a similar extent, as does the intact polypeptide. For example, a polypeptide fragment can comprise a recognizable structural motif or functional domain such as a DNA binding domain that activates transcription, e.g., by binding to a specific DNA promoter region an activation domain, or a domain for protein-protein interactions.

Production of Transgenic Plants

Modification of Traits

The polynucleotides of the invention are favorably employed to produce transgenic plants with various traits, or characteristics, that have been modified in a desirable manner, e.g., to improve the seed characteristics of a plant. For example, alteration of expression levels or patterns (e.g., spatial or temporal expression patterns) of one or more of the transcription factors (or transcription factor homologs) of the invention, as compared with the levels of the same protein found in a wild-type plant, can be used to modify a plant's traits. An illustrative example of trait modification, improved characteristics, by altering expression levels of a particular transcription factor is described further in the Examples and the Sequence Listing.

Arabidopsis as a model system

Arabidopsis thaliana is the object of rapidly growing attention as a model for genetics and metabolism in plants. Arabidopsis has a small genome, and well-documented studies are available. It is easy to grow in large numbers and mutants defining important genetically controlled mechanisms are either available, or can readily be obtained. Various methods to introduce and express isolated homologous genes are available (see Koncz et al., eds., Methods in Arabidopsis Research (1992) World Scientific, New Jersey, N.J., in “Preface”). Because of its small size, short life cycle, obligate autogamy and high fertility, Arabidopsis is also a choice organism for the isolation of mutants and studies in morphogenetic and development pathways, and control of these pathways by transcription factors (Koncz supra, p. 72). A number of studies introducing transcription factors into A. thaliana have demonstrated the utility of this plant for understanding the mechanisms of gene regulation and trait alteration in plants. (See, for example, Koncz supra, and U.S. Pat. No. 6,417,428).

Arabidopsis Genes in Transgenic Plants.

Expression of genes which encode transcription factors modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997 Genes and Development 11: 3194-3205) and Peng et al. (1999 Nature 400: 256-261). In addition, many others have demonstrated that an Arabidopsis transcription factor expressed in an exogenous plant species elicits the same or very similar phenotypic response. See, for example, Fu et al. (2001 Plant Cell 13: 1791-1802); Nandi et al. (2000 Curr. Biol. 10: 215-218); Coupland (1995 Nature 377: 482-483); and Weigel and Nilsson (1995, Nature 377: 482-500).

Homologous Genes Introduced into Transgenic Plants.

Homologous genes that may be derived from any plant, or from any source whether natural, synthetic, semi-synthetic or recombinant, and that share significant sequence identity or similarity to those provided by the present invention, may be introduced into plants, for example, crop plants, to confer desirable or improved traits. Consequently, transgenic plants may be produced that comprise a recombinant expression vector or cassette with a promoter operably linked to one or more sequences homologous to presently disclosed sequences. The promoter may be, for example, a plant or viral promoter.

The invention thus provides for methods for preparing transgenic plants, and for modifying plant traits. These methods include introducing into a plant a recombinant expression vector or cassette comprising a functional promoter operably linked to one or more sequences homologous to presently disclosed sequences. Plants and kits for producing these plants that result from the application of these methods are also encompassed by the present invention.

Transcription Factors of Interest for the Modification of Plant Traits

Currently, the existence of a series of maturity groups for different latitudes represents a major barrier to the introduction of new valuable traits. Any trait (e.g. disease resistance) has to be bred into each of the different maturity groups separately, a laborious and costly exercise. The availability of single strain, which could be grown at any latitude, would therefore greatly increase the potential for introducing new traits to crop species such as soybean and cotton.

For the specific effects, traits and utilities conferred to plants, one or more transcription factor genes of the present invention may be used to increase or decrease, or improve or prove deleterious to a given trait. For example, knocking out a transcription factor gene that naturally occurs in a plant, or suppressing the gene (with, for example, antisense suppression), may cause decreased tolerance to a drought stress relative to non-transformed or wild-type plants. By overexpressing this gene, the plant may experience increased tolerance to the same stress. More than one transcription factor gene may be introduced into a plant, either by transforming the plant with one or more vectors comprising two or more transcription factors, or by selective breeding of plants to yield hybrid crosses that comprise more than one introduced transcription factor.

Genes, Traits and Utilities that Affect Plant Characteristics

Plant transcription factors can modulate gene expression, and, in turn, be modulated by the environmental experience of a plant. Significant alterations in a plant's environment invariably result in a change in the plant's transcription factor gene expression pattern. Altered transcription factor expression patterns generally result in phenotypic changes in the plant. Transcription factor gene product(s) in transgenic plants then differ(s) in amounts or proportions from that found in wild-type or non-transformed plants, and those transcription factors likely represent polypeptides that are used to alter the response to the environmental change. By way of example, it is well accepted in the art that analytical methods based on altered expression patterns may be used to screen for phenotypic changes in a plant far more effectively than can be achieved using traditional methods.

Sugar Sensing.

In addition to their important role as an energy source and structural component of the plant cell, sugars are central regulatory molecules that control several aspects of plant physiology, metabolism and development (Hsieh et al. (1998) Proc. Natl. Acad. Sci. 95: 13965-13970). It is thought that this control is achieved by regulating gene expression and, in higher plants, sugars have been shown to repress or activate plant genes involved in many essential processes such as photosynthesis, glyoxylate metabolism, respiration, starch and sucrose synthesis and degradation, pathogen response, wounding response, cell cycle regulation, pigmentation, flowering and senescence. The mechanisms by which sugars control gene expression are not understood.

Several sugar sensing mutants have turned out to be allelic to abscisic acid (ABA) and ethylene mutants. ABA is found in all photosynthetic organisms and acts as a key regulator of transpiration, stress responses, embryogenesis, and seed germination. Most ABA effects are related to the compound acting as a signal of decreased water availability, whereby it triggers a reduction in water loss, slows growth, and mediates adaptive responses. However, ABA also influences plant growth and development via interactions with other phytohormones. Physiological and molecular studies indicate that maize and Arabidopsis have almost identical pathways with regard to ABA biosynthesis and signal transduction. For further review, see Finkelstein and Rock ((2002) Abscisic acid biosynthesis and response (In The Arabidopsis Book, Editors: Somerville and Meyerowitz (American Society of Plant Biologists, Rockville, Md.).

This potentially implicates the sequences of the invention that, when overexpressed, confer a sugar sensing or hormone signaling phenotype in plants. On the other hand, the sucrose treatment used in these experiments (9.4% w/v) could also be an osmotic stress. Therefore, one could interpret these data as an indication that these transgenic lines are more tolerant to osmotic stress. However, it is well known that plant responses to ABA, osmotic and other stress may be linked, and these different treatments may even act in a synergistic manner to increase the degree of a response. For example, Xiong, Ishitani, and Zhu ((1999) Plant Physiol. 119: 205-212) have shown that genetic and molecular studies may be used to show extensive interaction between osmotic stress, temperature stress, and ABA responses in plants. These investigators analyzed the expression of RD29A-LUC in response to various treatment regimes in Arabidopsis. The RD29A promoter contains both the ABA-responsive and the dehydration-responsive element—also termed the C-repeat—and can be activated by osmotic stress, low temperature, or ABA treatment; transcription of the RD29A gene in response to osmotic and cold stresses is mediated by both ABA-dependent and ABA-independent pathways (Xiong, Ishitani, and Zhu (1999) supra). LUC refers to the firefly luciferase coding sequence, which, in this case, was driven by the stress responsive RD29A promoter. The results revealed both positive and negative interactions, depending on the nature and duration of the treatments. Low temperature stress was found to impair osmotic signaling but moderate heat stress strongly enhanced osmotic stress induction, thus acting synergistically with osmotic signaling pathways. In this study, the authors reported that osmotic stress and ABA can act synergistically by showing that the treatments simultaneously induced transgene and endogenous gene expression. Similar results were reported by Bostock and Quatrano ((1992) Plant Physiol. 98: 1356-1363), who found that osmotic stress and ABA act synergistically and induce maize Em gene expression. Ishitani et al (1997) Plant Cell 9: 1935-1949) isolated a group of Arabidopsis single-gene mutations that confer enhanced responses to both osmotic stress and ABA. The nature of the recovery of these mutants from osmotic stress and ABA treatment suggested that although separate signaling pathways exist for osmotic stress and ABA, the pathways share a number of components; these common components may mediate synergistic interactions between osmotic stress and ABA. Thus, contrary to the previously-held belief that ABA-dependent and ABA-independent stress signaling pathways act in a parallel manner, our data reveal that these pathways cross-talk and converge to activate stress gene expression.

Because sugars are important signaling molecules, the ability to control either the concentration of a signaling sugar or how the plant perceives or responds to a signaling sugar could be used to control plant development, physiology or metabolism. For example, the flux of sucrose (a disaccharide sugar used for systemically transporting carbon and energy in most plants) has been shown to affect gene expression and alter storage compound accumulation in seeds. Manipulation of the sucrose signaling pathway in seeds may therefore cause seeds to have more protein, oil or carbohydrate, depending on the type of manipulation. Similarly, in tubers, sucrose is converted to starch which is used as an energy store. It is thought that sugar signaling pathways may partially determine the levels of starch synthesized in the tubers. The manipulation of sugar signaling in tubers could lead to tubers with a higher starch content.

Thus, altering the expression of the presently disclosed transcription factor genes that manipulate the sugar signal transduction pathway, including, for example, GI 75, G303, G354, G481, G916, G922, G1069, G1073, G1820, G2053, G2701, G2789, G2839, G2854, along with their equivalogs, or that exhibit an osmotic stress phenotype, including, for example, G47, G482, G489 or G1069, G1073, as evidenced by their tolerance to, for example, high mannitol, salt or PEG, may be used to produce plants with desirable traits, including increased drought tolerance. In particular, manipulation of sugar signal transduction pathways could be used to alter source-sink relationships in seeds, tubers, roots and other storage organs leading to increase in yield.

Abiotic stress: drought and low humidity tolerance. Exposure to dehydration invokes similar survival strategies in plants as does freezing stress (see, for example, Yelenosky (1989) Plant Physiol 89: 444-451) and drought stress induces freezing tolerance (see, for example, Siminovitch et al. (1982) Plant Physiol 69: 250-255; and Guy et al. (1992) Planta 188: 265-270). In addition to the induction of cold-acclimation proteins, strategies that allow plants to survive in low water conditions may include, for example, reduced surface area, or surface oil or wax production. Modifying the expression of the presently disclosed transcription factor genes, including G2133, G1274, G922, G2999, G3086, G354, G1792, G2053, G975, G1069, G916, G1820, G2701, G47, G2854, G2789, G634, G175, G2839, G1452, G3083, G489, G303, G2992, and G682, and their equivalogs, may be used to increase a plant's tolerance to low water conditions and provide the benefits of improved survival, increased yield and an extended geographic and temporal planting range.

Osmotic stress. Modification of the expression of a number of presently disclosed transcription factor genes, e.g., G47, G482, G489 or G1069, G2053 and their equivalogs, may be used to increase germination rate or growth under adverse osmotic conditions, which could impact survival and yield of seeds and plants. Osmotic stresses may be regulated by specific molecular control mechanisms that include genes controlling water and ion movements, functional and structural-stress-induced proteins, signal perception and transduction, and free radical scavenging, and many others (Wang et al. (2001) Acta Hort. (ISHS) 560: 285-292). Instigators of osmotic stress include freezing, drought and high salinity, each of which are discussed in more detail below.

In many ways, freezing, high salt and drought have similar effects on plants, not the least of which is the induction of common polypeptides that respond to these different stresses. For example, freezing is similar to water deficit in that freezing reduces the amount of water available to a plant. Exposure to freezing temperatures may lead to cellular dehydration as water leaves cells and forms ice crystals in intercellular spaces (Buchanan, supra). As with high salt concentration and freezing, the problems for plants caused by low water availability include mechanical stresses caused by the withdrawal of cellular water. Thus, the incorporation of transcription factors that modify a plant's response to osmotic stress into, for example, a crop or ornamental plant, may be useful in reducing damage or loss. Specific effects caused by freezing, high salt and drought are addressed below.

The relationship between Salt, Drought and Freezing Tolerance

Plants are subject to a range of environmental challenges. Several of these, including drought stress, have the ability to impact whole plant and cellular water availability. Not surprisingly, then, plant responses to this collection of stresses are related. In a recent review, Zhu notes that “most studies on water stress signaling have focused on salt stress primarily because plant responses to salt and drought are closely related and the mechanisms overlap” (Zhu (2002) Ann. Rev. Plant Biol. 53: 247-273). Many examples of similar responses and pathways to this set of stresses have been documented. For example, the CBF transcription factors have been shown to condition resistance to salt, freezing and drought (Kasuga et al. (1999) Nature Biotech. 17: 287-291). The Arabidopsis rd29B gene is induced in response to both salt and dehydration stress, a process that is mediated largely through an ABA signal transduction process (Uno et al. (2000) Proc. Natl. Acad. Sci. USA 97: 11632-11637), resulting in altered activity of transcription factors that bind to an upstream element within the rd29B promoter. In Mesembryanthemum crystallinum (ice plant), Patharker and Cushman have shown that a calcium-dependent protein kinase (McCDPK1) is induced by exposure to both drought and salt stresses (Patharker and Cushman (2000) Plant J. 24: 679-691). The stress-induced kinase was also shown to phosphorylate a transcription factor, presumably altering its activity, although transcript levels of the target transcription factor are not altered in response to salt or drought stress. Similarly, Saijo et al. demonstrated that a rice salt/drought-induced calmodulin-dependent protein kinase (OsCDPK7) conferred increased salt and drought tolerance to rice when overexpressed (Saijo et al. (2000) Plant J. 23: 319-327).

Exposure to dehydration invokes similar survival strategies in plants as does freezing stress (see, for example, Yelenosky (1989) Plant Physiol 89: 444-451) and drought stress induces freezing tolerance (see, for example, Siminovitch et al. (1982) Plant Physiol 69: 250-255; and Guy et al. (1992) Planta 188: 265-270). In addition to the induction of cold-acclimation proteins, strategies that allow plants to survive in low water conditions may include, for example, reduced surface area, or surface oil or wax production.

Consequently, one skilled in the art would expect that some pathways involved in resistance to one of these stresses, and hence regulated by an individual transcription factor, will also be involved in resistance to another of these stresses, regulated by the same or homologous transcription factors. Of course, the overall resistance pathways are related, not identical, and therefore not all transcription factors controlling resistance to one stress will control resistance to the other stresses. Nonetheless, if a transcription factor conditions resistance to one of these stresses, it would be apparent to one skilled in the art to test for resistance to these related stresses.

Thus, the genes of the sequence listing, including, for example, G175, G922, G1452, G1820, G2701, G2999, G3086, and their equivalogs, that provide tolerance to salt may be used to engineer salt tolerant crops and trees that can flourish in soils with high saline content or under drought conditions. In particular, increased salt tolerance during the germination stage of a plant enhances survival and yield. Presently disclosed transcription factor genes that provide increased salt tolerance during germination, the seedling stage, and throughout a plant's life cycle, would find particular value for imparting survival and yield in areas where a particular crop would not normally prosper.

Summary of altered plant characteristics. The clades of structurally and functionally related sequences that derive from a wide range of plants, including the polynucleotides of the invention (for example, SEQ ID 1, 11, 87, 89, 91, 93, 95, 97, and 99, polynucleotides that encode polypeptide SEQ ID NOs: 2, 12, 88, 90, 92, 94, 96, 98, 100, fragments thereof, paralogs, orthologs, equivalogs, and fragments thereof, is provided. These sequences have been shown in laboratory and field experiments to confer altered size and abiotic stress tolerance phenotypes in plants. The invention also provides polypeptides comprising SEQ ID NOs: 2, 12, 88, 90, 92, 94, 96, 98, and 100, and fragments thereof, conserved domains thereof, paralogs, orthologs, equivalogs, and fragments thereof. Plants that overexpress these sequences have been observed to exhibit a sugar sensing phenotype and/or be more tolerant to a wide variety of abiotic stresses, including drought and high salt stress. Many of the orthologs of these sequences are listed in the Sequence Listing, and due to the high degree of structural similarity to the sequences of the invention, it is expected that these sequences will also function to increase drought stress tolerance. The invention also encompasses the complements of the polynucleotides. The polynucleotides are useful for screening libraries of molecules or compounds for specific binding and for creating transgenic plants having increased drought stress tolerance.

Antisense and Co-Suppression

In addition to expression of the nucleic acids of the invention as gene replacement or plant phenotype modification nucleic acids, the nucleic acids are also useful for sense and anti-sense suppression of expression, e.g. to down-regulate expression of a nucleic acid of the invention, e.g. as a further mechanism for modulating plant phenotype. That is, the nucleic acids of the invention, or subsequences or anti-sense sequences thereof, can be used to block expression of naturally occurring homologous nucleic acids. A variety of sense and anti-sense technologies are known in the art, e.g. as set forth in Lichtenstein and Nellen (1997) Antisense Technology: A Practical Approach IRL Press at Oxford University Press, Oxford, U.K. Antisense regulation is also described in Crowley et al. (1985) Cell 43: 633-641; Rosenberg et al. (1985) Nature 313: 703-706; Preiss et al. (1985) Nature 313: 27-32; Melton (1985) Proc. Natl. Acad. Sci. 82: 144-148; Izant and Weintraub (1985) Science 229: 345-352; and Kim and Wold (1985) Cell 42: 129-138. Additional methods for antisense regulation are known in the art. Antisense regulation has been used to reduce or inhibit expression of plant genes in, for example in European Patent Publication No. 271988. Antisense RNA may be used to reduce gene expression to produce a visible or biochemical phenotypic change in a plant (Smith et al. (1988) Nature, 334: 724-726; Smith et al. (1990) Plant Mol. Biol. 14: 369-379). In general, sense or anti-sense sequences are introduced into a cell, where they are optionally amplified, e.g. by transcription. Such sequences include both simple oligonucleotide sequences and catalytic sequences such as ribozymes.

For example, a reduction or elimination of expression (i.e., a “knock-out”) of a transcription factor or transcription factor homolog polypeptide in a transgenic plant, e.g., to modify a plant trait, can be obtained by introducing an antisense construct corresponding to the polypeptide of interest as a cDNA. For antisense suppression, the transcription factor or homolog cDNA is arranged in reverse orientation (with respect to the coding sequence) relative to the promoter sequence in the expression vector. The introduced sequence need not be the full length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous transcription factor gene in the plant cell.

Suppression of endogenous transcription factor gene expression can also be achieved using RNA interference, or RNAi. RNAi is a post-transcriptional, targeted gene-silencing technique that uses double-stranded RNA (dsRNA) to incite degradation of messenger RNA (mRNA) containing the same sequence as the dsRNA (Constans, (2002) The Scientist 16:36). Small interfering RNAs, or siRNAs are produced in at least two steps: an endogenous ribonuclease cleaves longer dsRNA into shorter, 21-23 nucleotide-long RNAs. The siRNA segments then mediate the degradation of the target mRNA (Zamore, (2001) Nature Struct. Biol., 8:746-50). RNAi has been used for gene function determination in a manner similar to antisense oligonucleotides (Constans, (2002) The Scientist 16:36). Expression vectors that continually express siRNAs in transiently and stably transfected have been engineered to express small hairpin RNAs (shRNAs), which get processed in vivo into siRNAs-like molecules capable of carrying out gene-specific silencing (Brummelkamp et al., (2002) Science 296:550-553, and Paddison, et al. (2002) Genes & Dev. 16:948-958). Post-transcriptional gene silencing by double-stranded RNA is discussed in further detail by Hammond et al. (2001) Nature Rev Gen 2: 110-119, Fire et al. (1998) Nature 391: 806-811 and Timmons and Fire (1998) Nature 395: 854. Vectors in which RNA encoded by a transcription factor or transcription factor homolog cDNA is over-expressed can also be used to obtain co-suppression of a corresponding endogenous gene, e.g., in the manner described in U.S. Pat. No. 5,231,020 to Jorgensen. Such co-suppression (also termed sense suppression) does not require that the entire transcription factor cDNA be introduced into the plant cells, nor does it require that the introduced sequence be exactly identical to the endogenous transcription factor gene of interest. However, as with antisense suppression, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of the transcription factor mRNA, e.g., sequences comprising one or more stop codon, or nonsense mutation) can also be used to suppress expression of an endogenous transcription factor, thereby reducing or eliminating its activity and modifying one or more traits. Methods for producing such constructs are described in U.S. Pat. No. 5,583,021. Preferably, such constructs are made by introducing a premature stop codon into the transcription factor gene. Alternatively, a plant trait can be modified by gene silencing using double-strand RNA (Sharp (1999) Genes and Development 13: 139-141). Another method for abolishing the expression of a gene is by insertion mutagenesis using the T-DNA of Agrobacterium tumefaciens. After generating the insertion mutants, the mutants can be screened to identify those containing the insertion in a transcription factor or transcription factor homolog gene. Plants containing a single transgene insertion event at the desired gene can be crossed to generate homozygous plants for the mutation. Such methods are well known to those of skill in the art (See for example Koncz et al. (1992) Methods in Arabidopsis Research, World Scientific Publishing Co. Pte. Ltd., River Edge, N.J.).

Alternatively, a plant phenotype can be altered by eliminating an endogenous gene, such as a transcription factor or transcription factor homolog, e.g., by homologous recombination (Kempin et al. (1997) Nature 389: 802-803).

A plant trait can also be modified by using the Cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The polynucleotides and polypeptides of this invention can also be expressed in a plant in the absence of an expression cassette by manipulating the activity or expression level of the endogenous gene by other means, such as, for example, by ectopically expressing a gene by T-DNA activation tagging (Ichikawa et al. (1997) Nature 390 698-701; Kakimoto et al. (1996) Science 274: 982-985). This method entails transforming a plant with a gene tag containing multiple transcriptional enhancers and once the tag has inserted into the genome, expression of a flanking gene coding sequence becomes deregulated. In another example, the transcriptional machinery in a plant can be modified so as to increase transcription levels of a polynucleotide of the invention (See, e.g., PCT Publications WO 96/06166 and WO 98/53057 which describe the modification of the DNA-binding specificity of zinc finger proteins by changing particular amino acids in the DNA-binding motif).

The transgenic plant can also include the machinery necessary for expressing or altering the activity of a polypeptide encoded by an endogenous gene, for example, by altering the phosphorylation state of the polypeptide to maintain it in an activated state.

Transgenic plants (or plant cells, or plant explants, or plant tissues) incorporating the polynucleotides of the invention and/or expressing the polypeptides of the invention can be produced by a variety of well established techniques as described above. Following construction of a vector, most typically an expression cassette, including a polynucleotide, e.g., encoding a transcription factor or transcription factor homolog, of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al., eds., (1984) Handbook of Plant Cell Culture—Crop Species, Macmillan Publ. Co., New York, N.Y.; Shimamoto et al. (1989) Nature 338: 274-276; Fromm et al. (1990) Bio/Technol. 8: 833-839; and Vasil et al. (1990) Bio/Technol. 8: 429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,619,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified. The modified trait can be any of those traits described above. Additionally, to confirm that the modified trait is due to changes in expression levels or activity of the polypeptide or polynucleotide of the invention can be determined by analyzing mRNA expression using Northern blots, RT-PCR or microarrays, or protein expression using immunoblots or Western blots or gel shift assays.

Integrated Systems—Sequence Identity

Additionally, the present invention may be an integrated system, computer or computer readable medium that comprises an instruction set for determining the identity of one or more sequences in a database. In addition, the instruction set can be used to generate or identify sequences that meet any specified criteria. Furthermore, the instruction set may be used to associate or link certain functional benefits, such improved characteristics, with one or more identified sequence.

For example, the instruction set can include, e.g., a sequence comparison or other alignment program, e.g., an available program such as, for example, the Wisconsin Package Version 10.0, such as BLAST, FASTA, PILEUP, FINDPATTERNS or the like (GCG, Madison, Wis.). Public sequence databases such as GenBank, EMBL, Swiss-Prot and PIR or private sequence databases such as PHYTOSEQ sequence database (Incyte Genomics, Palo Alto, Calif.) can be searched.

Alignment of sequences for comparison can be conducted by the local homology algorithm of Smith and Waterman (1981) Adv. Appl. Math. 2: 482-489, by the homology alignment algorithm of Needleman and Wunsch (1970) J. Mol. Biol. 48: 443-453, by the search for similarity method of Pearson and Lipman (1988) Proc. Natl. Acad. Sci. 85: 2444-2448, by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window can be a segment of at least about 20 contiguous positions, usually about 50 to about 200, more usually about 100 to about 150 contiguous positions. A description of the method is provided in Ausubel et al. supra.

A variety of methods for determining sequence relationships can be used, including manual alignment and computer assisted sequence alignment and analysis. This later approach is a preferred approach in the present invention, due to the increased throughput afforded by computer assisted methods. As noted above, a variety of computer programs for performing sequence alignment are available, or can be produced by one of skill.

One example algorithm that is suitable for determining percent sequence identity and sequence similarity is the BLAST algorithm, which is described in Altschul et al. (1990) J. Mol. Biol. 215: 403-410. Software for performing BLAST analyses is publicly available, e.g., through the National Library of Medicine's National Center for Biotechnology Information (ncbi.nlm.nih; see at world wide web (www) National Institutes of Health US government (gov) website). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1992) Proc. Natl. Acad. Sci. 89: 10915-10919). Unless otherwise indicated, “sequence identity” here refers to the % sequence identity generated from a tblastx using the NCBI version of the algorithm at the default settings using gapped alignments with the filter “off” (see, for example, NIH NLM NCBI website at ncbi.nlm.nih, supra).

In addition to calculating percent sequence identity, the BLAST algorithm also performs a statistical analysis of the similarity between two sequences (see, e.g. Karlin and Altschul (1993) Proc. Natl. Acad. Sci. 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001. An additional example of a useful sequence alignment algorithm is PILEUP. PILEUP creates a multiple sequence alignment from a group of related sequences using progressive, pairwise alignments. The program can align, e.g., up to 300 sequences of a maximum length of 5,000 letters.

The integrated system, or computer typically includes a user input interface allowing a user to selectively view one or more sequence records corresponding to the one or more character strings, as well as an instruction set which aligns the one or more character strings with each other or with an additional character string to identify one or more region of sequence similarity. The system may include a link of one or more character strings with a particular phenotype or gene function. Typically, the system includes a user readable output element that displays an alignment produced by the alignment instruction set.

The methods of this invention can be implemented in a localized or distributed computing environment. In a distributed environment, the methods may implemented on a single computer comprising multiple processors or on a multiplicity of computers. The computers can be linked, e.g. through a common bus, but more preferably the computer(s) are nodes on a network. The network can be a generalized or a dedicated local or wide-area network and, in certain preferred embodiments, the computers may be components of an intra-net or an internet.

Thus, the invention provides methods for identifying a sequence similar or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an inter or intra net) and a query is made against the sequence database using the relevant sequences herein and associated plant phenotypes or gene functions.

Any sequence herein can be entered into the database, before or after querying the database. This provides for both expansion of the database and, if done before the querying step, for insertion of control sequences into the database. The control sequences can be detected by the query to ensure the general integrity of both the database and the query. As noted, the query can be performed using a web browser based interface. For example, the database can be a centralized public database such as those noted herein, and the querying can be done from a remote terminal or computer across an internet or intranet.

Any sequence herein can be used to identify a similar, homologous, paralogous, or orthologous sequence in another plant. This provides means for identifying endogenous sequences in other plants that may be useful to alter a trait of progeny plants, which results from crossing two plants of different strain. For example, sequences that encode an ortholog of any of the sequences herein that naturally occur in a plant with a desired trait can be identified using the sequences disclosed herein. The plant is then crossed with a second plant of the same species but which does not have the desired trait to produce progeny which can then be used in further crossing experiments to produce the desired trait in the second plant. Therefore the resulting progeny plant contains no transgenes; expression of the endogenous sequence may also be regulated by treatment with a particular chemical or other means, such as EMR. Some examples of such compounds well known in the art include: ethylene; cytokinins; phenolic compounds, which stimulate the transcription of the gene needed for infection; specific monosaccharides and acidic environments which potentiate vir gene induction; acidic polysaccharides which induce one or more chromosomal genes; and opines; other mechanisms include light or dark treatment (for a review of examples of such treatments, see, Winans (1992) Microbiol. Rev. 56: 12-31; Eyal et al. (1992) Plant Mol. Biol. 19: 589-599; Chrispeels et al. (2000) Plant Mol. Biol. 42: 279-290; Piazza et al. (2002) Plant Physiol. 128: 1077-1086).

Table 6 lists sequences within the UniGene database determined to be orthologous to a number of transcription factor sequences of the present invention. The column headings include the transcription factors listed by (a) the Clade Identifier (the Reference Arabidopsis sequence used to identify each clade); (b) the SEQ ID NO: of each Clade Identifier; (c) the AGI Identifier for each Clade Identifier; (d) the UniGene identifier for each orthologous sequence identified in this study; (e) the species from which the orthologs to the transcription factors are derived;; and (f) the smallest sum probability relationship of the homologous sequence to Arabidopsis Clade Identifier sequence in a given row, determined by BLAST analysis.

TABLE 6
Orthologs of Representative Arabidopsis Transcription Factor Genes Identified Using BLAST

| Clade | Clade | | | | | |
|---|---|---|---|---|---|---|
| Identifier | Identifier | | | | | |
| (Arabidopsis | SEQ ID | AGI Identifier for | | Ortholog | | |
| GID) | NO: | Clade Identifier | UniGene Identifier | SEQ ID NO: | Species | p-Value |
| 223 | G175 | AT4G26440 | Les_S5295446 | 464 | Lycopersicon | 1.00E-174 |
| | | | | | esculentum | |
| 223 | G175 | AT4G26440 | Os_S121030 | 421 | Oryza sativa | 2.00E-77 |
| 223 | G175 | AT4G26440 | SGN-UNIGENE- | 468 | Lycopersicon | 1.00E-75 |
| | | | 57877 | | esculentum | |
| 223 | G175 | AT4G26440 | Zm_S11524014 | 450 | Zea mays | 9.00E-50 |
| 223 | G175 | AT4G26440 | SGN-UNIGENE- | 467 | Lycopersicon | 7.00E-40 |
| | | | 52888 | | esculentum | |
| 223 | G175 | AT4G26440 | SGN-UNIGENE- | 466 | Lycopersicon | 6.00E-36 |
| | | | 50193 | esculentum | | |
| 223 | G175 | AT4G26440 | Os_50781 | 422 | Oryza sativa | 3.00E-19 |
| 255 | G184 | AT4G22070 | SGN-UNIGENE- | 474 | Lycopersicon | 1.00E-104 |
| | | | 47543 | | esculentum | |
| 255 | G184 | AT4G22070 | SGN-UNIGENE- | 473 | Lycopersicon | 1.00E-100 |
| | | | 47034 | | esculentum | |
| 255 | G184 | AT4G22070 | Gma_S6668474 | 435 | Glycine max | 2.00E-77 |
| 255 | G184 | AT4G22070 | SGN-UNIGENE- | 476 | Lycopersicon | 2.00E-71 |
| | | | SINGLET-18500 | | esculentum | |
| 255 | G184 | AT4G22070 | SGN-UNIGENE- | 477 | Lycopersicon | 5.00E-50 |
| | | | SINGLET-1941 | | esculentum | |
| 255 | G184 | AT4G22070 | SGN-UNIGENE- | 478 | Lycopersicon | 8.00E-37 |
| | | | SINGLET-20683 | | esculentum | |
| 255 | G184 | AT4G22070 | SGN-UNIGENE- | 475 | Lycopersicon | 5.00E-24 |
| | | | 52279 | | esculentum | |
| 255 | G184 | AT4G22070 | Gma_S4878547 | 434 | Glycine max | 2.00E-12 |
| 255 | G184 | AT4G22070 | SGN-UNIGENE- | 494 | Lycopersicon | 2.00E-11 |
| | | | SINGLET-2301 | | esculentum | |
| 255 | G184 | AT4G22070 | Hv_S119532 | 444 | Hordeum vulgare | 2.00E-10 |
| 255 | G184 | AT4G22070 | Zm_S11388469 | 452 | Zea mays | 2.00E-06 |
| 257 | G186 | AT1G62300 | SGN-UNIGENE- | 474 | Lycopersicon | 1.00E-104 |
| | | | 47543 | | esculentum | |
| 257 | G186 | AT1G62300 | SGN-UNIGENE- | 473 | Lycopersicon | 1.00E-100 |
| | | | 47034 | | esculentum | |
| 257 | G186 | AT1G62300 | Gma_S6668474 | 435 | Glycine max | 2.00E-77 |
| 257 | G186 | AT1G62300 | SGN-UNIGENE- | 476 | Lycopersicon | 2.00E-71 |
| | | | SINGLET-18500 | | esculentum | |
| 257 | G186 | AT1G62300 | SGN-UNIGENE- | 477 | Lycopersicon | 5.00E-50 |
| | | | SINGLET-1941 | | esculentum | |
| 257 | G186 | AT1G62300 | SGN-UNIGENE- | 478 | Lycopersicon | 8.00E-37 |
| | | | SINGLET-20683 | | esculentum | |
| 257 | G186 | AT1G62300 | SGN-UNIGENE- | 475 | Lycopersicon | 5.00E-24 |
| | | | 52279 | | esculentum | |
| 257 | G186 | AT1G62300 | Gma_S4878547 | 434 | Glycine max | 2.00E-12 |
| 257 | G186 | AT1G62300 | SGN-UNIGENE- | 494 | Lycopersicon | 2.00E-11 |
| | | | SINGLET-2301 | | esculentum | |
| 257 | G186 | AT1G62300 | Hv_S119532 | 444 | Hordeum vulgare | 2.00E-10 |
| 257 | G186 | AT1G62300 | Zm_S11388469 | 452 | Zea mays | 2.00E-06 |
| 259 | G353 | AT5G59820 | SGN-UNIGENE- | 470 | Lycopersicon | 6.00E-32 |
| | | | 56766 | | esculentum | |
| 259 | G353 | AT5G59820 | Gma_S4898433 | 431 | Glycine max | 3.00E-26 |
| 259 | G353 | AT5G59820 | Ta_S200273 | 456 | Triticum aestivum | 1.00E-24 |
| 259 | G353 | AT5G59820 | Os_S109163 | 423 | Oryza sativa | 2.00E-20 |
| 259 | G353 | AT5G59820 | Gma_S4973977 | 432 | Glycine max | 9.00E-17 |
| 259 | G353 | AT5G59820 | Ta_S111267 | 455 | Triticum aestivum | 3.00E-16 |
| 259 | G353 | AT5G59820 | Mtr_S5397852 | 439 | Medicago | 2.00E-14 |
| | | | | | truncatula | |
| 259 | G353 | AT5G59820 | Hv_S207187 | 443 | Hordeum vulgare | 5.00E-10 |
| 259 | G353 | AT5G59820 | Ta_S296415 | 457 | Triticum aestivum | 1.00E-05 |
| 227 | G354 | AT3G46090 | SGN-UNIGENE- | 470 | Lycopersicon | 6.00E-32 |
| | | | 56766 | | esculentum | |
| 227 | G354 | AT3G46090 | Gma_S4898433 | 431 | Glycine max | 3.00E-26 |
| 227 | G354 | AT3G46090 | Ta_S200273 | 456 | Triticum aestivum | 1 .00E-24 |
| 227 | G354 | AT3G46090 | Os_S109163 | 423 | Oryza sativa | 2.00E-20 |
| 227 | G354 | AT3G46090 | Gma_S4973977 | 432 | Glycine max | 9.00E-17 |
| 227 | G354 | AT3G46090 | Ta_S111267 | 455 | Triticum aestivum | 3.00E-16 |
| 227 | G354 | AT3G46090 | Mtr_S5397852 | 439 | Medicago | 2.00E-14 |
| | | | | | truncatula | |
| 227 | G354 | AT3G46090 | Hv_S207187 | 443 | Hordeum vulgare | 5.00E-10 |
| 227 | G354 | AT3G46090 | Ta_S296415 | 457 | Triticum aestivum | 1.00E-05 |
| 229 | G489 | AT1G08970 | Vvi_S16526885 | 498 | Vitis vinifera | 1.00E-77 |
| 229 | G489 | AT1G08970 | SGN-UNIGENE- | 471 | Lycopersicon | 4.00E-75 |
| | | | 45265 | | esculentum | |
| 229 | G489 | AT1G08970 | Mtr_S5463839 | 440 | Medicago | 6.00E-73 |
| | | | | | truncatula | |
| 229 | G489 | AT1G08970 | Les_S5293479 | 465 | Lycopersicon | 2.00E-69 |
| | | | | | esculentum | |
| 229 | G489 | AT1G08970 | Mtr_57092400 | 441 | Medicago | 9.00E-66 |
| | | | | | truncatula | |
| 229 | G489 | AT1G08970 | Pta_517047341 | 505 | Pinus taeda | 7.00E-48 |
| 229 | G489 | AT1G08970 | SGN-UNIGENE- | 472 | Lycopersicon | 2.00E-36 |
| | | | 45266 | | esculentum | |
| 229 | G489 | AT1G08970 | Os_S37232 | 424 | Oryza sativa | 5.00E-09 |
| 229 | G489 | AT1G08970 | Vvi_515374122 | 497 | Vitis vinifera | 2.00E-08 |
| 263 | G596 | AT2G45430 | Pta_S16786360 | 508 | Pinus taeda | 2.00E-70 |
| 263 | G596 | AT2G45430 | Gma_S4935598 | 436 | Glycine max | 2.00E-67 |
| 263 | G596 | AT2G45430 | Pta_S16788492 | 509 | Pinus taeda | 7.00E-63 |
| 263 | G596 | AT2G45430 | Pta_S16802054 | 510 | Pinus taeda | 1.00E-57 |
| 263 | G596 | AT2G45430 | Pta S15799222 | 507 | Pinus taeda | 6.00E-43 |
| 231 | G634 | AT1G33240 | Pta_S17050439 | 506 | Pinus taeda | 3.00E-39 |
| 231 | G634 | AT1G33240 | Zm_S11449298 | 451 | Zea mays | 3.00E-35 |
| 233 | G682 | AT4G01060 | Vvi_S15356289 | 499 | Vitis vinifera | 2.00E-30 |
| 233 | G682 | AT4G01060 | Ta_S45274 | 458 | Triticum aestivum | 3.00E-14 |
| 233 | G682 | AT4G01060 | Vvi_S16820566 | 500 | Vitis vinifera | 3.00E-12 |
| 233 | G682 | AT4G01060 | Gma_S4901946 | 433 | Glycine max | 0.004 |
| 265 | G714 | AT1G54830 | Vvi_S16526885 | 498 | Vitis vinifera | 1.00E-77 |
| 265 | G714 | AT1G54830 | SGN-UNIGENE- | 471 | Lycopersicon | 4.00E-75 |
| | | | 45265 | | esculentum | |
| 265 | G714 | AT1G54830 | Mtr_S5463839 | 440 | Medicago | 6.00E-73 |
| | | | | | truncatula | |
| 265 | G714 | AT1G54830 | Les_S5293479 | 465 | Lycopersicon | 2.00E-69 |
| | | | | | | esculentum |
| 265 | G714 | AT1G54830 | Mtr_57092400 | 441 | Medicago | 9.00E-66 |
| | | | | | truncatula | |
| 265 | G714 | AT1G54830 | Pta_517047341 | 505 | Pinus taeda | 7.00E-48 |
| 265 | G714 | AT1G54830 | SGN-UNIGENE- | 472 | Lycopersicon | 2.00E-36 |
| | | | 45266 | esculentum | | |
| 265 | G714 | AT1G54830 | Os_S37232 | 424 | Oryza sativa | 5.00E-09 |
| 267 | G877 | AT5G56270 | Les_S5295446 | 464 | Lycopersicon | 1.00E-174 |
| | | | | | esculentum | |
| 267 | G877 | AT5G56270 | Os_S121030 | 421 | Oryza sativa | 2.00E-77 |
| 267 | G877 | AT5G56270 | SGN-UNIGENE- | 468 | Lycopersicon | 1.00E-75 |
| | | | 57877 | | esculentum | |
| 267 | G877 | AT5G56270 | Zm_S11524014 | 450 | Zea mays | 9.00E-50 |
| 267 | G877 | AT5G56270 | SGN-UNIGENE- | 467 | Lycopersicon | 7.00E-40 |
| | | | 52888 | | esculentum | |
| 267 | G877 | AT5G56270 | SGN-UNIGENE- | 466 | Lycopersicon | 6.00E-36 |
| | | | 50193 | | esculentum | |
| 267 | G877 | AT5G56270 | Os_S50781 | 422 | Oryza sativa | 3.00E-19 |
| 267 | G877 | AT5G56270 | SGN-UNIGENE- | 496 | Lycopersicon | 7.00E-10 |
| | | | 56707 | | esculentum | |
| 235 | G916 | AT4G04450 | SGN-UNIGENE- | 474 | Lycopersicon | 1.00E-104 |
| | | | 47543 | | esculenturn | |
| 235 | G916 | AT4G04450 | SGN-UNIGENE- | 473 | Lycopersicon | 1.00E-100 |
| | | | 47034 | | esculentum | |
| 235 | G916 | AT4G04450 | Gma_S6668474 | 435 | Glycine max | 2.00E-77 |
| 235 | G916 | AT4G04450 | SGN-UNIGENE- | 476 | Lycopersicon | 2.00E-71 |
| | | | SINGLET-18500 | | esculentum | |
| 235 | G916 | AT4G04450 | SGN-UNIGENE- | 477 | Lycopersicon | 5.00E-50 |
| | | | SINGLET-1941 | | esculentum | |
| 235 | G916 | AT4G04450 | SGN-UNIGENE- | 478 | Lycopersicon | 8.00E-37 |
| | | | SINGLET-20683 | | esculentum | |
| 235 | G916 | AT4G04450 | SGN-UNIGENE- | 475 | Lycopersicon | 5.00E-24 |
| | | | 52279 | | esculentum | |
| 235 | G916 | AT4G04450 | Gma_S4878547 | 434 | Glycine max | 2.00E-12 |
| 235 | G916 | AT4G04450 | Hv_S119532 | 444 | Hordeum vulgare | 2.00E-10 |
| 235 | G916 | AT4G04450 | Zm_S11388469 | 452 | Zea mays | 2.00E-06 |
| 237 | G975 | AT1G15360 | SGN-UNIGENE- | 482 | Lycopersicon | 9.00E-59 |
| | | | SINGLET-335836 | | esculentum | |
| 237 | G975 | AT1G15360 | SGN-UNIGENE- | 480 | Lycopersicon | 2.00E-52 |
| | | | SiNGLET-14957 | | esculentum | |
| 239 | G1069 | AT4G14465 | SGN-UNIGENE- | 483 | Lycopersicon | 6.00E-55 |
| | | | 59076 | | esculentum | |
| 239 | G1069 | AT4G14465 | Vvi_S16805621 | 501 | Vitis vinifera | 1.00E-04 |
| 271 | G1387 | AT5G25390 | SGN-UNIGENE- | 482 | Lycopersicon | 9.00E-59 |
| | | | SINGLET-335836 | | esculentum | |
| 271 | G1387 | AT5G25390 | SGN-UNIGENE- | 480 | Lycopersicon | 2.00E-52 |
| | | | SINGLET-14957 | | esculentum | |
| 273 | G1634 | AT5G05790 | Vvi_S16872328 | 502 | Vitis vinifera | 4.00E-63 |
| 273 | G1634 | AT5G05790 | SGN-UNIGENE- | 486 | Lycopersicon | 5.00E-34 |
| | | | SINGLET-48341 | | esculentum | |
| 273 | G1634 | AT5G05790 | SGN-UNIGENE- | 485 | Lycopersicon | 4.00E-12 |
| | | | SINGLET-41892 | | esculentum | |
| 275 | G1889 | AT2G28710 | SGN-UNIGENE- | 470 | Lycopersicon | 6.00E-32 |
| | | | 56766 | | esculentum | |
| 275 | G1889 | AT2G28710 | Gma_S4898433 | 431 | Glycine max | 3.00E-26 |
| 275 | G1889 | AT2G28710 | Ta_S200273 | 456 | Triticum aestivum | 1.00E-24 |
| 275 | G1889 | AT2G28710 | Os_S109163 | 423 | Oryza sativa | 2.00E-20 |
| 275 | G1889 | AT2G28710 | Gma_S4973977 | 432 | Glycine max | 9.00E-17 |
| 275 | G1889 | AT2G28710 | Ta_S111267 | 455 | Triticum aestivum | 3.00E-16 |
| 275 | G1889 | AT2G28710 | Mtr_S5397852 | 439 | Medicago | 2.00E-14 |
| | | | | | truncatula | |
| 275 | G1889 | AT2G28710 | Hv_S207187 | 443 | Hordeum vulgare | 5.00E-10 |
| 277 | G1940 | AT5G54900 | SGN-UNIGENE- | 487 | Lycopersicon | 1.00E-144 |
| | | | 44207 | | esculentum | |
| 277 | G1940 | AT5G54900 | Zm_S11525357 | 454 | Zea mays | 1.00E-130 |
| 277 | G1940 | AT5G54900 | Zm_S11522955 | 453 | Zea mays | 1.00E-100 |
| 277 | G1940 | AT5GS4900 | Vvi_S16865171 | 503 | Vitis vinifera | 1.00E-85 |
| 277 | G1940 | AT5G54900 | Hv_S153237 | 446 | Hordeum vulgare | 9.00E-72 |
| 277 | G1940 | AT5G54900 | Ta_S152820 | 461 | Triticum aestivum | 1.00E-66 |
| 277 | G1940 | AT5G54900 | SGN-UNIGENE- | 491 | Lycopersicon | 3.00E-55 |
| | | | SINGLET-396174 | | esculentum | |
| 277 | G1940 | AT5G54900 | SGN-UNIGENE- | 490 | Lycopersicon | 4.00E-53 |
| | | | SINGLET-333119 | | esculentum | |
| 277 | G1940 | AT5GS4900 | Gma_S4975207 | 437 | Glycine max | 6.00E-51 |
| 277 | G1940 | AT5GS4900 | SGN-UNIGENE- | 489 | Lycopersicon | 1.00E-51 |
| | | | SiNGLET-17539 | | esculentum | |
| 277 | G1940 | AT5G54900 | Hv_S63965 | 447 | Hordeum vulgare | 400.E-43 |
| 277 | G1940 | AT5G54900 | SGN-UNIGENE | 448 | Lycopersicon | 200.E-43 |
| | | | 56600 | | esculentum | |
| 277 | G1940 | AT5GS4900 | Os_S32676 | 426 | Oryza sativa | 2.00E-31 |
| 277 | G1940 | AT5G54900 | Ta_S125786 | 460 | Triticum aestivum | 6.00E-26 |
| 277 | G1940 | AT5G54900 | Ta_S267457 | 462 | Triticum aestivum | 5.00E-24 |
| 277 | G1940 | AT5G54900 | Vvi_S16866336 | 504 | Vitis vinifera | 7.00E-18 |
| 277 | G1940 | AT5G54900 | Os_S75860 | 427 | Oryza sativa | 4.00E-11 |
| 277 | G1940 | AT5G54900 | SGN-UNIGENE- | 492 | Lycopersicon | 2.00E-04 |
| | | | SINGLET-49629 | | esculentum | |
| 279 | G1974 | AT3G46070 | SGN-UNIGENE- | 470 | Lycopersicon | 6.00E-32 |
| | | | 56766 | | esculentum | |
| 279 | G1974 | AT3G46070 | Gma_S4898433 | 431 | Glycine max | 3.00E-26 |
| 279 | G1974 | AT3G46070 | Ta_S200273 | 456 | Triticum aestivum | 1.00E-24 |
| 279 | G1974 | AT3G46070 | Os_S109163 | 423 | Oryza sativa | 2.00E-20 |
| 279 | G1974 | AT3G46070 | Gma_S4973977 | 432 | Glycine max | 9.00E-17 |
| 279 | G1974 | AT3G46070 | Ta_S111267 | 455 | Triticum aestivum | 3.00E-16 |
| 279 | G1974 | AT3G46070 | Mtr_S5397852 | 439 | Medicago | 2.00E-14 |
| | | | | | truncatula | |
| 279 | G1974 | AT3G46070 | Hv_S207187 | 443 | Hordeum vulgare | 5.00E-1O |
| 279 | G1974 | AT3G46070 | Ta_S296415 | 457 | Triticum aestivum | 1.00E-05 |
| 281 | G2153 | AT3G04570 | SGN-UNIGENE- | 483 | Lycopersicon | 6.00E-55 |
| | | | 59076 | | esculentum | |
| 281 | G2153 | AT3G04570 | Mtr_S5308977 | 442 | Medicago | 2.00E-31 |
| | | | | | truncatula | |
| 281 | G2153 | AT3G04570 | Hv_S52928 | 449 | Hordeum vulgare | 5 |
| 283 | G2583 | AT5G11190 | SGN-UNIGENE- | 482 | Lycopersicon | 9.00E-59 |
| | | | SINGLET-335836 | | esculentum | |
| 283 | G2583 | AT5G11190 | SGN-UNIGENE- | 480 | Lycopersicon | 2.00E-52 |
| | | | SINGLET-14957 | | esculentum | |
| 245 | G2701 | AT3G11280 | Vvi_S16872328 | 502 | Vitis vinifera | 4.00E-63 |
| 245 | G2701 | AT3G11280 | SGN-UNIGENE- | 486 | Lycopersicon | 5.00E-34 |
| | | | SINGLET-48341 | | esculentum | |
| 245 | G2701 | AT3G11280 | SGN-UNIGENE- | 485 | Lycopersicon | 4.00E-12 |
| | | | SINGLET-41892 | | esculentum | |
| 247 | G2789 | AT3G60870 | Pta_S16786360 | 508 | Pinus taeda | 2.00E-70 |
| 247 | G2789 | AT3G60870 | Gma_S4935598 | 436 | Glycine max | 2.00E-67 |
| 247 | G2789 | AT3G60870 | Pta_S16788492 | 509 | Pinus taeda | 7.00E-63 |
| 247 | G2789 | AT3G60870 | Pta_S16802054 | 510 | Pinustaeda | 1.00E-57 |
| 247 | G2789 | AT3G60870 | Pta_S15799222 | 507 | Pinus taeda | 6.00E-43 |
| 249 | G2839 | AT3G46080 | SGN-UNIGENE- | 470 | Lycopersicon | 6.00E-32 |
| | | | 56766 | | esculentum | |
| 249 | G2839 | AT3G46080 | Gma_S4898433 | 431 | Glycine max | 3.00E-26 |
| 249 | G2839 | AT3G46080 | Ta_S200273 | 456 | Triticum aestivum | 1.00E-24 |
| 249 | G2839 | AT3G46080 | Os_S109163 | 423 | Oryza sativa | 2.00E-20 |
| 249 | G2839 | AT3G46080 | Gma_S4973977 | 432 | Glycine max | 9.00E-17 |
| 249 | G2839 | AT3G46080 | Ta_S111267 | 455 | Triticum aestivum | 3.00E-16 |
| 249 | G2839 | AT3G46080 | Mtr_S5397852 | 439 | Medicago | 2.00E-14 |
| | | | | | truncatula | |
| 249 | G2839 | AT3G46080 | Hv_S207187 | 443 | Hordeum vulgare | 5.00E-10 |
| 249 | G2839 | AT3G46080 | Ta_S296415 | 457 | Triticum aestivum | 1.00E-05 |
| 251 | G2854 | AT4G27000 | SGN-UNIGENE- | 487 | Lycopersicon | 1.00E-144 |
| | | | 44207 | | esculentum | |
| 251 | G2854 | AT4G27000 | Zm_S11525357 | 454 | Zea mays | 1.00E-130 |
| 251 | G2854 | AT4G27000 | Zm_S11522955 | 453 | Zea mays | 1.00E-100 |
| 251 | G2854 | AT4G27000 | Vvi_S1686S171 | 503 | Vitis vinifera | 1.00E-85 |
| 251 | G2854 | AT4G27000 | Hv_S153237 | 446 | Hordeum vulgare | 9.00E-72 |
| 251 | G2854 | AT4G27000 | Ta_5152820 | 461 | Triticum aestivum | 1.00E-66 |
| 251 | G2854 | AT4G27000 | SGN-UNIGENE- | 491 | Lycopersicon | 3.00E-55 |
| | | | SINGLET-396174 | | esculentum | |
| 251 | G2854 | AT4G27000 | SGN-UNIGENE- | 490 | Lycopersicon | 4.00E-53 |
| | | | SINGLET-333119 | | esculentum | |
| 251 | G2854 | AT4G27000 | Gma_S4975207 | 437 | Glycine max | 6.00E-51 |
| 251 | G2854 | AT4G27000 | SGN-UNIGENE- | 489 | Lycopersicon | 1 .00E-5 1 |
| | | | SINGLET-17539 | | esculentum | |
| 251 | G2854 | AT4G27000 | Hv_S63965 | 447 | Hordeum vulgare | 4.00E-43 |
| 251 | G2854 | AT4G27000 | SGN-UNIGENE- | 488 | Lycopersicon | 2.00E-43 |
| | | | 56600 | | esculentum | |
| 251 | G2854 | AT4G27000 | Os_S32676 | 426 | Oryza sativa | 2.00E-31 |
| 251 | G2854 | AT4G27000 | Ta_S125786 | 460 | Triticum aestivum | 6.00E-26 |
| 251 | G2854 | AT4G27000 | Ta S267457 | 462 | Triticum aestivum | 5.00E-24 |
| 251 | G2854 | AT4G27000 | Vvi_516866336 | 504 | Vitis vinifera | 7.00E-18 |
| 251 | G2854 | AT4G27000 | Os_S75860 | 427 | Oryza sativa | 4.00E-1 1 |
| 251 | G2854 | AT4G27000 | SGN-UNIGENE- | 492 | Lycopersicon | 2.00E-04 |
| | | | SINGLET-49629 | | esculentum | |
| 253 | G3083 | AT3G14880 | Gma_S4880456 | 438 | Glycine max | 1.00E-25 |
| 253 | G3083 | AT3G14880 | Ta_S179586 | 463 | Triticum aestivum | 1.00E-13 |
| 253 | G3083 | AT3G14880 | Os_S54214 | 428 | Oryza sativa | 5.00E-08 |
| 253 | G3083 | AT3G14880 | Hv_S60182 | 448 | Hordeum vulgare | 3.00E-06 |

Table 7 lists the gene identification number (GID) and homologous relationships found using analyses according to the Examples for the sequences of the Sequence Listing. Those sequences listed as “reference sequences” were originally determined by experimentation to confer drought tolerance when their expression was altered. Generally, each reference sequence was used to identify the clade in which functionally related homologous sequences may be found.

TABLE 7
Homologs and Other Related Genes of Representative Arabidopsis
Transcription Factor Genes Identified using BLAST

| | | Polynucleotide | Species from Which | |
|---|---|---|---|---|
| | | (DNA) or | Homologous Sequence is | Relationship of SEQ ID NO: to |
| SEQ ID NO: | GID No: | polypeptide (PRT) | Derived | Other Genes |
| 1 | G47 | DNA | Arabidopsis thaliana | Reference sequence; predicted |
| | | | | polypeptide sequence is |
| | | | | paralogous to G2133 |
| 2 | G47 | PRT | Arabidopsis thaliana | Reference sequence; |
| | | | | paralogous to G2133 |
| 3 | G922 | DNA | Arabidopsis thaliana | Reference sequence |
| 4 | G922 | PRT | Arabidopsis thaliana | Reference sequence |
| 5 | G1274 | DNA | Arabidopsis thaliana | Reference sequence |
| 6 | G1274 | PRT | Arabidopsis thaliana | Reference sequence |
| 7 | G1792 | DNA | Arabidopsis thaliana | Reference sequence |
| 8 | G1792 | PRT | Arabidopsis thaliana | Reference sequence |
| 9 | G2053 | DNA | Arabidopsis thaliana | Reference sequence |
| 10 | G2053 | PRT | Arabidopsis thaliana | Reference sequence |
| 11 | G2133 | DNA | Arabidopsis thaliana | Reference sequence; predicted |
| | | | | polypeptide sequence is |
| | | | | paralogous to G47 |
| 12 | G2133 | PRT | Arabidopsis thaliana | Reference sequence; |
| | | | | paralogous to G47 |
| 13 | G2999 | DNA | Arabidopsis thaliana | Reference sequence |
| 14 | G2999 | PRT | Arabidopsis thaliana | Reference sequence |
| 15 | G3086 | DNA | Arabidopsis thaliana | Reference sequence |
| 16 | G3086 | PRT | Arabidopsis thaliana | Reference sequence |
| 17 | G30 | DNA | Arabidopsis thaliana | Predicted polypeptide sequence |
| | | | | is paralogous to G1792 |
| 18 | G30 | PRT | Arabidopsis thaliana | Paralogous to G1792 |
| 19 | G515 | DNA | Arabidopsis thaliana | Predicted polypeptide sequence |
| | | | | is paralogous to G2053 |
| 20 | G515 | PRT | Arabidopsis thaliana | Paralogous to G2053 |
| 21 | G516 | DNA | Arabidopsis thaliana | Predicted polypeptide sequence |
| | | | | is paralogous to G2053 |
| 22 | G516 | PRT | Arabidopsis thaliana | Paralogous to G2053 |
| 23 | G517 | DNA | Arabidopsis thaliana | Predicted polypeptide sequence |
| | | | | is paralogous to G2053 |
| 24 | G517 | PRT | Arabidopsis thaliana | Paralogous to G2053 |
| 25 | G592 | DNA | Arabidopsis thaliana | Predicted polypeptide sequence |
| | | | | is paralogous to G3086 |
| 26 | G592 | PRT | Arabidopsis thaliana | Paralogous to G3086 |
| 27 | G1134 | DNA | Arabidopsis thaliana | Predicted polypeptide sequence |
| | | | | is paralogous to G3086 |
| 28 | G1134 | PRT | Arabidopsis thaliana | Paralogous to G3086 |
| 29 | G1275 | DNA | Arabidopsis thaliana | Predicted polypeptide sequence |
| | | | | is paralogous to G1274 |
| 30 | G1275 | PRT | Arabidopsis thaliana | Paralogous to G1274 |
| 31 | G1758 | DNA | Arabidopsis thaliana | Predicted polypeptide sequence |
| | | | | is paralogous to G1274 |
| 32 | G1758 | PRT | Arabidopsis thaliana | Paralogous to G1274 |
| 33 | G1791 | DNA | Arabidopsis thaliana | Predicted polypeptide sequence |
| | | | | is paralogous to G1792 |
| 34 | G1791 | PRT | Arabidopsis thaliana | Paralogous to G1792 |
| 35 | G1795 | DNA | Arabidopsis thaliana | Predicted polypeptide sequence |
| | | | | is paralogous to G1792 |
| 36 | G1795 | PRT | Arabidopsis thaliana | Paralogous to G1792 |
| 37 | G2149 | DNA | Arabidopsis thaliana | Predicted polypeptide sequence |
| | | | | is paralogous to G3086 |
| 38 | G2149 | PRT | Arabidopsis thaliana | Paralogous to G3086 |
| 39 | G2555 | DNA | Arabidopsis thaliana | Predicted polypeptide sequence |
| | | | | is paralogous to G3086 |
| 40 | G2555 | PRT | Arabidopsis thaliana | Paralogous to G3086 |
| 41 | G2766 | DNA | Arabidopsis thaliana | Predicted polypeptide sequence |
| | | | | is paralogous to G3086 |
| 42 | G2766 | PRT | Arabidopsis thaliana | Paralogous to G3086 |
| 43 | G2989 | DNA | Arabidopsis thaliana | Predicted polypeptide sequence |
| | | | | is paralogous to G2999 |
| 44 | G2989 | PRT | Arabidopsis thaliana | Paralogous to G2999 |
| 45 | G2990 | DNA | Arabidopsis thaliana | Predicted polypeptide sequence |
| | | | | is paralogous to G2999 |
| 46 | G2990 | PRT | Arabidopsis thaliana | Paralogous to G2999 |
| 47 | G2991 | DNA | Arabidopsis thaliana | Predicted polypeptide sequence |
| | | | | is paralogous to G2999 |
| 48 | G2991 | PRT | Arabidopsis thaliana | Paralogous to G2999 |
| 49 | G2992 | DNA | Arabidopsis thaliana | Reference sequence; predicted |
| | | | | polypeptide sequence is |
| | | | | paralogous to G2999 |
| 50 | G2992 | PRT | Arabidopsis thaliana | Reference sequence; |
| | | | | paralogous to G2999 |
| 51 | G2993 | DNA | Arabidopsis thaliana | Predicted polypeptide sequence |
| | | | | is paralogous to G2999 |
| 52 | G2993 | PRT | Arabidopsis thaliana | Paralogous to G2999 |
| 53 | G2994 | DNA | Arabidopsis thaliana | Predicted polypeptide sequence |
| | | | | is paralogous to G2999 |
| 54 | G2994 | PRT | Arabidopsis thaliana | Paralogous to G2999 |
| 55 | G2995 | DNA | Arabidopsis thaliana | Predicted polypeptide sequence |
| | | | | is paralogous to G2999 |
| 56 | G2995 | PRT | Arabidopsis thaliana | Paralogous to G2999 |
| 57 | G2996 | DNA | Arabidopsis thaliana | Predicted polypeptide sequence |
| | | | | is paralogous to G2999 |
| 58 | G2996 | PRT | Arabidopsis thaliana | Paralogous to G2999 |
| 59 | G2997 | DNA | Arabidopsis thaliana | Predicted polypeptide sequence |
| | | | | is paralogous to G2999 |
| 60 | G2997 | PRT | Arabidopsis thaliana | Paralogous to G2999 |
| 61 | G2998 | DNA | Arabidopsis thaliana | Predicted polypeptide sequence |
| | | | | is paralogous to G2999 |
| 62 | G2998 | PRT | Arabidopsis thaliana | Paralogous to G2999 |
| 63 | G3000 | DNA | Arabidopsis thaliana | Predicted polypeptide sequence |
| | | | | is paralogous to G2999 |
| 64 | G3000 | PRT | Arabidopsis thaliana | Paralogous to G2999 |
| 65 | G3001 | DNA | Arabidopsis thaliana | Predicted polypeptide sequence |
| | | | | is paralogous to G2999 |
| 66 | G3001 | PRT | Arabidopsis thaliana | Paralogous to G2999 |
| 67 | G3002 | DNA | Arabidopsis thaliana | Predicted polypeptide sequence |
| | | | | is paralogous to G2999 |
| 68 | G3002 | PRT | Arabidopsis thaliana | Paralogous to G2999 |
| 69 | G3380 | DNA | Oryza sativa (japonica | Predicted polypeptide sequence |
| | | | cultivar-group) | is orthologous to G1792 |
| 70 | G3380 | PRT | Oryza sativa (japonica | Orthologous to G1792 |
| | | | cultivar-group) | |
| 71 | G3381 | DNA | Oryza sativa (japonica | Predicted polypeptide sequence |
| | | | cultivar-group) | is orthologous to G1792 |
| 72 | G3381 | PRT | Oryza sativa (japonica | Orthologous to G1792 |
| | | | cultivar-group) | |
| 73 | G3383 | DNA | Oryza sativa (japonica | Predicted polypeptide sequence |
| | | | cultivar-group) | is orthologous to G1792 |
| 74 | G3383 | PRT | Oryza sativa (japonica | Orthologous to G1792 |
| | | | cultivar-group) | |
| 75 | G3515 | DNA | Oryza sativa (japonica | Predicted polypeptide sequence |
| | | | cultivar-group) | is orthologous to G1792 |
| 76 | G3515 | PRT | Oryza sativa (japonica | Orthologous to G1792 |
| | | | cultivar-group) | |
| 77 | G3516 | DNA | Zea mays | Predicted polypeptide sequence |
| | | | | is orthologous to G1792 |
| 78 | G3516 | PRT | Zea mays | Orthologous to G1792 |
| 79 | G3517 | DNA | Zea mays | Predicted polypeptide sequence |
| | | | | is orthologous to G1792 |
| 80 | G3517 | PRT | Zea mays | Orthologous to G1792 |
| 81 | G3518 | DNA | Glycine max | Predicted polypeptide sequence |
| | | | | is orthologous to G1792 |
| 82 | G3518 | PRT | Glycine max | Orthologous to G1792 |
| 83 | G3519 | DNA | Glycine max | Predicted polypeptide sequence |
| | | | | is orthologous to G1792 |
| 84 | G3519 | PRT | Glycine max | Orthologous to G1792 |
| 85 | G3520 | DNA | Glycine max | Predicted polypeptide sequence |
| | | | | is orthologous to G1792 |
| 86 | G3520 | PRT | Glycine max | Orthologous to G1792 |
| 87 | G3643 | DNA | Glycine max | Predicted polypeptide sequence |
| | | | | is orthologous to G47 |
| 88 | G3643 | PRT | Glycine max | Orthologous to G47 |
| 89 | G3644 | DNA | Oryza sativa (japonica | Predicted polypeptide sequence |
| | | | cultivar-group) | is orthologous to G47 |
| 90 | G3644 | PRT | Oryza sativa (japonica | Orthologous to G47 |
| | | | cultivar-group) | |
| 91 | G3645 | DNA | Brassica rapa subsp. | Predicted polypeptide sequence |
| | | | Pekinensis | is orthologous to G47 |
| 92 | G3645 | PRT | Brassica rapa subsp. | Orthologous to G47 |
| | | | Pekinensis | |
| 93 | G3646 | DNA | Brassica oleracea | Predicted polypeptide sequence |
| | | | | is orthologous to G47 |
| 94 | G3646 | PRT | Brassica oleracea | Orthologous to G47 |
| 95 | G3647 | DNA | Zinnia elegans | Predicted polypeptide sequence |
| | | | | is orthologous to G47 |
| 96 | G3647 | PRT | Zinnia elegans | Orthologous to G47 |
| 97 | G3649 | DNA | Oryza sativa (japonica | Predicted polypeptide sequence |
| | | | cultivar-group) | is orthologous to G47 |
| 98 | G3649 | PRT | Oryza sativa (japonica | Orthologous to G47 |
| | | | cultivar-group) | |
| 99 | G3651 | DNA | Oryza sativa (japonica | Predicted polypeptide sequence |
| | | | cultivar-group) | is orthologous to G47 |
| 100 | G3651 | PRT | Oryza sativa (japonica | Orthologous to G47 |
| | | | cultivar-group) | |
| 101 | G3663 | DNA | Lotus corniculatus var. | Predicted polypeptide sequence |
| | | | japonicus | is orthologous to G2999 |
| 102 | G3663 | PRT | Lotus corniculatus var. | Orthologous to G2999 |
| | | | japonicus | |
| 103 | G3668 | DNA | Flaveria bidentis | Predicted polypeptide sequence |
| | | | | is orthologous to G2999 |
| 104 | G3668 | PRT | Flaveria bidentis | Orthologous to G2999 |
| 105 | G3670 | DNA | Lotus corniculatus var. | Predicted polypeptide sequence |
| | | | japonicus | is orthologous to G2999 |
| 106 | G3670 | PRT | Lotus corniculatus var. | Orthologous to G2999 |
| | | | japonicus | |
| 107 | G3671 | DNA | Oryza sativa (japonica | Predicted polypeptide sequence |
| | | | cultivar-group) | is orthologous to G2999 |
| 108 | G3671 | PRT | Oryza sativa (japonica | Orthologous to G2999 |
| | | | cultivar-group) | |
| 109 | G3674 | DNA | Oryza sativa (indica | Predicted polypeptide sequence |
| | | | cultivar-group) | is orthologous to G2999 |
| 110 | G3674 | PRT | Oryza sativa (indica | Orthologous to G2999 |
| | | | cultivar-group) | |
| 111 | G3675 | DNA | Brassica napus | Predicted polypeptide sequence |
| | | | | is orthologous to G2999 |
| 112 | G3675 | PRT | Brassica napus | Orthologous to G2999 |
| 113 | G3680 | DNA | Zea mays | Predicted polypeptide sequence |
| | | | | is orthologous to G2999 |
| 114 | G3680 | PRT | Zea mays | Orthologous to G2999 |
| 115 | G3683 | DNA | Oryza sativa (japonica | Predicted polypeptide sequence |
| | | | cultivar-group) | is orthologous to G2999 |
| 116 | G3683 | PRT | Oryza sativa (japonica | Orthologous to G2999 |
| | | | cultivar-group) | |
| 117 | G3685 | DNA | Oryza sativa (japonica | Predicted polypeptide sequence |
| | | | cultivar-group) | is orthologous to G2999 |
| 118 | G3685 | PRT | Oryza sativa (japonica | Orthologous to G2999 |
| | | | cultivar-group) | |
| 119 | G3686 | DNA | Oryza sativa (indica | Predicted polypeptide sequence |
| | | | cultivar-group) | is orthologous to G2999 |
| 120 | G3686 | PRT | Oryza sativa (indica | Orthologous to G2999 |
| | | | cultivar-group) | |
| 121 | G3690 | DNA | Oryza sativa (japonica | Predicted polypeptide sequence |
| | | | cultivar-group) | is orthologous to G2999 |
| 122 | G3690 | PRT | Oryza sativa (japonica | Orthologous to G2999 |
| | | | cultivar-group) | |
| 123 | G3692 | DNA | Oryza sativa (japonica | Predicted polypeptide sequence |
| | | | cultivar-group) | is orthologous to G2999 |
| 124 | G3692 | PRT | Oryza sativa (japonica | Orthologous to G2999 |
| | | | cultivar-group) | |
| 125 | G3694 | DNA | Oryza sativa (japonica | Predicted polypeptide sequence |
| | | | cultivar-group) | is orthologous to G2999 |
| 126 | G3694 | PRT | Oryza sativa (japonica | Orthologous to G2999 |
| | | | cultivar-group) | |
| 127 | G3695 | DNA | Oryza sativa (japonica | Predicted polypeptide sequence |
| | | | cultivar-group) | is orthologous to G2999 |
| 128 | G3695 | PRT | Oryza sativa (japonica | Orthologous to G2999 |
| | | | cultivar-group) | |
| 129 | G3719 | DNA | Zea mays | Predicted polypeptide sequence |
| | | | | is orthologous to G1274 |
| 130 | G3719 | PRT | Zea mays | Orthologous to G1274 |
| 131 | G3720 | DNA | Zea mays | Predicted polypeptide sequence |
| | | | | is orthologous to G1274 |
| 132 | G3720 | PRT | Zea mays | Orthologous to G1274 |
| 133 | G3721 | DNA | Oryza sativa (japonica | Predicted polypeptide sequence |
| | | | cultivar-group) | is orthologous to G1274 |
| 134 | G3721 | PRT | Oryza sativa (japonica | Orthologous to G1274 |
| | | | cultivar-group) | |
| 135 | G3722 | DNA | Zea mays | Predicted polypeptide sequence |
| | | | | is orthologous to G1274 |
| 136 | G3722 | PRT | Zea mays | Orthologous to G1274 |
| 137 | G3723 | DNA | Glycine max | Predicted polypeptide sequence |
| | | | | is orthologous to G1274 |
| 138 | G3723 | PRT | Glycine max | Orthologous to G1274 |
| 139 | G3724 | DNA | Glycine max | Predicted polypeptide sequence |
| | | | | is orthologous to G1274 |
| 140 | G3724 | PRT | Glycine max | Orthologous to G1274 |
| 141 | G3725 | DNA | Oryza sativa (japonica | Predicted polypeptide sequence |
| | | | cultivar-group) | is orthologous to G1274 |
| 142 | G3725 | PRT | Oryza sativa (japonica | Orthologous to G1274 |
| | | | cultivar-group) | |
| 143 | G3726 | DNA | Oryza sativa (japonica | Predicted polypeptide sequence |
| | | | cultivar-group) | is orthologous to G1274 |
| 144 | G3726 | PRT | Oryza sativa (japonica | Orthologous to G1274 |
| | | | cultivar-group) | |
| 145 | G3727 | DNA | Zea mays | Predicted polypeptide sequence |
| | | | | is orthologous to G1274 |
| 146 | G3727 | PRT | Zea mays | Orthologous to G1274 |
| 147 | G3728 | DNA | Zea mays | Predicted polypeptide sequence |
| | | | | is orthologous to G1274 |
| 148 | G3728 | PRT | Zea mays | Orthologous to G1274 |
| 149 | G3729 | DNA | Oryza sativa (japonica | Predicted polypeptide sequence |
| | | | cultivar-group) | is orthologous to G1274 |
| 150 | G3729 | PRT | Oryza sativa (japonica | Orthologous to G1274 |
| | | | cultivar-group) | |
| 151 | G3730 | DNA | Oryza sativa (japonica | Predicted polypeptide sequence |
| | | | cultivar-group) | is orthologous to G1274 |
| 152 | G3730 | PRT | Oryza sativa (japonica | Orthologous to G1274 |
| | | | cultivar-group) | |
| 153 | G3731 | DNA | Lycopersicon esculentum | Predicted polypeptide sequence |
| | | | | is orthologous to G1274 |
| 154 | G3731 | PRT | Lycopersicon esculentum | Orthologous to G1274 |
| 155 | G3732 | DNA | Solanum tuberosum | Predicted polypeptide sequence |
| | | | | is orthologous to G1274 |
| 156 | G3732 | PRT | Solanum tuberosum | Orthologous to G1274 |
| 157 | G3733 | DNA | Hordeum vulgare | Predicted polypeptide sequence |
| | | | | is orthologous to G1274 |
| 158 | G3733 | PRT | Hordeum vulgare | Orthologous to G1274 |
| 159 | G3735 | DNA | Medicago truncatula | Predicted polypeptide sequence |
| | | | | is orthologous to G1792 |
| 160 | G3735 | PRT | Medicago truncatula | Orthologous to G1792 |
| 161 | G3736 | DNA | Triticum aestivum | Predicted polypeptide sequence |
| | | | | is orthologous to G1792 |
| 162 | G3736 | PRT | Triticum aestivum | Orthologous to G1792 |
| 163 | G3737 | DNA | Oryza sativa (japonica | Predicted polypeptide sequence |
| | | | cultivar-group) | is orthologous to G1792 |
| 164 | G3737 | PRT | Oryza sativa (japonica | Orthologous to G1792 |
| | | | cultivar-group) | |
| 165 | G3739 | DNA | Zea mays | Predicted polypeptide sequence |
| | | | | is orthologous to G1792 |
| 166 | G3739 | PRT | Zea mays | Orthologous to G1792 |
| 167 | G3740 | DNA | Oryza sativa (japonica | Predicted polypeptide sequence |
| | | | cultivar-group) | is orthologous to G3086 |
| 168 | G3740 | PRT | Oryza sativa (japonica | Orthologous to G3086 |
| | | | cultivar-group) | |
| 169 | G3741 | DNA | Oryza sativa (japonica | Predicted polypeptide sequence |
| | | | cultivar-group) | is orthologous to G3086 |
| 170 | G3741 | PRT | Oryza sativa (japonica | Orthologous to G3086 |
| | | | cultivar-group) | |
| 171 | G3742 | DNA | Oryza sativa (japonica | Predicted polypeptide sequence |
| | | | cultivar-group) | is orthologous to G3086 |
| 172 | G3742 | PRT | Oryza sativa (japonica | Orthologous to G3086 |
| | | | cultivar-group) | |
| 173 | G3744 | DNA | Oryza sativa (japonica | Predicted polypeptide sequence |
| | | | cultivar-group) | is orthologous to G3086 |
| 174 | G3744 | PRT | Oryza sativa (japonica | Orthologous to G3086 |
| | | | cultivar-group) | |
| 175 | G3746 | DNA | Oryza sativa (japonica | Predicted polypeptide sequence |
| | | | cultivar-group) | is orthologous to G3086 |
| 176 | G3746 | PRT | Oryza sativa (japonica | Orthologous to G3086 |
| | | | cultivar-group) | |
| 177 | G3755 | DNA | Zea mays | Predicted polypeptide sequence |
| | | | | is orthologous to G3086 |
| 178 | G3755 | PRT | Zea mays | Orthologous to G3086 |
| 179 | G3763 | DNA | Glycine max | Predicted polypeptide sequence |
| | | | | is orthologous to G3086 |
| 180 | G3763 | PRT | Glycine max | Orthologous to G3086 |
| 181 | G3764 | DNA | Glycine max | Predicted polypeptide sequence |
| | | | | is orthologous to G3086 |
| 182 | G3764 | PRT | Glycine max | Orthologous to G3086 |
| 183 | G3765 | DNA | Glycine max | Predicted polypeptide sequence |
| | | | | is orthologous to G3086 |
| 184 | G3765 | PRT | Glycine max | Orthologous to G3086 |
| 185 | G3766 | DNA | Glycine max | Predicted polypeptide sequence |
| | | | | is orthologous to G3086 |
| 186 | G3766 | PRT | Glycine max | Orthologous to G3086 |
| 187 | G3767 | DNA | Glycine max | Predicted polypeptide sequence |
| | | | | is orthologous to G3086 |
| 188 | G3767 | PRT | Glycine max | Orthologous to G3086 |
| 189 | G3768 | DNA | Glycine max | Predicted polypeptide sequence |
| | | | | is orthologous to G3086 |
| 190 | G3768 | PRT | Glycine max | Orthologous to G3086 |
| 191 | G3769 | DNA | Glycine max | Predicted polypeptide sequence |
| | | | | is orthologous to G3086 |
| 192 | G3769 | PRT | Glycine max | Orthologous to G3086 |
| 193 | G3771 | DNA | Glycine max | Predicted polypeptide sequence |
| | | | | is orthologous to G3086 |
| 194 | G3771 | PRT | Glycine max | Orthologous to G3086 |
| 195 | G3772 | DNA | Glycine max | Predicted polypeptide sequence |
| | | | | is orthologous to G3086 |
| 196 | G3772 | PRT | Glycine max | Orthologous to G3086 |
| 197 | G3782 | DNA | Pinus taeda | Predicted polypeptide sequence |
| | | | | is orthologous to G3086 |
| 198 | G3782 | PRT | Pinus taeda | Orthologous to G3086 |
| 199 | G3794 | DNA | Zea mays | Predicted polypeptide sequence |
| | | | | is orthologous to G1792 |
| 200 | G3794 | PRT | Zea mays | Orthologous to G1792 |
| 201 | G3795 | DNA | Capsicum annuum | Predicted polypeptide sequence |
| | | | | is orthologous to G1274 |
| 202 | G3795 | PRT | Capsicum annuum | Orthologous to G1274 |
| 203 | G3797 | DNA | Lactuca sativa | Predicted polypeptide sequence |
| | | | | is orthologous to G1274 |
| 204 | G3797 | PRT | Lactuca sativa | Orthologous to G1274 |
| 205 | G3802 | DNA | Sorghum bicolor | Predicted polypeptide sequence |
| | | | | is orthologous to G1274 |
| 206 | G3802 | PRT | Sorghum bicolor | Orthologous to G1274 |
| 207 | G3803 | DNA | Glycine max | Predicted polypeptide sequence |
| | | | | is orthologous to G1274 |
| 208 | G3803 | PRT | Glycine max | Orthologous to G1274 |
| 209 | G3804 | DNA | Zea mays | Predicted polypeptide sequence |
| | | | | is orthologous to G1274 |
| 210 | G3804 | PRT | Zea mays | Orthologous to G1274 |
| 211 | G3810 | DNA | Glycine max | Predicted polypeptide sequence |
| | | | | is orthologous to G922 |
| 212 | G3810 | PRT | Glycine max | Orthologous to G922 |
| 213 | G3811 | DNA | Glycine max | Predicted polypeptide sequence |
| | | | | is orthologous to G922 |
| 214 | G3811 | PRT | Glycine max | Orthologous to G922 |
| 215 | G3813 | DNA | Oryza sativa (japonica | Predicted polypeptide sequence |
| | | | cultivar-group) | is orthologous to G922 |
| 216 | G3813 | PRT | Oryza sativa (japonica | Orthologous to G922 |
| | | | cultivar-group) | |
| 217 | G3814 | DNA | Oryza sativa (japonica | Predicted polypeptide sequence |
| | | | cultivar-group) | is orthologous to G922 |
| 218 | G3814 | PRT | Oryza sativa (japonica | Orthologous to G922 |
| | | | cultivar-group) | |
| 219 | G3824 | DNA | Lycopersicon esculentum | Predicted polypeptide sequence |
| | | | | is orthologous to G922 |
| 220 | G3824 | PRT | Lycopersicon esculentum | Orthologous to G922 |
| 221 | G3827 | DNA | Oryza sativa (japonica | Predicted polypeptide sequence |
| | | | cultivar-group) | is orthologous to G922 |
| 222 | G3827 | PRT | Oryza sativa (japonica | Orthologous to G922 |
| | | | cultivar-group) | |
| 223 | G175 | DNA | Arabidopsis thaliana | Reference sequence |
| 224 | G175 | PRT | Arabidopsis thaliana | Reference sequence |
| 225 | G303 | DNA | Arabidopsis thaliana | Reference sequence |
| 226 | G303 | PRT | Arabidopsis thaliana | Reference sequence |
| 227 | G354 | DNA | Arabidopsis thaliana | Reference sequence |
| 228 | G354 | PRT | Arabidopsis thaliana | Reference sequence |
| 229 | G489 | DNA | Arabidopsis thaliana | Reference sequence |
| 230 | G489 | PRT | Arabidopsis thaliana | Reference sequence |
| 231 | G634 | DNA | Arabidopsis thaliana | Reference sequence |
| 232 | G634 | PRT | Arabidopsis thaliana | Reference sequence |
| 233 | G682 | DNA | Arabidopsis thaliana | Reference sequence |
| 234 | G682 | PRT | Arabidopsis thaliana | Reference sequence |
| 235 | G916 | DNA | Arabidopsis thaliana | Reference sequence |
| 236 | G916 | PRT | Arabidopsis thaliana | Reference sequence |
| 237 | G975 | DNA | Arabidopsis thaliana | Reference sequence; predicted |
| | | | | polypeptide sequence is |
| | | | | paralogous to G2583 |
| 238 | G975 | PRT | Arabidopsis thaliana | Reference sequence; |
| | | | | paralogous to G2583 |
| 239 | G1069 | DNA | Arabidopsis thaliana | Reference sequence; |
| | | | | functionally related, |
| | | | | homologous to G1073 |
| 240 | G1069 | PRT | Arabidopsis thaliana | Reference sequence; |
| | | | | functionally related, |
| | | | | homologous to G1073 |
| 241 | G1452 | DNA | Arabidopsis thaliana | Reference sequence; |
| | | | | functionally related, |
| | | | | homologous to G512 |
| 242 | G1452 | PRT | Arabidopsis thaliana | Reference sequence; |
| | | | | functionally related, |
| | | | | homologous to G512 |
| 243 | G1820 | DNA | Arabidopsis thaliana | Reference sequence |
| 244 | G1820 | PRT | Arabidopsis thaliana | Reference sequence |
| 245 | G2701 | DNA | Arabidopsis thaliana | Reference sequence; predicted |
| | | | | polypeptide sequence is |
| | | | | paralogous to G1634 |
| 246 | G2701 | PRT | Arabidopsis thaliana | Reference sequence; |
| | | | | paralogous to G1634 |
| 247 | G2789 | DNA | Arabidopsis thaliana | Reference sequence; predicted |
| | | | | polypeptide sequence is |
| | | | | paralogous to G596 |
| 248 | G2789 | PRT | Arabidopsis thaliana | Reference sequence; |
| | | | | paralogous to G596 |
| 249 | G2839 | DNA | Arabidopsis thaliana | Reference sequence; predicted |
| | | | | polypeptide sequence is |
| | | | | paralogous to G354 |
| 250 | G2839 | PRT | Arabidopsis thaliana | Reference sequence; |
| | | | | paralogous to G354 |
| 251 | G2854 | DNA | Arabidopsis thaliana | Reference sequence; predicted |
| | | | | polypeptide sequence is |
| | | | | paralogous to G1940 |
| 252 | G2854 | PRT | Arabidopsis thaliana | Reference sequence; |
| | | | | paralogous to G1940 |
| 253 | G3083 | DNA | Arabidopsis thaliana | Reference sequence |
| 254 | G3083 | PRT | Arabidopsis thaliana | Reference sequence |
| 255 | G184 | DNA | Arabidopsis thaliana | Predicted polypeptide sequence |
| | | | | is paralogous to G916 |
| 256 | G184 | PRT | Arabidopsis thaliana | Paralogous to G916 |
| 257 | G186 | DNA | Arabidopsis thaliana | Predicted polypeptide sequence |
| | | | | is paralogous to G916 |
| 258 | G186 | PRT | Arabidopsis thaliana | Paralogous to G916 |
| 259 | G353 | DNA | Arabidopsis thaliana | Predicted polypeptide sequence |
| | | | | is paralogous to G354 |
| 260 | G353 | PRT | Arabidopsis thaliana | Paralogous to G354 |
| 261 | G512 | DNA | Arabidopsis thaliana | Predicted polypeptide sequence |
| | | | | is paralogous to G1452 |
| 262 | G512 | PRT | Arabidopsis thaliana | Paralogous to G1452 |
| 263 | G596 | DNA | Arabidopsis thaliana | Predicted polypeptide sequence |
| | | | | is paralogous to G2789 |
| 264 | G596 | PRT | Arabidopsis thaliana | Paralogous to G2789 |
| 265 | G714 | DNA | Arabidopsis thaliana | Predicted polypeptide sequence |
| | | | | is paralogous to G489 |
| 266 | G714 | PRT | Arabidopsis thaliana | Paralogous to G489 |
| 267 | G877 | DNA | Arabidopsis thaliana | Predicted polypeptide sequence |
| | | | | is paralogous to G175 |
| 268 | G877 | PRT | Arabidopsis thaliana | Paralogous to G175 |
| 269 | G1357 | DNA | Arabidopsis thaliana | Predicted polypeptide sequence |
| | | | | is paralogous to G1452 |
| 270 | G1357 | PRT | Arabidopsis thaliana | Paralogous to G1452 |
| 271 | G1387 | DNA | Arabidopsis thaliana | Predicted polypeptide sequence |
| | | | | is paralogous to G975 |
| 272 | G1387 | PRT | Arabidopsis thaliana | Paralogous to G975 |
| 273 | G1634 | DNA | Arabidopsis thaliana | Predicted polypeptide sequence |
| | | | | is paralogous to G2701 |
| 274 | G1634 | PRT | Arabidopsis thaliana | Paralogous to G2701 |
| 275 | G1889 | DNA | Arabidopsis thaliana | Predicted polypeptide sequence |
| | | | | is paralogous to G354 |
| 276 | G1889 | PRT | Arabidopsis thaliana | Paralogous to G354 |
| 277 | G1940 | DNA | Arabidopsis thaliana | Predicted polypeptide sequence |
| | | | | is paralogous to G2854 |
| 278 | G1940 | PRT | Arabidopsis thaliana | Paralogous to G2854 |
| 279 | G1974 | DNA | Arabidopsis thaliana | Predicted polypeptide sequence |
| | | | | is paralogous to G354 |
| 280 | G1974 | PRT | Arabidopsis thaliana | Paralogous to G354 |
| 281 | G2153 | DNA | Arabidopsis thaliana | Predicted polypeptide sequence |
| | | | | is paralogous to G1073 |
| 282 | G2153 | PRT | Arabidopsis thaliana | Paralogous to G1073 |
| 283 | G2583 | DNA | Arabidopsis thaliana | Predicted polypeptide sequence |
| | | | | is paralogous to G975 |
| 284 | G2583 | PRT | Arabidopsis thaliana | Paralogous to G975 |
| 287 | G226 | DNA | Arabidopsis thaliana | Reference sequence; predicted |
| | | | | polypeptide sequence is |
| | | | | paralogous to G682 |
| 288 | G226 | PRT | Arabidopsis thaliana | Reference sequence; |
| | | | | paralogous to G682 |
| 289 | G481 | DNA | Arabidopsis thaliana | Reference sequence; predicted |
| | | | | polypeptide sequence is |
| | | | | paralogous to G482 |
| 290 | G481 | PRT | Arabidopsis thaliana | Reference sequence; |
| | | | | paralogous to G482 |
| 291 | G482 | DNA | Arabidopsis thaliana | Reference sequence; predicted |
| | | | | polypeptide sequence is |
| | | | | paralogous to G481 |
| 292 | G482 | PRT | Arabidopsis thaliana | Reference sequence; |
| | | | | paralogous to G481 |
| 293 | G485 | DNA | Arabidopsis thaliana | Predicted polypeptide sequence |
| | | | | is paralogous to G481 and |
| | | | | G482 |
| 294 | G485 | PRT | Arabidopsis thaliana | Paralogous to G481 and G482 |
| 295 | G486 | DNA | Arabidopsis thaliana | Functionally related and |
| | | | | homologous to G481 and G482 |
| 296 | G486 | PRT | Arabidopsis thaliana | Functionally related and |
| | | | | homologous to G481 and G482 |
| 297 | G1067 | DNA | Arabidopsis thaliana | Predicted polypeptide sequence |
| | | | | is paralogous to G1073 |
| 298 | G1067 | PRT | Arabidopsis thaliana | Paralogous to G1073 |
| 299 | G1070 | DNA | Arabidopsis thaliana | Functionally related and |
| | | | | homologous to G1073 |
| 300 | G1070 | PRT | Arabidopsis thaliana | Functionally related and |
| | | | | homologous to G1073 |
| 301 | G1073 | DNA | Arabidopsis thaliana | Reference sequence |
| 302 | G1073 | PRT | Arabidopsis thaliana | Reference sequence |
| 303 | G1075 | DNA | Arabidopsis thaliana | Functionally related and |
| | | | | homologous to G1073 |
| 304 | G1075 | PRT | Arabidopsis thaliana | Functionally related and |
| | | | | homologous to G1073 |
| 305 | G1076 | DNA | Arabidopsis thaliana | Functionally related and |
| | | | | homologous to G1073 |
| 306 | G1076 | PRT | Arabidopsis thaliana | Functionally related and |
| | | | | homologous to G1073 |
| 307 | G1248 | DNA | Arabidopsis thaliana | Functionally related and |
| | | | | homologous to G481 and G482 |
| 308 | G1248 | PRT | Arabidopsis thaliana | Functionally related and |
| | | | | homologous to G481 and G482 |
| 309 | G1364 | DNA | Arabidopsis thaliana | Predicted polypeptide sequence |
| | | | | is paralogous to G481 and |
| | | | | G482 |
| 310 | G1364 | PRT | Arabidopsis thaliana | Paralogous to G481 and G482 |
| 311 | G1781 | DNA | Arabidopsis thaliana | Functionally related and |
| | | | | homologous to G481 and G482 |
| 312 | G1781 | PRT | Arabidopsis thaliana | Functionally related and |
| | | | | homologous to G481 and G482 |
| 313 | G1816 | DNA | Arabidopsis thaliana | Predicted polypeptide sequence |
| | | | | is paralogous to G226 and |
| | | | | G682 |
| 314 | G1816 | PRT | Arabidopsis thaliana | Paralogous to G226 and G682 |
| 315 | G1945 | DNA | Arabidopsis thaliana | Functionally related and |
| | | | | homologous to G1073 |
| 316 | G1945 | PRT | Arabidopsis thaliana | Functionally related and |
| | | | | homologous to G1073 |
| 317 | G2155 | DNA | Arabidopsis thaliana | Functionally related and |
| | | | | homologous to G1073 |
| 318 | G2155 | PRT | Arabidopsis thaliana | Functionally related and |
| | | | | homologous to G1073 |
| 319 | G2156 | DNA | Arabidopsis thaliana | Predicted polypeptide sequence |
| | | | | is paralogous to G1073 |
| 320 | G2156 | PRT | Arabidopsis thaliana | Paralogous to G1073 |
| 321 | G2345 | DNA | Arabidopsis thaliana | Predicted polypeptide sequence |
| | | | | is paralogous to G481 and |
| | | | | G482 |
| 322 | G2345 | PRT | Arabidopsis thaliana | Paralogous to G481 and G482 |
| 323 | G2657 | DNA | Arabidopsis thaliana | Functionally related and |
| | | | | homologous to G1073 |
| 324 | G2657 | PRT | Arabidopsis thaliana | Functionally related and |
| | | | | homologous to G1073 |
| 325 | G2718 | DNA | Arabidopsis thaliana | Predicted polypeptide sequence |
| | | | | is paralogous to G481 and |
| | | | | G482 |
| 326 | G2718 | PRT | Arabidopsis thaliana | Paralogous to G481 and G482 |
| 327 | G3392 | DNA | Oryza sativa (japonica | Predicted polypeptide sequence |
| | | | cultivar-group) | is orthologous to G682 |
| 328 | G3392 | PRT | Oryza sativa (japonica | Orthologous to G682 |
| | | | cultivar-group) | |
| 329 | G3393 | DNA | Oryza sativa (japonica | Predicted polypeptide sequence |
| | | | cultivar-group) | is orthologous to G682 |
| 330 | G3393 | PRT | Oryza sativa (japonica | Orthologous to G682 |
| | | | cultivar-group) | |
| 331 | G3394 | DNA | Oryza sativa (japonica | Predicted polypeptide sequence |
| | | | cultivar-group) | is orthologous to G481 and |
| | | | | G482 |
| 332 | G3394 | PRT | Oryza sativa (japonica | Orthologous to G481 and G482 |
| | | | cultivar-group) | |
| 333 | G3395 | DNA | Oryza sativa (japonica | Predicted polypeptide sequence |
| | | | cultivar-group) | is orthologous to G481 and |
| | | | | G482 |
| 334 | G3395 | PRT | Oryza sativa (japonica | Orthologous to G481 and G482 |
| | | | cultivar-group) | |
| 335 | G3396 | DNA | Oryza sativa (japonica | Predicted polypeptide sequence |
| | | | cultivar-group) | is orthologous to G481 and |
| | | | | G482 |
| 336 | G3396 | PRT | Oryza sativa (japonica | Orthologous to G481 and G482 |
| | | | cultivar-group) | |
| 337 | G3397 | DNA | Oryza sativa (japonica | Predicted polypeptide sequence |
| | | | cultivar-group) | is orthologous to G481 and |
| | | | | G482 |
| 338 | G3397 | PRT | Oryza sativa (japonica | Orthologous to G481 and G482 |
| | | | cultivar-group) | |
| 339 | G3398 | DNA | Oryza sativa (japonica | Predicted polypeptide sequence |
| | | | cultivar-group) | is orthologous to G481 and |
| | | | | G482 |
| 340 | G3398 | PRT | Oryza sativa (japonica | Orthologous to G481 and G482 |
| | | | cultivar-group) | |
| 341 | G3399 | DNA | Oryza sativa (japonica | Predicted polypeptide sequence |
| | | | cultivar-group) | is orthologous to G1073 |
| 342 | G3399 | PRT | Oryza sativa (japonica | Orthologous to G1073 |
| | | | cultivar-group) | |
| 343 | G3400 | DNA | Oryza sativa (japonica | Predicted polypeptide sequence |
| | | | cultivar-group) | is orthologous to G1073 |
| 344 | G3400 | PRT | Oryza sativa (japonica | Orthologous to G1073 |
| | | | cultivar-group) | |
| 345 | G3401 | DNA | Oryza sativa (japonica | Predicted polypeptide sequence |
| | | | cultivar-group) | is orthologous to G1073 |
| 346 | G3401 | PRT | Oryza sativa (japonica | Orthologous to G1073 |
| | | | cultivar-group) | |
| 347 | G3403 | DNA | Oryza sativa (japonica | Predicted polypeptide sequence |
| | | | cultivar-group) | is orthologous to G1073 |
| 348 | G3403 | PRT | Oryza sativa (japonica | Orthologous to G1073 |
| | | | cultivar-group) | |
| 349 | G3404 | DNA | Oryza sativa (japonica | Functionally related and |
| | | | cultivar-group) | homologous to G1073 |
| 350 | G3404 | PRT | Oryza sativa (japonica | Functionally related and |
| | | | cultivar-group) | homologous to G1073 |
| 351 | G3405 | DNA | Oryza sativa (japonica | Functionally related and |
| | | | cultivar-group) | homologous to G1073 |
| 352 | G3405 | PRT | Oryza sativa (japonica | Functionally related and |
| | | | cultivar-group) | homologous to G1073 |
| 353 | G3406 | DNA | Oryza sativa (japonica | Functionally related and |
| | | | cultivar-group) | homologous to G1073 |
| 354 | G3406 | PRT | Oryza sativa (japonica | Functionally related and |
| | | | cultivar-group) | homologous to G1073 |
| 355 | G3407 | DNA | Oryza sativa (japonica | Functionally related and |
| | | | cultivar-group) | homologous to G1073 |
| 356 | G3407 | PRT | Oryza sativa (japonica | Functionally related and |
| | | | cultivar-group) | homologous to G1073 |
| 357 | G3408 | DNA | Oryza sativa (japonica | Functionally related and |
| | | | cultivar-group) | homologous to G1073 |
| 358 | G3408 | PRT | Oryza sativa (japonica | Functionally related and |
| | | | cultivar-group) | homologous to G1073 |
| 359 | G3429 | DNA | Oryza sativa (japonica | Predicted polypeptide sequence |
| | | | cultivar-group) | is orthologous to G481 and |
| | | | | G482 |
| 360 | G3429 | PRT | Oryza sativa (japonica | Orthologous to G481 and G482 |
| | | | cultivar-group) | |
| 361 | G3431 | DNA | Zea mays | Predicted polypeptide sequence |
| | | | | is orthologous to G682 |
| 362 | G3431 | PRT | Zea mays | Orthologous to G682 |
| 363 | G3434 | DNA | Zea mays | Predicted polypeptide sequence |
| | | | | is orthologous to G481 and |
| | | | | G482 |
| 364 | G3434 | PRT | Zea mays | Orthologous to G481 and G482 |
| 365 | G3435 | DNA | Zea mays | Predicted polypeptide sequence |
| | | | | is orthologous to G481 and |
| | | | | G482 |
| 366 | G3435 | PRT | Zea mays | Orthologous to G481 and G482 |
| 367 | G3436 | DNA | Zea mays | Predicted polypeptide sequence |
| | | | | is orthologous to G481 and |
| | | | | G482 |
| 368 | G3436 | PRT | Zea mays | Orthologous to G481 and G482 |
| 369 | G3437 | DNA | Zea mays | Predicted polypeptide sequence |
| | | | | is orthologous to G481 and |
| | | | | G482 |
| 370 | G3437 | PRT | Zea mays | Orthologous to G481 and G482 |
| 371 | G3444 | DNA | Zea mays | Predicted polypeptide sequence |
| | | | | is orthologous to G682 |
| 372 | G3444 | PRT | Zea mays | Orthologous to G682 |
| 373 | G3445 | DNA | Glycine max | Predicted polypeptide sequence |
| | | | | is orthologous to G682 |
| 374 | G3445 | PRT | Glycine max | Orthologous to G682 |
| 375 | G3446 | DNA | Glycine max | Predicted polypeptide sequence |
| | | | | is orthologous to G682 |
| 376 | G3446 | PRT | Glycine max | Orthologous to G682 |
| 377 | G3447 | DNA | Glycine max | Predicted polypeptide sequence |
| | | | | is orthologous to G682 |
| 378 | G3447 | PRT | Glycine max | Orthologous to G682 |
| 379 | G3448 | DNA | Glycine max | Predicted polypeptide sequence |
| | | | | is orthologous to G682 |
| 380 | G3448 | PRT | Glycine max | Orthologous to G682 |
| 381 | G3449 | DNA | Glycine max | Predicted polypeptide sequence |
| | | | | is orthologous to G682 |
| 382 | G3449 | PRT | Glycine max | Orthologous to G682 |
| 383 | G3450 | DNA | Glycine max | Predicted polypeptide sequence |
| | | | | is orthologous to G682 |
| 384 | G3450 | PRT | Glycine max | Orthologous to G682 |
| 385 | G3456 | DNA | Glycine max | Predicted polypeptide sequence |
| | | | | is orthologous to G1073 |
| 386 | G3456 | PRT | Glycine max | Orthologous to G1073 |
| 387 | G3458 | DNA | Glycine max | Functionally related and |
| | | | | homologous to G1073 |
| 388 | G3458 | PRT | Glycine max | Functionally related and |
| | | | | homologous to G1073 |
| 389 | G3459 | DNA | Glycine max | Predicted polypeptide sequence |
| | | | | is functionally related and |
| | | | | homologous to G1073 |
| 390 | G3459 | PRT | Glycine max | Functionally related and |
| | | | | homologous to G1073 |
| 391 | G3460 | DNA | Glycine max | Predicted polypeptide sequence |
| | | | | is functionally related and |
| | | | | homologous to G1073 |
| 392 | G3460 | PRT | Glycine max | Functionally related and |
| | | | | homologous to G1073 |
| 393 | G3462 | DNA | Glycine max | Predicted polypeptide sequence |
| | | | | is orthologous to G1073 |
| 394 | G3462 | PRT | Glycine max | Orthologous to G1073 |
| 395 | G3470 | DNA | Glycine max | Predicted polypeptide sequence |
| | | | | is orthologous to G481 and |
| | | | | G482 |
| 396 | G3470 | PRT | Glycine max | Orthologous to G481 and G482 |
| 397 | G3471 | DNA | Glycine max | Predicted polypeptide sequence |
| | | | | is orthologous to G481 and |
| | | | | G482 |
| 398 | G3471 | PRT | Glycine max | Orthologous to G481 and G482 |
| 399 | G3472 | DNA | Glycine max | Predicted polypeptide sequence |
| | | | | is orthologous to G481 and |
| | | | | G482 |
| 400 | G3472 | PRT | Glycine max | Orthologous to G481 and G482 |
| 401 | G3473 | DNA | Glycine max | Predicted polypeptide sequence |
| | | | | is orthologous to G481 and |
| | | | | G482 |
| 402 | G3473 | PRT | Glycine max | Orthologous to G481 and G482 |
| 403 | G3474 | DNA | Glycine max | Predicted polypeptide sequence |
| | | | | is orthologous to G481 and |
| | | | | G482 |
| 404 | G3474 | PRT | Glycine max | Orthologous to G481 and G482 |
| 405 | G3475 | DNA | Glycine max | Predicted polypeptide sequence |
| | | | | is orthologous to G481 and |
| | | | | G482 |
| 406 | G3475 | PRT | Glycine max | Orthologous to G481 and G482 |
| 407 | G3476 | DNA | Glycine max | Predicted polypeptide sequence |
| | | | | is orthologous to G481 and |
| | | | | G482 |
| 408 | G3476 | PRT | Glycine max | Orthologous to G481 and G482 |
| 409 | G3477 | DNA | Glycine max | Predicted polypeptide sequence |
| | | | | is orthologous to G481 and |
| | | | | G482 |
| 410 | G3477 | PRT | Glycine max | Orthologous to G481 and G482 |
| 411 | G3478 | DNA | Glycine max | Predicted polypeptide sequence |
| | | | | is orthologous to G481 and |
| | | | | G482 |
| 412 | G3478 | PRT | Glycine max | Orthologous to G481 and G482 |
| 413 | G3556 | DNA | Oryza sativa (japonica | Predicted polypeptide sequence |
| | | | cultivar-group) | is orthologous to G1073 |
| 414 | G3556 | PRT | Oryza sativa (japonica | Orthologous to G1073 |
| | | | cultivar-group) | |
| 415 | G3835 | DNA | Oryza sativa (japonica | Predicted polypeptide sequence |
| | | | cultivar-group) | is orthologous to G481 and |
| | | | | G482 |
| 416 | G3835 | PRT | Oryza sativa (japonica | Orthologous to G481 and G482 |
| | | | cultivar-group) | |
| 417 | G3836 | DNA | Oryza sativa (japonica | Predicted polypeptide sequence |
| | | | cultivar-group) | is orthologous to G481 and |
| | | | | G482 |
| 418 | G3836 | PRT | Oryza sativa (japonica | Orthologous to G481 and G482 |
| | | | cultivar-group) | |
| 419 | G3837 | DNA | Glycine max | Predicted polypeptide sequence |
| | | | | is orthologous to G481 and |
| | | | | G482 |
| 420 | G3837 | PRT | Glycine max | Orthologous to G481 and G482 |

Molecular Modeling

Another means that may be used to confirm the utility and function of transcription factor sequences that are orthologous or paralogous to presently disclosed transcription factors is through the use of molecular modeling software. Molecular modeling is routinely used to predict polypeptide structure, and a variety of protein structure modeling programs, such as “Insight II” (Accelrys, Inc.) are commercially available for this purpose. Modeling can thus be used to predict which residues of a polypeptide can be changed without altering function (Crameri et al. (2003) U.S. Pat. No. 6,521,453). Thus, polypeptides that are sequentially similar can be shown to have a high likelihood of similar function by their structural similarity, which may, for example, be established by comparison of regions of superstructure. The relative tendencies of amino acids to form regions of superstructure (for example, helixes and _-sheets) are well established. For example, O'Neil et al. ((1990) Science 250: 646-651) have discussed in detail the helix forming tendencies of amino acids. Tables of relative structure forming activity for amino acids can be used as substitution tables to predict which residues can be functionally substituted in a given region, for example, in DNA-binding domains of known transcription factors and equivalogs. Homologs that are likely to be functionally similar can then be identified.

Of particular interest is the structure of a transcription factor in the region of its conserved domains, such as those identified in Table 1. Structural analyses may be performed by comparing the structure of the known transcription factor around its conserved domain with those of orthologs and paralogs. Analysis of a number of polypeptides within a transcription factor group or clade, including the functionally or sequentially similar polypeptides provided in the Sequence Listing, may also provide an understanding of structural elements required to regulate transcription within a given family.

### EXAMPLES

The invention, now being generally described, will be more readily understood by reference to the following examples, which are included merely for purposes of illustration of certain aspects and embodiments of the present invention and are not intended to limit the invention. It will be recognized by one of skill in the art that a transcription factor that is associated with a particular first trait may also be associated with at least one other, unrelated and inherent second trait which was not predicted by the first trait.

The complete descriptions of the traits associated with each polynucleotide of the invention are fully disclosed in Example IX. The complete description of the transcription factor gene family and identified conserved domains of the polypeptide encoded by the polynucleotide is fully disclosed in Table 1.

### Example 1

### Full Length Gene Identification and Cloning

Putative transcription factor sequences (genomic or ESTs) related to known transcription factors were identified in the Arabidopsis thaliana GenBank database using the tblastn sequence analysis program using default parameters and a P-value cutoff threshold of −4 or −5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Boehringer Mannheim Corp. (now Roche Diagnostics Corp., Indianapolis, Ind.). Purified radiolabelled probes were added to filters immersed in Church hybridization medium (0.5 M NaPO₄ pH 7.0, 7% SDS, 1% w/v bovine serum albumin) and hybridized overnight at 60° C. with shaking. Filters were washed two times for 45 to 60 minutes with lxSCC, 1% SDS at 60° C.

To identify additional sequence 5′ or 3′ of a partial cDNA sequence in a cDNA library, 5′ and 3′ rapid amplification of cDNA ends (RACE) was performed using the MARATHON cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the MARATHON Adaptor to the cDNA to form a library of adaptor-ligated ds cDNA.

Gene-specific primers were designed to be used along with adaptor specific primers for both 5′ and 3′ RACE reactions. Nested primers, rather than single primers, were used to increase PCR specificity. Using 5′ and 3′ RACE reactions, 5′ and 3′ RACE fragments were obtained, sequenced and cloned. The process can be repeated until 5′ and 3′ ends of the full-length gene were identified. Then the full-length cDNA was generated by PCR using primers specific to 5′ and 3′ ends of the gene by end-to-end PCR.

### Example II

### Construction of Expression Vectors

The sequence was amplified from a genomic or cDNA library using primers specific to sequences upstream and downstream of the coding region. The expression vector was pMEN20 or pMEN65, which are both derived from pMON316 (Sanders et al. (1987) Nucleic Acids Res. 15:1543-1558) and contain the CaMV 35S promoter to express transgenes. To clone the sequence into the vector, both pMEN20 and the amplified DNA fragment were digested separately with SalI and NotI restriction enzymes at 37° C. for 2 hours. The digestion products were subject to electrophoresis in a 0.8% agarose gel and visualized by ethidium bromide staining. The DNA fragments containing the sequence and the linearized plasmid were excised and purified by using a QIAQUICK gel extraction kit (Qiagen, Valencia Calif.). The fragments of interest were ligated at a ratio of 3:1 (vector to insert). Ligation reactions using T4 DNA ligase (New England Biolabs, Beverly Mass.) were carried out at 16° C. for 16 hours. The ligated DNAs were transformed into competent cells of the E. coli strain DH5alpha by using the heat shock method. The transformations were plated on LB plates containing 50 mg/l kanamycin (Sigma Chemical Co. St. Louis Mo.). Individual colonies were grown overnight in five milliliters of LB broth containing 50 mg/l kanamycin at 37° C. Plasmid DNA was purified by using Qiaquick Mini Prep kits (Qiagen).

### Example III

### Transformation of Agrobacterium With the Expression Vector

After the plasmid vector containing the gene was constructed, the vector was used to transform Agrobacterium tumefaciens cells expressing the gene products. The stock of Agrobacterium tumefaciens cells for transformation was made as described by Nagel et al. (1990) FEMS Microbiol Letts. 67: 325-328. Agrobacterium strain ABI was grown in 250 ml LB medium (Sigma) overnight at 28° C. with shaking until an absorbance over 1 cm at 600 nm (A₆₀₀) of 0.5-1.0 was reached. Cells were harvested by centrifugation at 4,000×g for 15 min at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with plasmids prepared as described above following the protocol described by,Nagel et al. (supra). For each DNA construct to be transformed, 50-100 ng DNA (generally resuspended in 10 mM Tris-HCl, 1 mM EDTA, pH 8.0) was mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 μg/ml spectinomycin (Sigma) and incubated for 24-48 hours at 28° C. Single colonies were then picked and inoculated in fresh medium. The presence of the plasmid construct was verified by PCR amplification and sequence analysis.

### Example IV

### Transformation of Arabidopsis Plants with Agrobacterium tumefaciens With Expression Vector

After transformation of Agrobacterium tumefaciens with plasmid vectors containing the gene, single Agrobacterium colonies were identified, propagated, and used to transform Arabidopsis plants. Briefly, 500 ml cultures of LB medium containing 50 mg/l kanamycin were inoculated with the colonies and grown at 28° C. with shaking for 2 days until an optical absorbance at 600 nm wavelength over 1 cm (A₆₀₀) of >2.0 is reached. Cells were then harvested by centrifugation at 4,000×g for 10 min, and resuspended in infiltration medium (½× Murashige and Skoog salts (Sigma), 1× Gamborg's B-5 vitamins (Sigma), 5.0% (w/v) sucrose (Sigma), 0.044 μM benzylamino purine (Sigma), 200 μl/l Silwet L-77 (Lehle Seeds) until an A₆₀₀ of 0.8 was reached.

Prior to transformation, Arabidopsis thaliana seeds (ecotype Columbia) were sown at a density of ˜10 plants per 4″ pot onto Pro-Mix BX potting medium (Hummert International) covered with fiberglass mesh (18 mm×16 mm). Plants were grown under continuous illumination (50-75 μE/m²/sec) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 sec, and placed on their sides to allow draining into a 1′×2′ flat surface covered with plastic wrap. After 24 h, the plastic wrap was removed and pots are turned upright. The immersion procedure was repeated one week later, for a total of two immersions per pot. Seeds were then collected from each transformation pot and analyzed following the protocol described below.

### Example V

### Identification of Arabidopsis Primary Transformants

Seeds collected from the transformation pots were sterilized essentially as follows. Seeds were dispersed into in a solution containing 0.1% (v/v) Triton X-100 (Sigma) and sterile water and washed by shaking the suspension for 20 min. The wash solution was then drained and replaced with fresh wash solution to wash the seeds for 20 min with shaking. After removal of the ethanol/detergent solution, a solution containing 0.1% (v/v) Triton X-100 and 30% (v/v) bleach (CLOROX; Clorox Corp. Oakland Calif.) was added to the seeds, and the suspension was shaken for 10 min. After removal of the bleach/detergent solution, seeds were then washed five times in sterile distilled water. The seeds were stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies), and 50 mg/l kanamycin). Seeds were germinated under continuous illumination (50-75 μE/m²/sec) at 22-23° C. After 7-10 days of g under these conditions, kanamycin resistant primary transformants (T1 generation) were visible and obtained. These seedlings were transferred first to fresh selection plates where the seedlings continued to grow for 3-5 more days, and then to soil (Pro-Mix BX potting medium).

Primary transformants were crossed and progeny seeds (T₂) collected; kanamycin resistant seedlings were selected and analyzed. The expression levels of the recombinant polynucleotides in the transformants varies from about a 5% expression level increase to a least a 100% expression level increase. Similar observations are made with respect to polypeptide level expression.

### Example VI

### Identification of Arabidopsis Plants with Transcription Factor Gene Knockouts

The screening of insertion mutagenized Arabidopsis collections for null mutants in a known target gene was essentially as described in Krysan et al. (1999) Plant Cell 11: 2283-2290. Briefly, gene-specific primers, nested by 5-250 base pairs to each other, were designed from the 5′ and 3′ regions of a known target gene. Similarly, nested sets of primers were also created specific to each of the T-DNA or transposon ends (the “right” and “left” borders). All possible combinations of gene specific and T-DNA/transposon primers were used to detect by PCR an insertion event within or close to the target gene. The amplified DNA fragments were then sequenced which allows the precise determination of the T-DNA/transposon insertion point relative to the target gene. Insertion events within the coding or intervening sequence of the genes were deconvoluted from a pool comprising a plurality of insertion events to a single unique mutant plant for functional characterization. The method is described in more detail in Yu and Adam, U.S. application Ser. No. 09/177,733 filed Oct. 23, 1998.

### Example VII

### Identification of Modified Phenotypes in Overexpression or Gene Knockout Plants

Experiments were performed to identify those transformants or knockouts that exhibited modified biochemical characteristics.

Calibration of NIRS response was performed using data obtained by wet chemical analysis of a population of Arabidopsis ecotypes that were expected to represent diversity of oil and protein levels.

Experiments were performed to identify those transformants or knockouts that exhibited modified sugar-sensing. For such studies, seeds from transformants were germinated on media containing 5% glucose or 9.4% sucrose which normally partially restrict hypocotyl elongation. Plants with altered sugar sensing may have either longer or shorter hypocotyls than normal plants when grown on this media. Additionally, other plant traits may be varied such as root mass.

In some instances, expression patterns of the stress-induced genes may be monitored by microarray experiments. In these experiments, cDNAs are generated by PCR and resuspended at a final concentration of about 100 ng/μl in 3×SSC or 150 mM Na-phosphate (Eisen and Brown (1999) Methods Enzymol. 303: 179-205). The cDNAs are spotted on microscope glass slides coated with polylysine. The prepared cDNAs are aliquoted into 384 well plates and spotted on the slides using, for example, an x-y-z gantry (OmniGrid) which may be purchased from GeneMachines (Menlo Park, Calif.) outfitted with quill type pins which may be purchased from Telechem International (Sunnyvale, Calif.). After spotting, the arrays are cured for a minimum of one week at room temperature, rehydrated and blocked following the protocol recommended by Eisen and Brown (1999) supra.

Sample total RNA (10 μg) samples are labeled using fluorescent Cy3 and Cy5 dyes. Labeled samples are resuspended in 4×SSC/0.03% SDS/4 μg salmon sperm DNA/2 μg tRNA/50 mM Na-pyrophosphate, heated for 95° C. for 2.5 minutes, spun down and placed on the array. The array is then covered with a glass coverslip and placed in a sealed chamber. The chamber is then kept in a water bath at 62° C. overnight. The arrays are washed as described in Eisen and Brown (1999, supra) and scanned on a General Scanning 3000 laser scanner. The resulting files are subsequently quantified using IMAGENE, software (BioDiscovery, Los Angeles Calif.).

RT-PCR experiments may be performed to identify those genes induced after exposure to drought stress. Generally, the gene expression patterns from ground plant leaf tissue is examined. Reverse transcriptase PCR was conducted using gene specific primers within the coding region for each sequence identified. The primers were designed near the 3′ region of each DNA binding sequence initially identified.

Total RNA from these ground leaf tissues was isolated using the CTAB extraction protocol. Once extracted total RNA was normalized in concentration across all the tissue types to ensure that the PCR reaction for each tissue received the same amount of cDNA template using the 28S band as reference. Poly(A+) RNA was purified using a modified protocol from the Qiagen OLIGOTEX purification kit batch protocol. cDNA was synthesized using standard protocols. After the first strand cDNA synthesis, primers for Actin 2 were used to normalize the concentration of cDNA across the tissue types. Actin 2 is found to be constitutively expressed in fairly equal levels across the tissue types we are investigating.

For RT PCR, cDNA template was mixed with corresponding primers and Taq DNA polymerase. Each reaction consisted of 0.2 μl cDNA template, 2 μl 10× Tricine buffer, 2 μl 10× Tricine buffer and 16.8 μl water, 0.05 μl Primer 1, 0.05 μl, Primer 2, 0.3 μl Taq DNA polymerase and 8.6 μl water.

The 96 well plate is covered with microfilm and set in the thermocycler to start the reaction cycle. By way of illustration, the reaction cycle may comprise the following steps:

Step 1: 93° C. for 3 min;

Step 2: 93° C. for 30 sec;

Step 3: 65° C. for 1 min;

Step 4: 72° C. for 2 min;

Steps 2, 3 and 4 are repeated for 28 cycles;

Step 5: 72° C. for 5 min; and

STEP 6 4° C.

To amplify more products, for example, to identify genes that have very low expression, additional steps may be performed: The following method illustrates a method that may be used in this regard. The PCR plate is placed back in the thermocycler for 8 more cycles of steps 2-4.

Step 2 93° C. for 30 sec;

Step 3 65° C. for 1 min;

Step 4 72° C. for 2 min, repeated for 8 cycles; and

Step 5 4° C.

Eight microliters of PCR product and 1.5 μl of loading dye are loaded on a 1.2% agarose gel for analysis after 28 cycles and 36 cycles. Expression levels of specific transcripts are considered low if they were only detectable after 36 cycles of PCR. Expression levels are considered medium or high depending on the levels of transcript compared with observed transcript levels for an internal control such as actin2. Transcript levels are determined in repeat experiments and compared to transcript levels in control (e.g., non-transformed) plants.

The sequences of the Sequence Listing, can be used to prepare transgenic plants and plants with altered drought stress tolerance. The specific transgenic plants listed below are produced from the sequences of the Sequence Listing, as noted.

### Example VIII

### Analysis Methods for Soil-Based Drought Assays

Soil-based drought screens were performed with Arabidopsis plants overexpressing the transcription factors listed in the Sequence Listing. Seeds from wild-type Arabidopsis plants, or plants overexpressing a polypeptide of the invention, were stratified for three days at 4° C. in 0.1% agarose. Fourteen seeds of each overexpressor or wild-type were then sown in three inch clay pots containing a 50:50 mix of vermiculite:perlite topped with a small layer of MetroMix 200 and grown for fifteen days under 24 hr light. Pots containing wild-type and overexpressing seedlings were placed in flats in random order. Drought stress was initiated by placing pots on absorbent paper for seven to eight days. The seedlings were considered to be sufficiently stressed when the majority of the pots containing wild-type seedlings within a flat had become severely wilted. Pots were then re-watered and survival was scored four to seven days later. Plants were ranked against wild-type controls for each of two criteria: tolerance to the drought conditions and recovery (survival) following re-watering

At the end of the initial drought period, each pot was assigned a numeric value score depending on the above criteria. Scores of 0-6 were assigned (Table 9), with a low value of “0” assigned to plants with an extremely poor appearance (i.e., the plants were uniformly brown) and a value of “6” given to plants that were rated very healthy in appearance (i.e., the plants were all green). After the plants were rewatered and incubated an additional four to seven days, the plants were reevaluated to indicate the degree of recovery from the water deprivation treatment.

An analysis was then conducted to determine which plants best survived water deprivation, identifying the transgenes that consistently conferred drought-tolerant phenotypes and their ability to recover from this treatment. The analysis was performed by comparing overall and within-flat tabulations with a set of statistical models to account for variations between batches. Several measures of survival were tabulated, including: (a) the average proportion of plants surviving relative to wild-type survival within the same flat; (b) the median proportion surviving relative to wild-type survival within the same flat; (c) the overall average survival (taken over all batches, flats, and pots); (d) the overall average survival relative to the overall wild-type survival; and (e) the average visual score of plant health before rewatering.

### Example IX

### Genes that Confer Significant Improvements to Plants

Examples of genes and homologs that confer significant improvements to knockout or overexpressing plants are noted below. Experimental observations made by us with regard to specific genes whose expression has been modified in overexpressing or knock-out plants, and potential applications based on these observations, are also presented.

This example provides experimental evidence for increased biomass and abiotic stress tolerance controlled by the transcription factor polypeptides and polypeptides of the invention.

Salt stress assays are intended to find genes that confer better germination, seedling vigor or growth in high salt. Evaporation from the soil surface causes upward water movement and salt accumulation in the upper soil layer where the seeds are placed. Thus, germination normally takes place at a salt concentration much higher than the mean salt concentration of the whole soil profile. Plants differ in their tolerance to NaCl depending on their stage of development, therefore seed germination, seedling vigor, and plant growth responses are evaluated.

Osmotic stress assays (including NaCl and mannitol assays) are intended to determine if an osmotic stress phenotype is NaCl-specific or if it is a general osmotic stress related phenotype. Plants tolerant to osmotic stress could also have more tolerance to drought and/or freezing.

Drought assays are intended to find genes that mediate better plant survival after short-term, severe water deprivation. Ion leakage is measured if needed. Osmotic stress tolerance also is predictive of a drought tolerant phenotype.

Sugar sensing assays are intended to find genes involved in sugar sensing by germinating seeds on high concentrations of sucrose and glucose and looking for degrees of hypocotyl elongation. The germination assay on mannitol controls for responses related to osmotic stress. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships). Glucose-specific hexose-sensing has also been described in plants and is implicated in cell division and repression of “famine” genes (photosynthetic or glyoxylate cycles).

Germination assays followed modifications of the same basic protocol. Sterile seeds were sown on the conditional media listed below. Plates were incubated at 22° C. under 24-hour light (120-130 μEin/m²/s) in a growth chamber. Evaluation of germination and seedling vigor was conducted 3 to 15 days after planting. The basal media was 80% Murashige-Skoog medium (MS)+vitamins.

For salt and osmotic stress germination experiments, the medium was supplemented with 150 mM NaCl or 300 mM mannitol. Growth regulator sensitivity assays were performed in MS media, vitamins, and either 0.3 μM ABA, 9.4% sucrose, or 5% glucose.

Results:

As noted below, overexpression of G2133, G1274, G922, G2999, G3086, G354, G1792, G2053, G975, G1069, G916, G1820, G2701, G47, G2854, G2789, G634, G175, G2839, G1452, G3083, G489, G303, G2992, and G682 was shown to increase drought stress tolerance in plants.

G2133 (SEQ ID NOs: 11 and 12)

Published Information

G2133 corresponds to gene F26A9.11 (AAF23336). No information is available about the function(s) of G2133.

Closely Related Genes from Other Species

G2133 does not show extensive sequence similarity with known genes from other plant species outside of the conserved AP2/EREBP domain.

Experimental Observations

The function of G2133 was studied using transgenic plants in which the gene was expressed under the control of the 35S promoter.

G2133 expression was detected in a variety of tissues: flower, leaf, embryo, and silique samples. Its expression might be altered by several conditions, including auxin treatment, osmotic stress, and Fusarium infection. Overexpression of G2133 caused a variety of alterations in plant growth and development: delayed flowering, altered inflorescence architecture, and a decrease in overall size and fertility.

At early stages, 35S::G2133 transformants were markedly smaller than controls and displayed curled, dark-green leaves. Most of these plants remained in a vegetative phase of development substantially longer than controls, and produced an increased number of leaves before bolting. In the most severely affected plants, bolting occurred more than a month later than in wild type (24-hour light). In addition, the plants displayed a reduction in apical dominance and formed large numbers of shoots simultaneously, from the axils of rosette leaves. These inflorescence stems had short internodes, and carried increased numbers of cauline leaf nodes, giving them a very leafy appearance. The fertility of 35S::G2133 plants was generally very low. In addition, G2133 overexpressing lines were found to be more resistant to the herbicide glyphosate in initial and repeat experiments.

No alterations were detected in 35S::G2133 plants in the biochemical analyses that were performed.

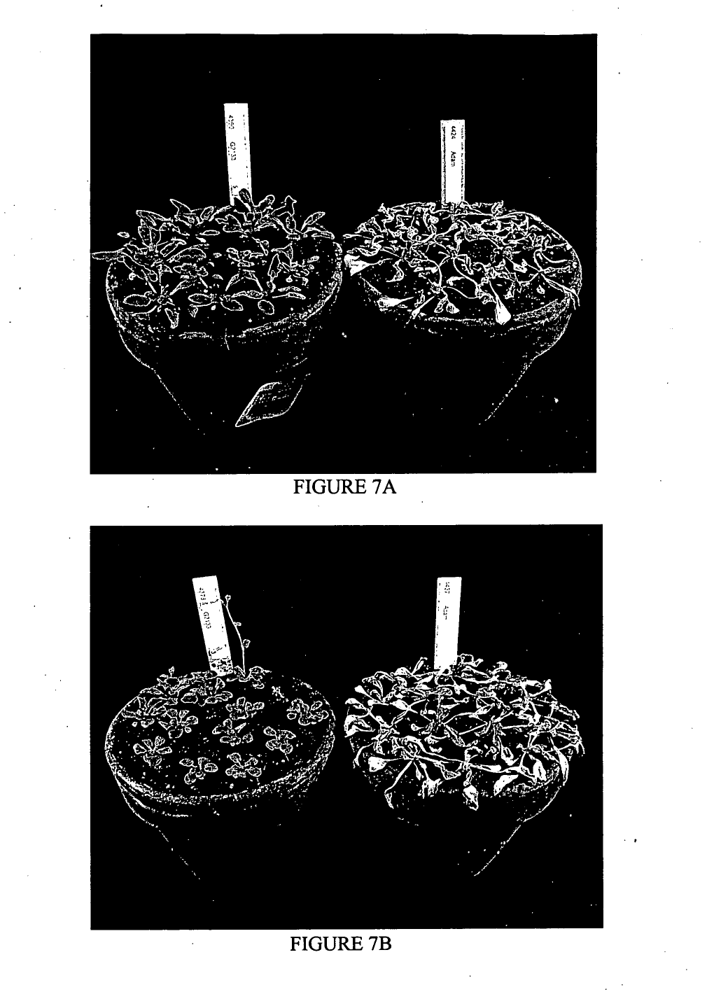

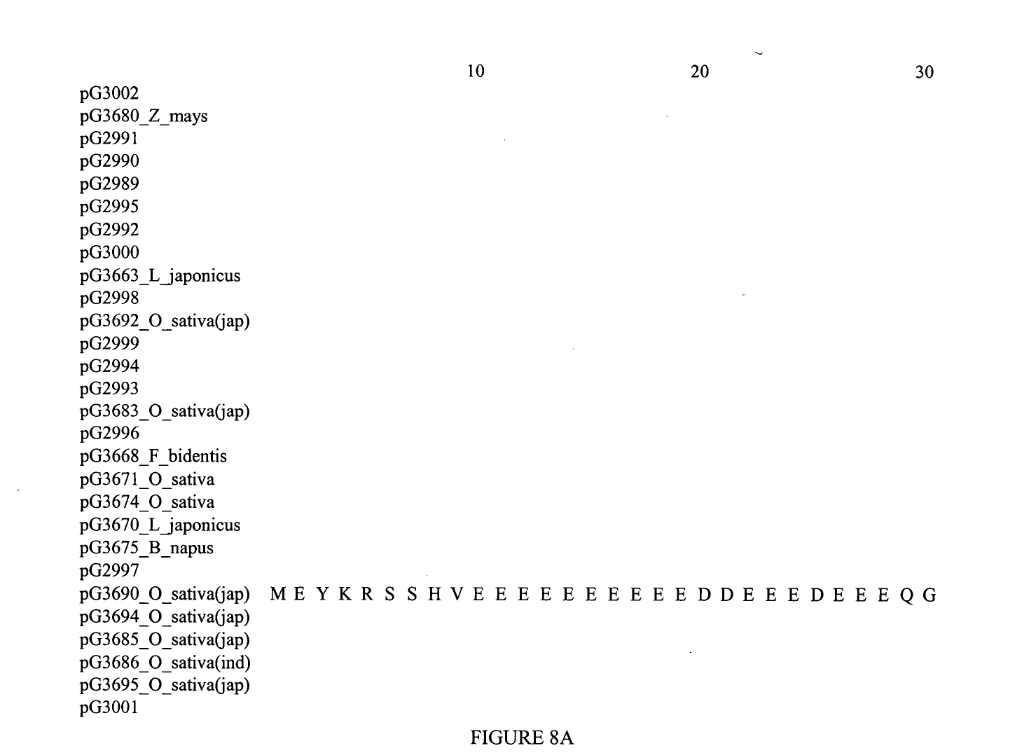

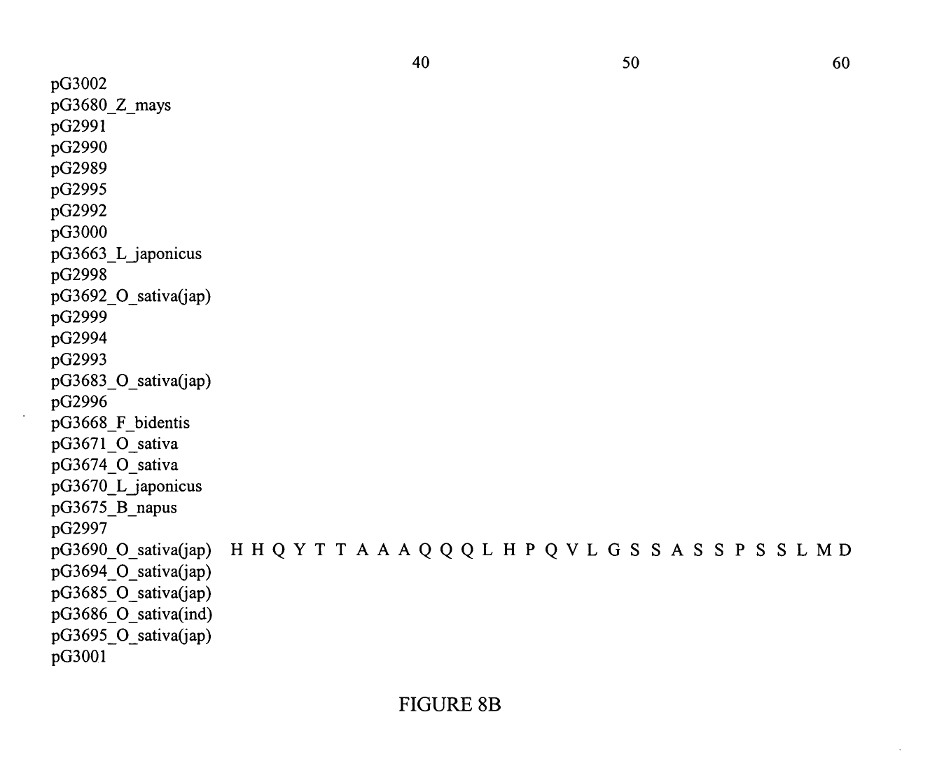

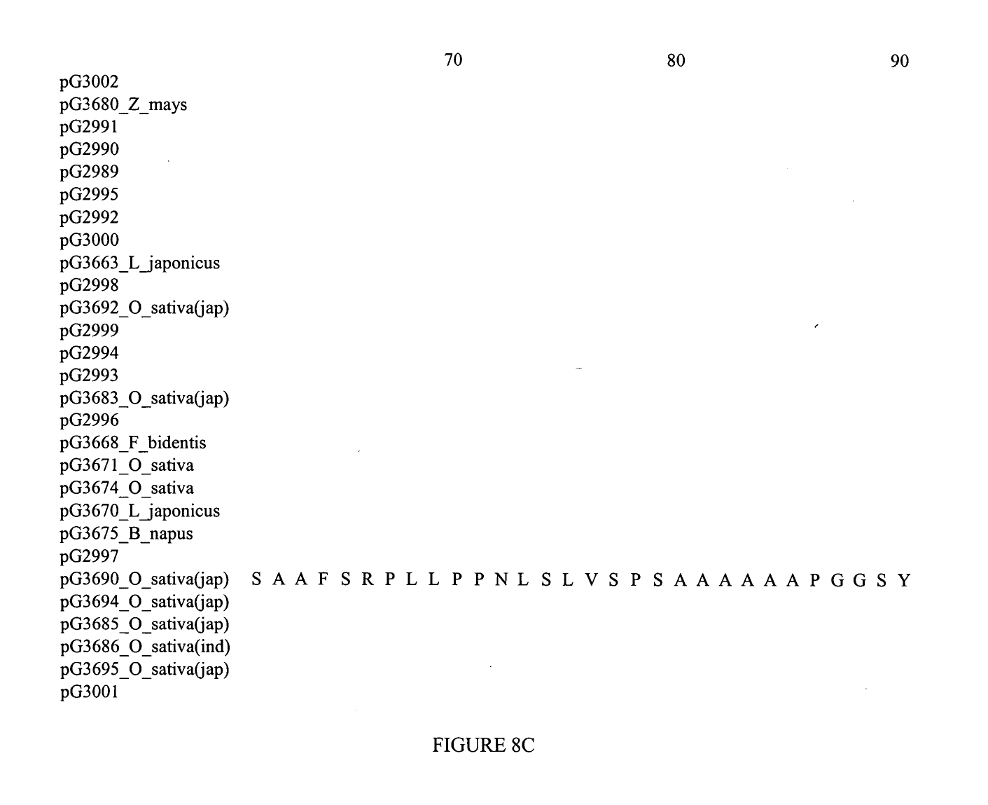

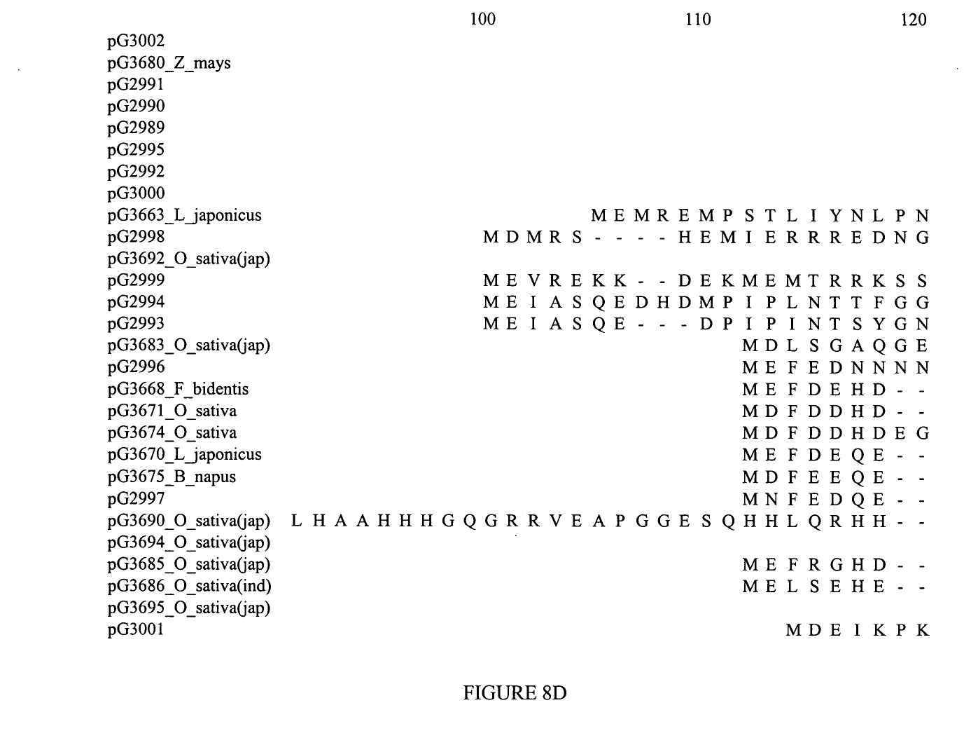

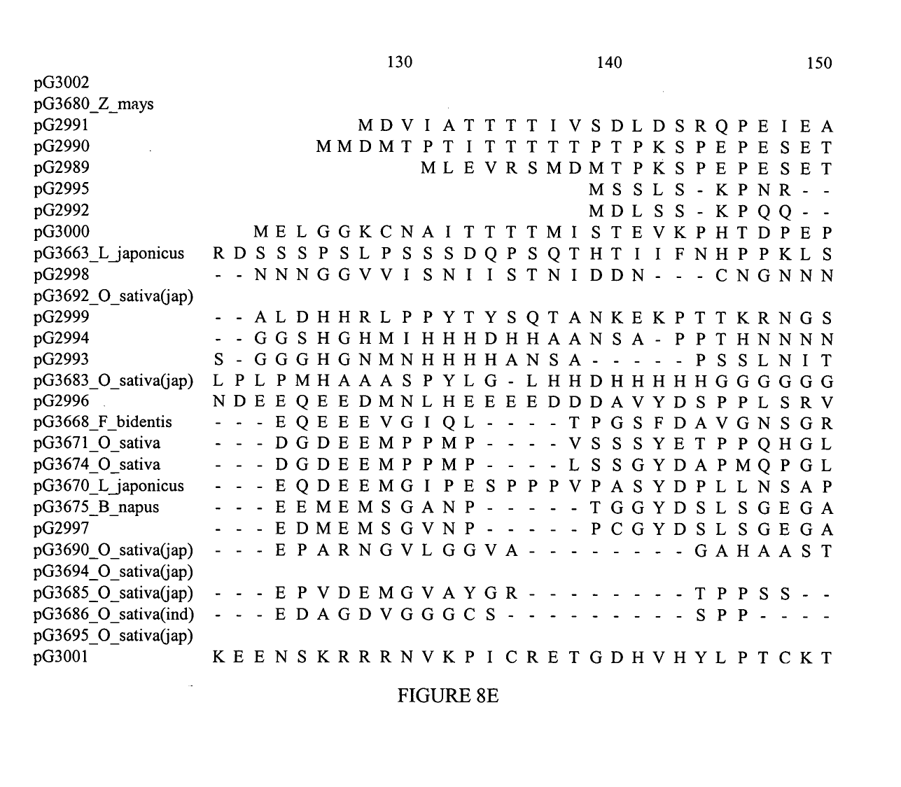

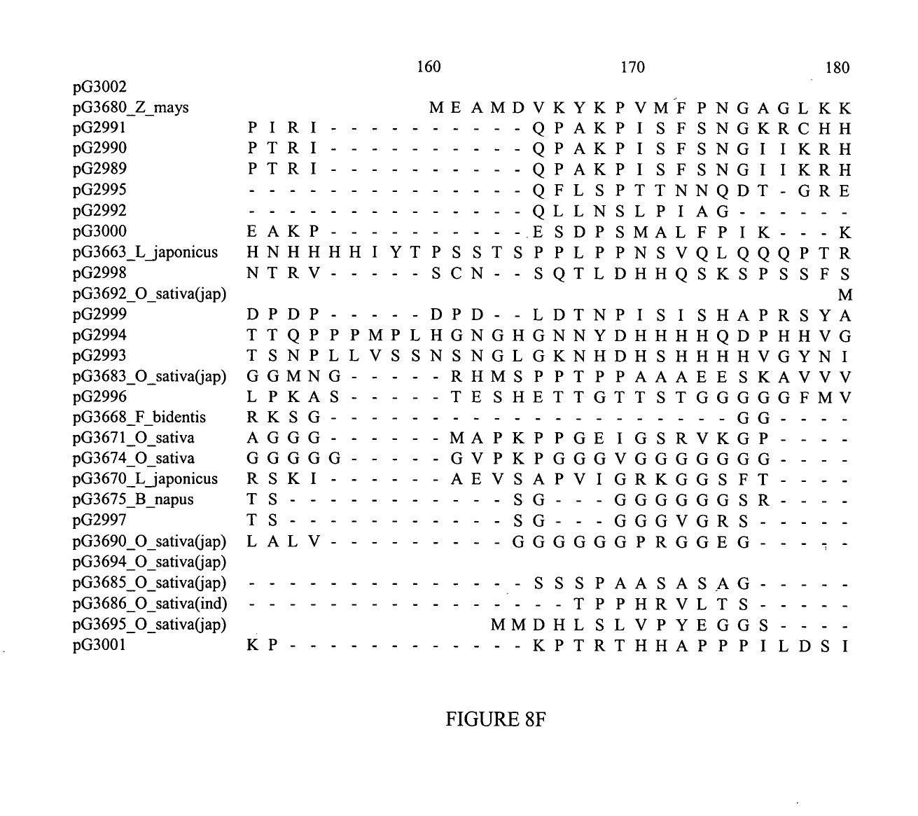

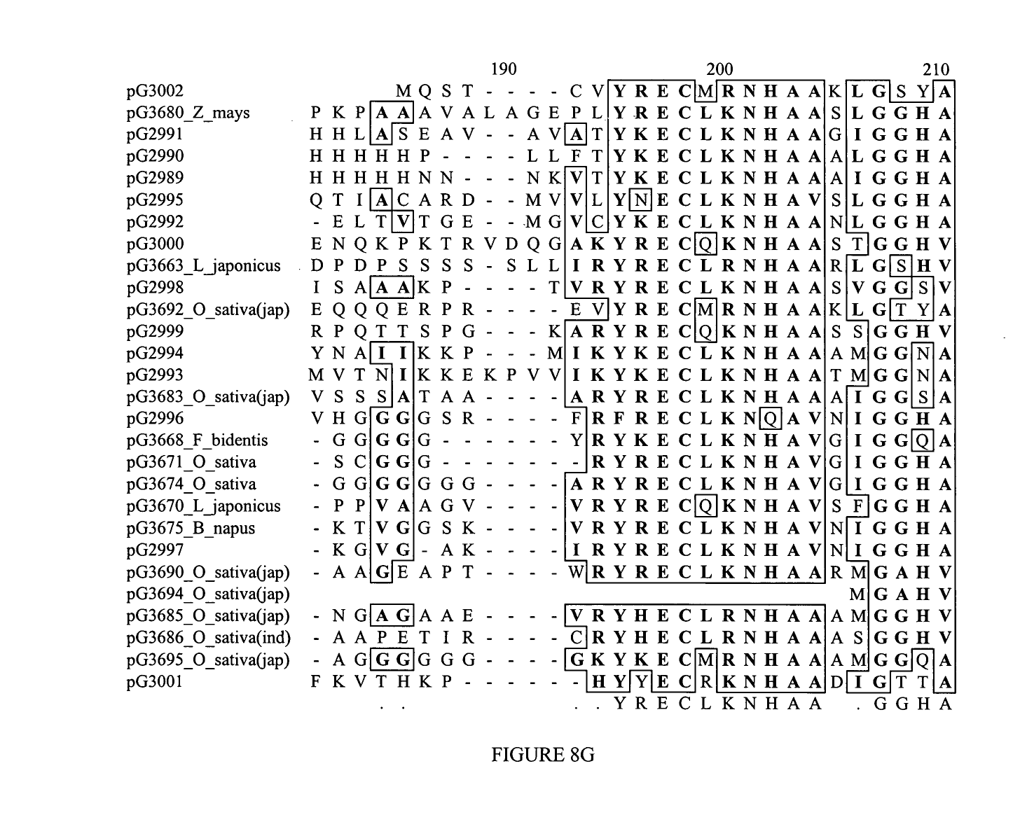

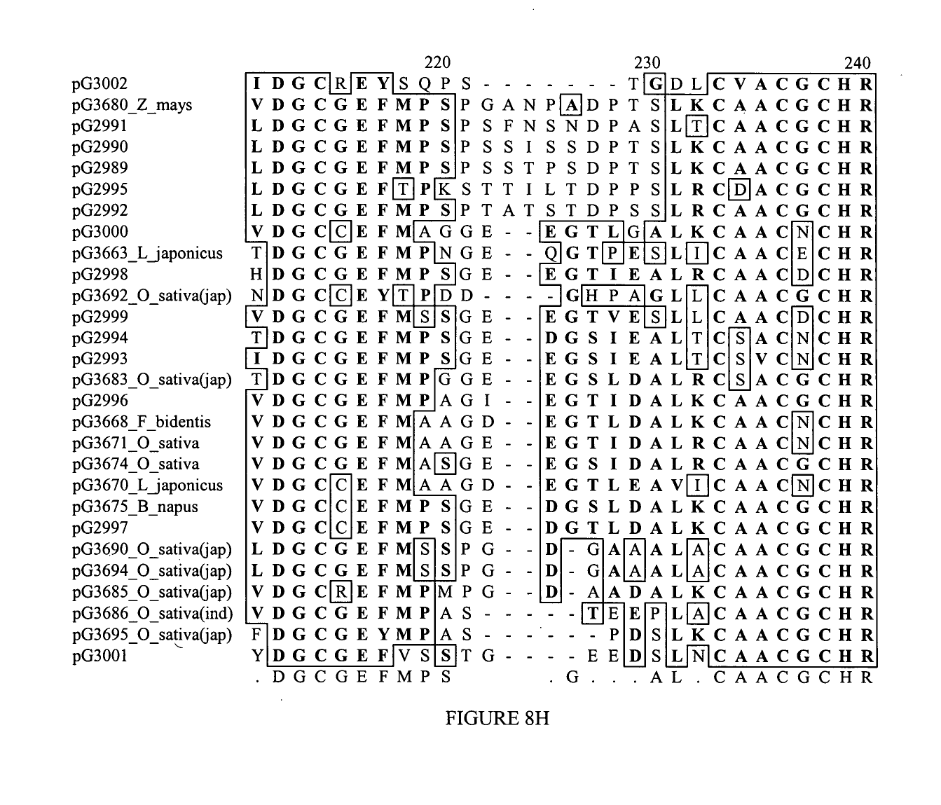

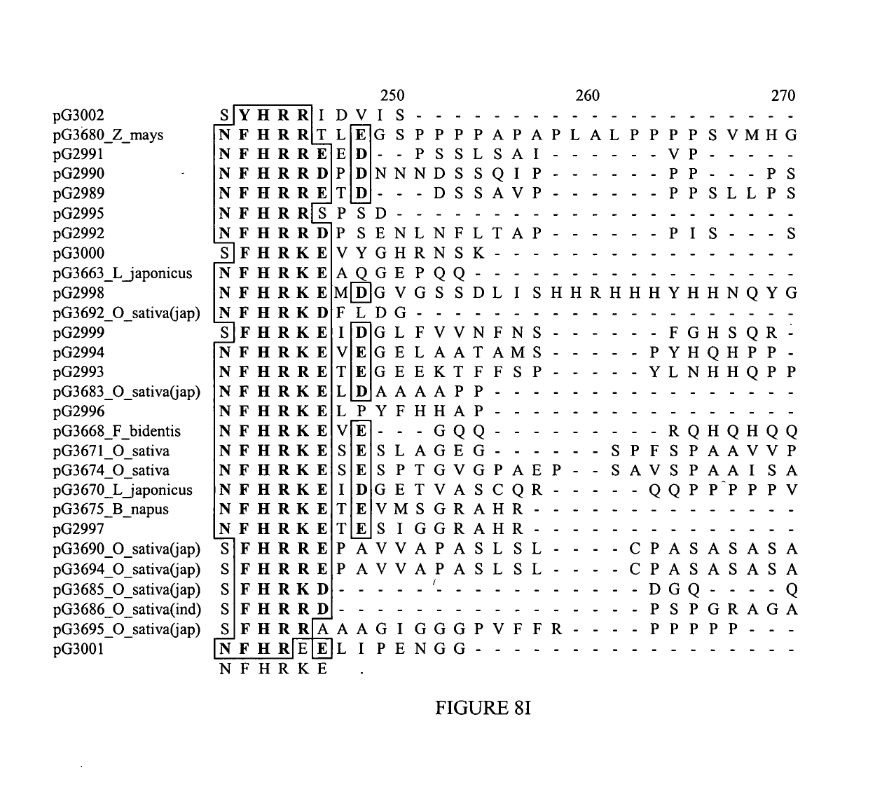

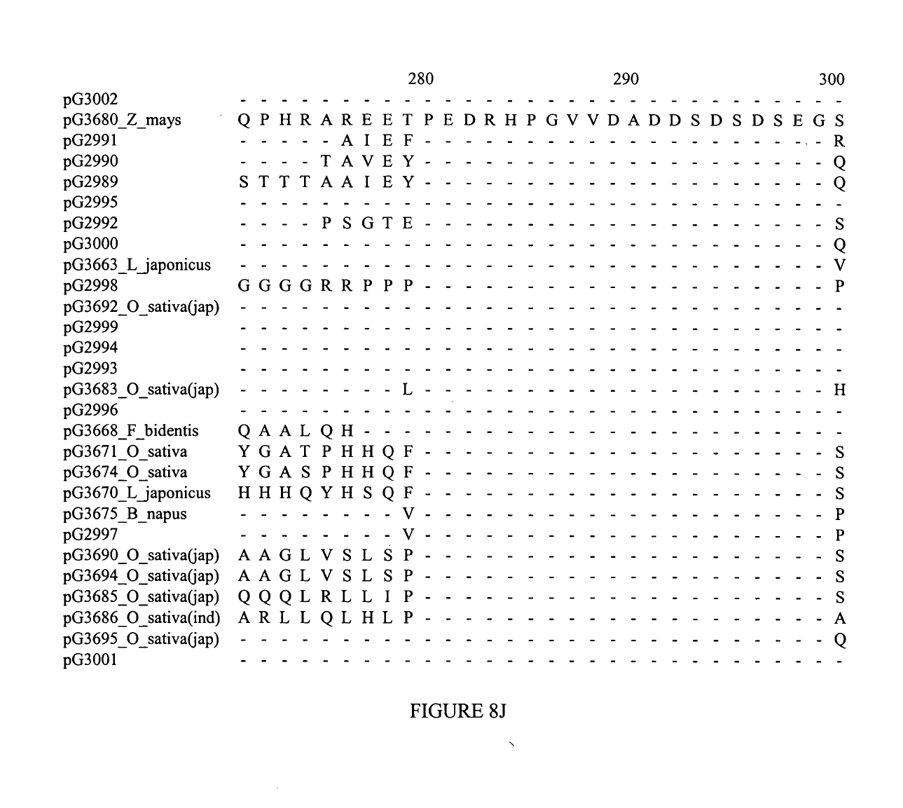

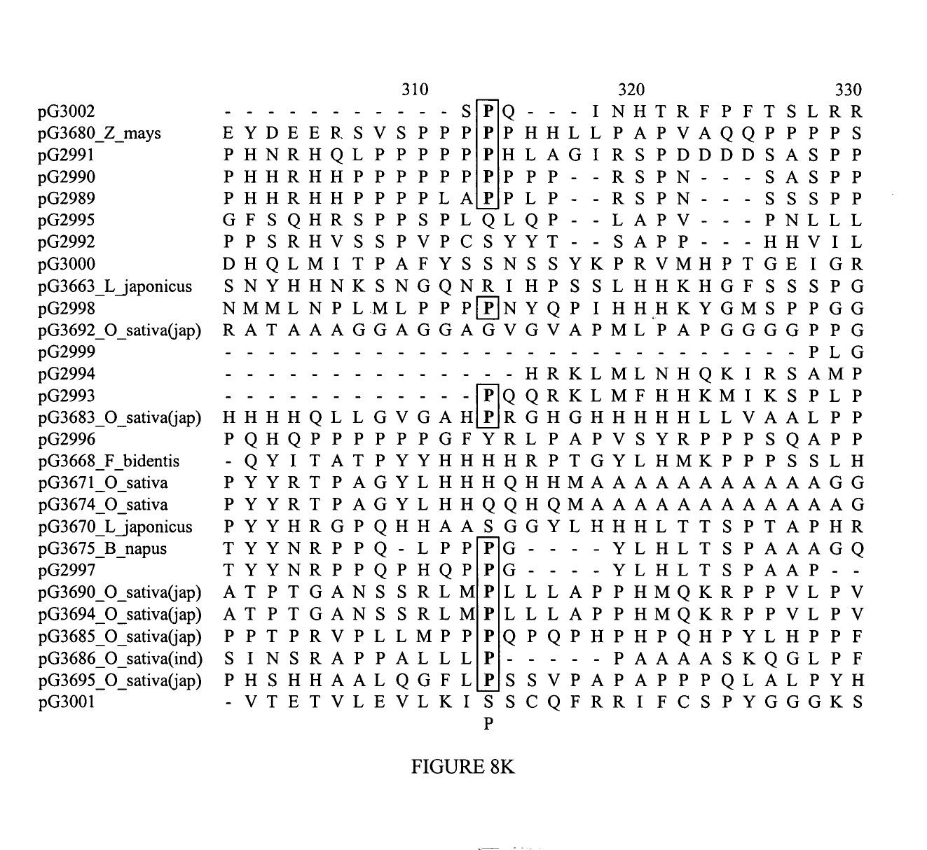

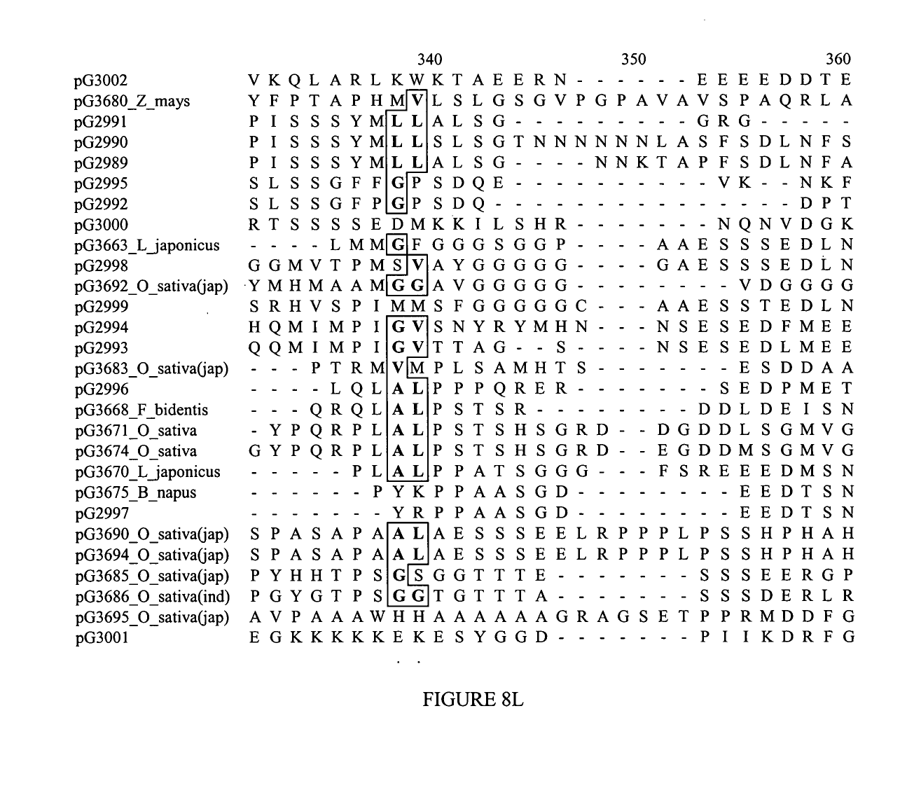

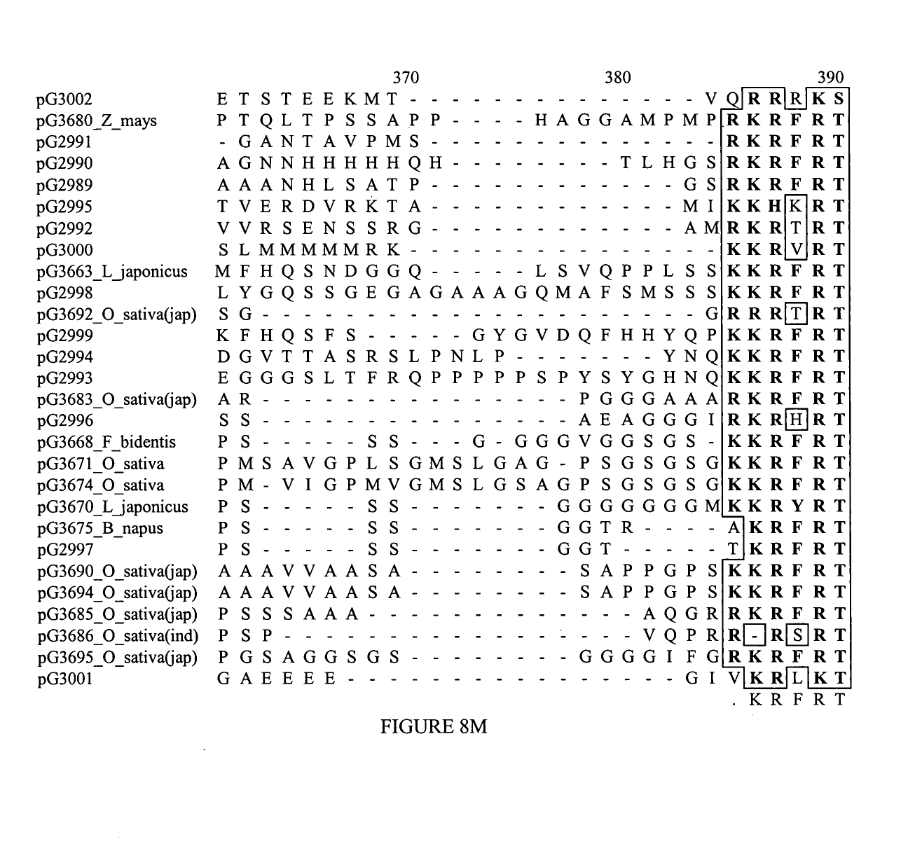

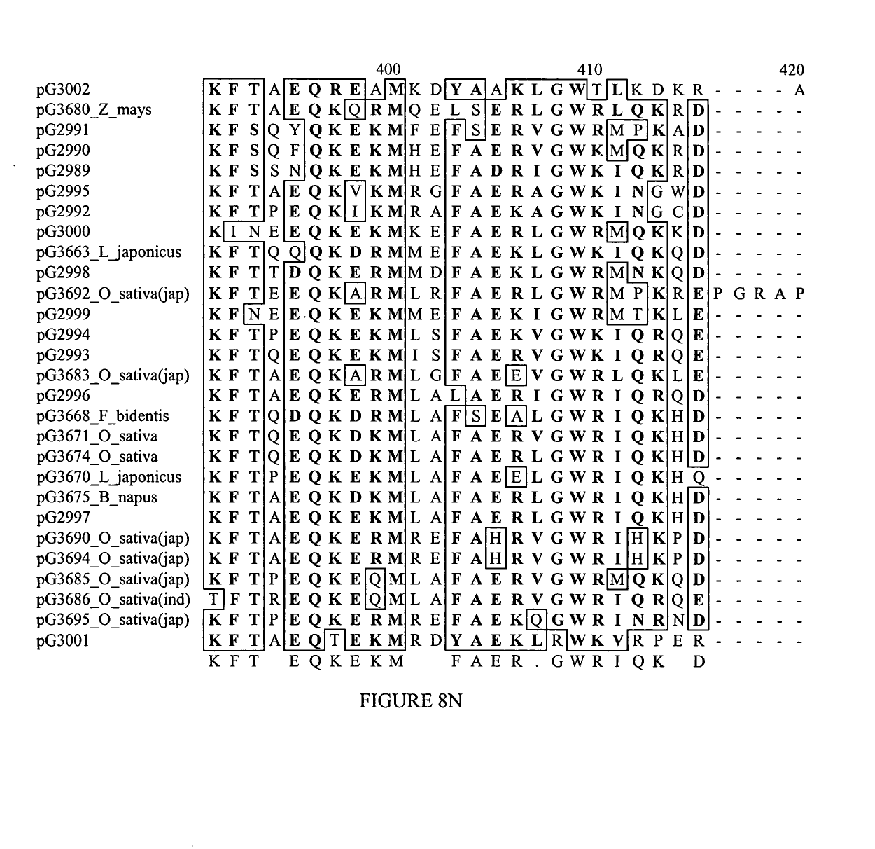

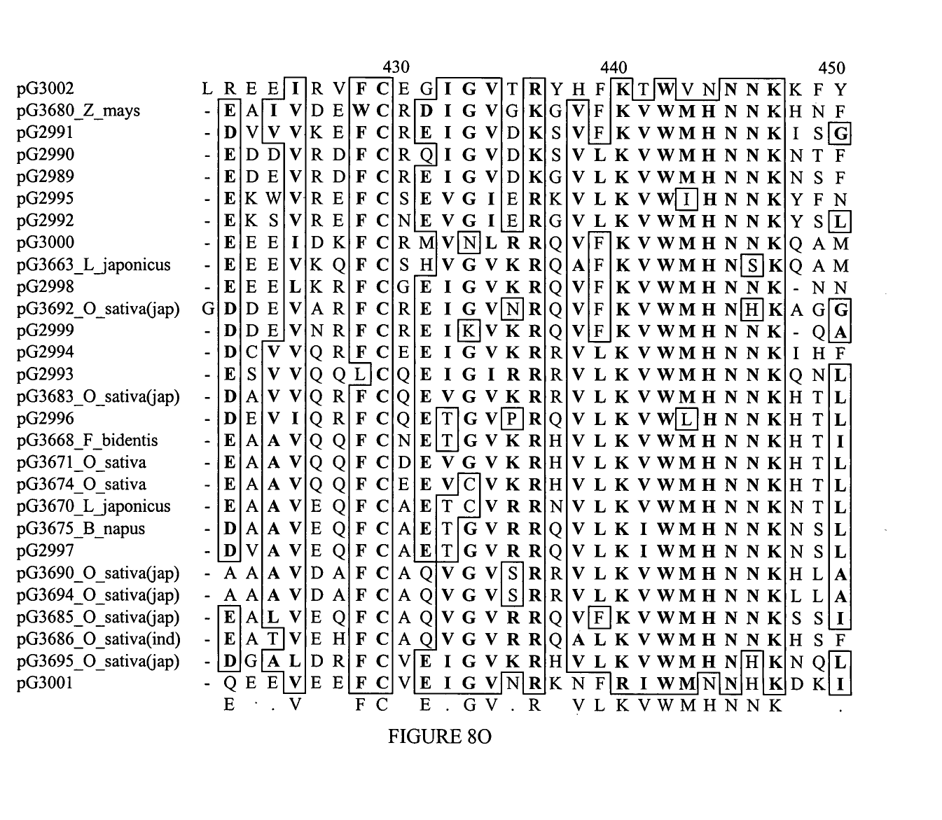

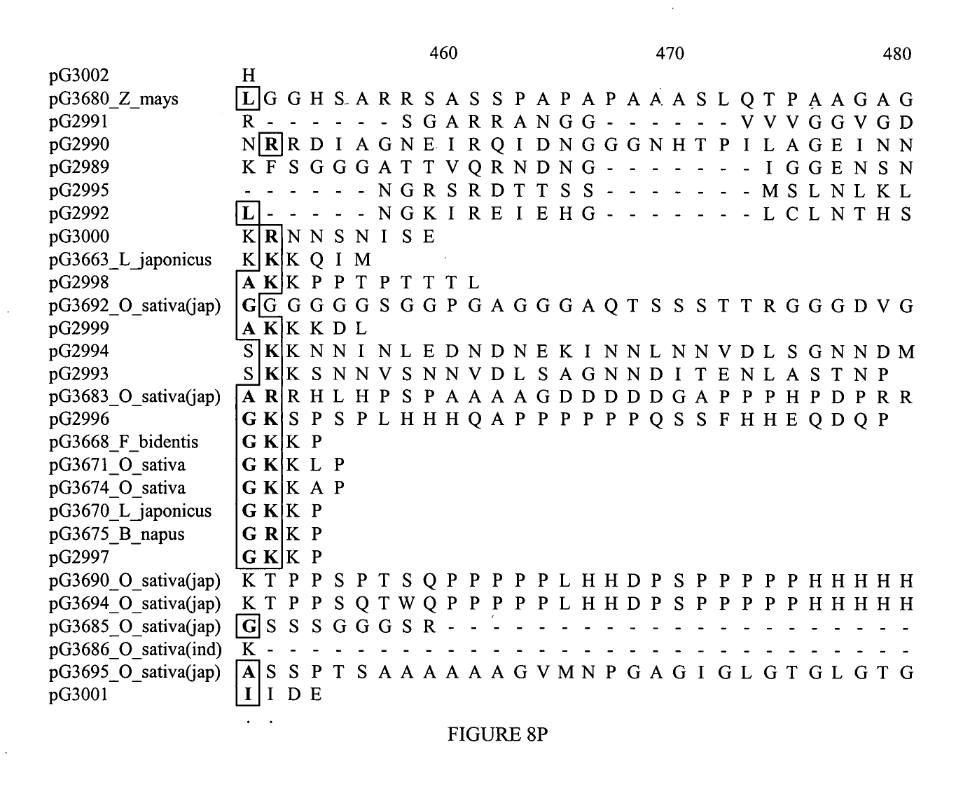

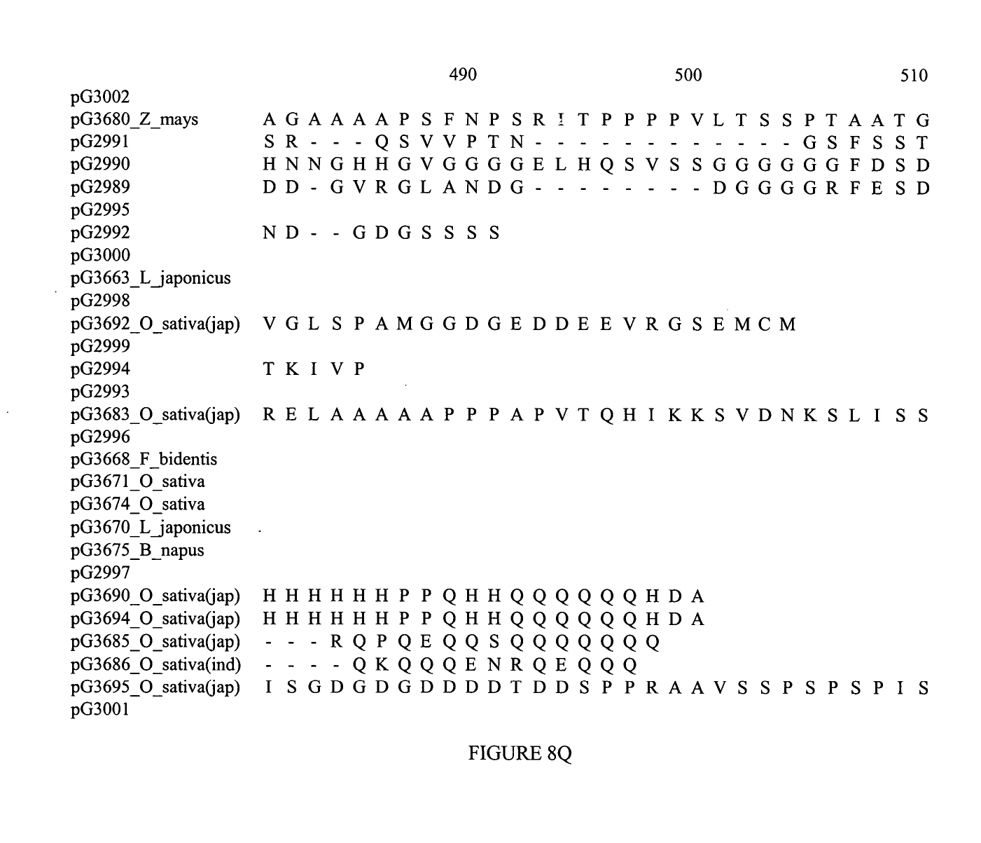

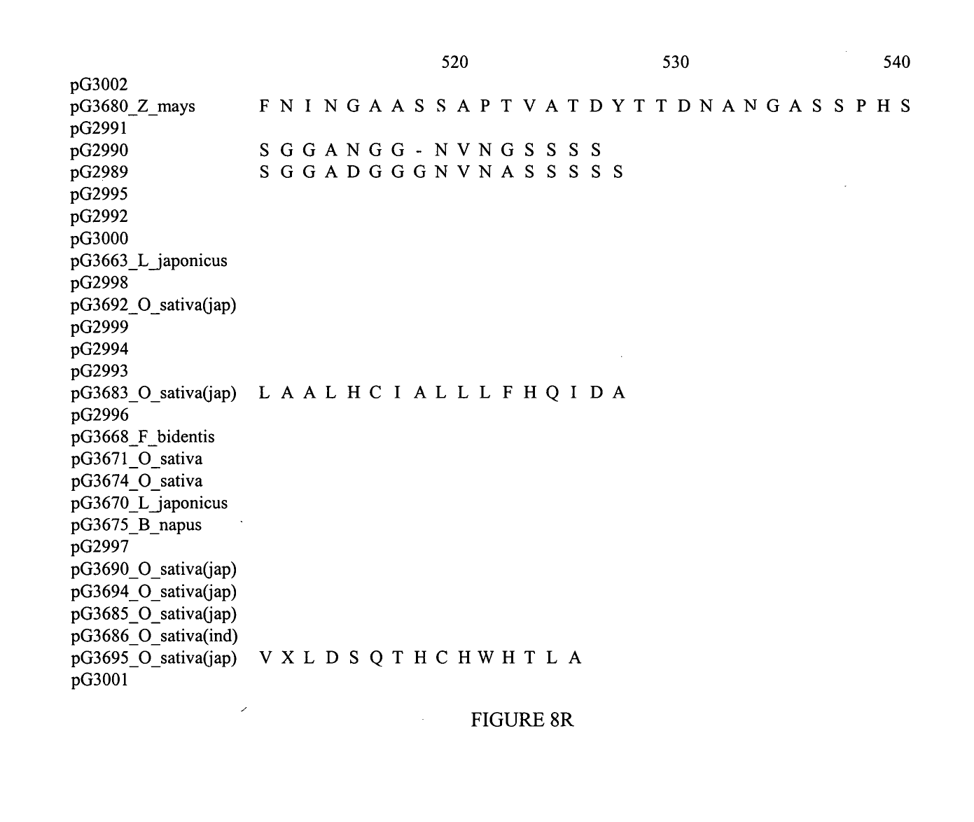

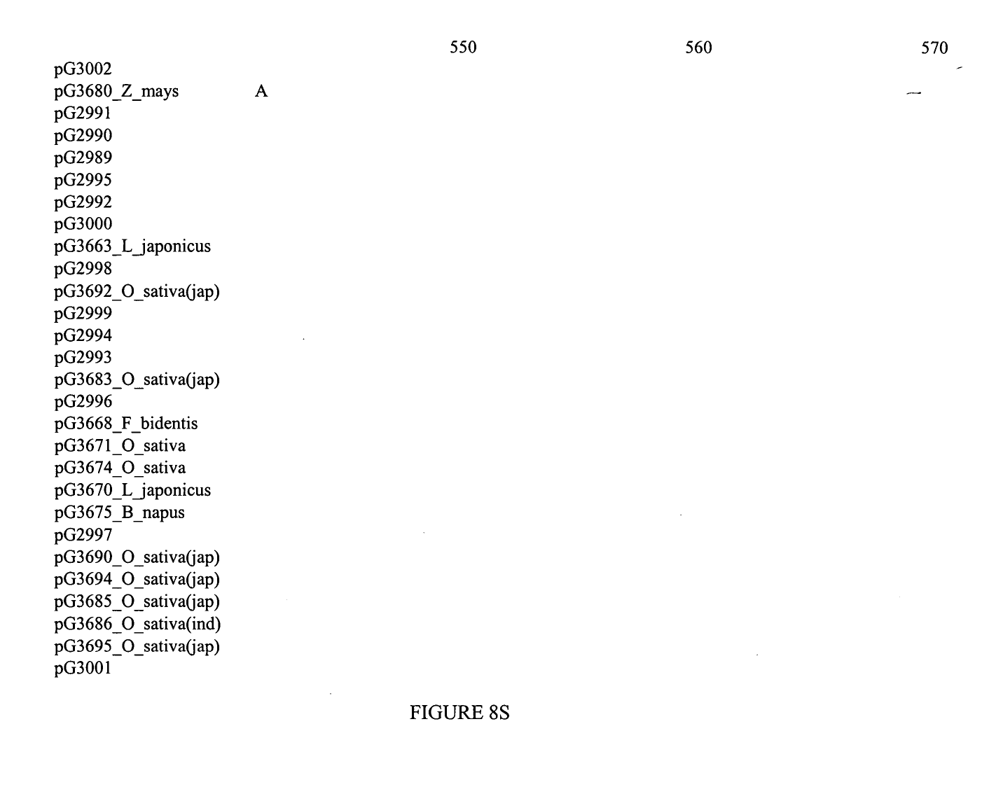

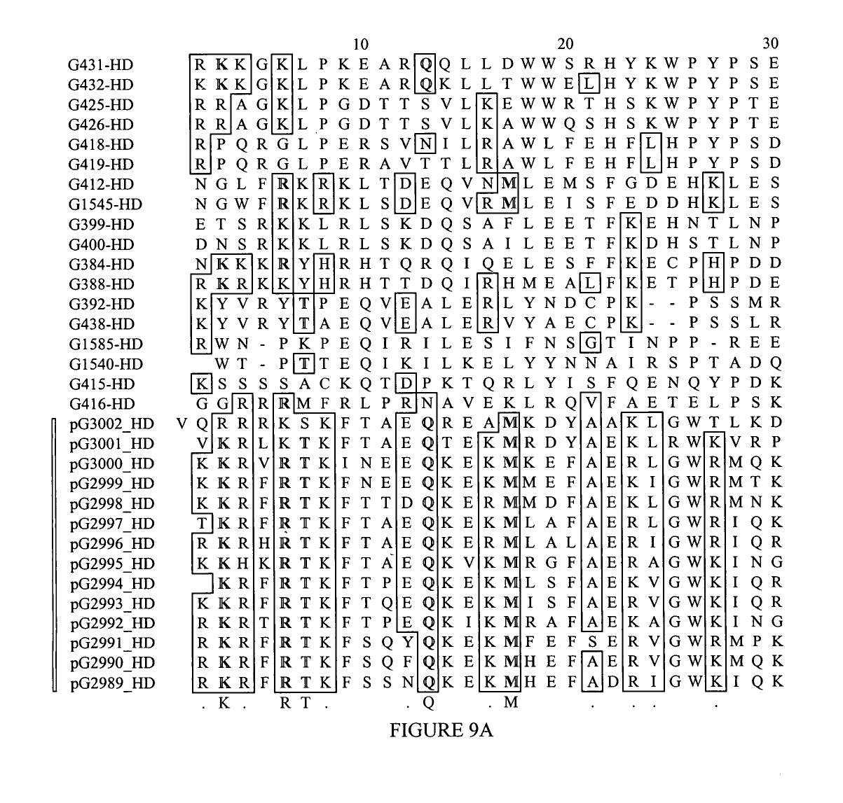

G2133 is a paralog of G47, the latter having been known from earlier studies to confer a drought tolerance phenotype when overexpressed. It was thus not surprising when G2133 was also shown to induce drought tolerance in a number of 35S::G2133 lines challenged in soil-based drought assays (Tables 9 and 10). Results with two of these lines are shown in FIGS. 7A and 7B, which compare the recovery of these lines from eight days of drought treatment with that of wild-type controls. After re-watering, all of the plants of both G2133 overexpressor lines became reinvigorated, and all of the control plants died or were severely affected by the drought treatment (Table 9).

Utilities

G2133 and its equivalogs can be used to increase the tolerance of plants to drought and to other osmotic stresses. G2133 could also be used for the generation of glyphosate resistant plants, and to increase plant resistance to oxidative stress. G2133 equivalogs include, for example, Arabidopsis thaliana SEQ ID NO: 2 (G47); Oryza sativa (japonica cultivar-group) SEQ ID NO: 98 (G3649), SEQ ID NO: 100 (G3651), and SEQ ID NO: 90 (G3644); Glycine max SEQ ID NO: 88 (G3643); Zinnia elegans SEQ ID NO: 96 (G3647); Brassica rapa subsp. Pekinensis SEQ ID NO: 92 (G3645); and Brassica oleracea SEQ ID NO: 94 (G3646).

G47 (SEQ ID NOs: 1 and 2)

Published Information

G47 corresponds to gene T22J18.2 (AAC25505). No information is available about the function(s) of G47.

Experimental Observations

The function of G47 was studied using transgenic plants in which the gene was expressed under the control of the 35S promoter. Overexpression of G47 resulted in a variety of morphological and physiological phenotypic alterations.

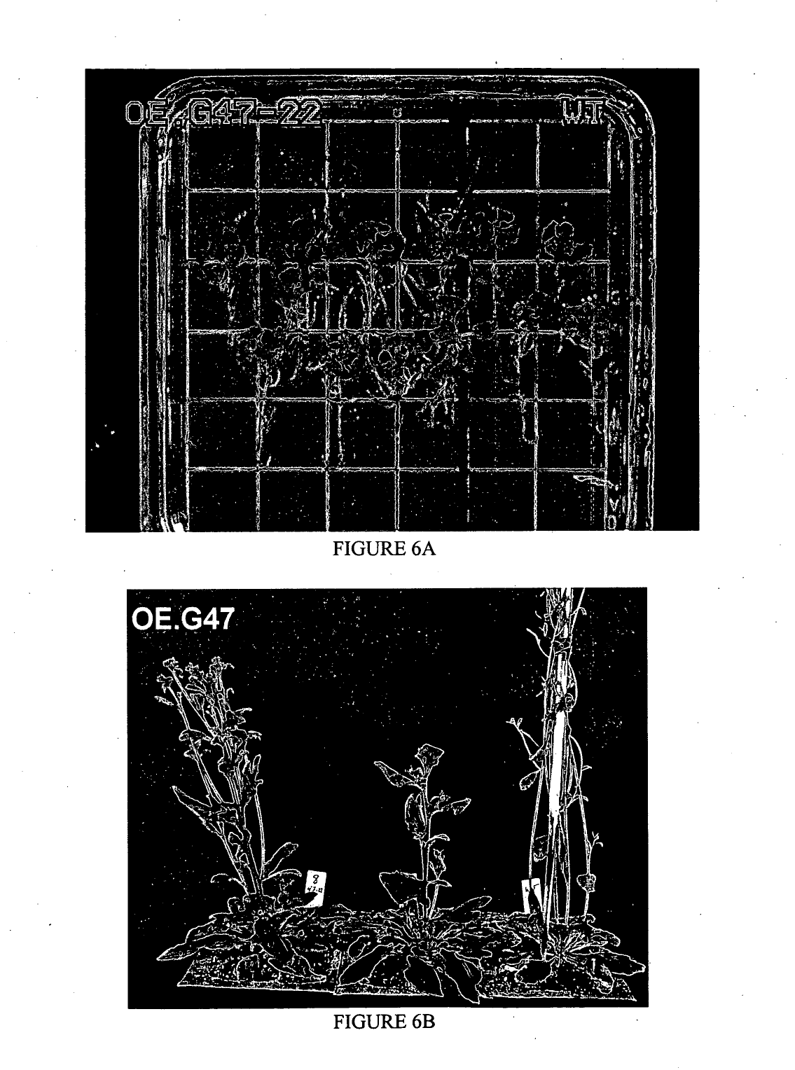

35S::G47 plants showed enhanced tolerance to osmotic stress. In a root growth assay on PEG containing media, G47 overexpressing transgenic seedlings were larger and had more root growth compared to the wild-type controls (FIG. 6A). Interestingly, G47 expression levels might be altered by environmental conditions, in particular reduced by salt and osmotic stresses. In addition to the phenotype observed in the osmotic stress assay, germination efficiency for the seeds from G47 overexpressors was low.

35S::G47 plants were also significantly larger and greener in soil-based drought assays than wild-type control plants (Tables 9 and 10).

Overexpression of G47 also produced a substantial delay in flowering time and caused a marked change in shoot architecture. 35S::G47 transformants were small at early stages and switched to flowering more than a week later than wild-type controls (continuous light conditions). Interestingly, the inflorescences from these plants appeared thick and fleshy, had reduced apical dominance, and exhibited reduced internode elongation leading to a short compact stature (FIG. 6B). The branching pattern of the stems also appeared abnormal, with the primary shoot becoming ‘kinked’ at each coflorescence node. Additionally, the plants showed slightly reduced fertility and formed rather small siliques that were borne on short pedicels and held vertically, close against the stem.

Additional alterations were detected in the inflorescence stems of 35S::G47 plants. Stem sections from T2-21 and T2-24 plants were of wider diameter, and had large irregular vascular bundles containing a much greater number of xylem vessels than wild type. Furthermore some of the xylem vessels within the bundles appeared narrow and were possibly more lignified than were those of controls.

G47 was expressed at higher levels in rosette leaves, and transcripts can be detected in other tissues (flower, embryo, silique, and germinating seedling), but apparently not in roots.

Utilities

G47 or its equivalogs can be used to increase the tolerance of plants to drought and to other osmotic stresses. G47 or its equivalogs could also be used to manipulate flowering time, to modify plant architecture and stem structure, including development of vascular tissues and lignin content. The use of G47 or its equivalogs from tree species could offer the potential for modulating lignin content. This might allow the quality of wood used for furniture or construction to be improved. G47 equivalogs include, for example, Arabidopsis thaliana SEQ ID NO: 12 (G2133); Oryza sativa (japonica cultivar-group) SEQ ID NOs: 98 (G3649), SEQ ID NO: 100 (G3651), and SEQ ID NO: 90 (G3644); Glycine max SEQ ID NO: 88 (G3643); Zinnia elegans SEQ ID NO: 96 (G3647); Brassica rapa subsp. Pekinensis SEQ ID NO: 92 (G3645); and Brassica oleracea SEQ ID NO: 94 (G3646).

G1274 (SEQ ID NOs: 5 and 6)

Published Information

G1274 is a member of the WRKY family of transcription factors. The gene corresponds to WRKY51 (At5g64810). No information is available about the function(s) of G 1274.

Exiperimental Observations

RT-PCR analysis was used to determine the endogenous expression pattern of G1274. Expression of G1274 was detected in leaf, root and flower tissues. The biotic stress related conditions, Erysiphe and SA treatment, induced expression of G1274 in leaf tissue. The gene also appeared to be slightly induced by osmotic and cold stress treatments and perhaps by auxin.

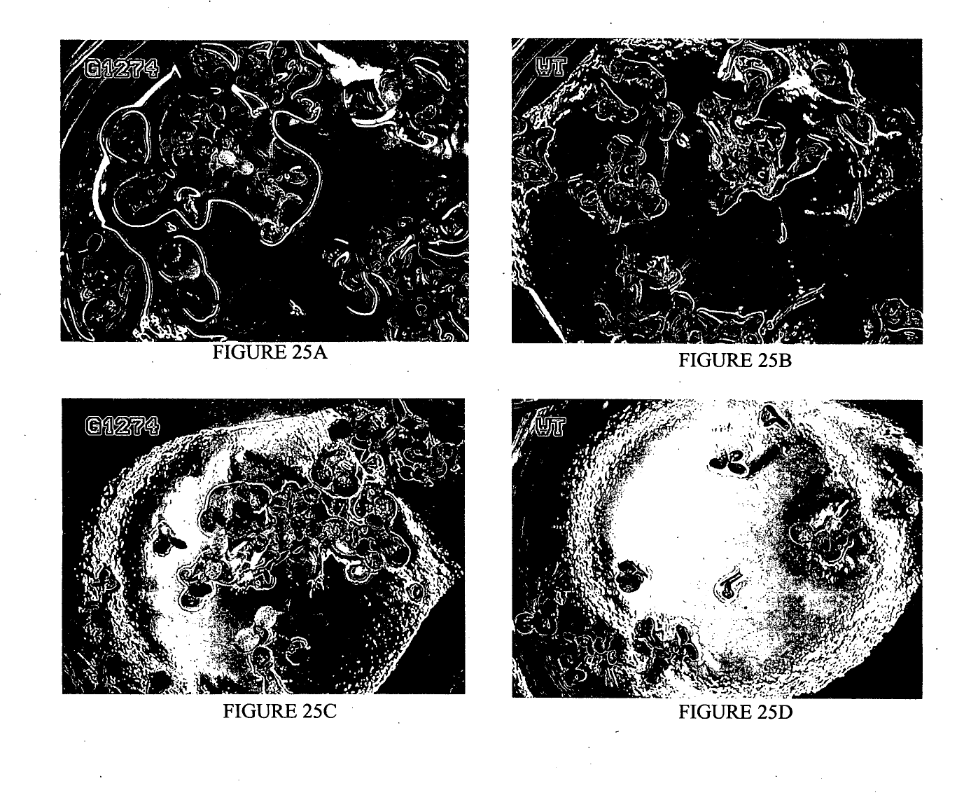

The function of G1274 was studied using transgenic plants in which the gene was expressed under the control of the 35S promoter. G1274 overexpressing lines were more tolerant to growth on low nitrogen containing media. In an assay intended to determine whether the transgene expression could alter C/N sensing, 35S::G1274 seedlings contained less anthocyanins (FIG. 25A) than wild-type controls (FIG. 25B) grown on high sucrose/N— and high sucrose/N/Gln plates. These data together indicated that overexpression of G1274 may alter a plant's ability to modulate carbon and/or nitrogen uptake and utilization.

G1274 overexpression and wild-type germination were also compared in a cold germination assay, the overexpressors appearing larger and greener (FIG. 25C) than the controls (FIG. 25D).

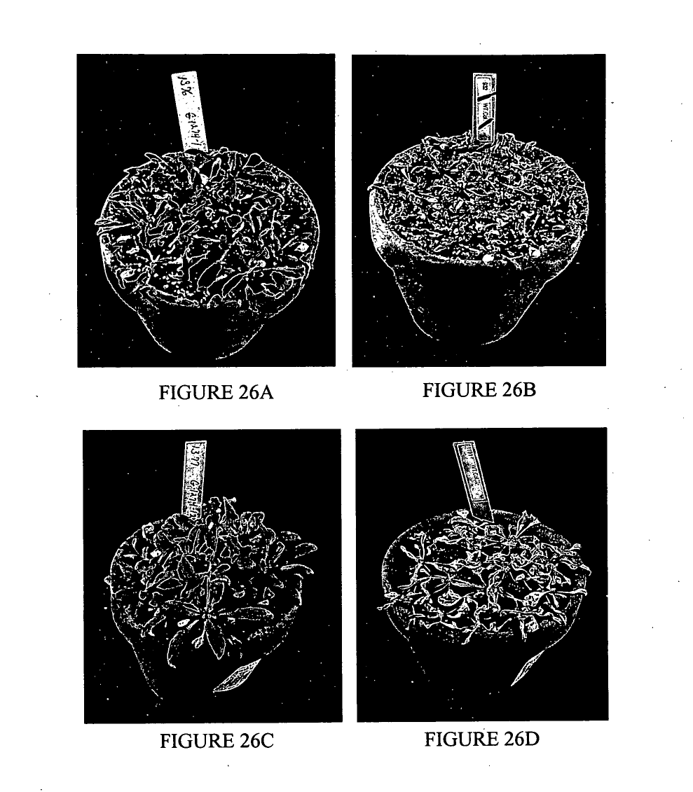

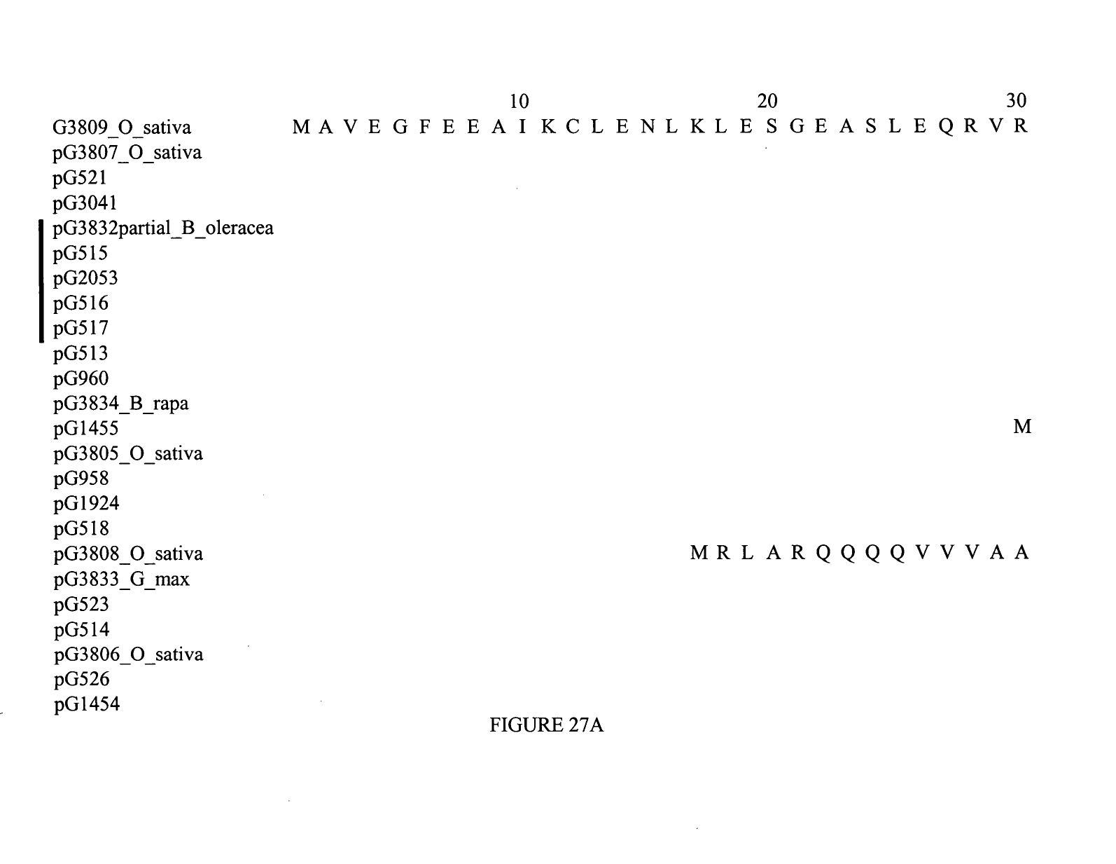

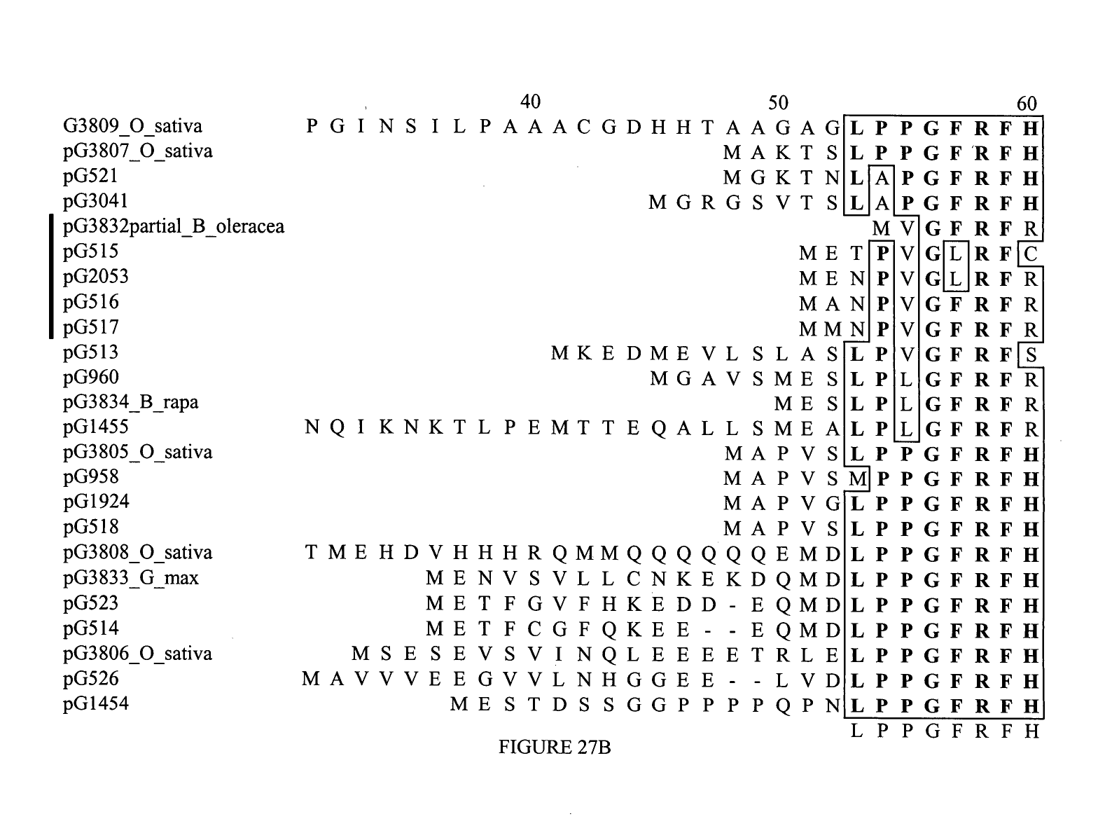

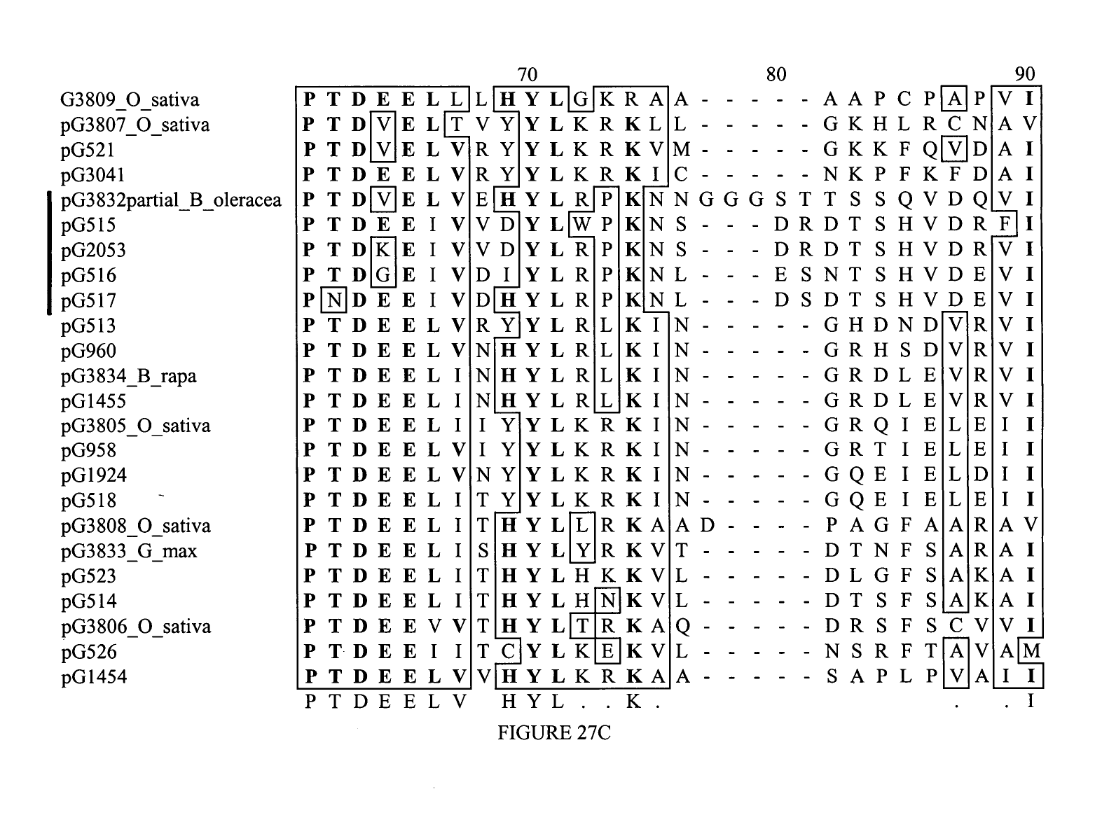

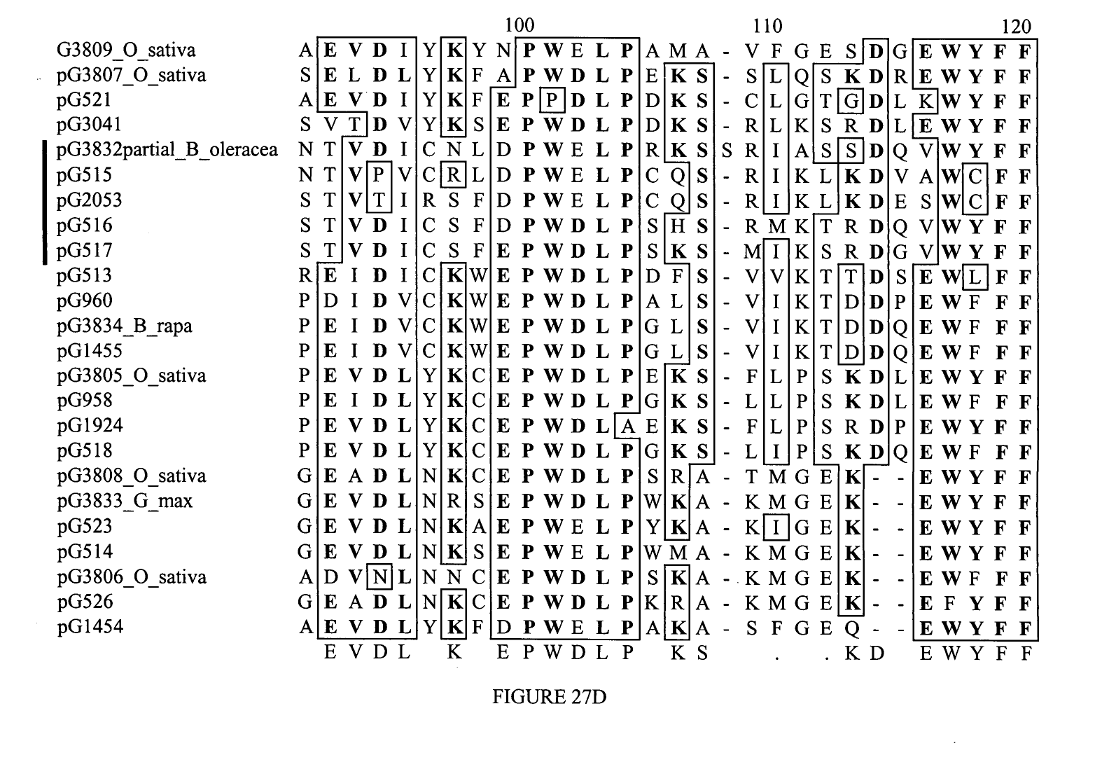

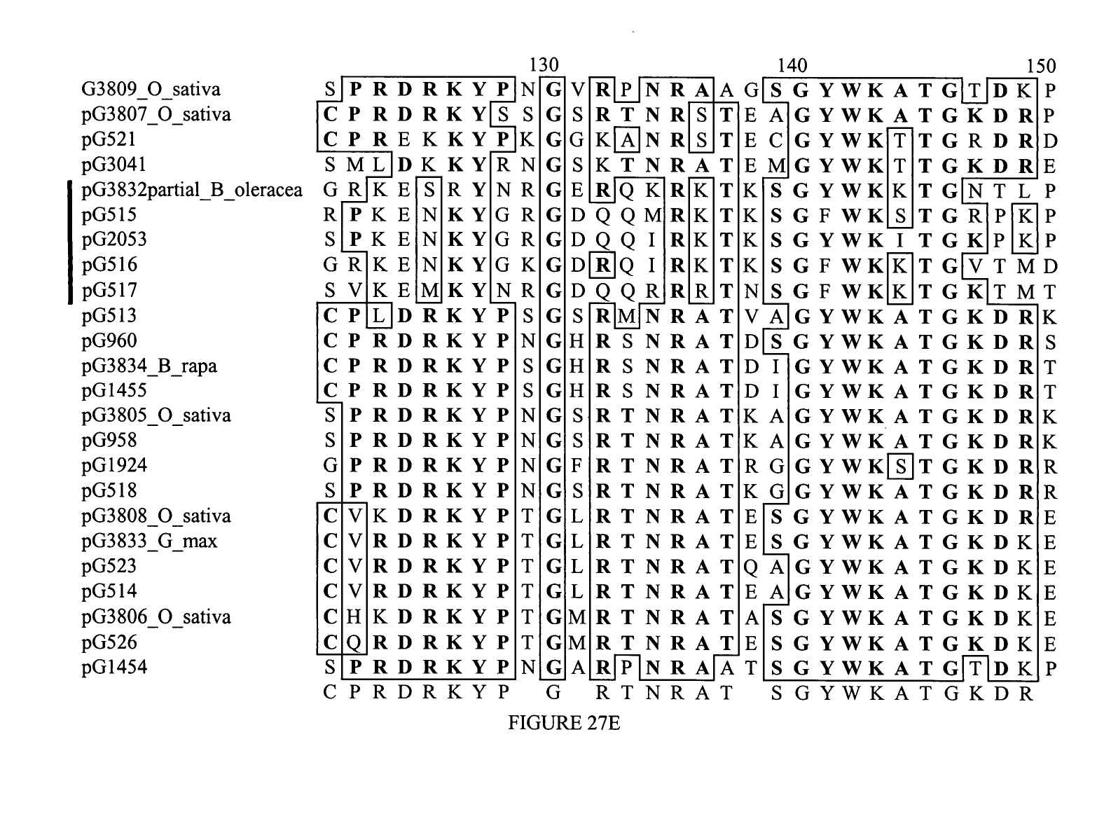

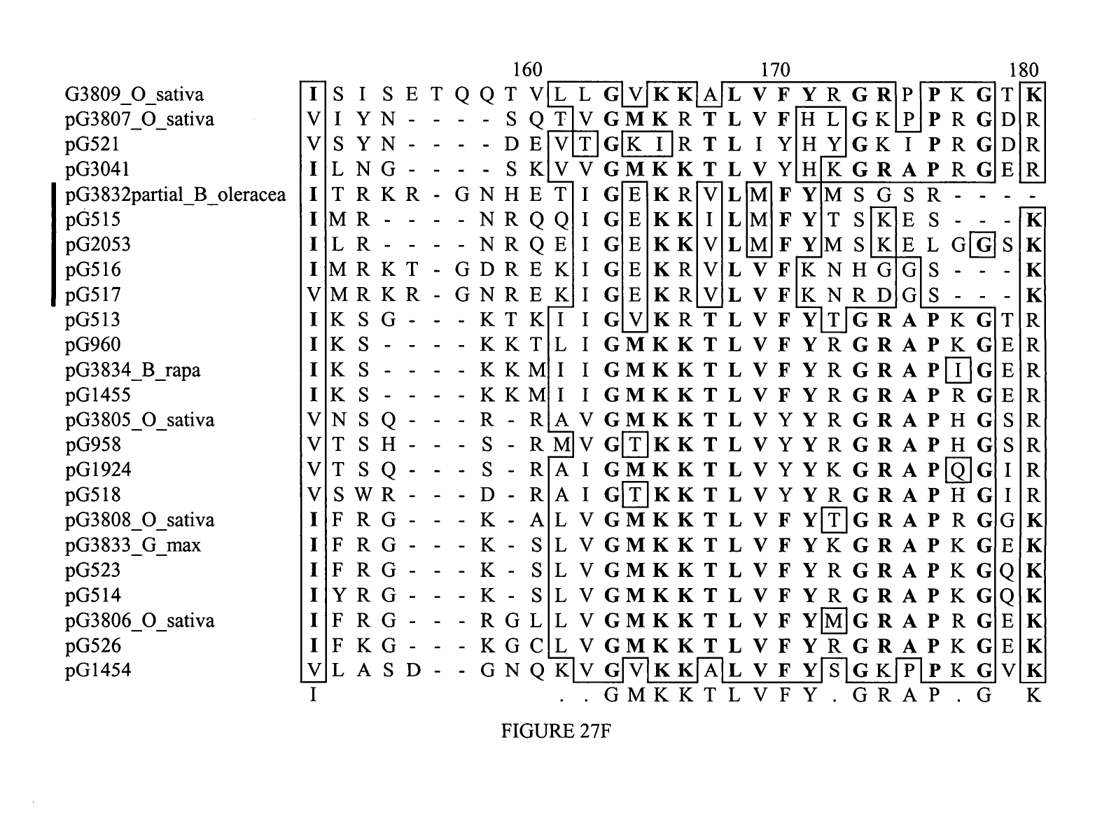

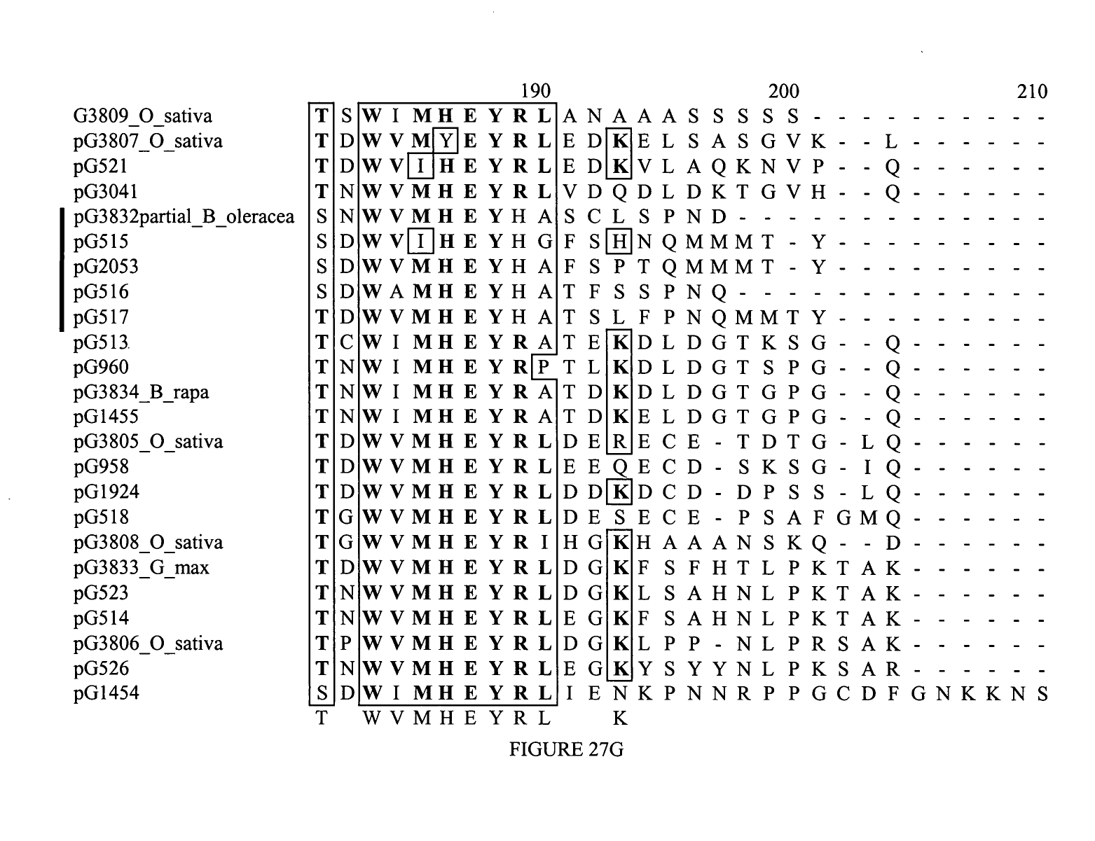

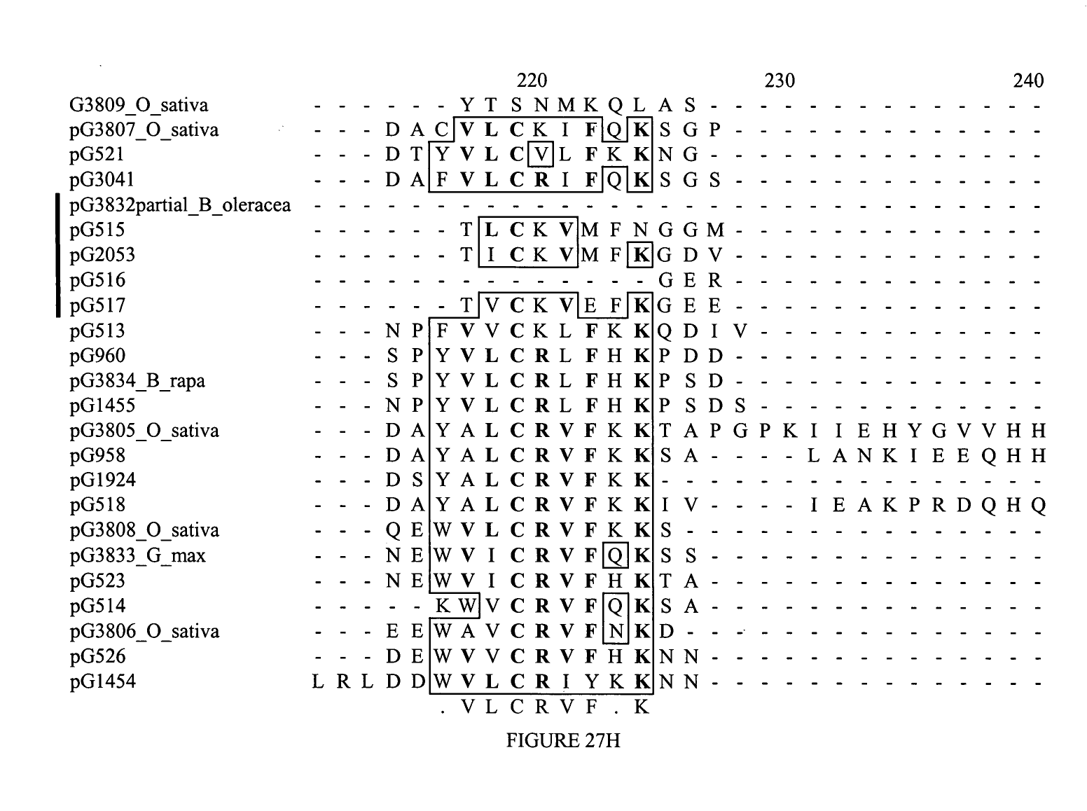

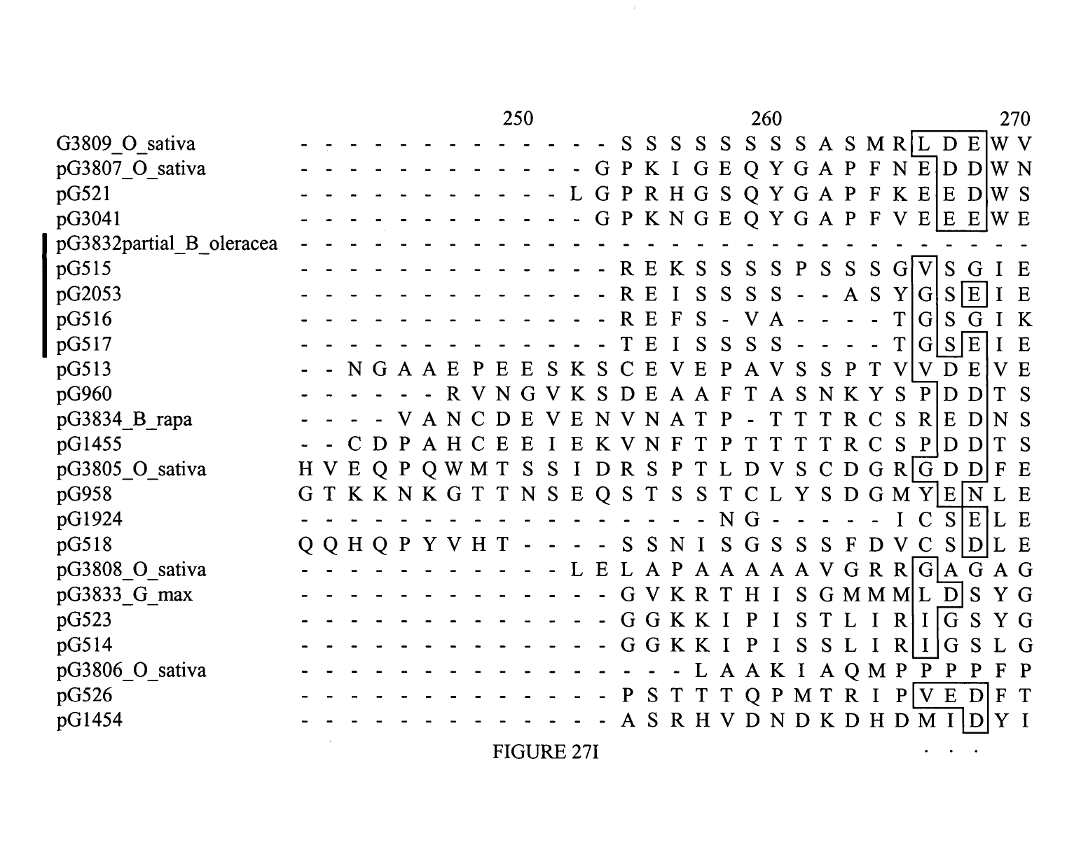

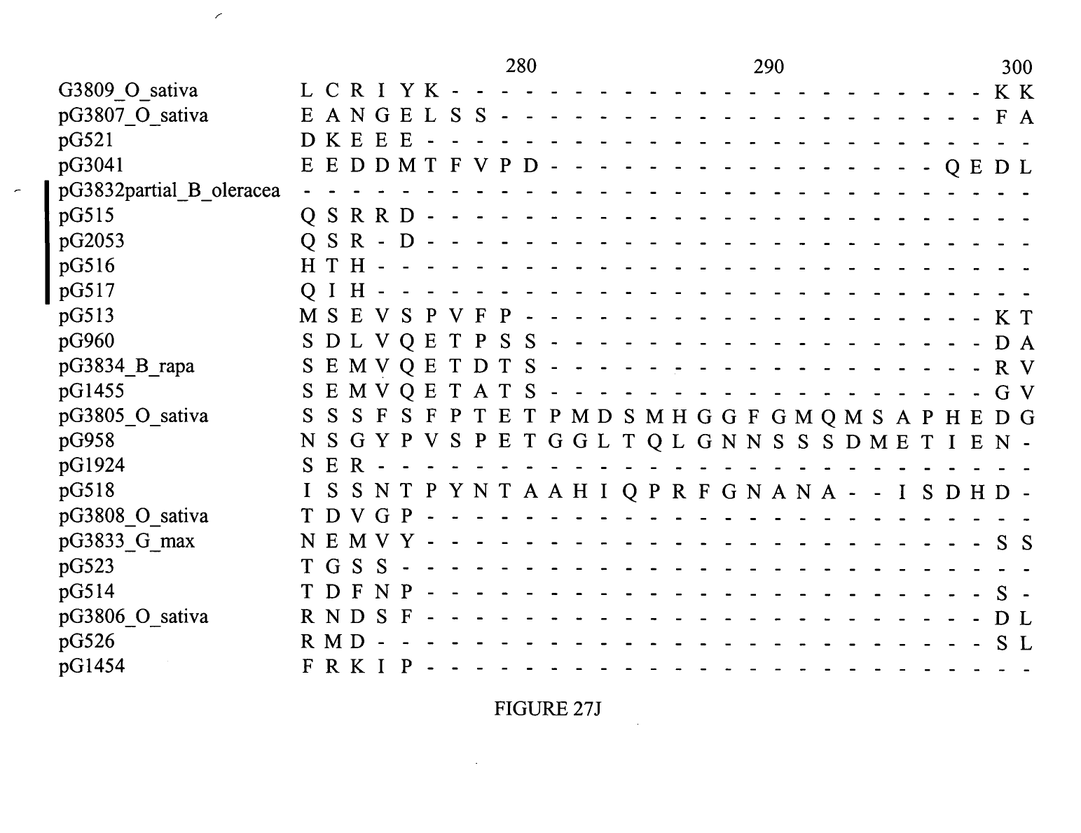

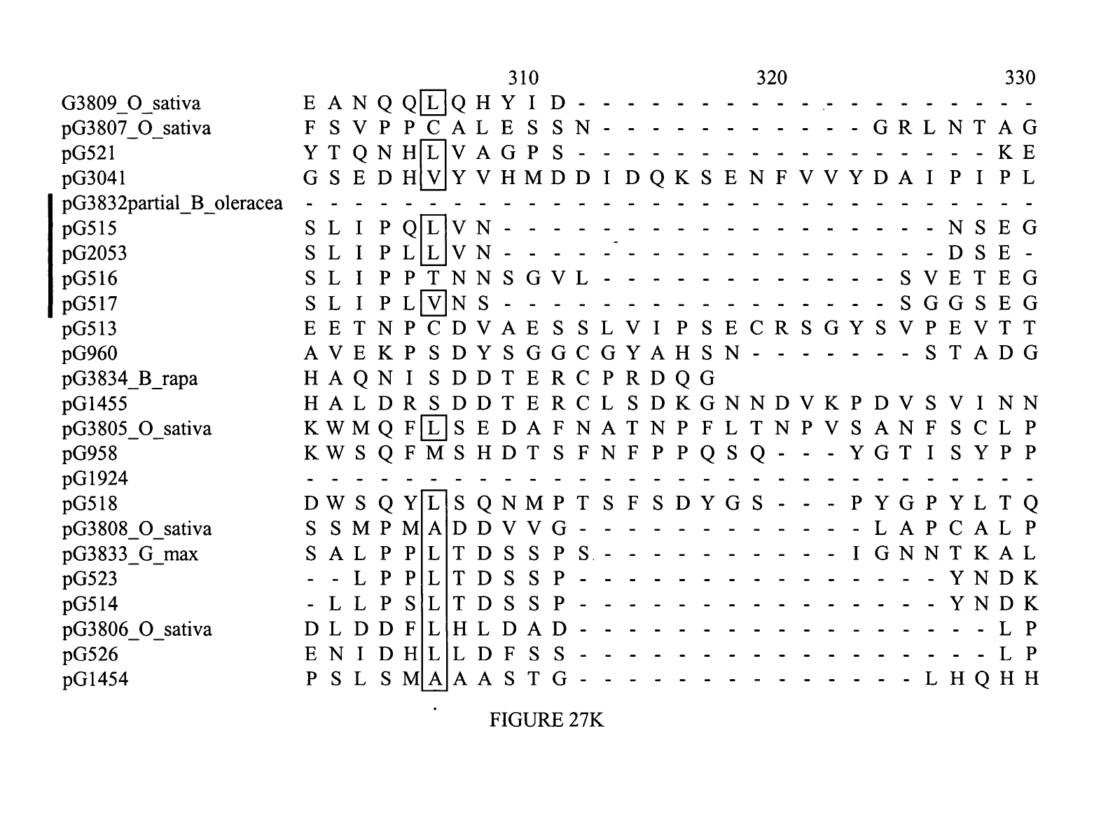

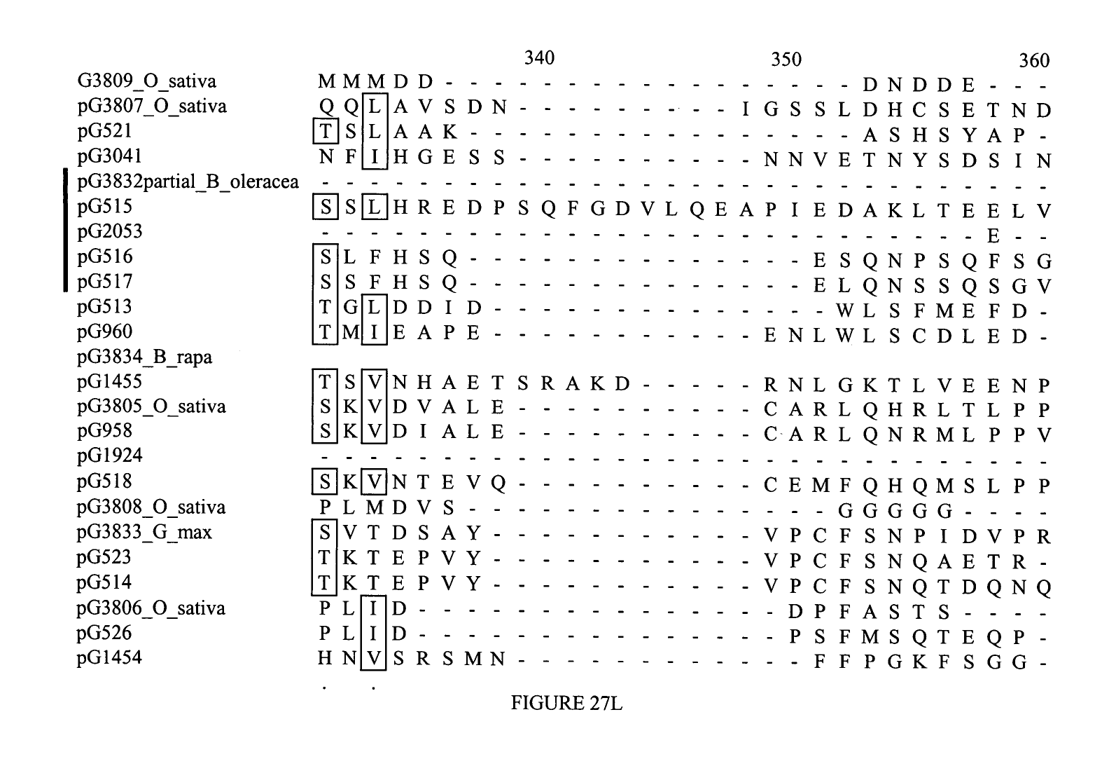

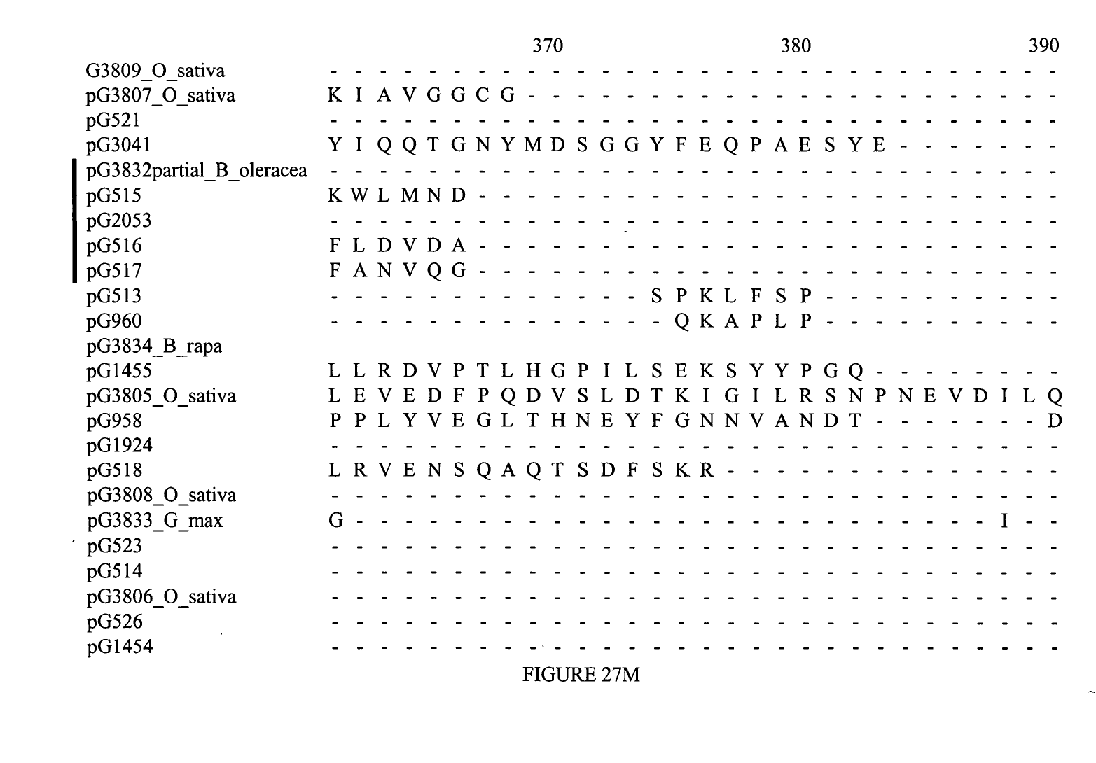

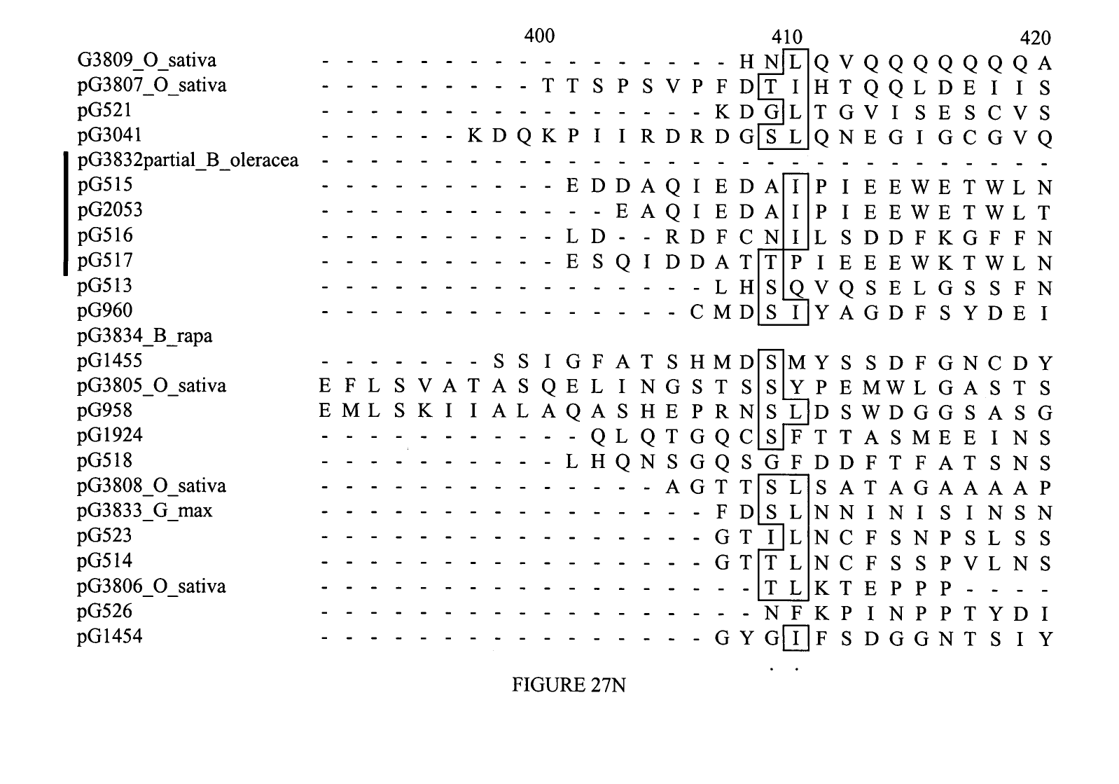

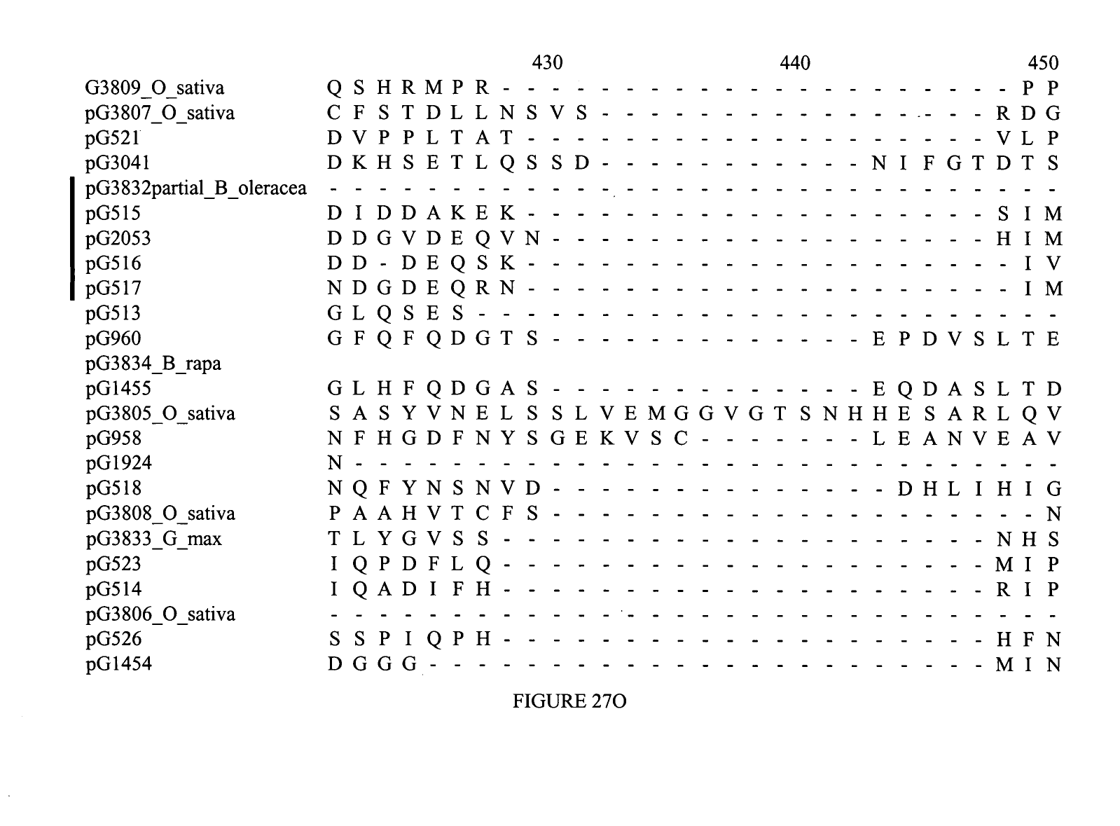

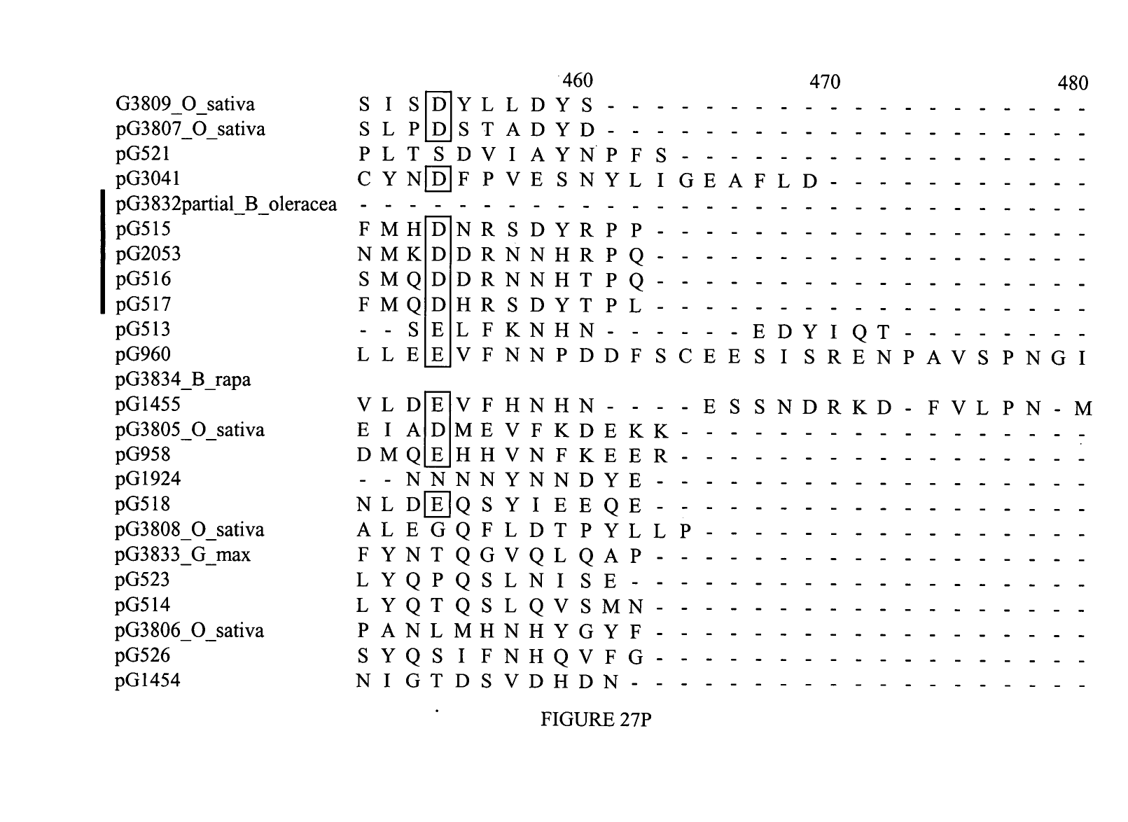

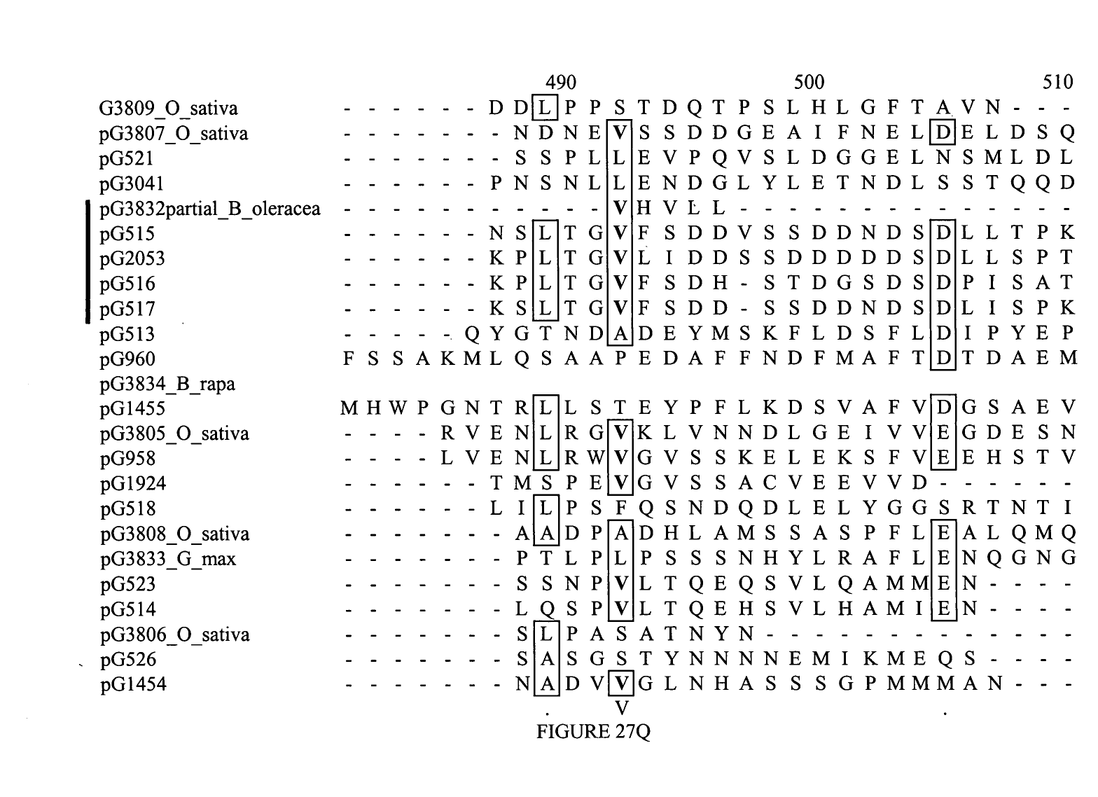

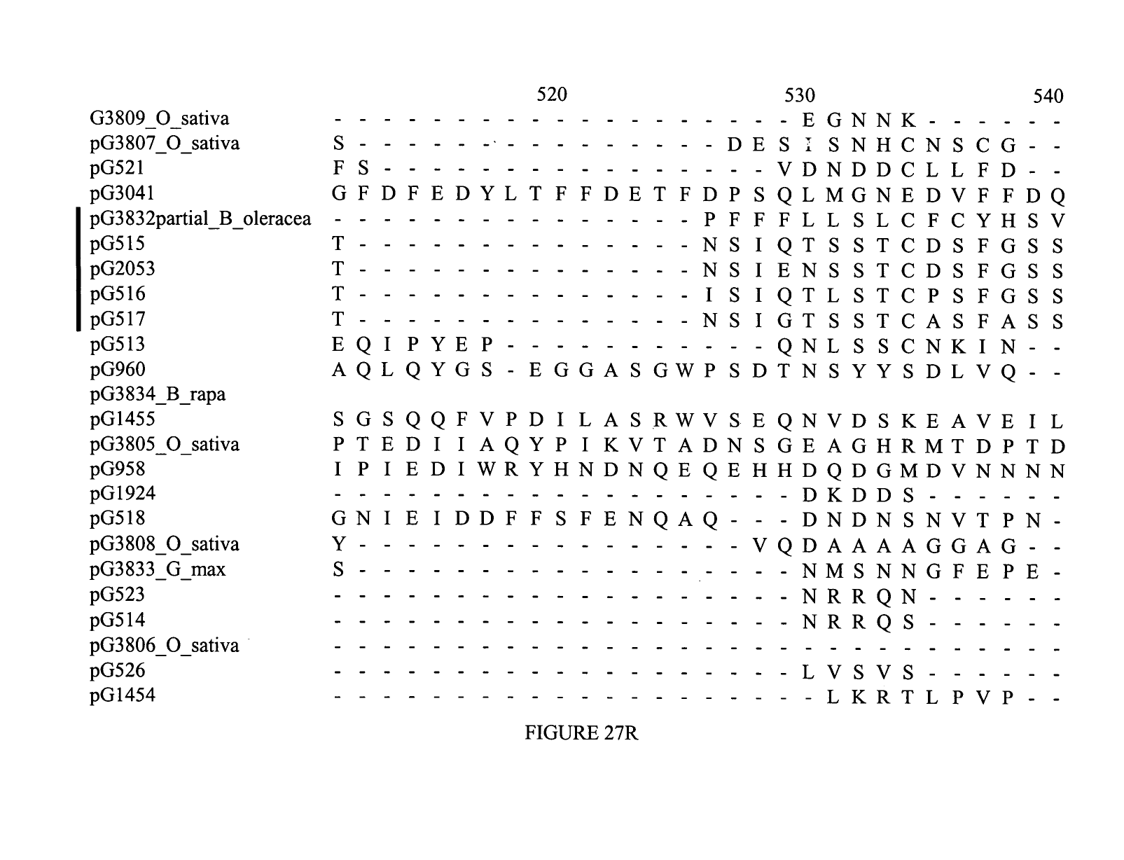

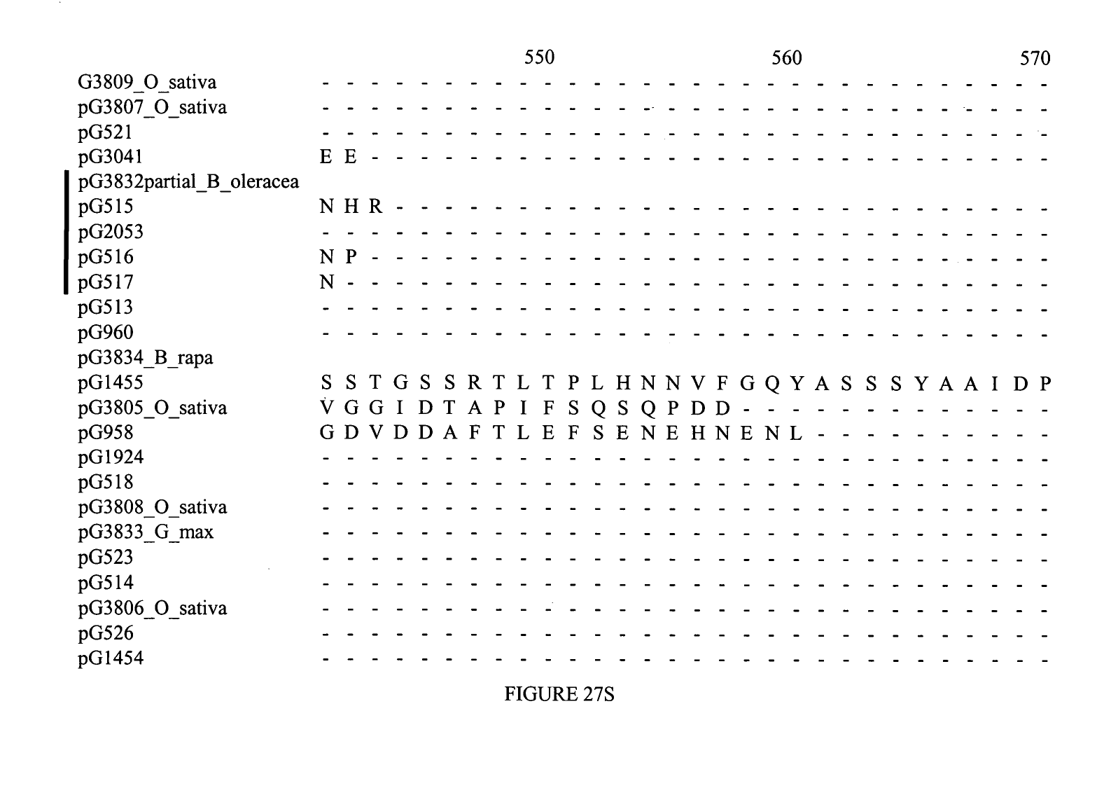

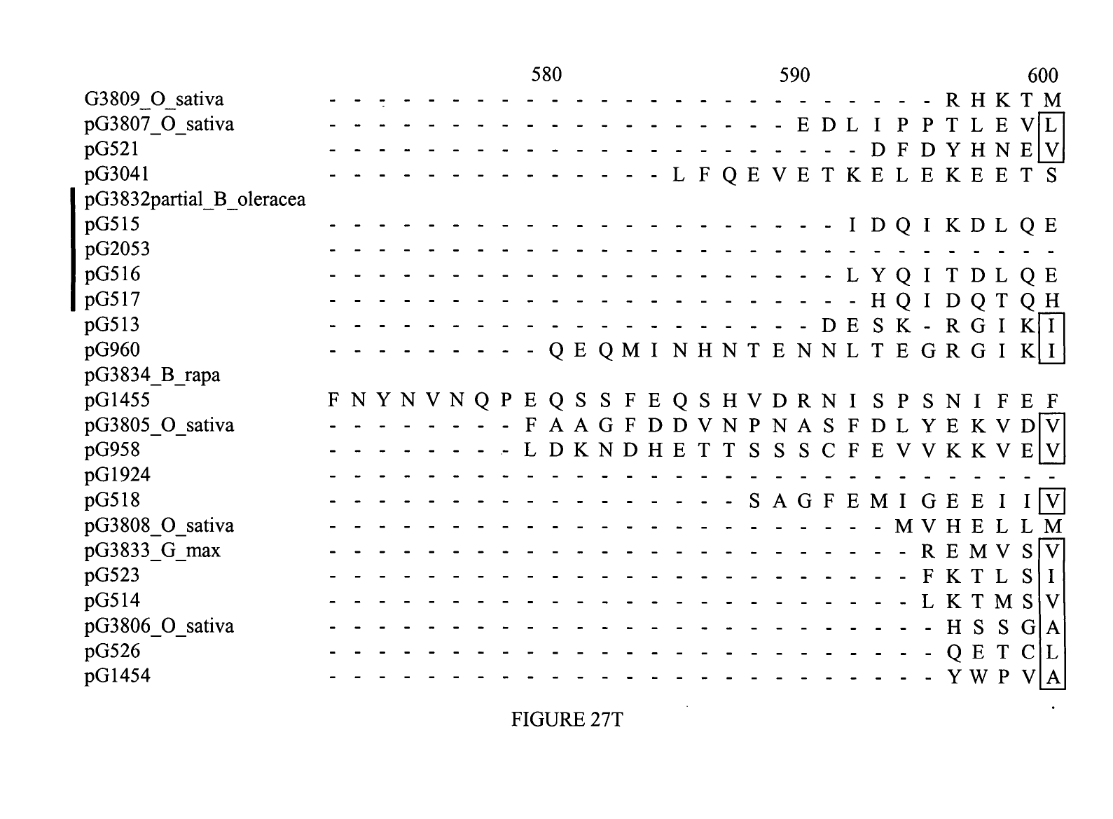

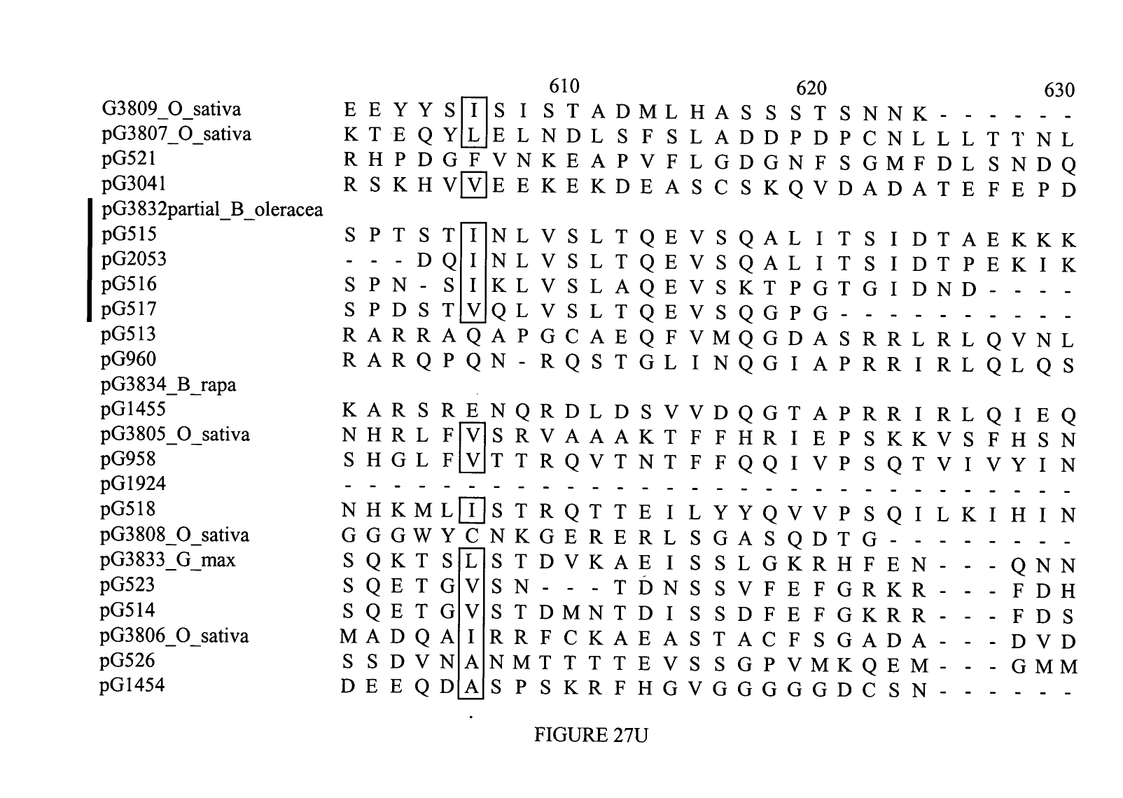

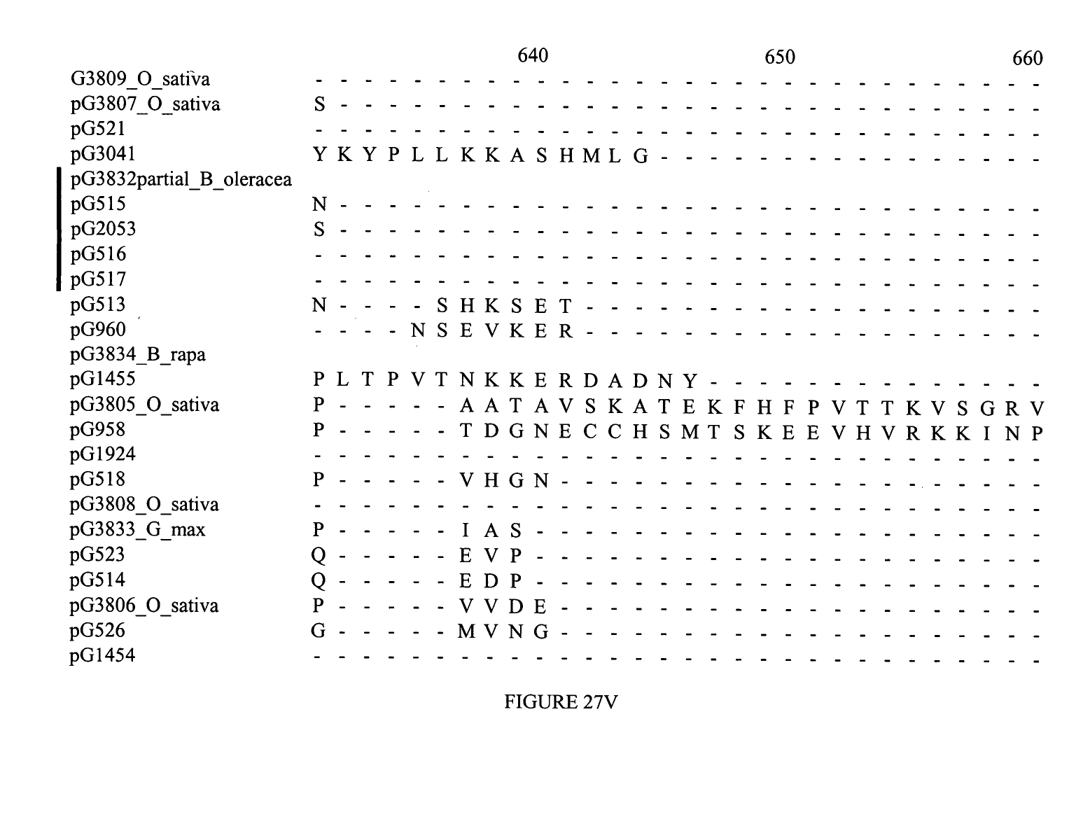

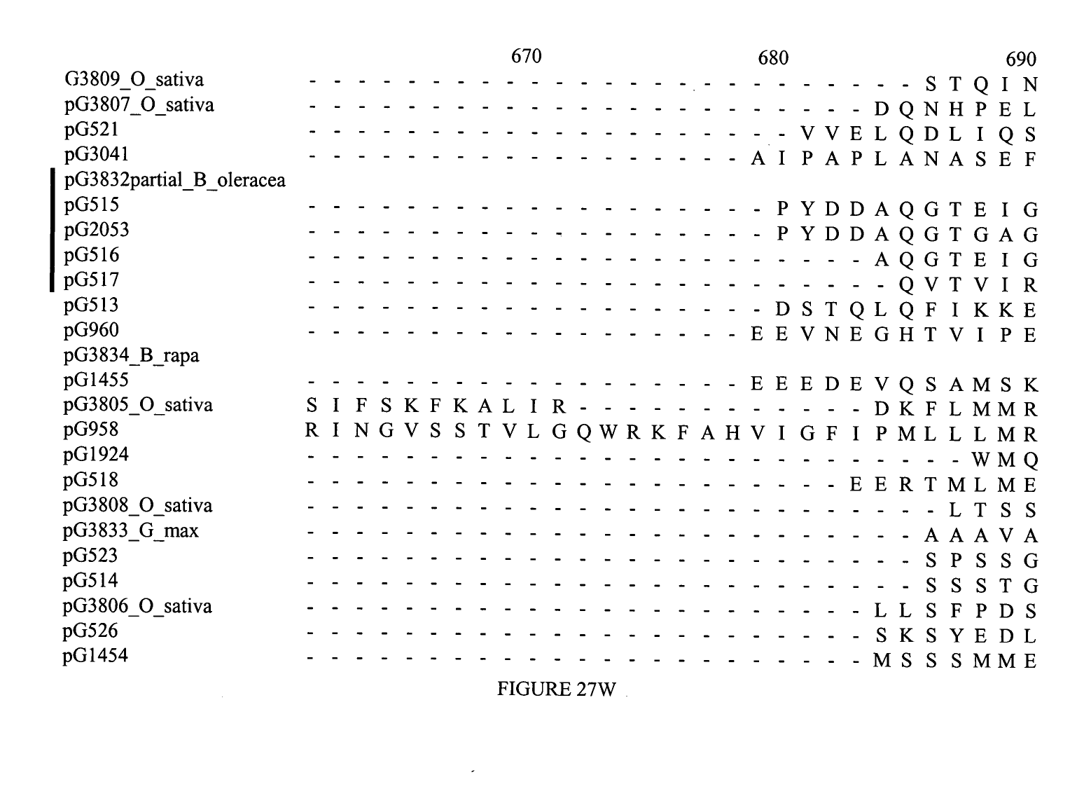

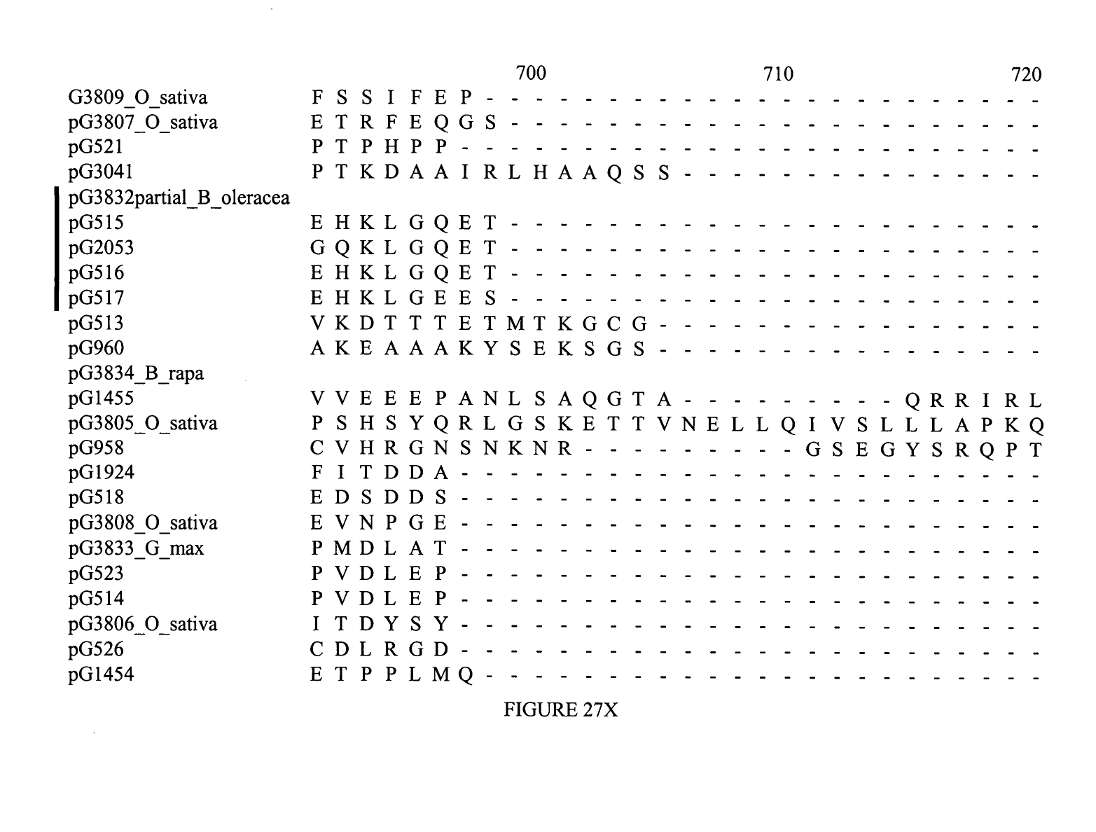

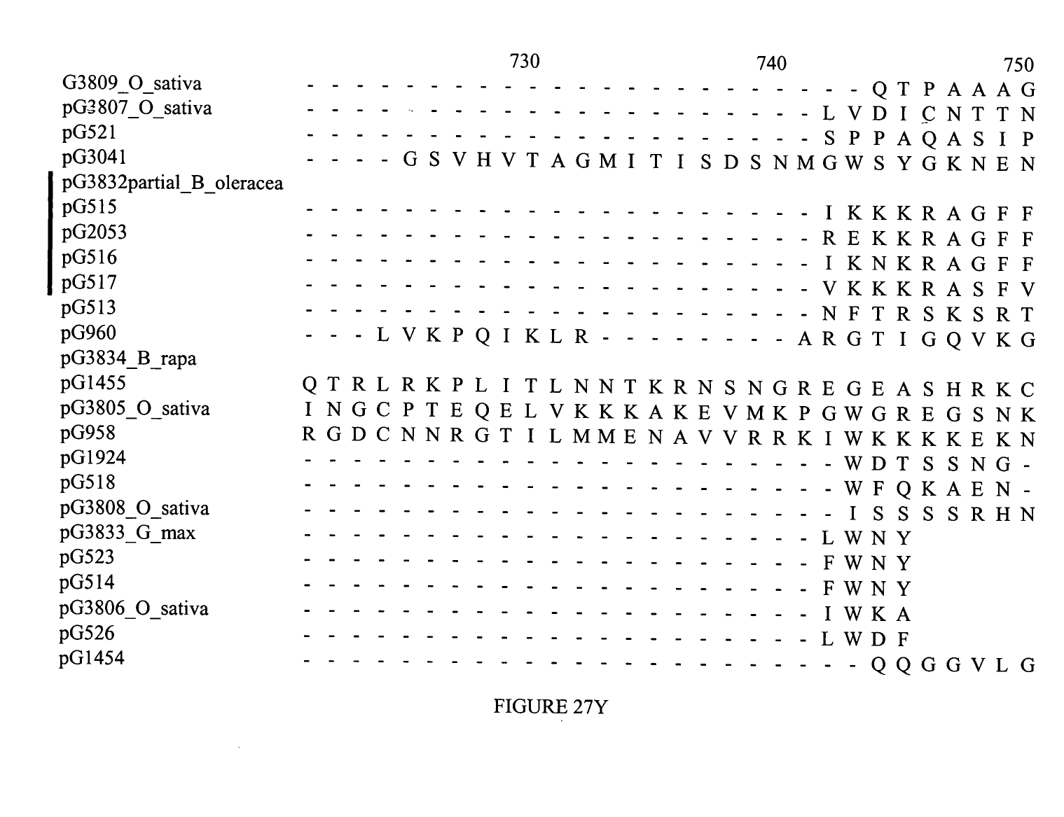

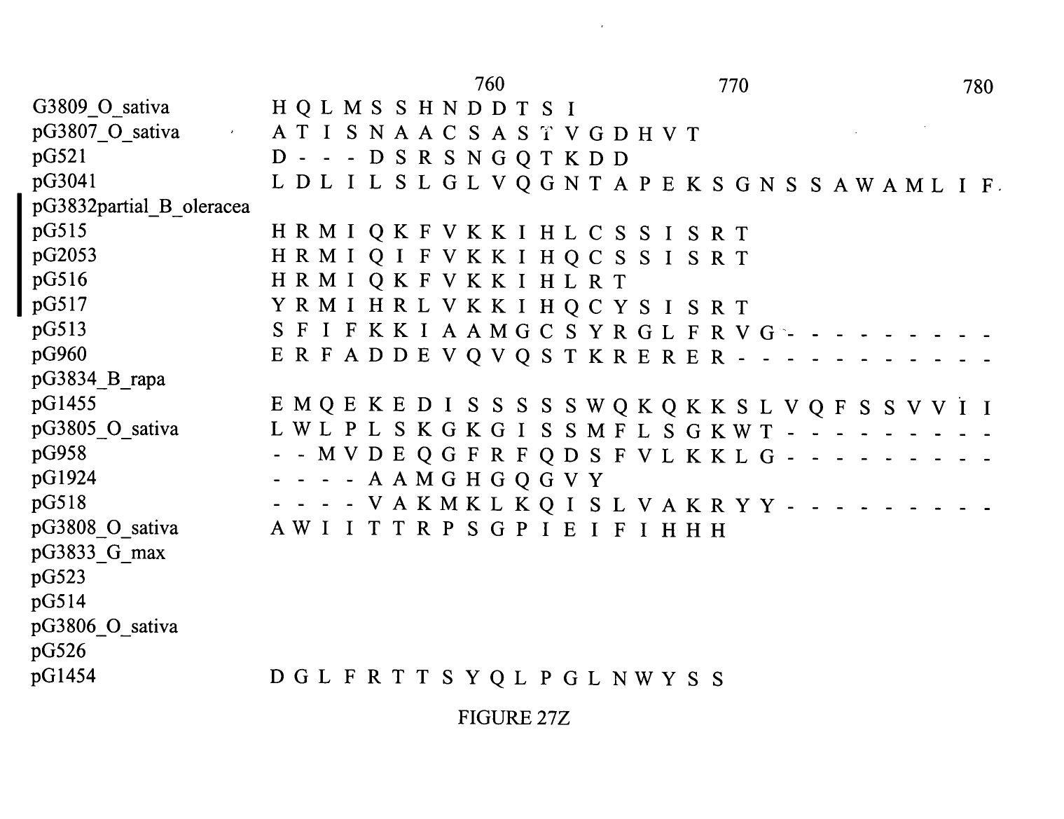

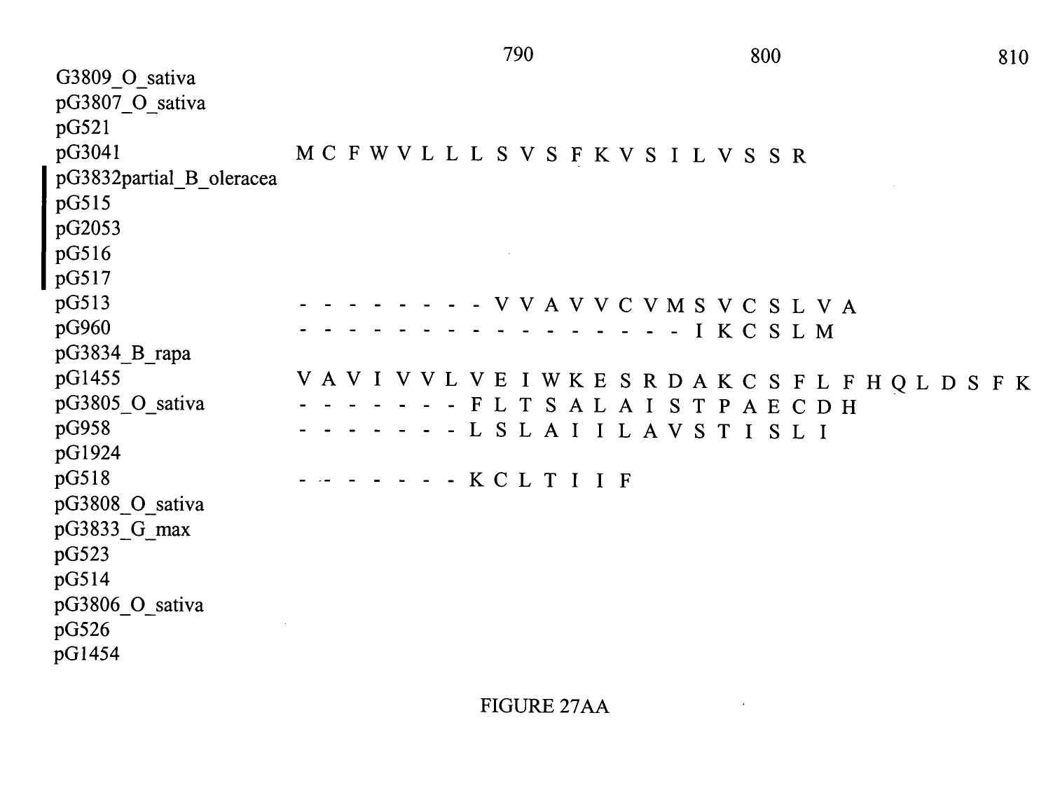

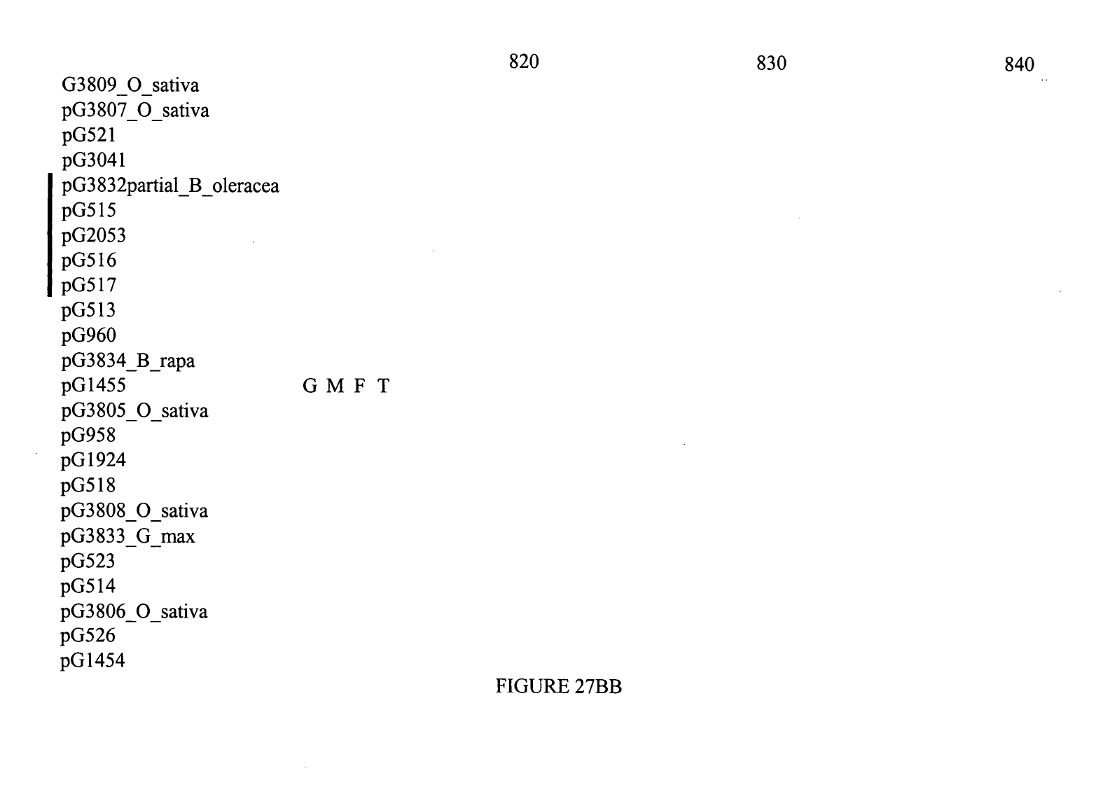

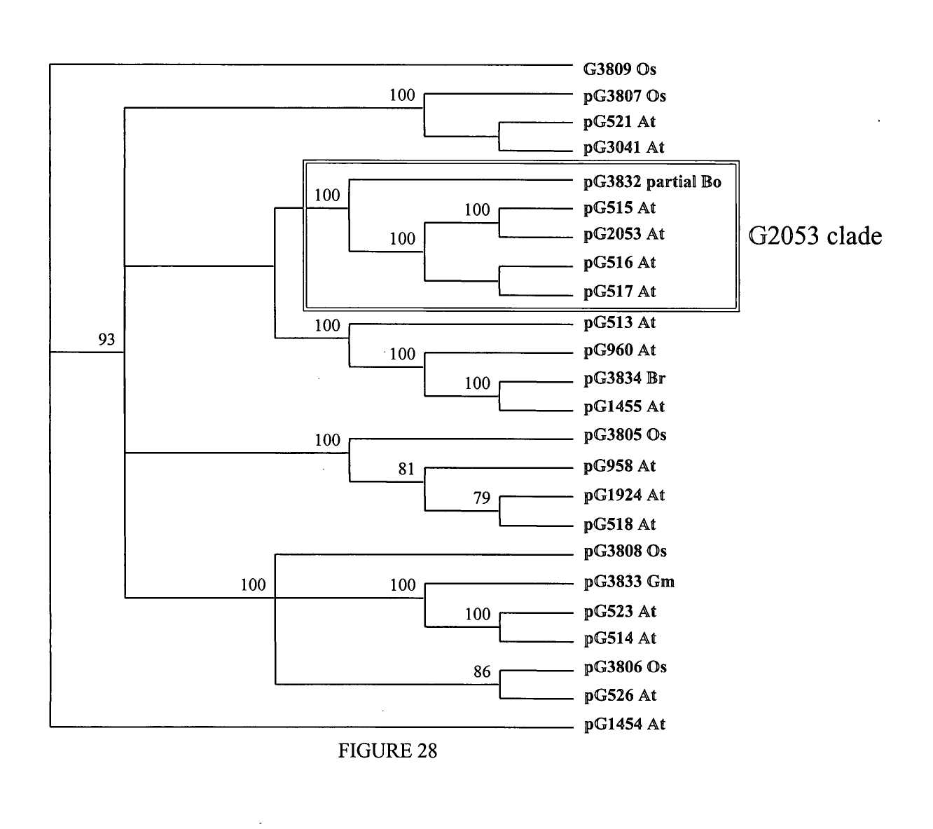

35S::G1274-overexpressing plants were significantly greener and larger than wild-type control plants in a soil-based drought assay (Tables 9 and 10). FIGS. 26A-26D compare soil-based drought assays for G1274 overexpressors and wild-type control plants, which confirms the results predicted after the performance of the plate-based osmotic stress assays. 35S::G1274 lines fared much better after a period of water deprivation (FIG. 26A) than control plants (FIG. 26B). This distinction was particularly evident in the overexpressor plants after once again being watered, said plants almost all fully recovered to a healthy and vigorous state in FIG. 26C. Conversely, none of the wild-type plants seen in FIG. 26D recovered after rewatering, as it was apparently too late for rehydration to rescue these plants (Table 10).

In addition, 35S::G1274 transgenic plants were more tolerant to chilling compared to the wild-type controls, in both germination as well as seedling growth assays.

Overexpression of G1274 produced alterations in leaf morphology and inflorescence architecture. Four out of eighteen 35S::G1274 primary transformants were slightly small and developed inflorescences that were short, and showed reduced internode elongation, leading to a bushier, more compact stature than in wild-type.

In an experiment using T2 populations, it was observed that the rosette leaves from many of the plants were distinctly broad and appeared to have a greater rosette biomass than in wild type.

A similar inflorescence phenotype was obtained from overexpression of a potentially related WRKY gene, G1275. However, G1275 also caused extreme dwarfing, which was not apparent when G1274 was overexpressed.

Utilities

The phenotypic effects of G1274 or equivalog overexpression could have several potential applications:

The enhanced performance of 35S::G1274 plants in a soil-based drought assay indicated that the gene or its equivalogs may be used to enhance drought tolerance in plants.

The enhanced performance of 35S::G1274 seedlings under chilling conditions indicates that the gene or its equivalogs might be applied to engineer crops that show better growth under cold conditions.

The morphological phenotype shown by 35S::G1274 lines indicate that the gene or its equivalogs might be used to alter inflorescence architecture, to produce more compact dwarf forms that might afford yield benefits.

The effects on leaf size that were observed as a result of G1274 or equivalog overexpression might also have commercial applications. Increased leaf size, or an extended period of leaf growth, could increase photosynthetic capacity, and biomass, and have a positive effect on yield. G1274 equivalogs include, for example, Arabidopsis thaliana SEQ ID NO: 30 (G1275) and SEQ ID NO: 32 (G1758); Oryza sativa (japonica cultivar-group) SEQ ID NO: 134 (G3721), SEQ ID NO: 142 (G3725), SEQ ID NO: 144 (G3726), SEQ ID NO: 150 (G3729), and SEQ ID NO: 152 (G3730); Glycine max SEQ ID NO: 138 (G3723), SEQ ID NO: 140 (G3724), and SEQ ID NO: 208 (G3803); Solanum tuberosum SEQ ID NO: 156 (G3732); Capsicum annuum SEQ ID NO: 202 (G3795); Lactuca sativa SEQ ID NO: 204 (G3797); Hordeum vulgare SEQ ID NO: 158 (G3733); Zea mays SEQ ID NO: 130 (G3719), SEQ ID NO: 132 (G3720), SEQ ID NO: 136 (G3722), SEQ ID NO: 146 (G3727), SEQ ID NO: 148 (G3728), and SEQ ID NO: 210 (G3804); Sorghum bicolor SEQ ID NO: 206 (G3802); and Lycopersicon esculentum SEQ ID NO: 154 (G373 1).

G922 (SEQ ID NOs: 3 and 4)

Published Information

G922 corresponds to Scarecrow-like 3 (SCL3) first described by Pysh et al. (GenBank accession number AF036301; (1999) Plant J. 18: 111-119). Northern blot analysis results show that G922 is expressed in siliques, roots, and to a lesser extent in shoot tissue from 14 day old seedlings. Pysh et al did not test any other tissues for G922 expression. In situ hybridization results showed that G922 was expressed predominantly in the endodermis in the root tissue. This pattern of expression was very similar to that of SCARECROW (SCR), G306. Experimental evidence indicated that the co-localization of the expression is not due to cross-hybridization of the G922 probe with G306. Pysh et al proposed that G922 may play a role in epidermal cell specification and that G922 may either regulate or be regulated by G306.

The sequence for G922 can also be found in the annotated BAC clone F11F12 from chromosome 1 (GenBank accession number AC012561). The sequence for F11F12 was submitted to GenBank by the DNA Sequencing and Technology Center at Stanford University.

Closely Related Genes from Other Species

The amino acid sequence for a region of the Oryza sativa chromosome I clone P0466H10 (GenBank accession number AP003259) is significantly identical to G922 outside of the SCR conserved domains. Therefore, the gene represented by this region of the rice clone may be the ortholog of G922.

Experimental Observations

The function of this gene was analyzed using transgenic plants in which G922 was expressed under the control of the 35S promoter.

Morphologically, plants overexpressing G922 had altered leaf morphology, coloration, fertility, and overall plant size. In wild-type plants, expression of G922 was induced by auxin, ABA, heat, and drought treatments. In non-induced wild-type plants, G922 was expressed constitutively at low levels.

Transgenic plants overexpressing G922 were more salt tolerant than wild-type plants as determined by a root growth assay on MS media supplemented with 150 mM NaCl. Plant overexpressing G922 also were more tolerant to osmotic stress as determined by germination assays in salt-containing (150 mM NaCl; FIG. 21A) and sucrose-containing (9.4%; FIG. 21C) media than wild-type controls grown in high salt and sucrose (FIGS. 21B and 21D, respectively).

The high salt assays suggested that this gene would confer drought tolerance, a supposition confirmed by soil-based assays, in which G922-overexpressing plants were significantly healthier after water deprivation treatment than wild-type control plants (Tables 9 and 10).

Utilities

Based upon results observed in plants overexpressing G922 or its equivalogs could be used to alter salt tolerance, tolerance to osmotic stress, and leaf morphology in other plant species. Evaporation from the soil surface causes upward water movement and salt accumulation in the upper soil layer where the seeds are placed. Thus, germination normally takes place at a salt concentration much higher than the mean salt concentration of in the whole soil profile. Increased salt tolerance during the germination stage of a crop plant would impact survivability and yield.

Altered leaf morphology conferred by overexpression of G922 or its equivalogs could be desirable in ornamental horticulture. G922 equivalogs include, for example, Oryza sativa (japonica cultivar-group) SEQ ID NO: 218 (G3814), SEQ ID NO: 216 (G3813), and SEQ ID NO: 222 (G3827); Lycopersicon esculentum SEQ ID NO: 220 (G3824); and Glycine max SEQ ID NO: 212 (G3810) and SEQ ID NO: 214 (G3811).

G2999 (SEQ ED NOs: 13 and 14)

Published Information

G2999 was identified within a sequence released by the Arabidopsis Genome Initiative (Chromosome 2, GenBank accession AC006439).

Experimental Observations

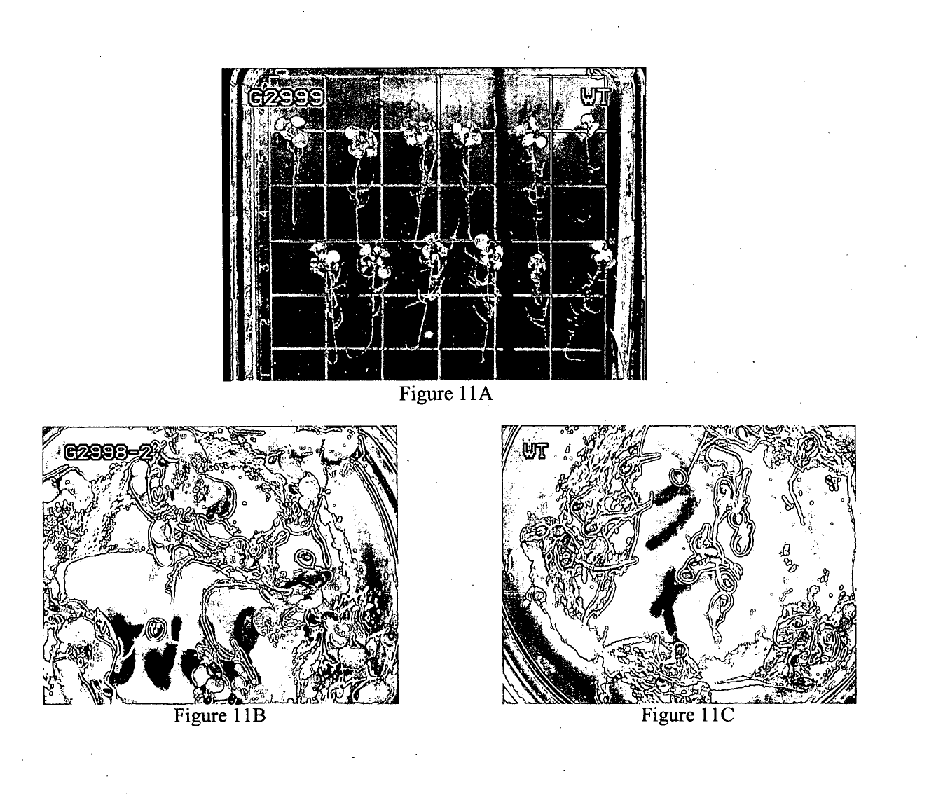

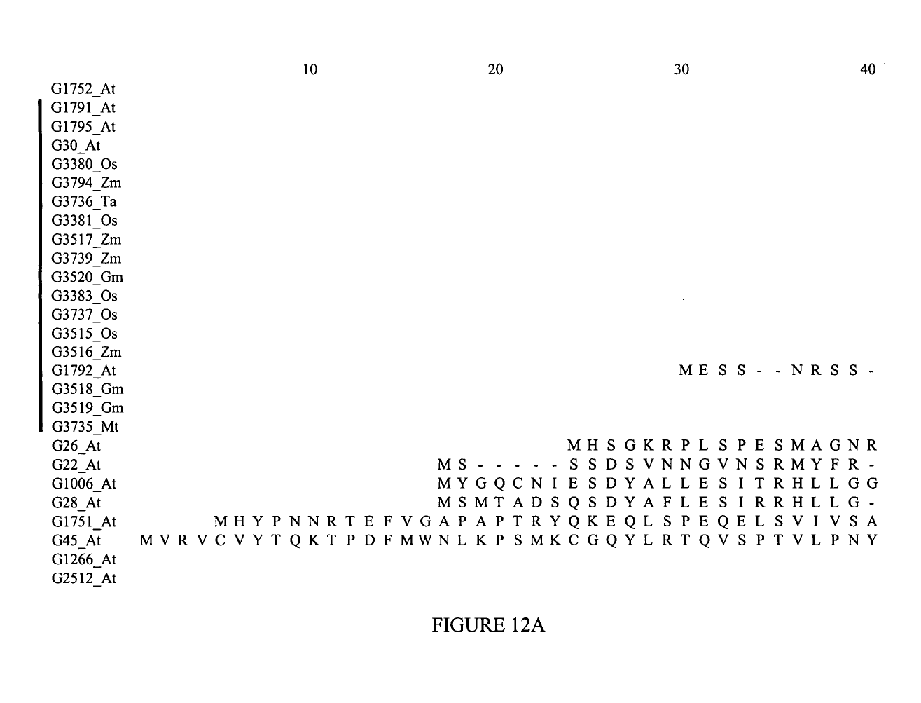

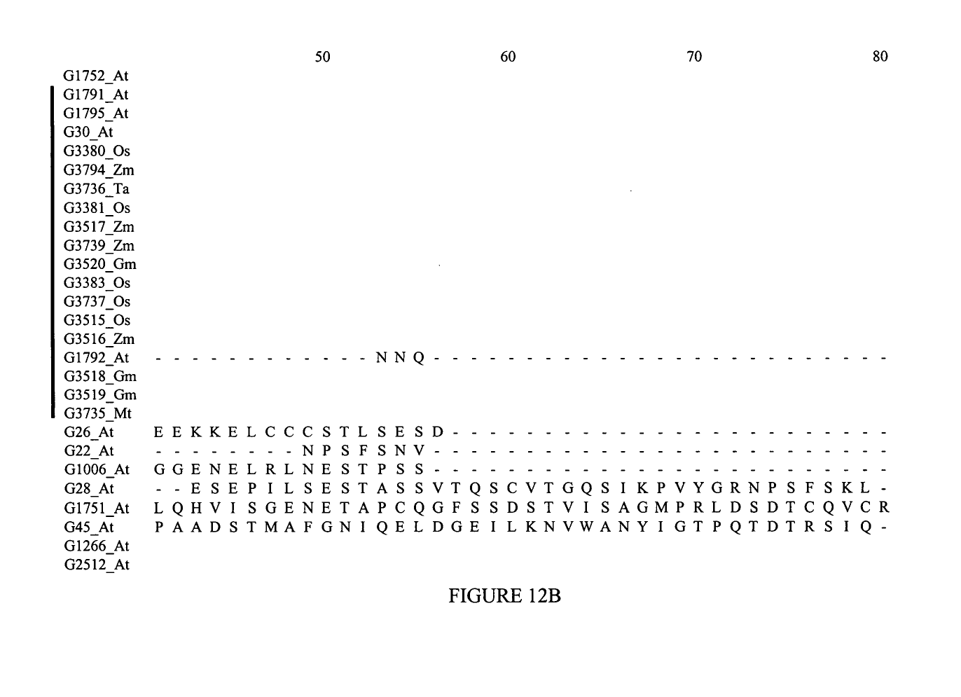

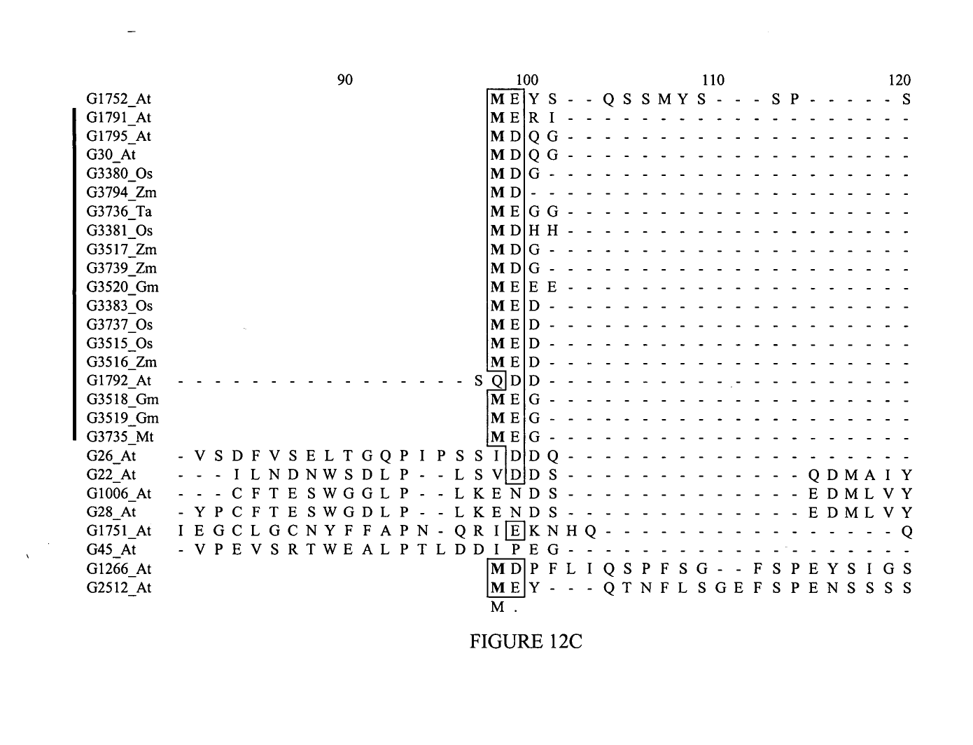

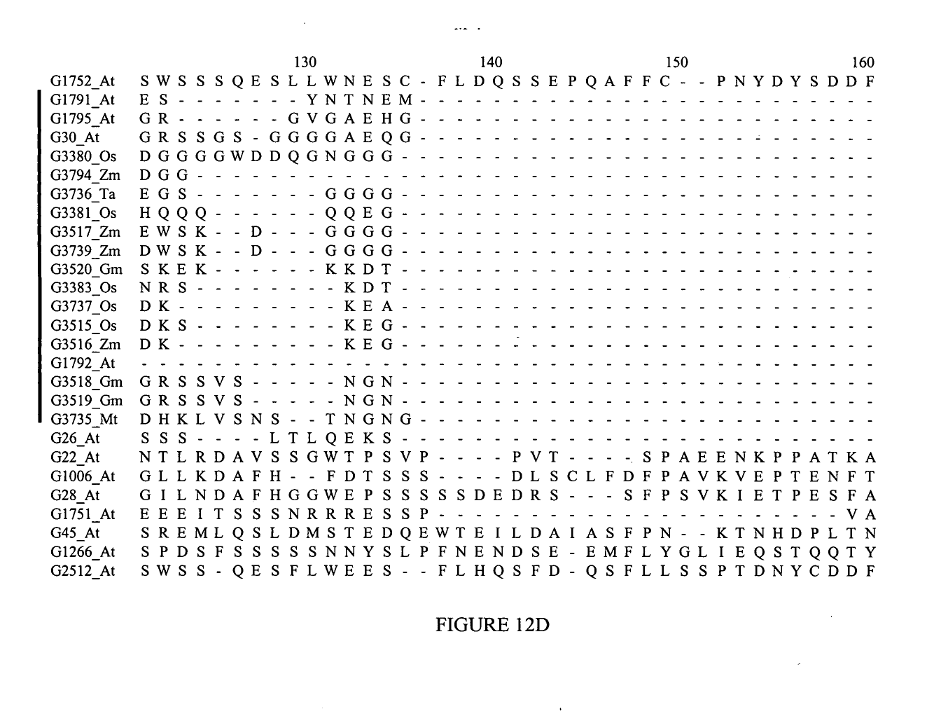

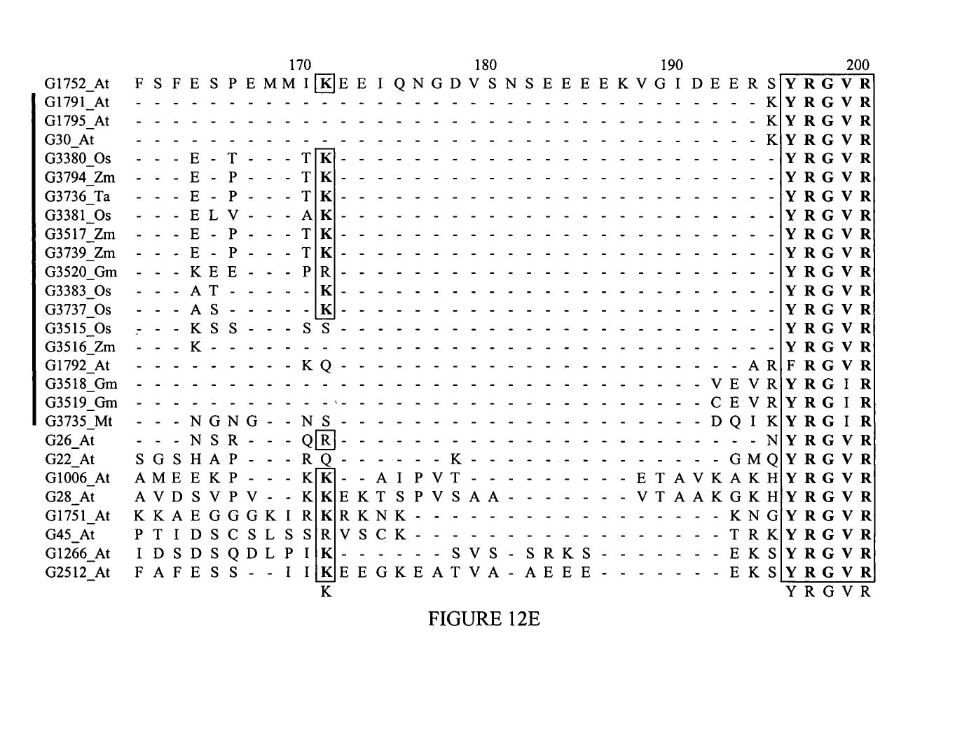

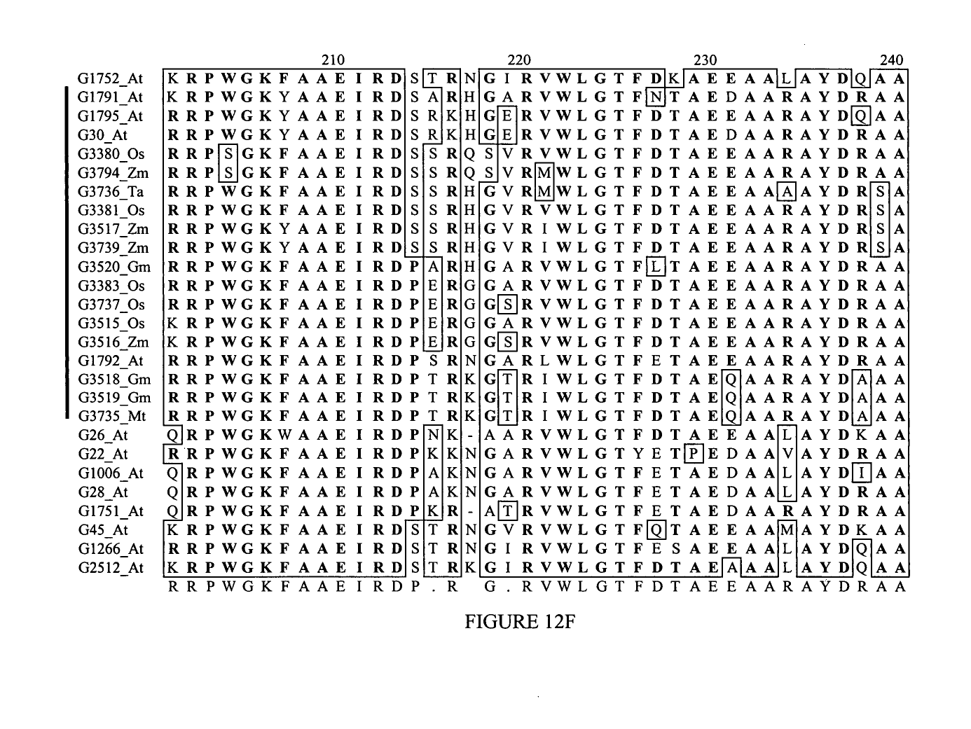

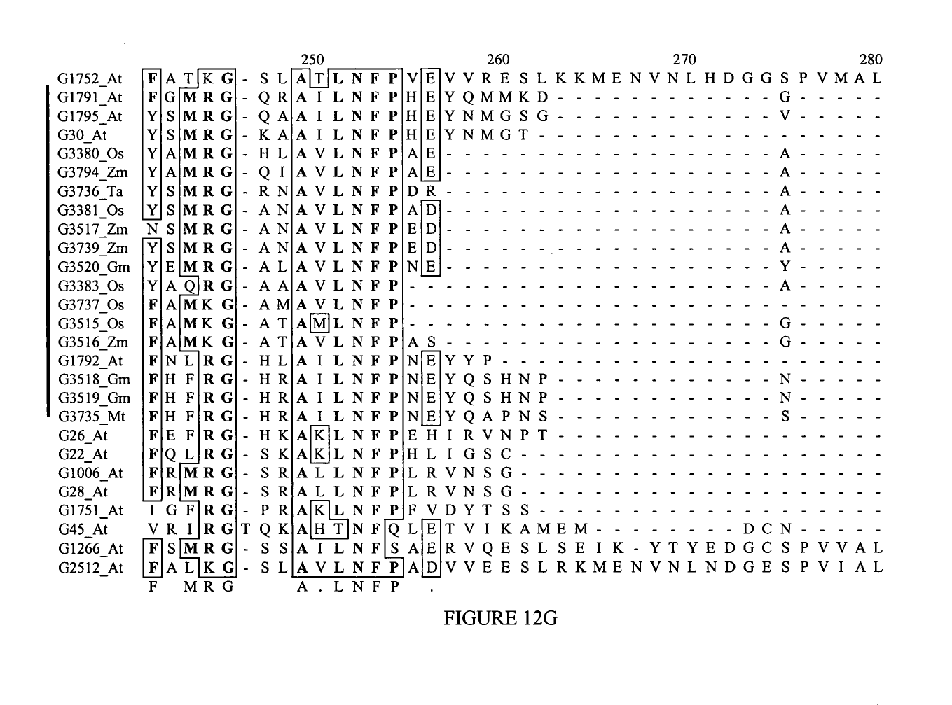

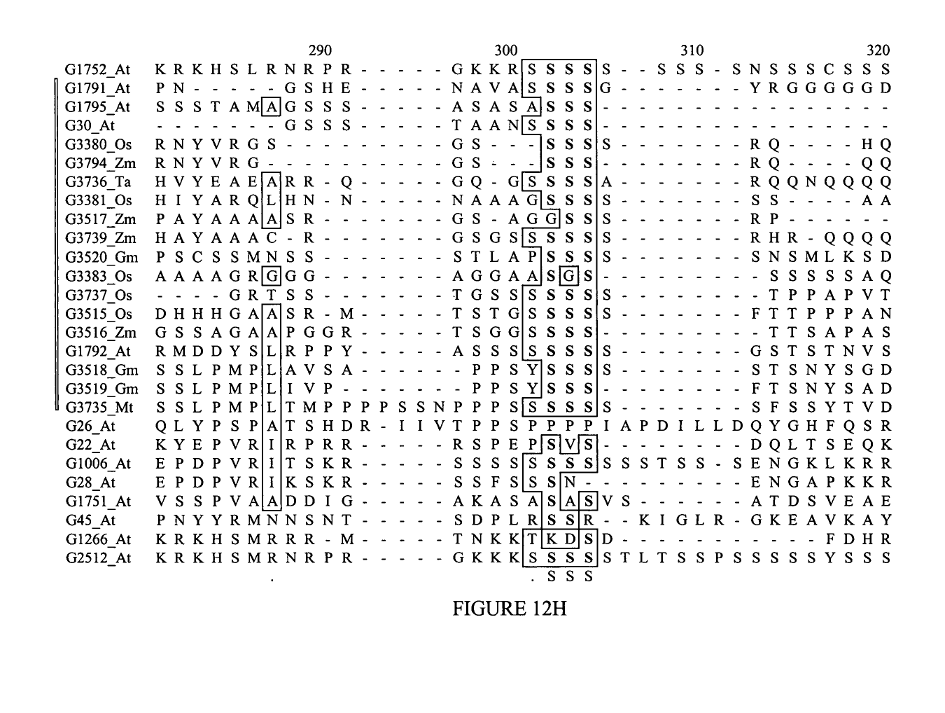

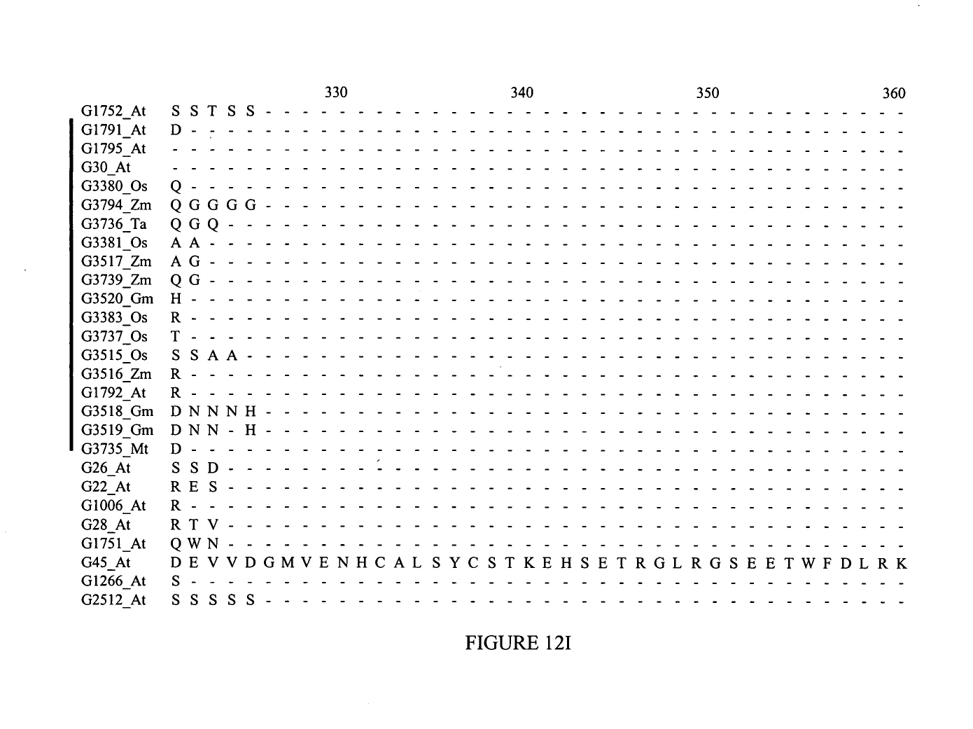

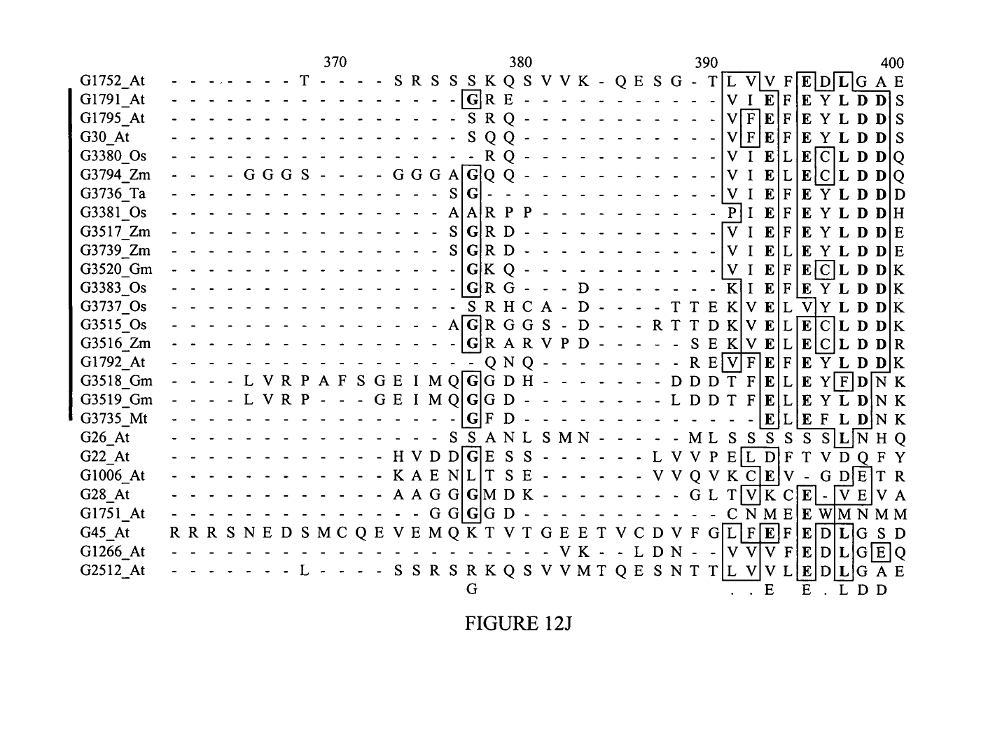

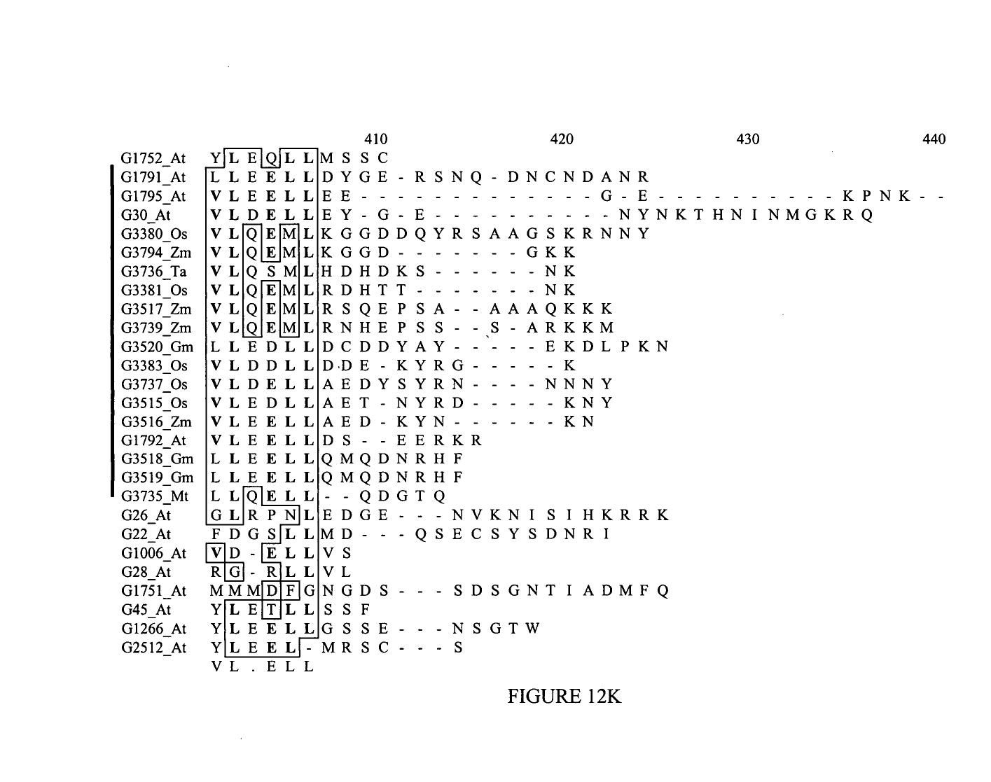

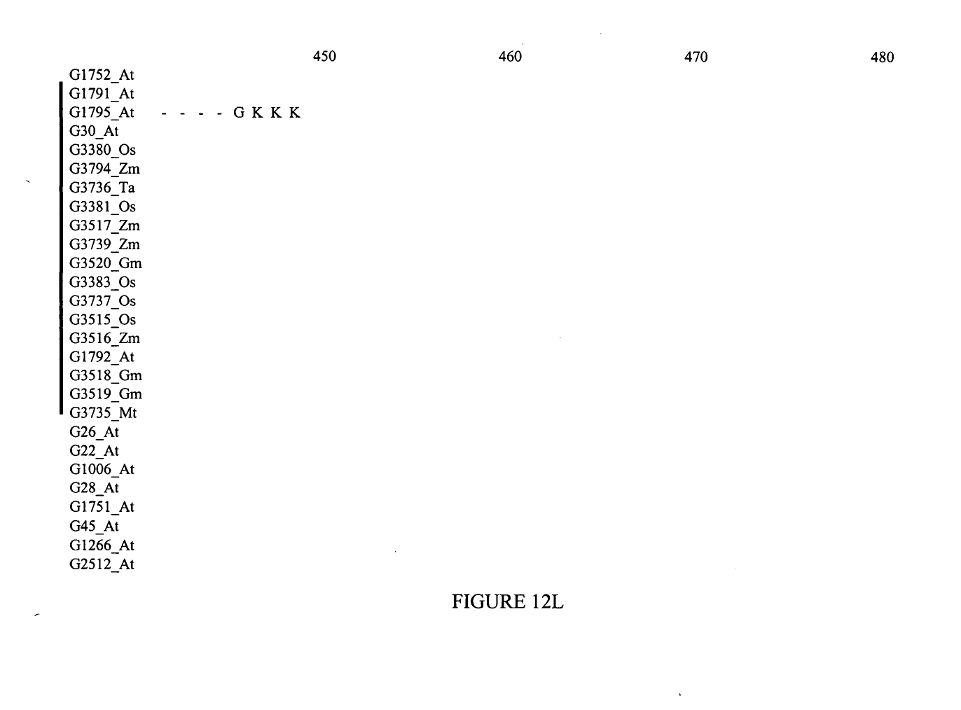

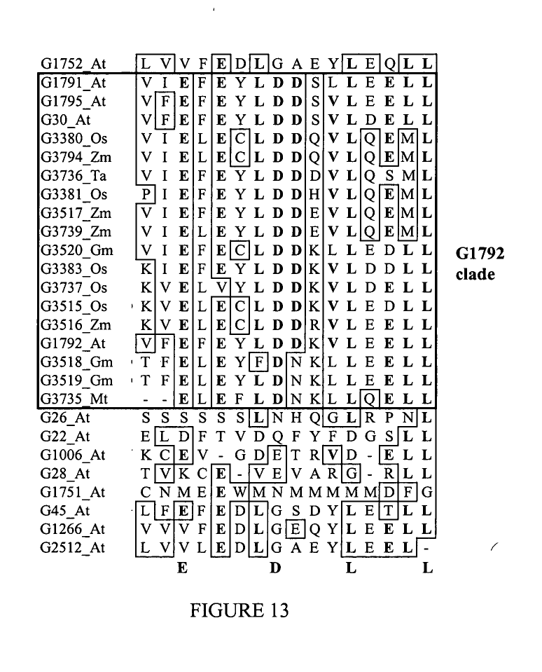

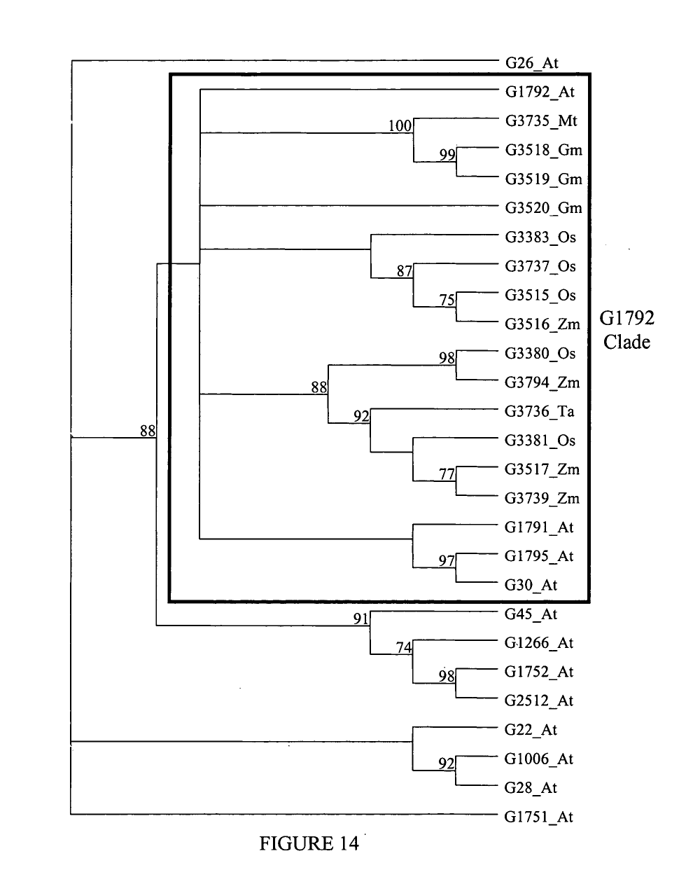

The boundaries of G2999 were determined by RACE experiments and a full-length clone was PCR-amplified out of cDNA derived from mixed tissues. The function of G2999 was then assessed by analysis of transgenic Arabidopsis lines in which the cDNA was constitutively expressed from a 35S CaMV promoter. 35S::G2999 transformants displayed wild-type morphology, but two of three T2 lines showed increased tolerance to salt stress in the physiology assays. Root growth assays with G2999 overexpressing seedlings and controls in a high sodium chloride medium showed that a majority of 35S::G2999 Arabidopsis seedlings appeared larger, greener, and had more root growth than the control seedlings on the right (FIG. 11A, four control seedlings are on the right). G2998, a paralogous Arabidopsis sequence, also showed a salt tolerance phenotype in a plate-based salt stress assay, where these overexpressors were greener and had more cotyledon expansion (FIG. 11B) than wild-type seedlings (FIG. 11C). Thus, G2998 and G2999 could act in the same pathways, and have a role in the response to abiotic stress.

The high salt assays suggested that this gene would confer drought tolerance, a supposition confirmed in a soil-based assay in which G2999 overexpressing-plants were significantly more drought tolerant than wild-type control plants (Tables 9 and 10).

Utilities

Given the salt resistance exhibited by 35S::G2999 transformants, the gene and its equivalogs can be used to engineer drought and salt tolerant crops and trees that can flourish in conditions of osmotic stress. G2999 equivalogs include, for example, Arabidopsis thaliana SEQ ID NO: 50 (G2992), SEQ ID NO: 48 (G2991), SEQ ID NO: 68 (G3002), SEQ ID NO: 66 (G3001), SEQ ID NO: 46 (G2990), SEQ ID NO: 44 (G2989), SEQ ID NO: 62 (G2998), SEQ ID NO: 64 (G3000), SEQ ID NO: 54 (G2994), SEQ ID NO: 52 (G2993), SEQ ID NO: 60 (G2997), SEQ ID NO: 58 (G2996), SEQ ID NO: 56 (G2995); Zea mays SEQ ID NO: 114 (G3680); Oryza sativa (japonica cultivar group) SEQ ID NO: 128 (G3695), SEQ ID NO: 126 (G3694), SEQ ID NO: 122 (G3690), SEQ ID NO: 118 (G3685), SEQ ID NO: 108 (G3671), SEQ ID NO: 116 (G3683), and SEQ ID NO: 124 (G3692); Oryza sativa (indica cultivar group) SEQ ID NO: 120 (G3686) and SEQ ID NO: 110 (G3674); Lotus corniculatus var. japonicus SEQ ID NO: 102 (G3663) and SEQ ID NO: 106 (G3670); Brassica napus SEQ ID NO: 112 (G3675); and Flaveria bidentis SEQ ID NO: 104 (G3668).

G3086 (SEQ ID NOs: 15 and 16)

Published Information

G3086 corresponds to gene AT1G51140, annotated by the Arabidopsis Genome Initiative. No information is available about the function(s) of G3086.

Experimental Observations

The function of G3086 was studied using transgenic plants in which the gene was expressed under the control of the 35S promoter. Overexpression of G3086 in Arabidopsis produced a pronounced acceleration in the onset of flowering. 35S::G3086 transformants produced visible flower buds 5-7 days early (in inductive 24-hour light conditions), and were markedly smaller than wild-type controls.

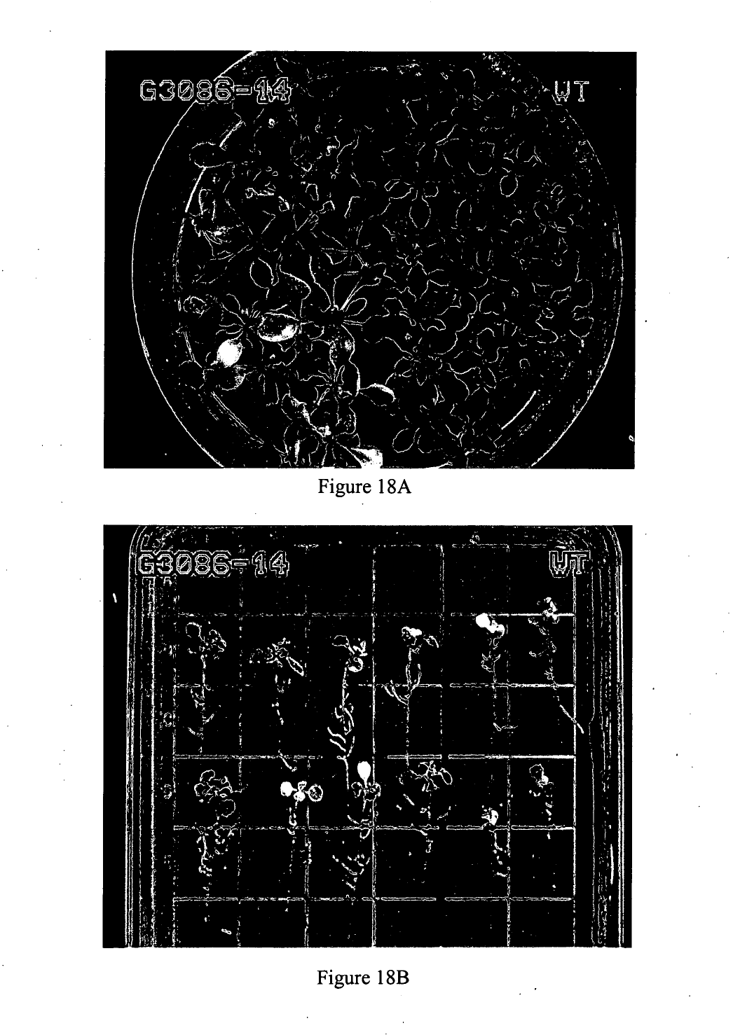

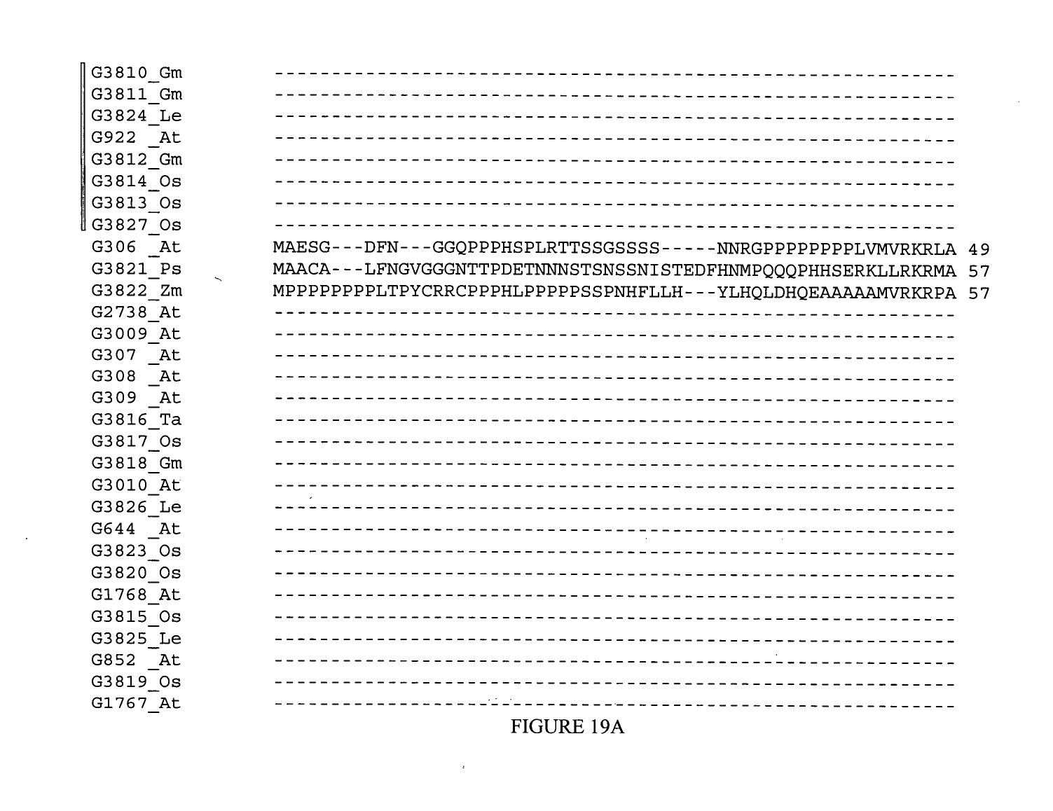

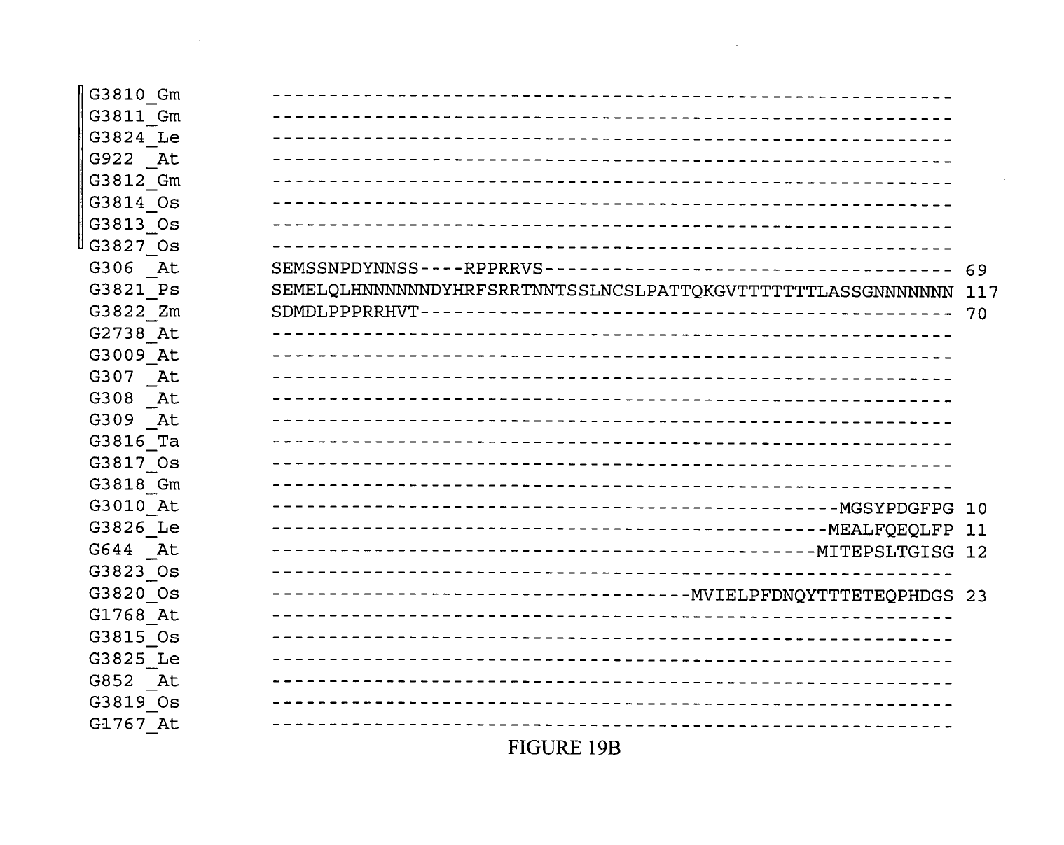

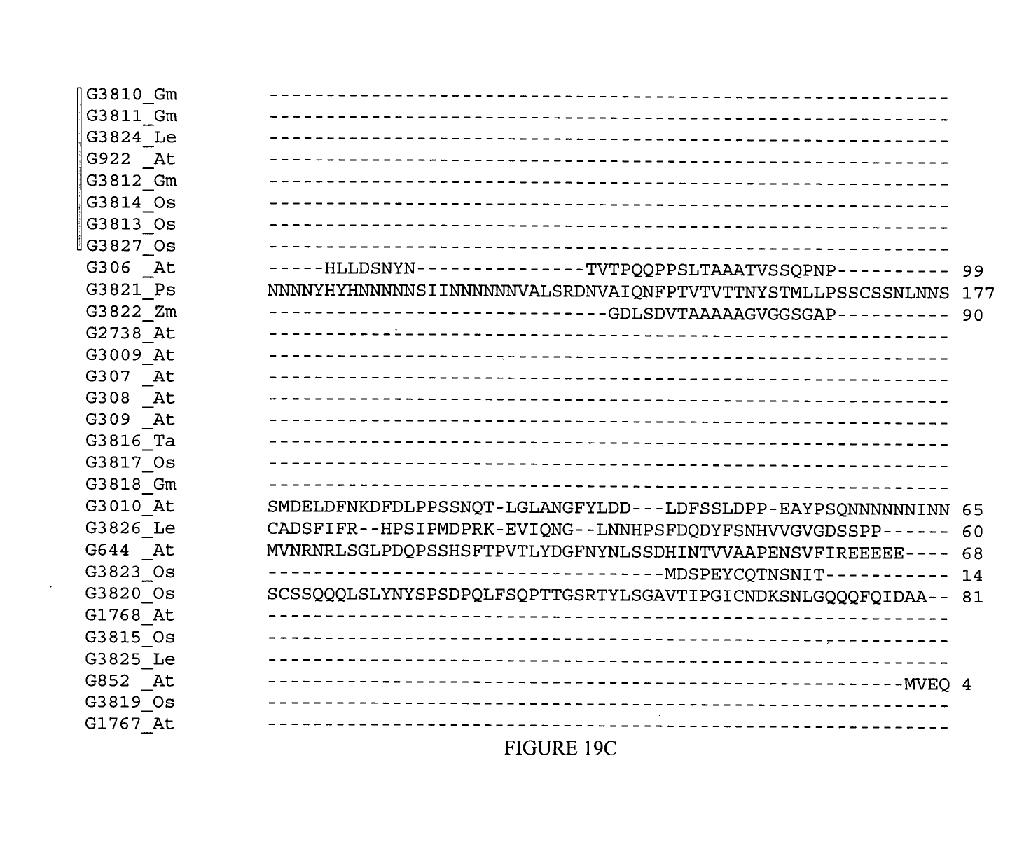

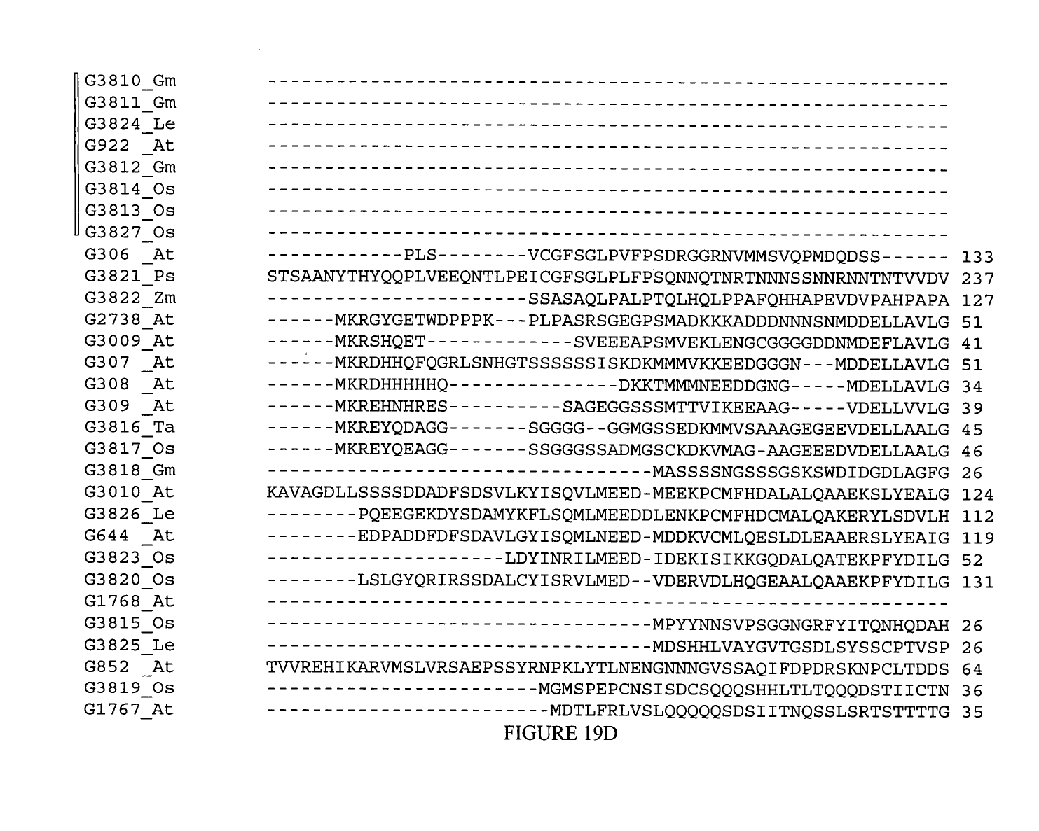

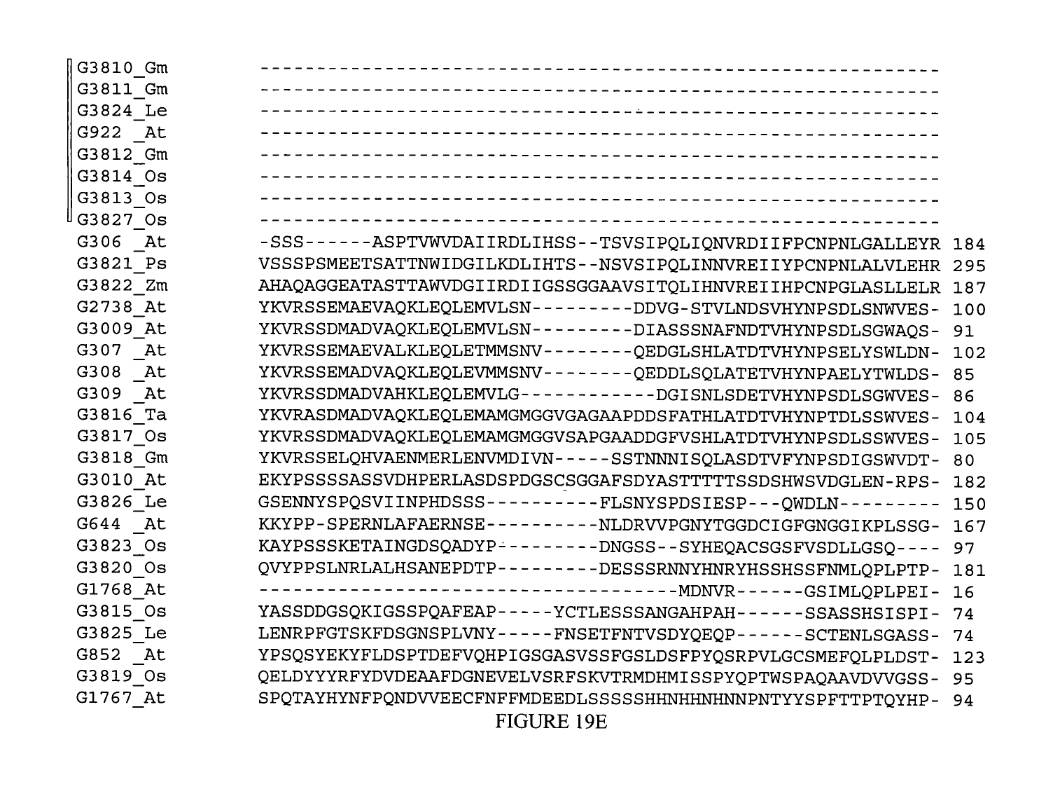

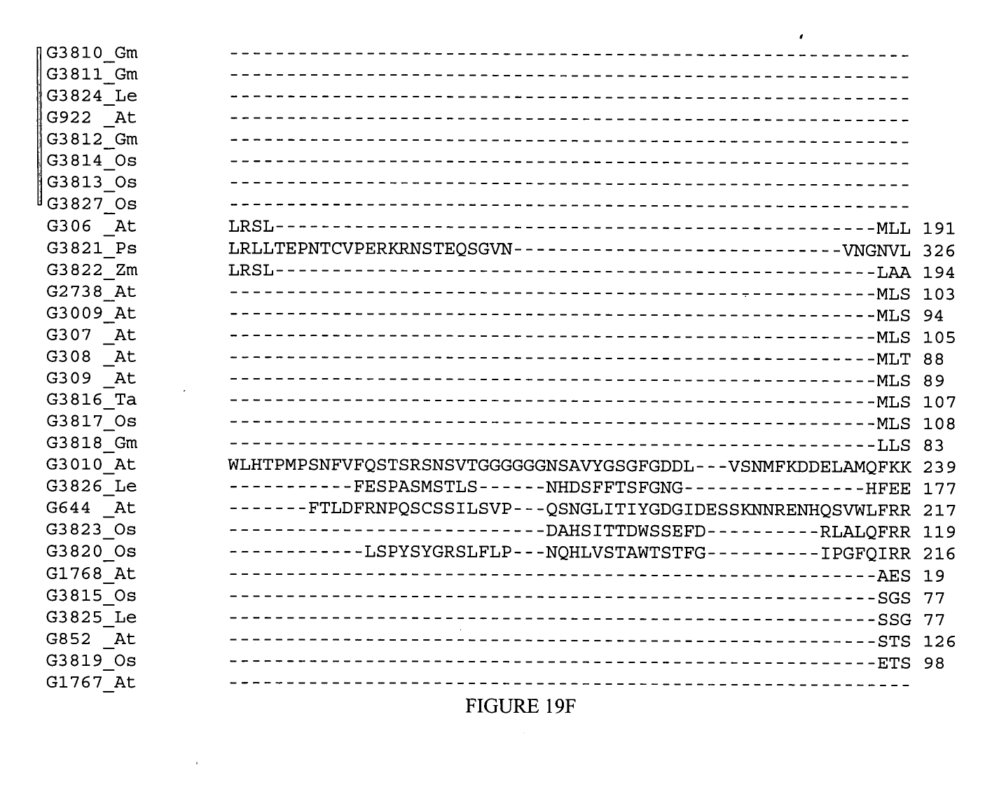

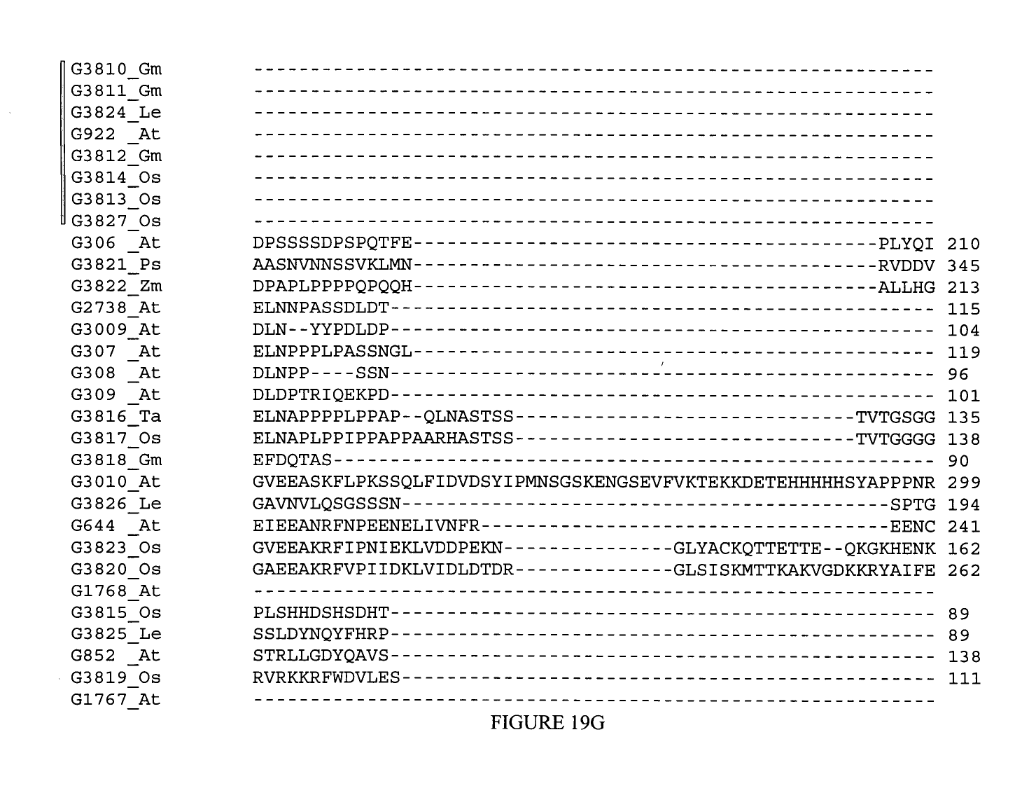

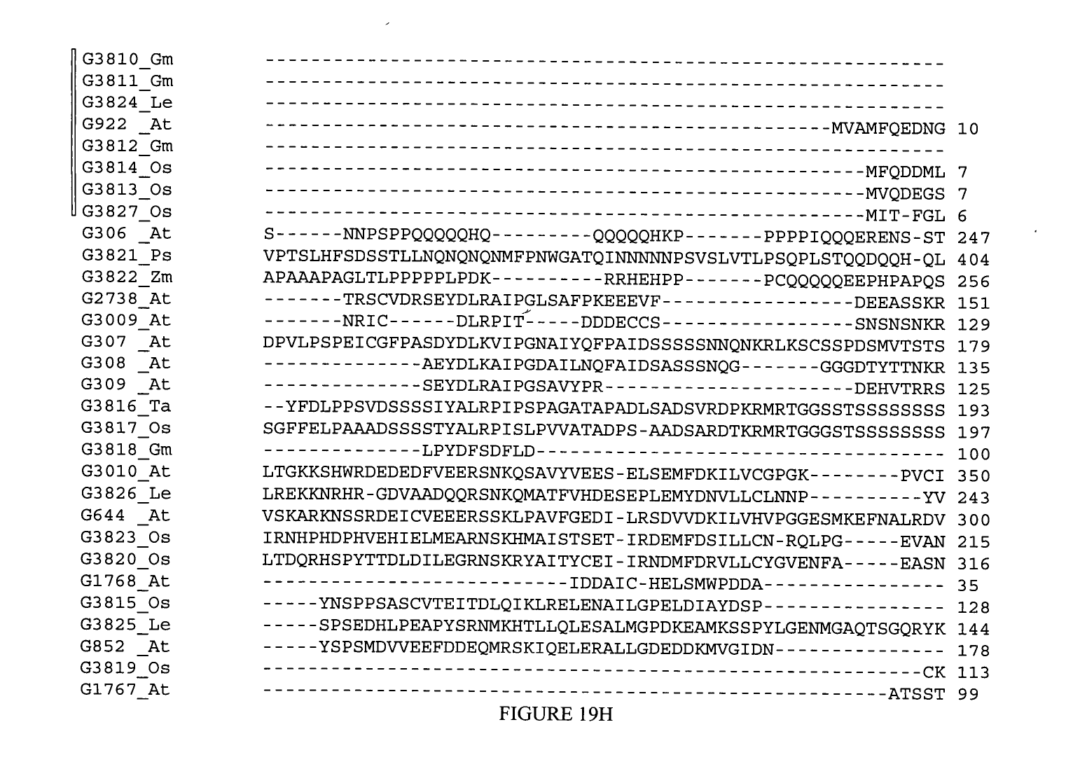

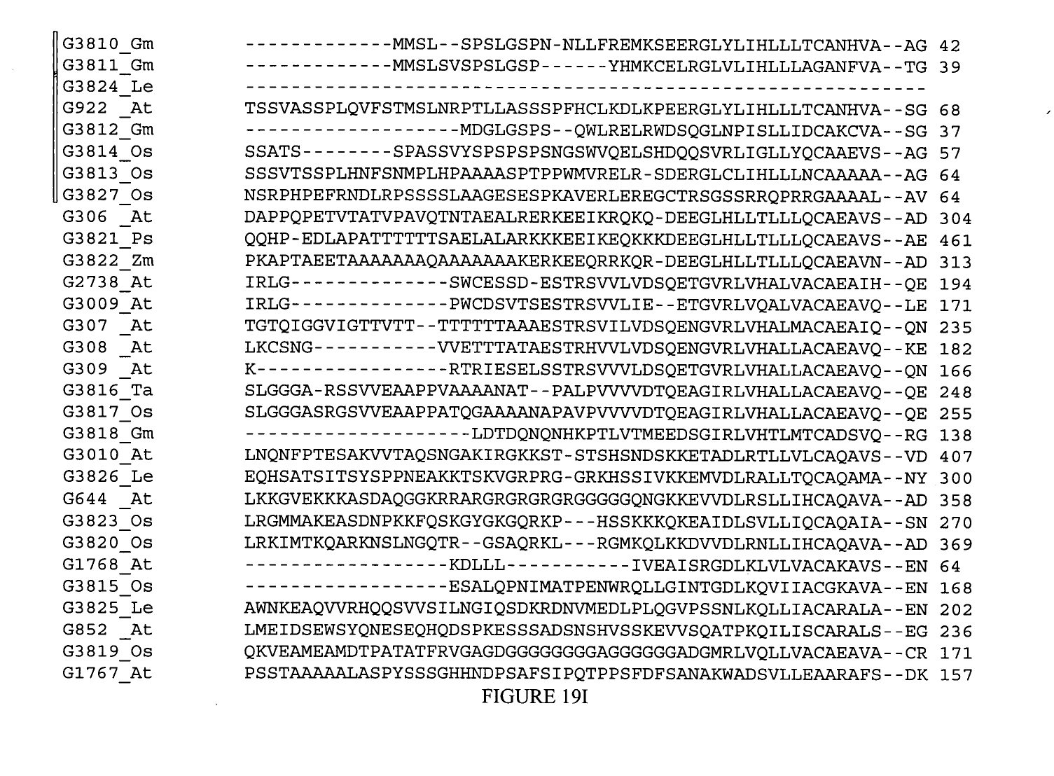

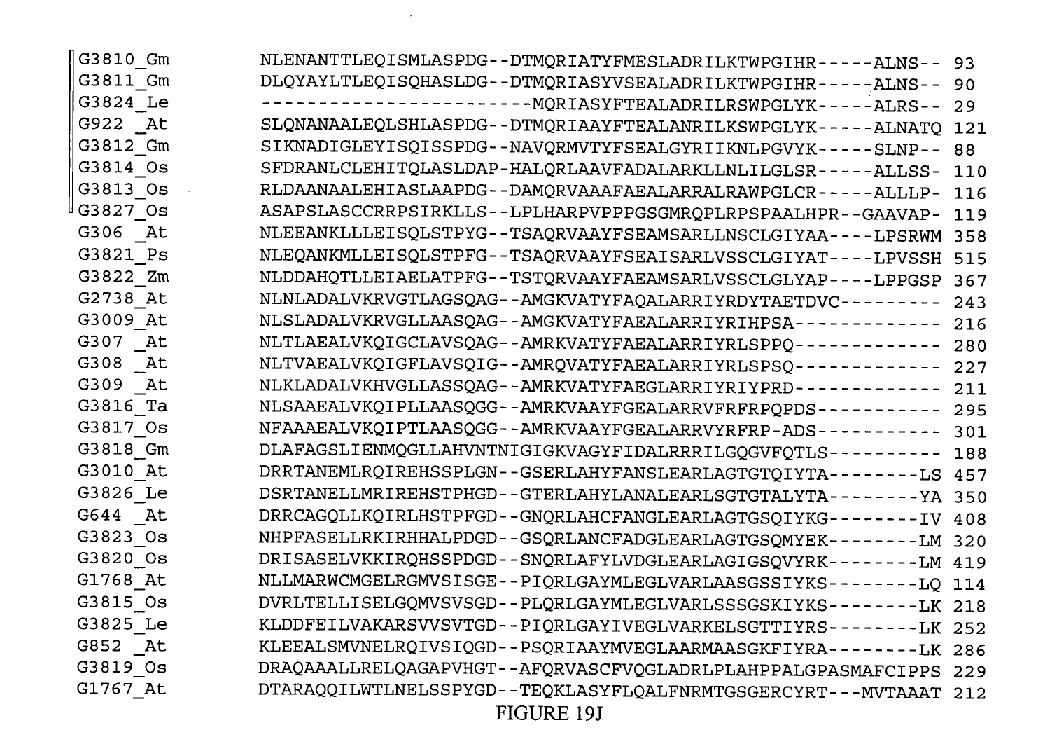

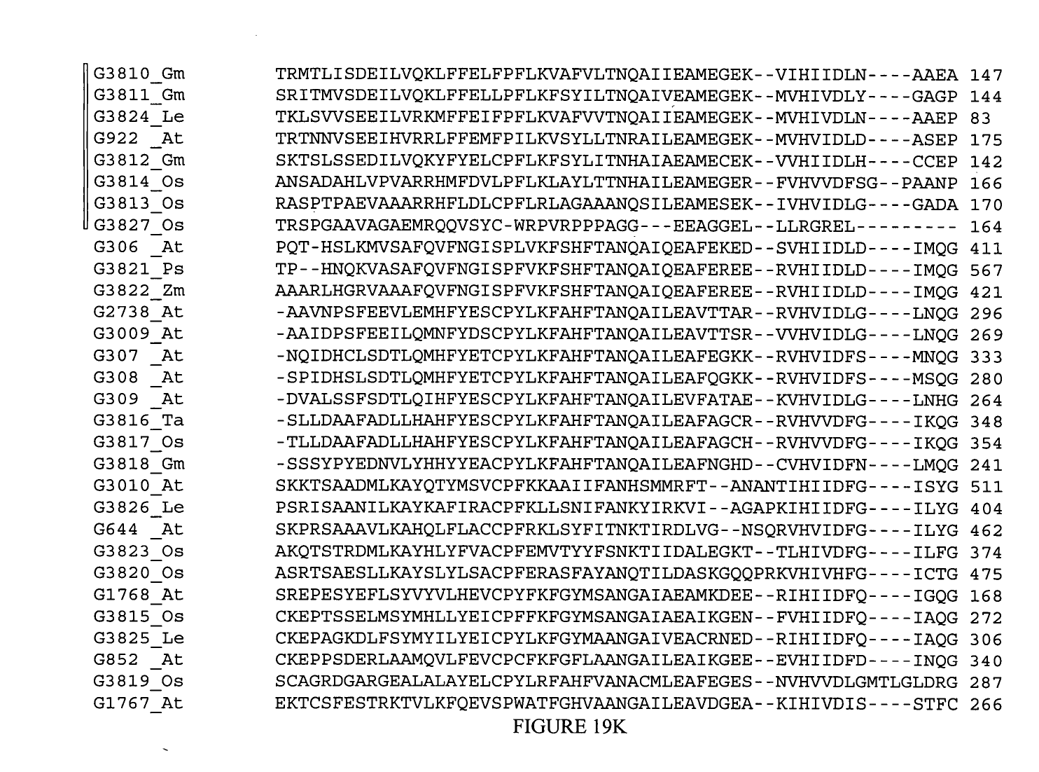

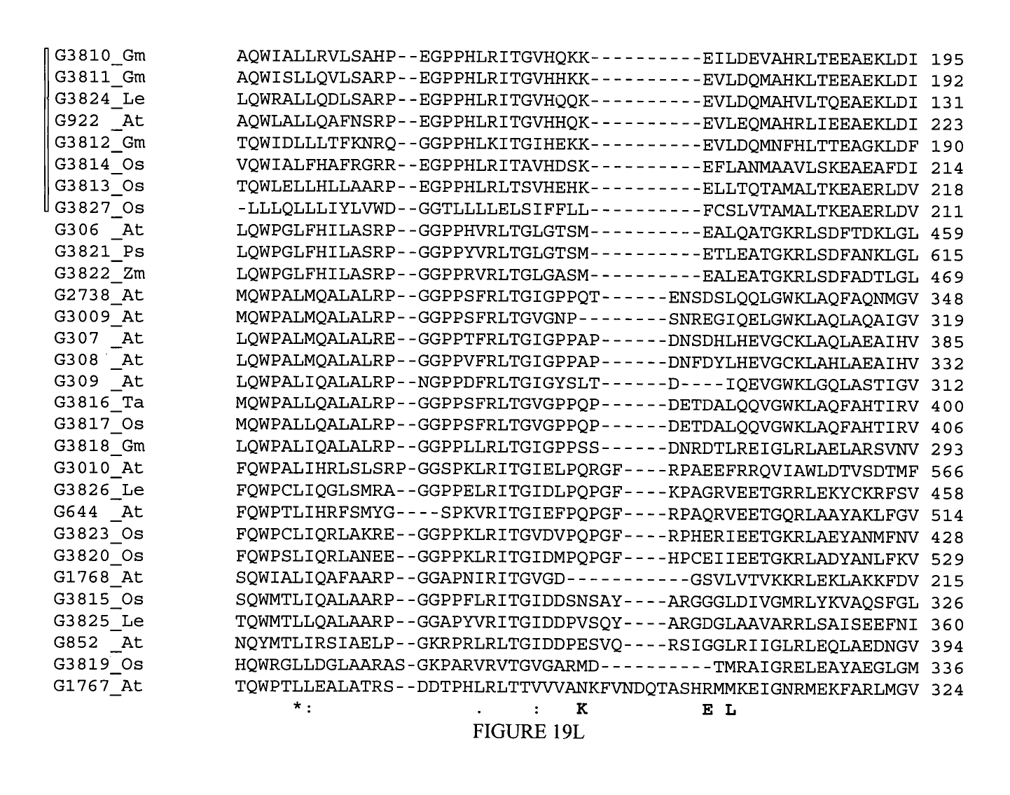

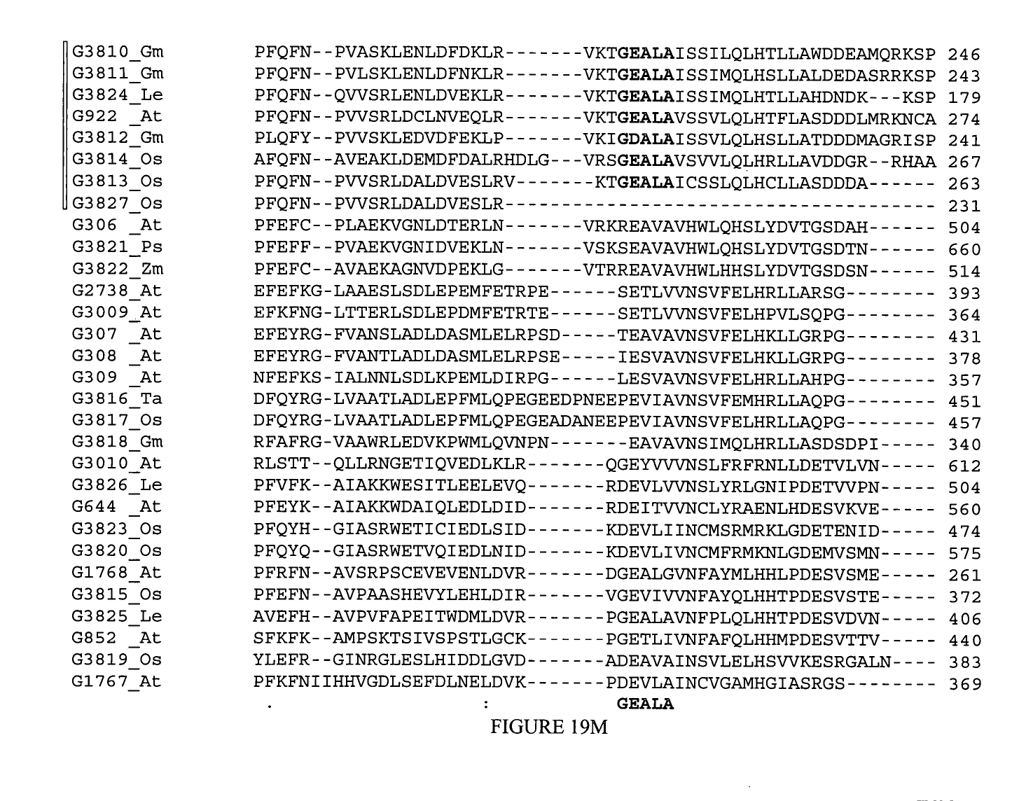

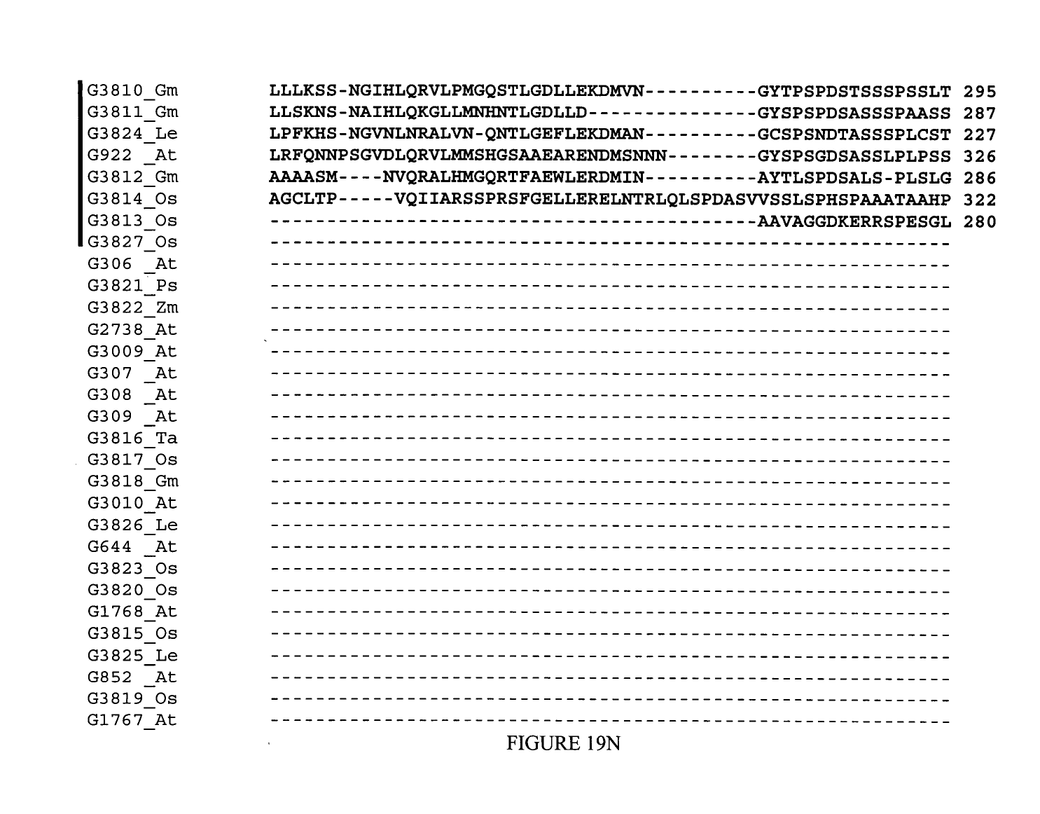

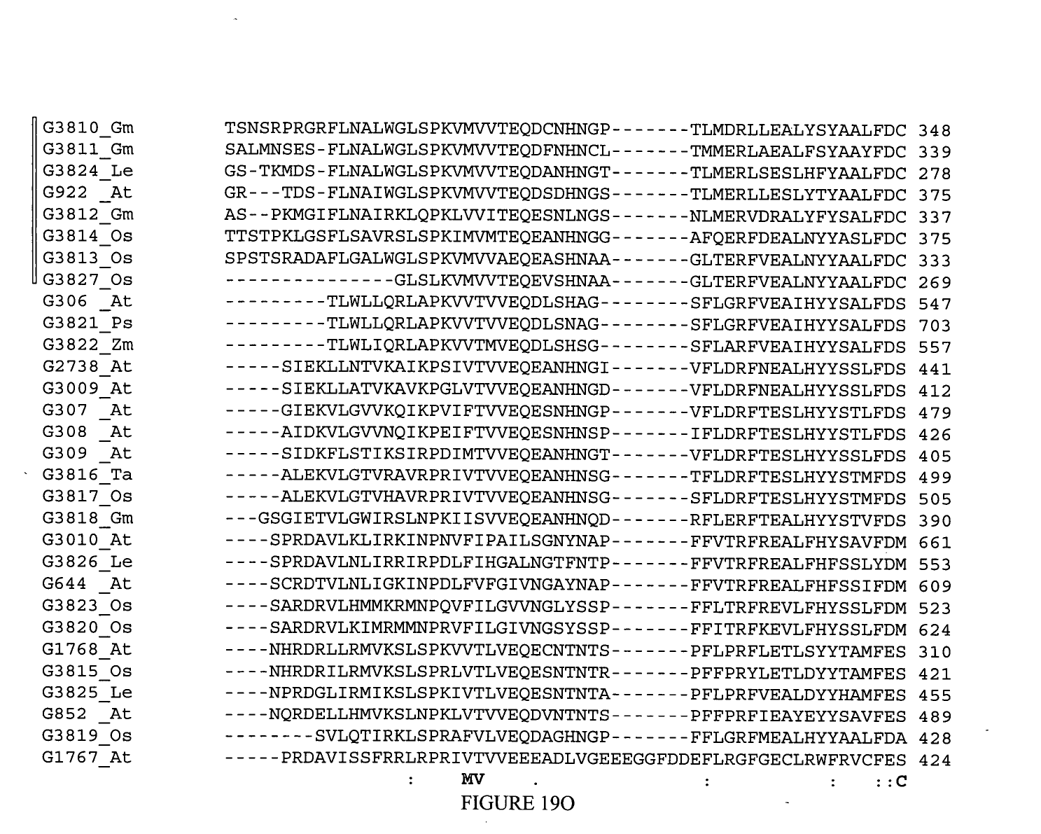

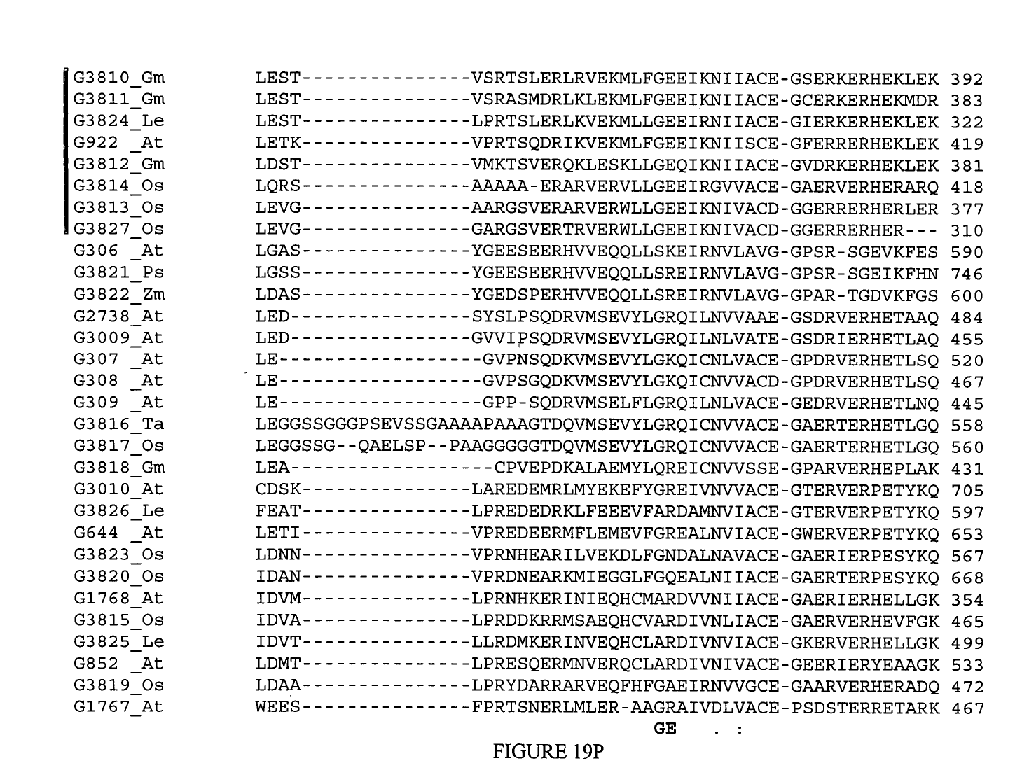

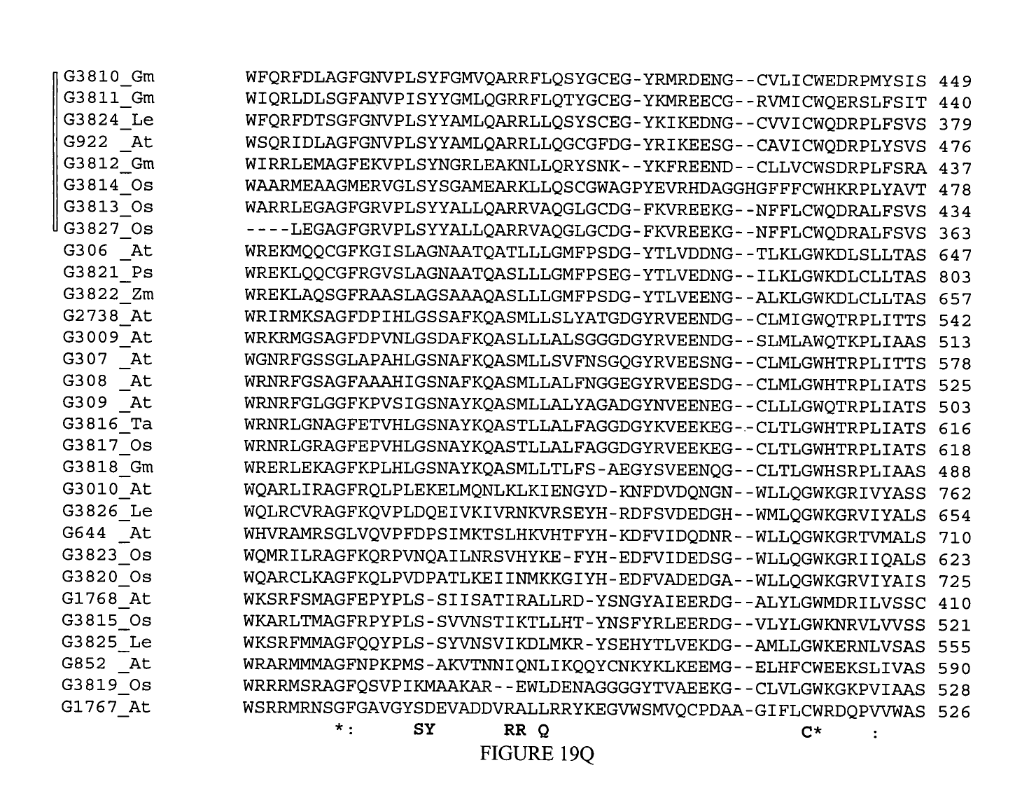

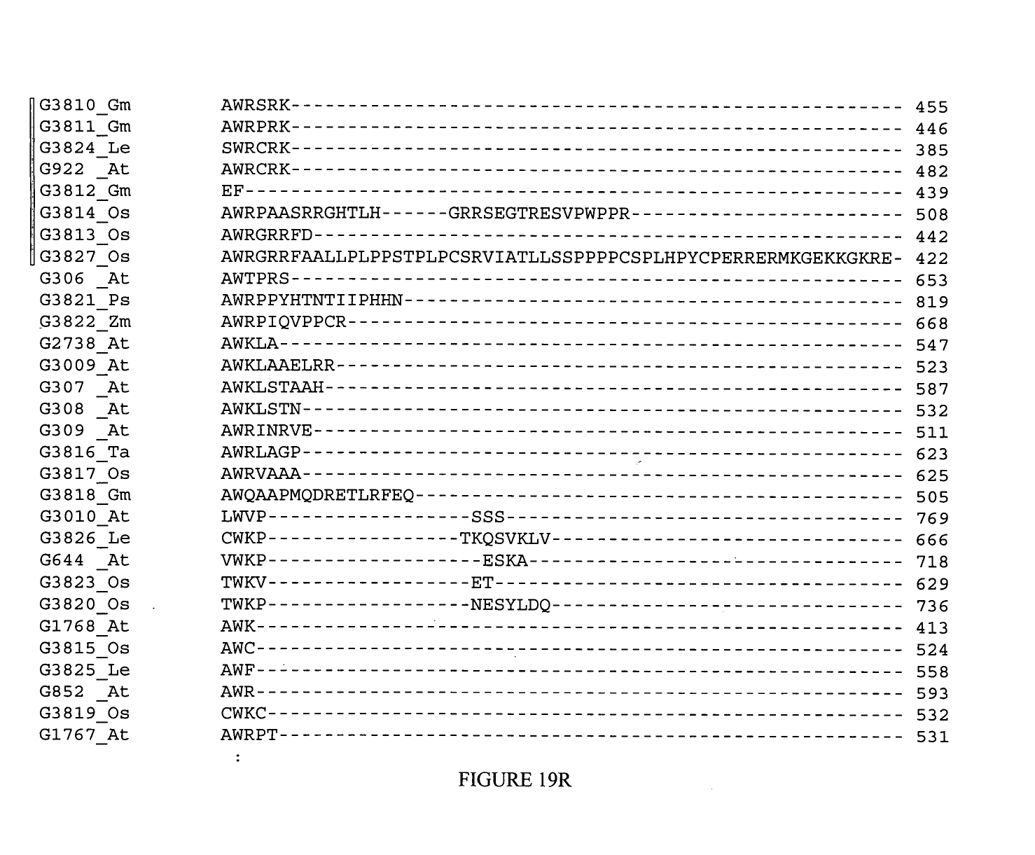

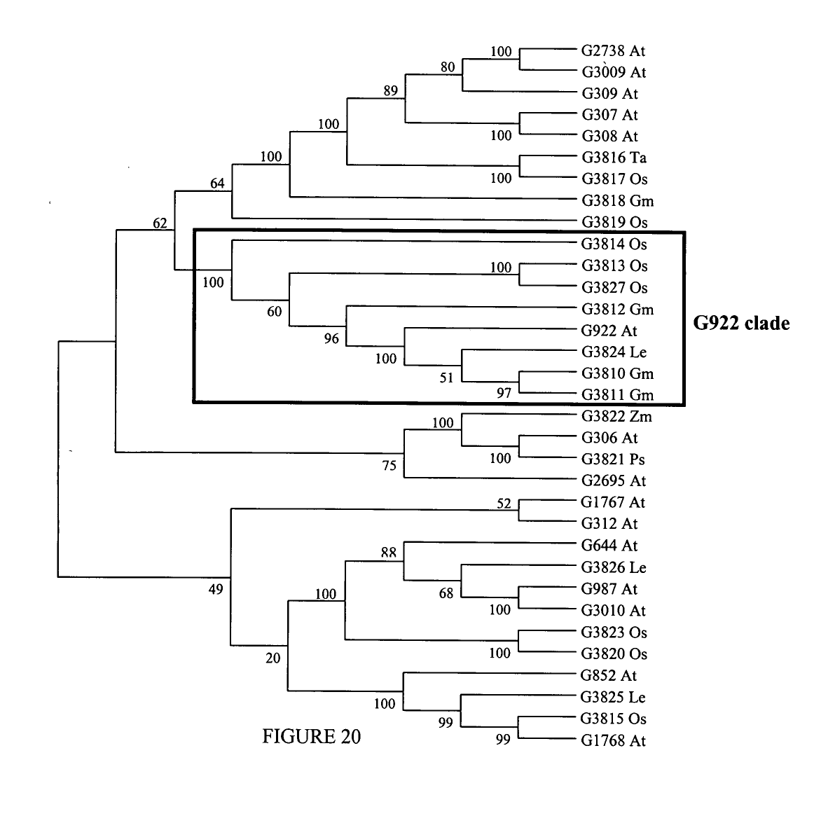

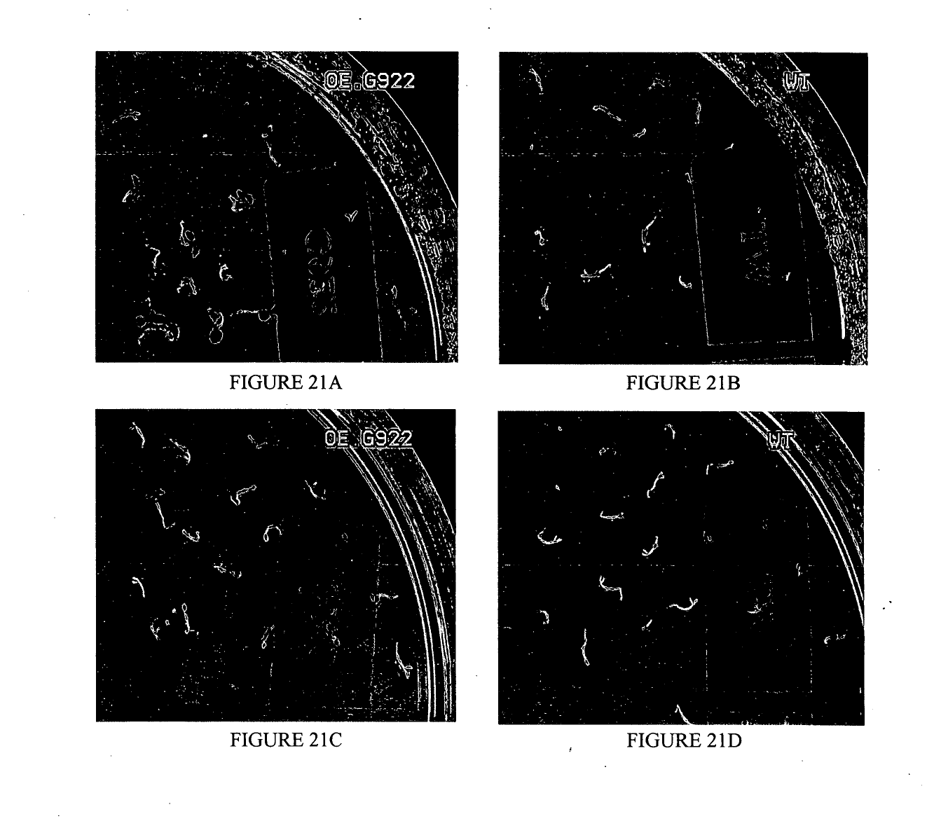

G3086 overexpressing lines were larger and more tolerant of heat stress. FIG. 18A shows the effects of a heat assay on Arabidopsis wild-type and G3086-overexpressing plants. The overexpressors on the left were generally larger, paler, and exhibited earlier bolting than the wild type plants seen on the right of this plate.

35S::G3086 transformants were also larger and displayed more root growth when grown under high salt conditions. G3086 overexpressors, as exemplified by the eight seedlings on the right of FIG. 18B, were larger, greener, and had more root growth than control plants, as exemplified by the four seedlings on the right in FIG. 18B.

The high salt assays suggested that this gene may confer drought tolerance, a supposition confirmed in a soil-based assay in which G3086 overexpressing-plants were significantly more tolerant of drought stress than control plants in soil-based drought assays (Tables 9 and 10).

Utilities

Based on the phenotypes observed in morphological and physiological assays, G3086 and its equivalogs might be have a number of utilities.

Given the salt resistance exhibited by 35S::G3086 transformants, the gene or its equivalogs might be used to engineer salt tolerant crops and trees that can flourish in saline soils, or under drought conditions.

Based on the response of 35S::G3086 lines to heat stress, the gene or its equivalogs might be used to engineer crop plants with increased tolerance to abiotic stresses such as high temperatures, a stress that often occurs simultaneously with other environmental stress conditions such as drought or salt stress.

The early flowering displayed by 35S::G3086 transformants indicated that the gene or its equivalogs might be used to accelerate the flowering of commercial species, or to eliminate any requirements for vernalization.

G3086 equivalogs include, for example, Arabidopsis thaliana SEQ ID NO: 26 (G592), SEQ ID NO: 28 (G1134), SEQ ID NO: 38 (G2149), SEQ ID NO: 40 (G2555); and SEQ ID NO: 42 (G2766); Oryza sativa (japonica cultivar-group) SEQ ID NO: 168 (G3740), SEQ ID NO: 170 (G3741), SEQ ID NO: 172 (G3742), SEQ ID NO: 174 (G3744), and SEQ ID NO: 176 (G3746); Glycine max SEQ ID NO: 180 (G3763), SEQ ID NO: 182 (G3764), SEQ ID NO: 184 (G3765), SEQ ID NO: 186 (G3766), SEQ ID NO: 188 (G3767), SEQ ID NO: 190 (G3768), SEQ ID NO: 192 (G3769), SEQ ID NO: 194 (G3771), and SEQ ID NO: 196 (G3772); Zea mays SEQ ID NO: 178 (G3755); and Pinus taeda SEQ ID NO: 197 (G3782).

G354 (SEQ ID NOs: 227 and 228)

Published Information

G354 was identified in the sequence of BAC clone F12M12, GenBank accession number AL355775, released by the Arabidopsis Genome Initiative. G354 corresponds to ZAT7 (Meissner and Michael (1997) Plant Mol. Biol. 33: 615-624).

Experimental Observations

The highest level of expression of G354 was observed in rosette leaves, embryos, and siliques. Some expression of G354 was also observed in flowers.

The function of this gene was analyzed using transgenic plants in which G353 was expressed under the control of the 35S promoter. 35S::G354 plants had a reduction in flower pedicel length, and downward pointing siliques. This phenotype was very similar to that described for the brevipedicellus (bp) mutant (Koornneef et al. (1983) J. Hered. 74: 265-272) and in overexpression of a related gene G353. Other morphological changes in shoots were also observed in 35S::G354 plants. Many 35S::G354 seedlings had abnormal cotyledons, elongated, thickened hypocotyls, and short roots. The majority of T1 plants had a very extreme phenotype, were tiny, and arrested development without forming inflorescences. T1 plants showing more moderate effects had poor seed yield.

Overexpression of G354 in Arabidopsis resulted in seedlings with an altered response to light. In a germination assay conducted in darkness, G354 seedlings failed to show an etiolation response, as can been seen in FIG. 30 which shows G354 overexpressing and wild-type seedlings germinated on MS plates in the dark. In some cases the phenotype was severe; overexpression of the transgene resulted in reduced open and greenish cotyledons.

G354 overexpressors were also shown to be tolerant to water deprivation in soil-based drought assays (Tables 9 and 10). Closely related paralogs of this gene, G353 and G2839, also showed an osmotic stress tolerance phenotype in a germination assay on media containing high sucrose; one line of 35S::G353 seedlings and several lines of 35S::G2839 were greener and had higher germination rates than controls. Thus, G354 and its paralogs G353 and G2839 appear to influence osmotic stress responses.

Utilities

G354 and its equivalogs can be could be used to increase a plant's tolerance to drought and other osmotic stress, and can be used alter inflorescence structure, which may have value in production of novel ornamental plants.

G1792 (SEQ ID NO: 7 and 8)

Published Information

G1792 was identified in the sequence of BAC clone K14B15 (AB025608, gene K14B15.14).

Closely Related Genes from Other Species

G1792 shows sequence similarity, outside the conserved AP2 domain, with a portion of a predicted protein from tomato, represented by EST sequence AI776626 (AI776626 EST257726 tomato resistant, Cornell Lycopersicon esculentum cDNA clone cLER19A14, mRNA sequence).

Experimental Observations

G1792 was studied using transgenic plants in which the gene was expressed under the control of the 35S promoter.

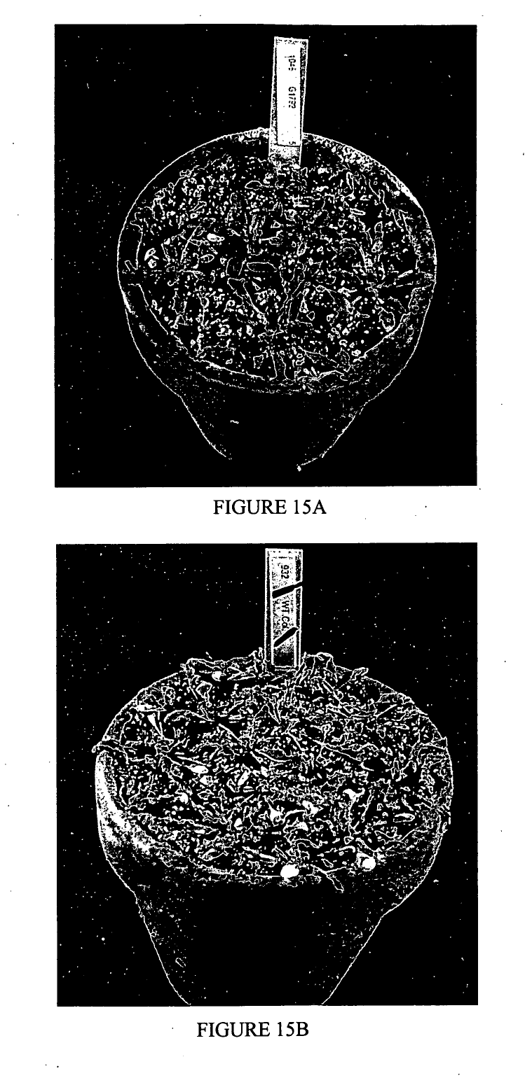

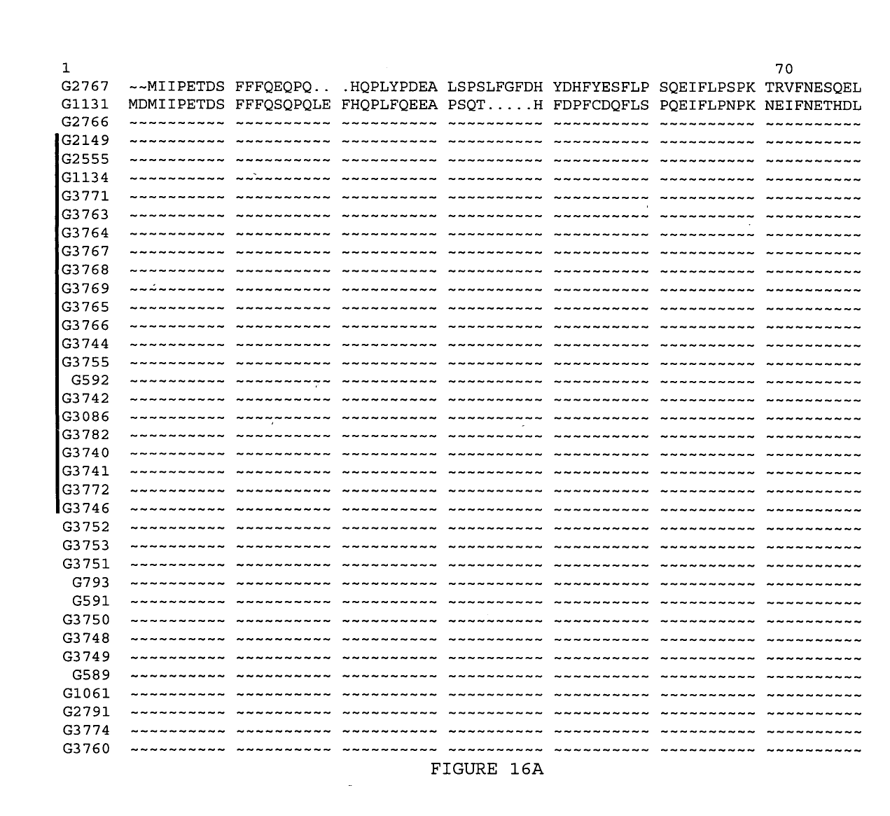

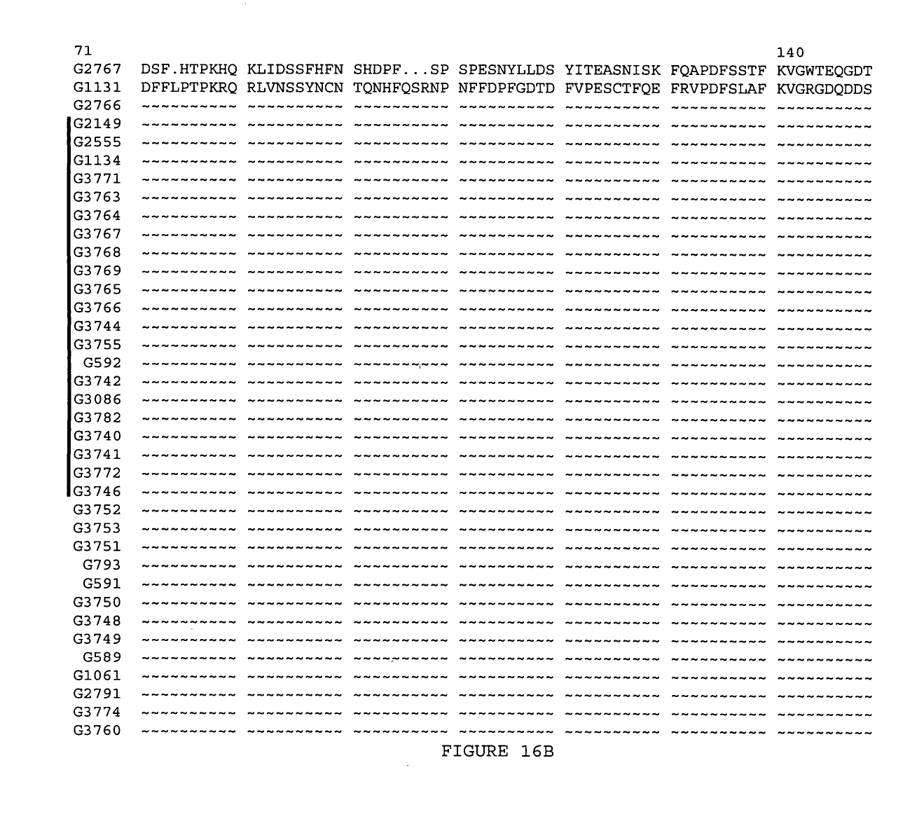

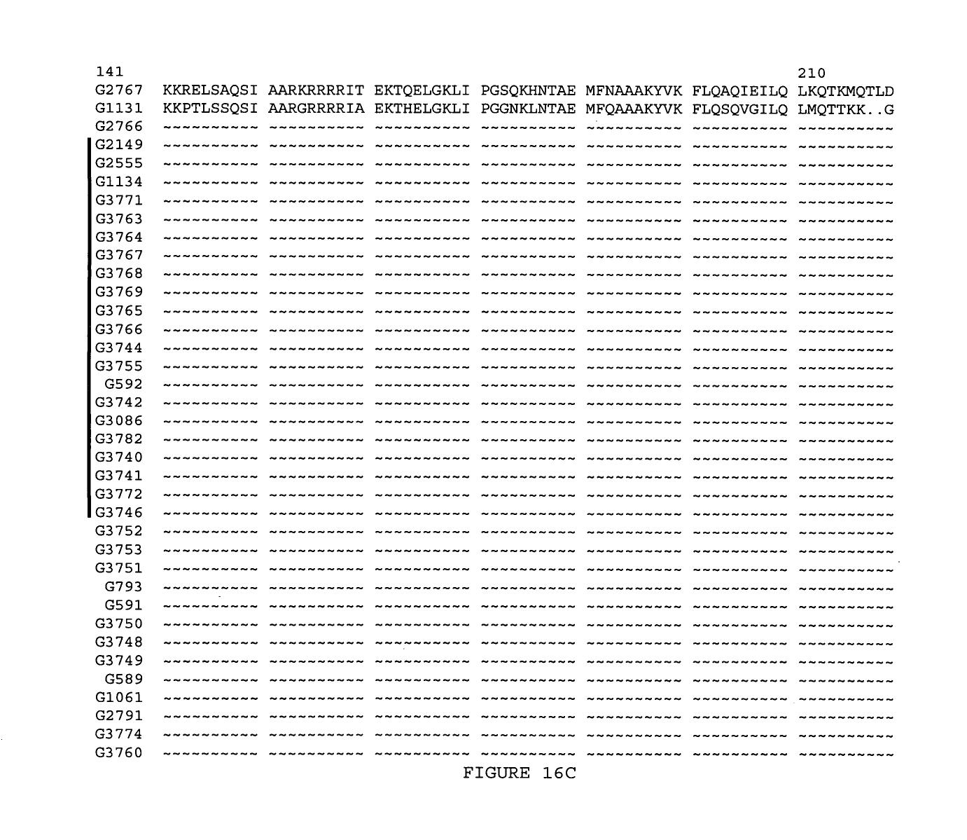

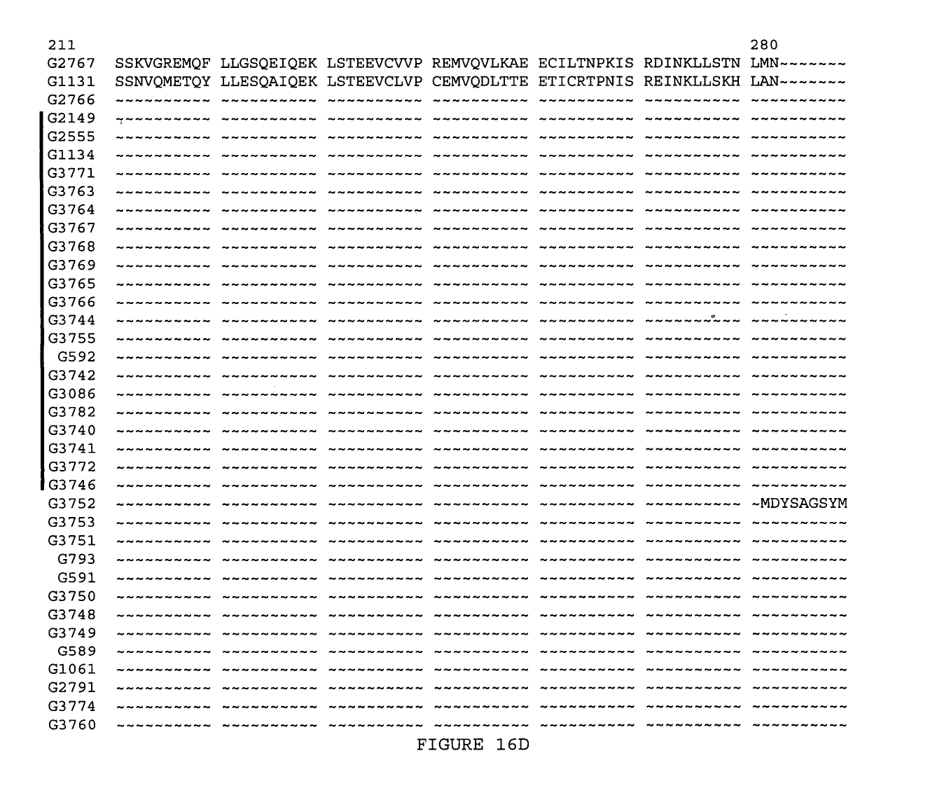

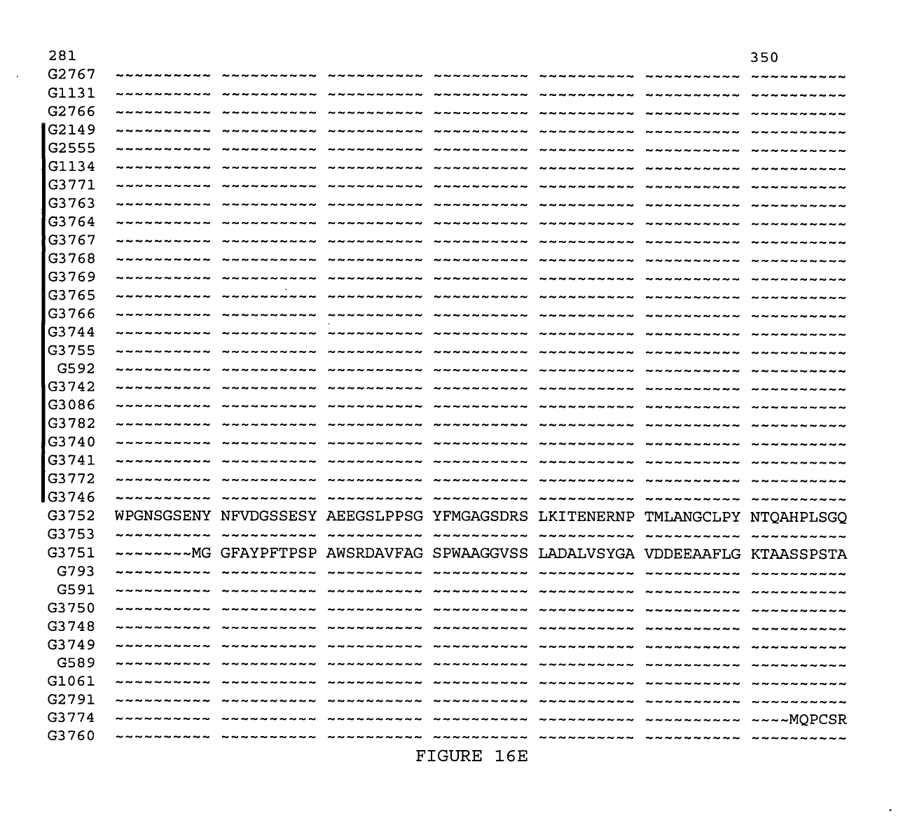

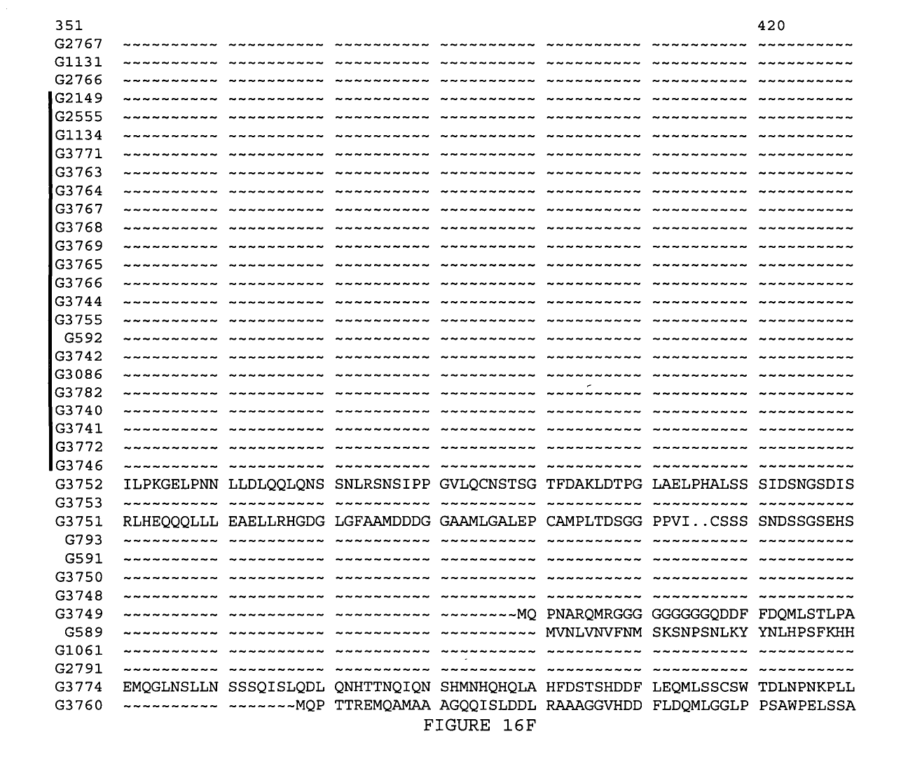

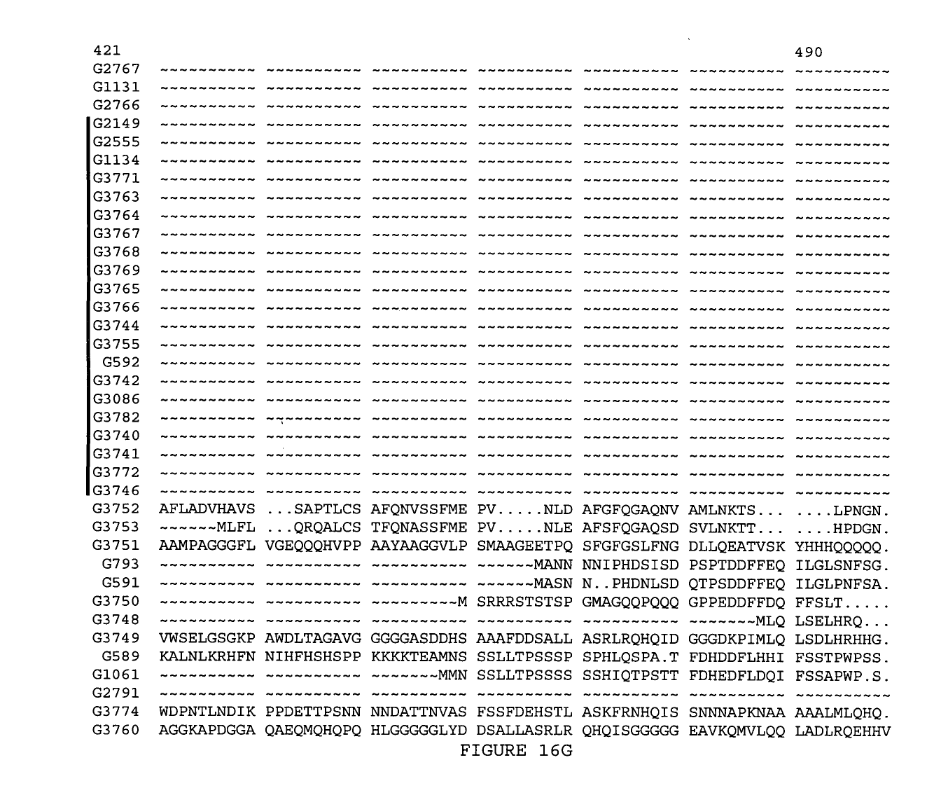

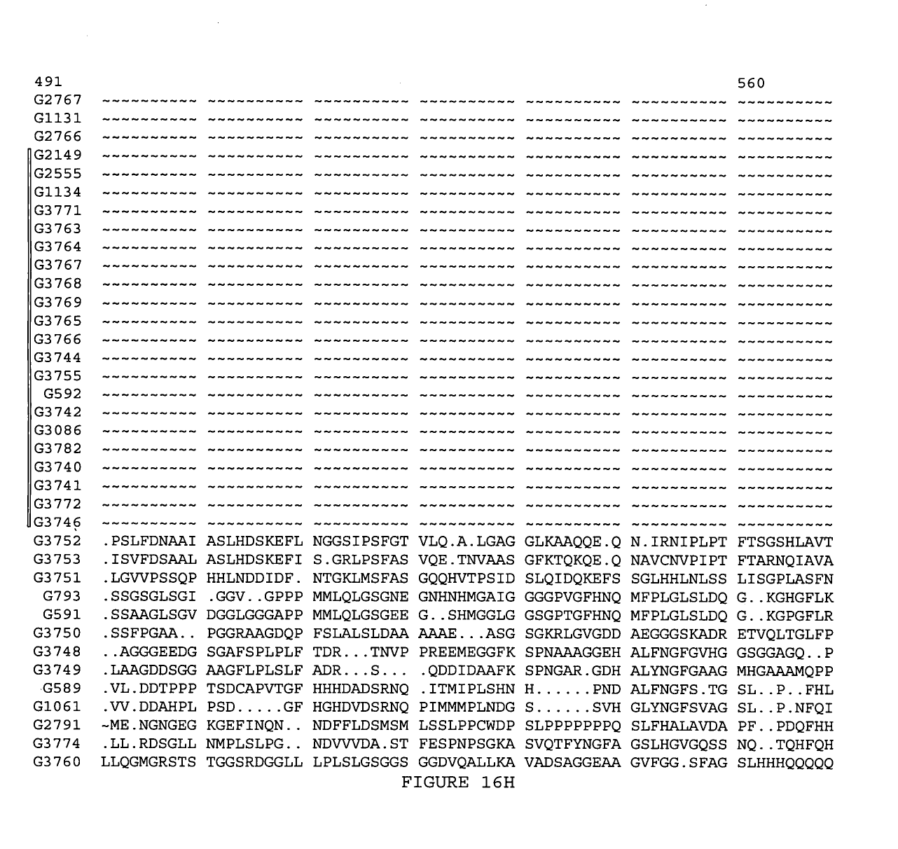

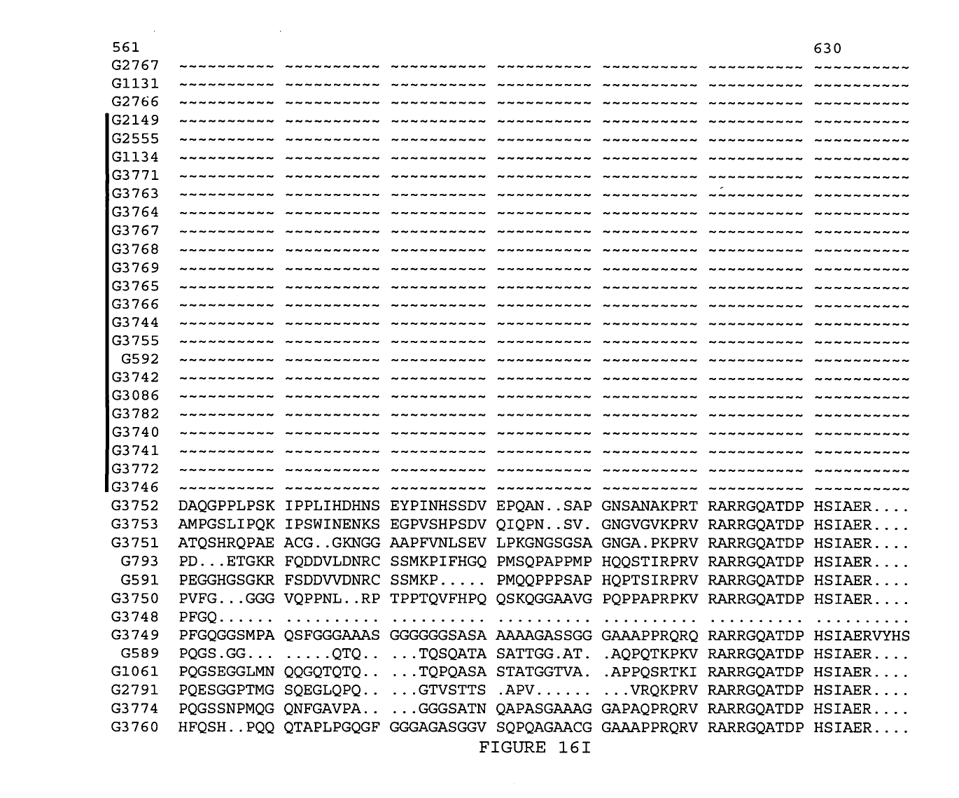

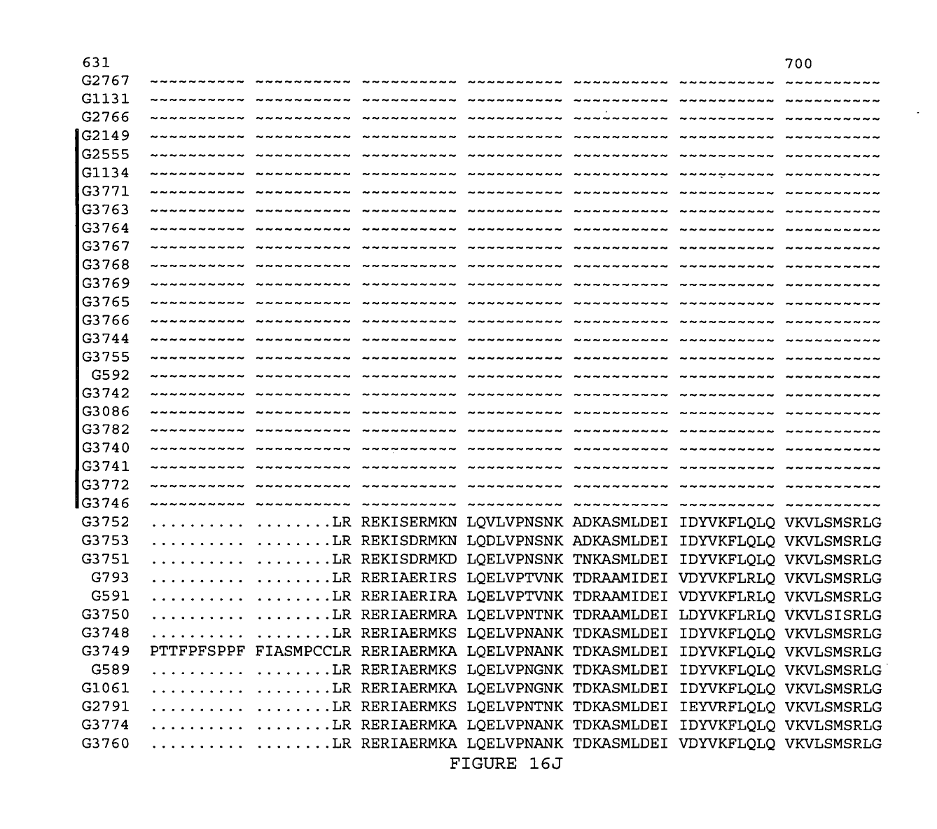

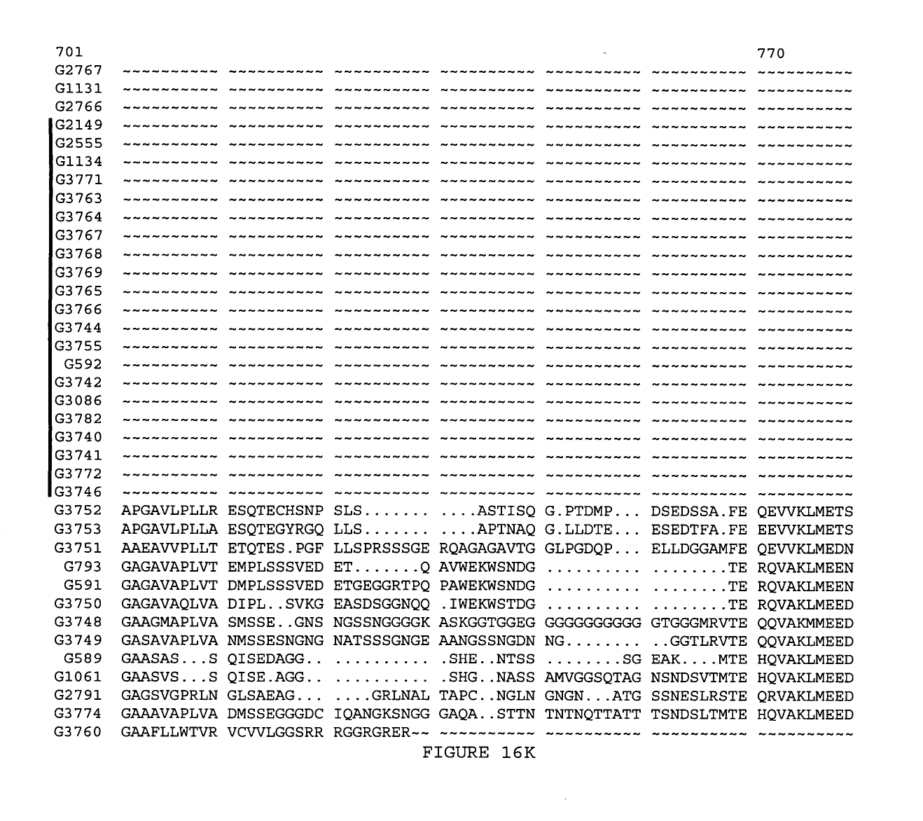

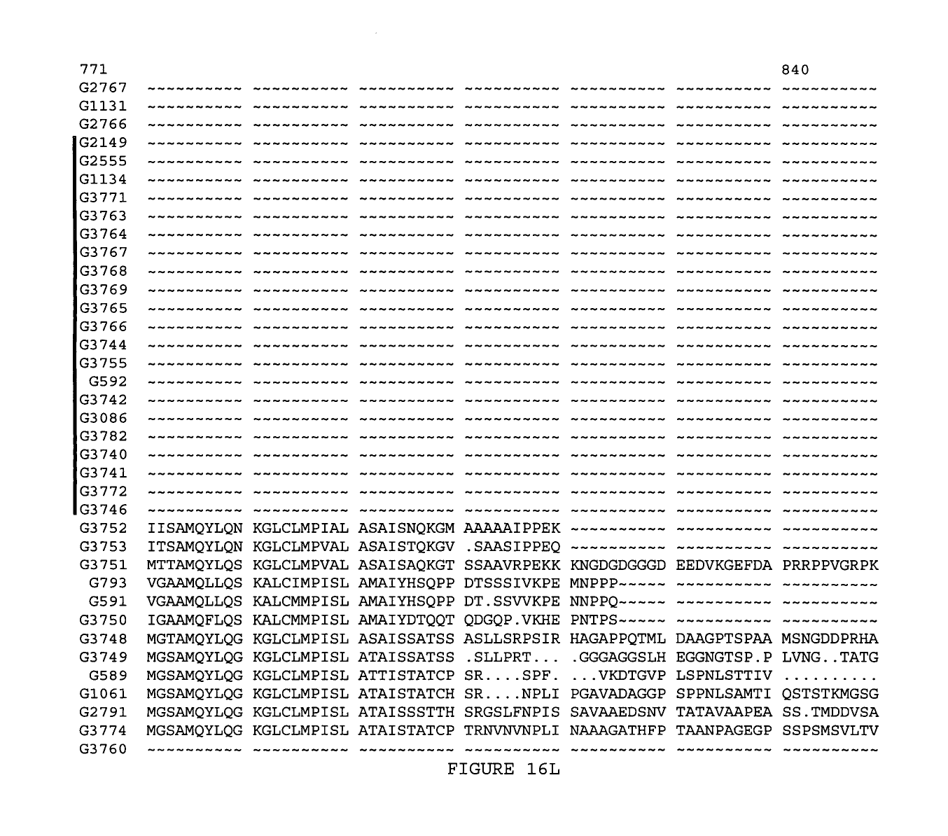

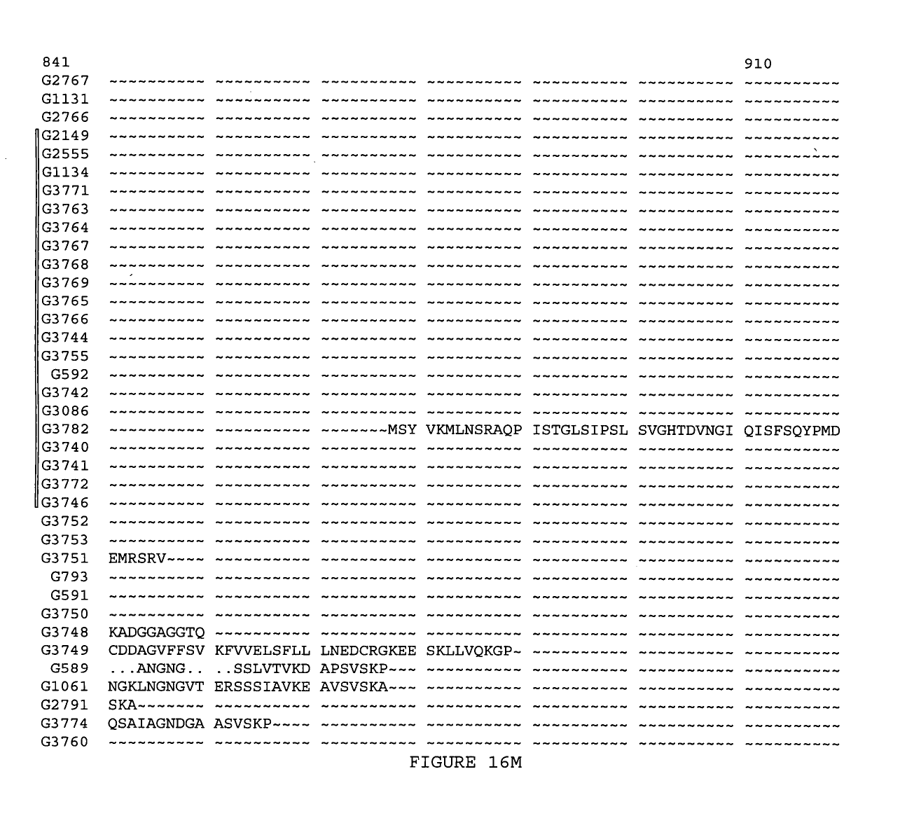

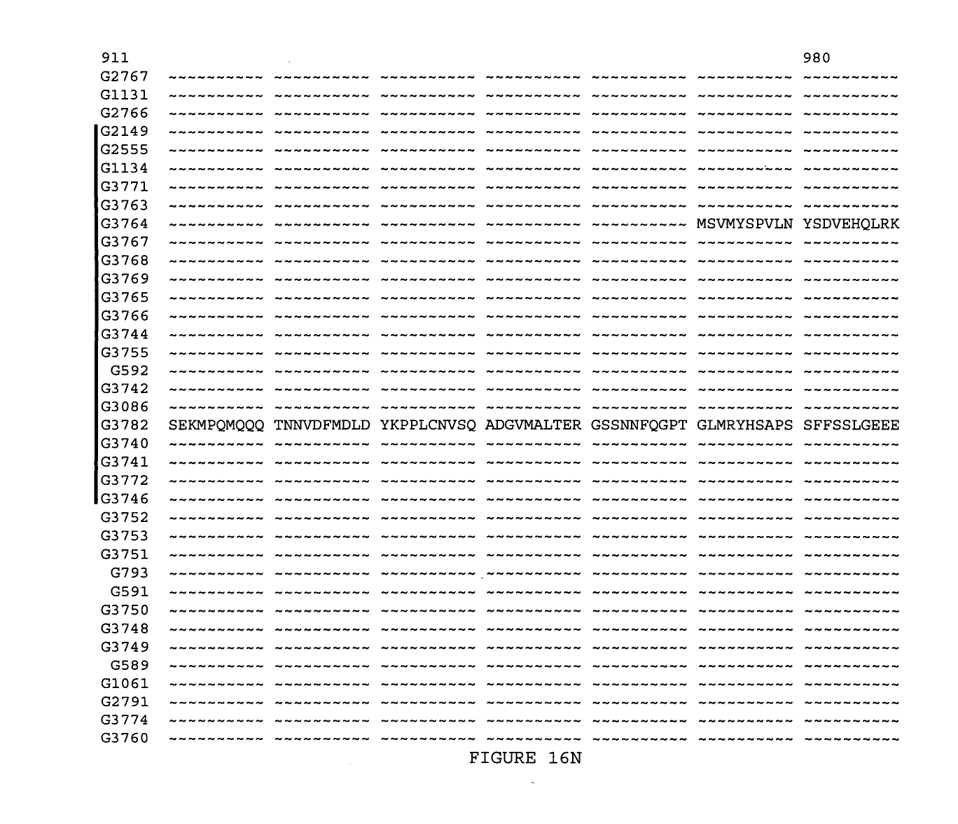

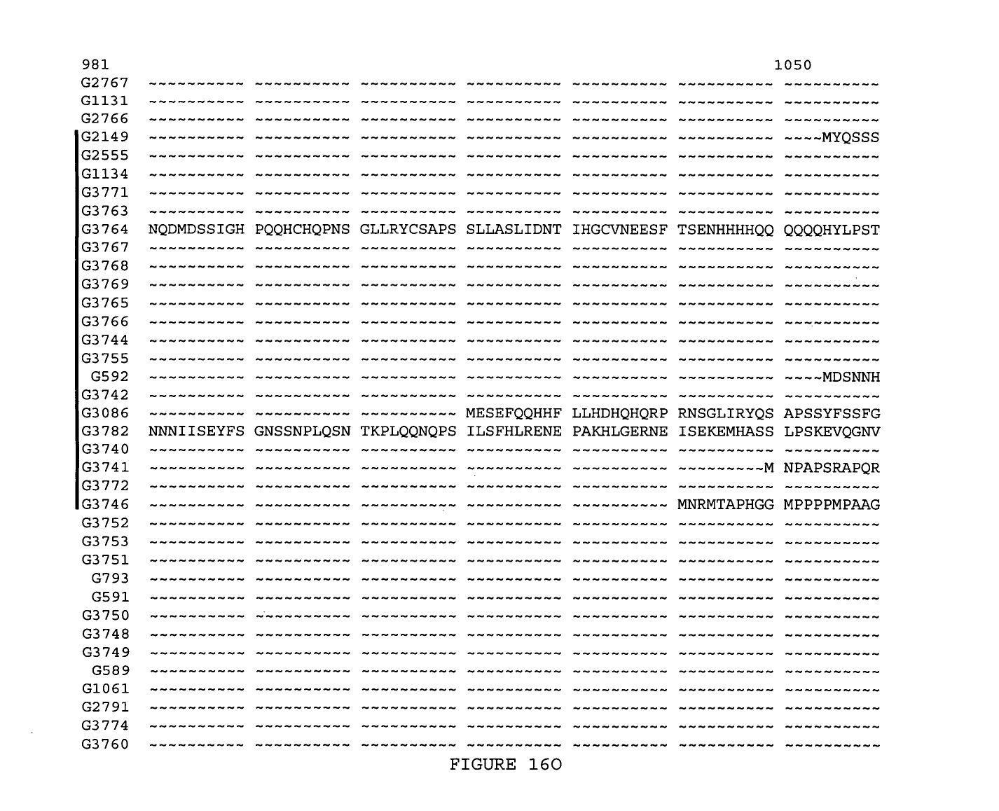

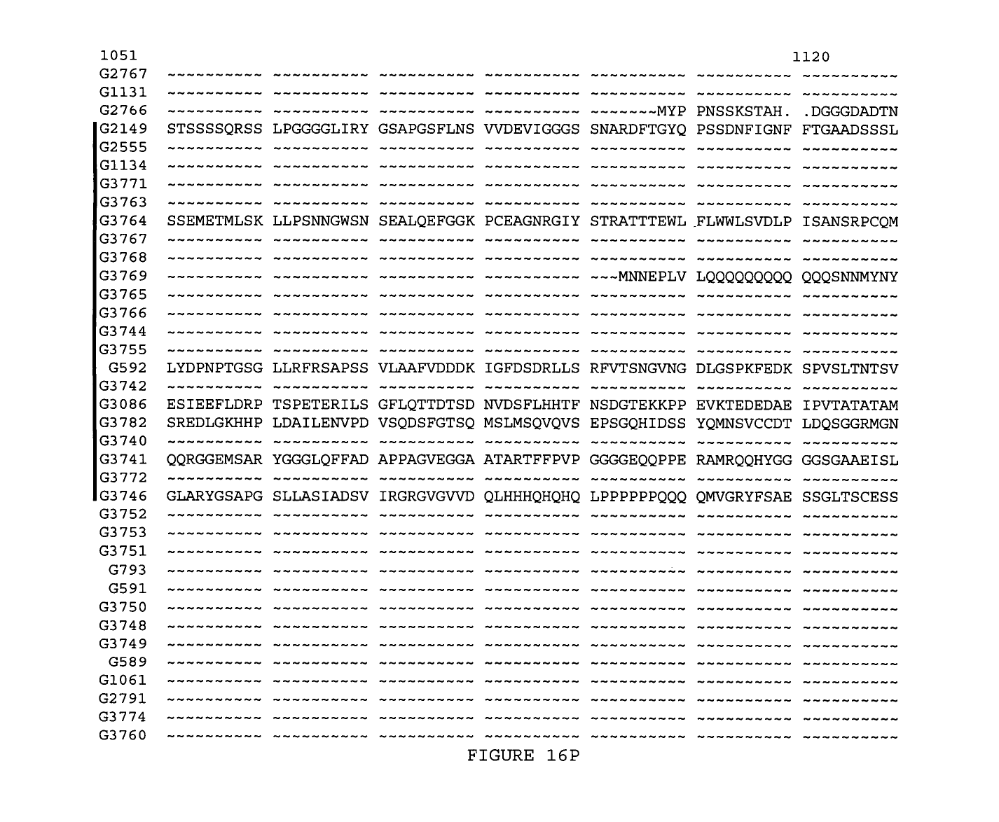

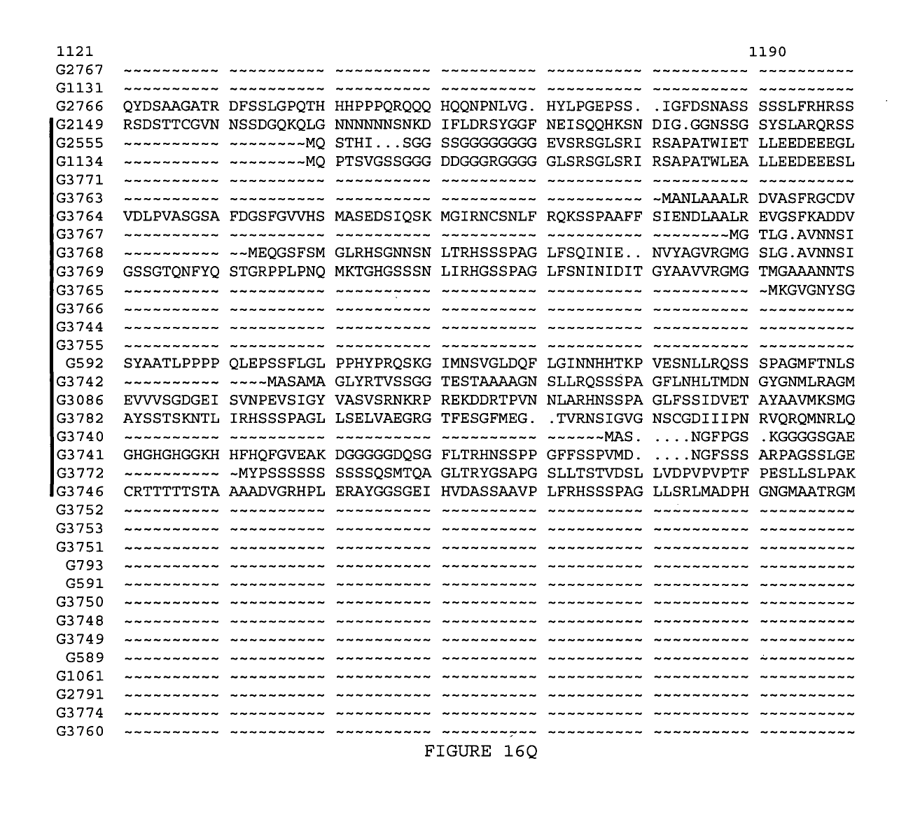

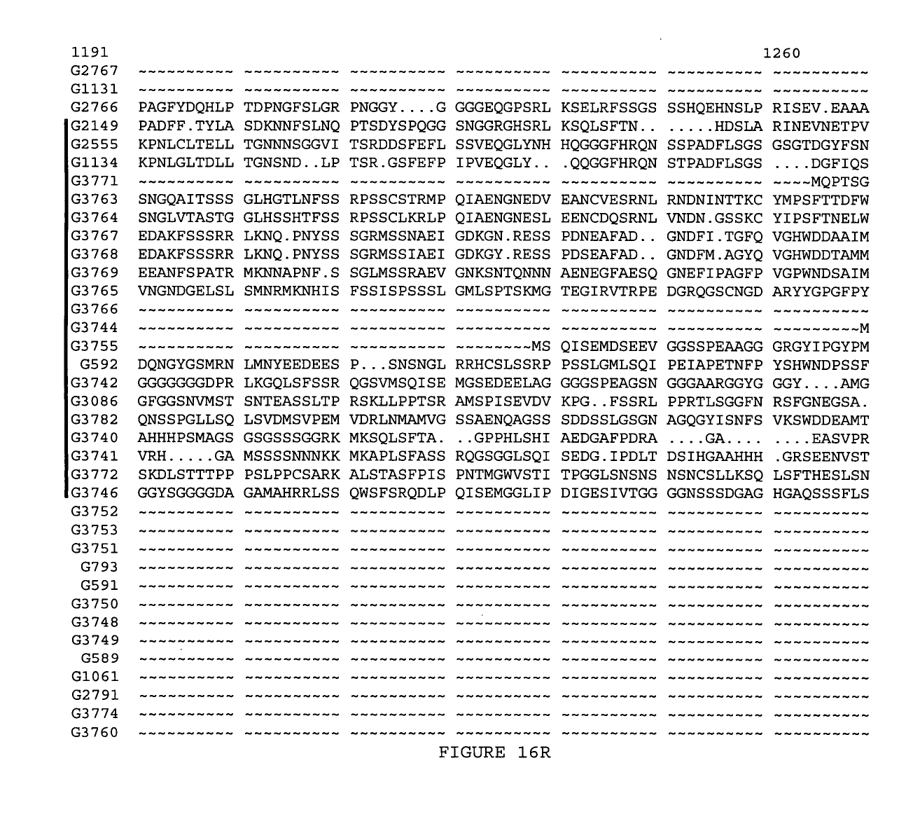

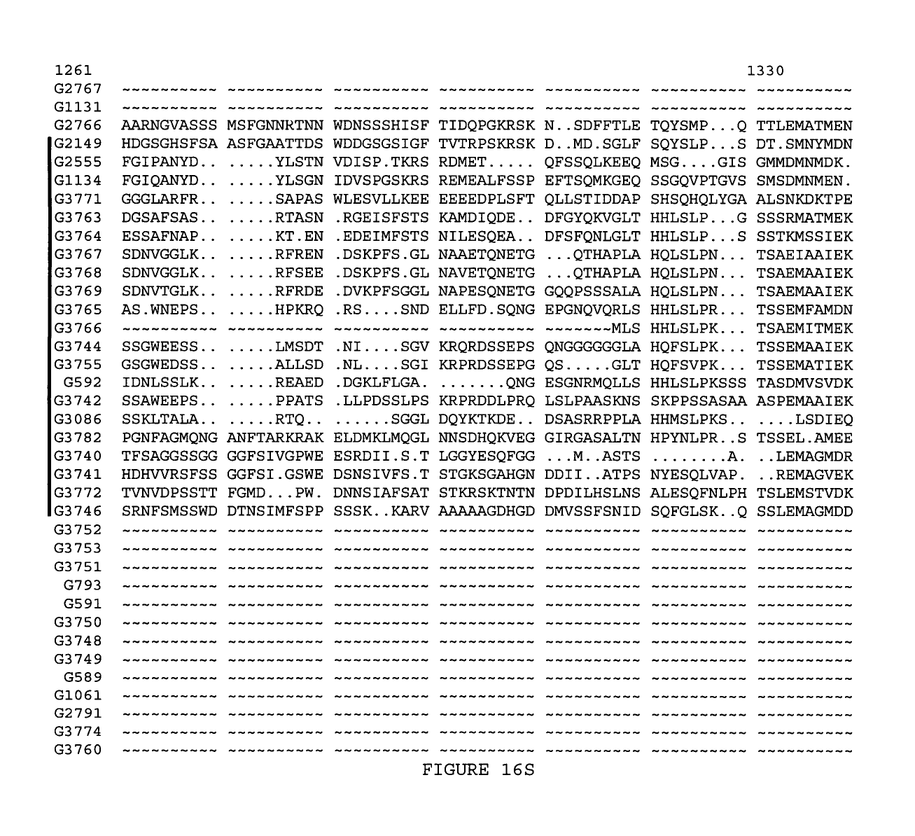

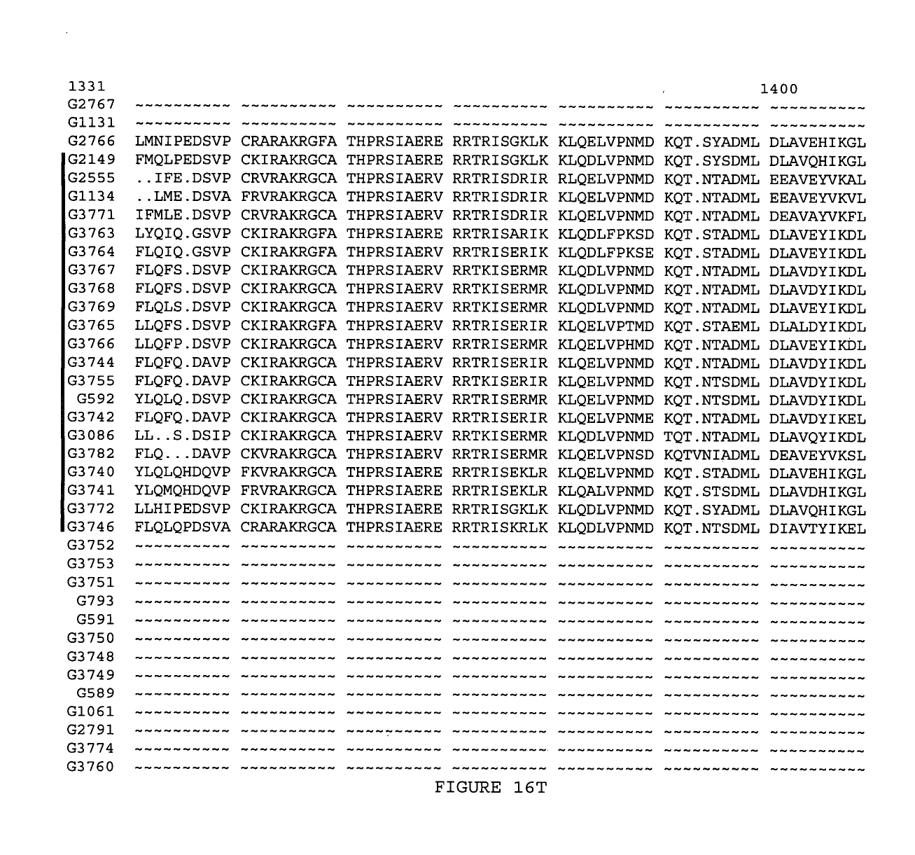

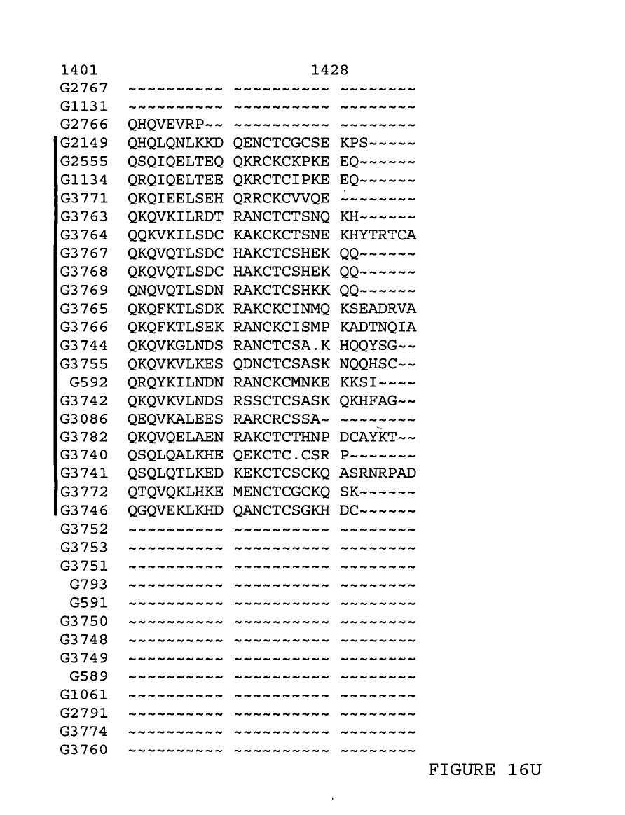

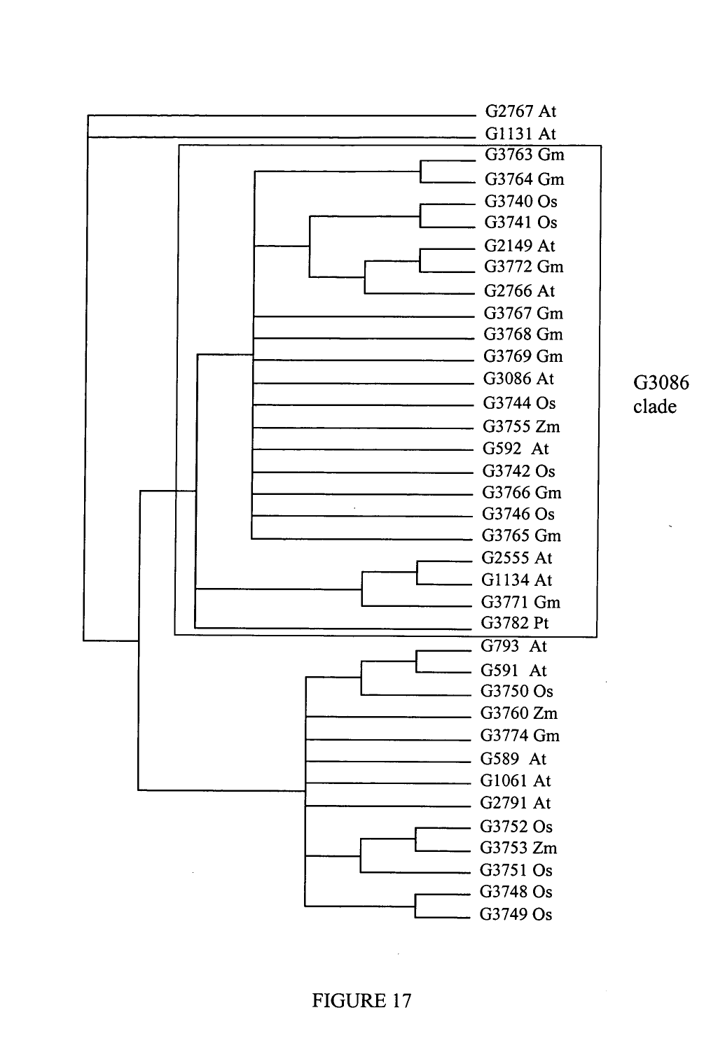

In soil-based assays, G1792 overexpressing plants were significantly more drought tolerant than wild-type control plants (FIGS. 15A and 15B, Tables 9 and 10).

35S::G1792 plants were more tolerant to the fungal pathogens Fusarium oxysporum and Botrytis cinerea and showed fewer symptoms after inoculation with a low dose of each pathogen. This result was confirmed using individual T2 lines. The effect of G1792 overexpression in increasing tolerance to pathogens received further, incidental confirmation. T2 plants of two 35S::G1792 lines had been growing in a room that suffered a serious powdery mildew infection. For each line, a pot of six plants was present in a flat containing nine other pots of lines from unrelated genes. In either of the two different flats, the only plants that were free from infection were those from the 35S::G1792 line. This observation suggested that G1792 overexpression might be used to increase resistance to powdery mildew. Additional experiments confirmed that 35S::G1792 plants showed increased tolerance to Erysiphe. G1792 was ubiquitously expressed, but appeared to be induced by salicylic acid.

35S::G1792 overexpressing plants also showed more tolerance to growth under nitrogen-limiting conditions. In a root growth assay under conditions of limiting N, 35S::G1792 lines were slightly less stunted. In a germination assay that monitored the effect of C on N signaling through anthocyanin production on high sucrose plus and minus glutamine the 35S::G1792 lines made less anthocyanin on high sucrose plus glutamine, suggesting that the gene can be involved in the plants ability to monitor their carbon and nitrogen status.

G1792 overexpressing plants showed several mild morphological alterations: leaves were dark green and shiny, and plants bolted, subsequently senesced, slightly later than wild-type controls. Among the T1 plants, additional morphological variation (not reproduced later in the T2 plants) was observed: many showed reductions in size as well as aberrations in leaf shape, phyllotaxy, and flower development.

Utilities

G1792 or its equivalogs can be used to improve drought and other osmotic stress tolerances, and engineer pathogen-resistant plants. In addition, it can also be used to improve seedling germination and performance under conditions of limited nitrogen.

Potential utilities of this gene or its equivalogs also include increasing chlorophyll content allowing more growth and productivity in conditions of low light. With a potentially higher photosynthetic rate, fruits could have higher sugar content. Increased carotenoid content could be used as a nutraceutical to produce foods with greater antioxidant capability.

G1792 or its equivalogs could be used to manipulate wax composition, amount, or distribution, which in turn could modify plant tolerance to drought and/or low humidity or resistance to insects, as well as plant appearance (shiny leaves). In particular, it would be interesting to see what the effect of increased wax deposition on leaves of a plant like cotton would do to drought resistance or water use efficiency. A possible application for this gene might be in reducing the wax coating on sunflower seeds (the wax fouls the oil extraction system during sunflower seed processing for oil). For this purpose, antisense or co-suppression of the gene in a tissue specific manner might be useful

G1792 equivalogs include, for example, Arabidopsis thaliana SEQ ID NO: 18 (G30), SEQ ID NO: 34 (G1791), and SEQ ID NO: 36 (G1795); Medicago truncatula SEQ ID NO: 160 (G3735); Glycine max SEQ ID NO: 82 (G3518), SEQ ID NO: 84 (G3519), SEQ ID NO: 86 (G3520); Oryza sativa (japonica cultivar-group) SEQ ID NO: 70 (G3380), SEQ ID NO: 72 (G3381), SEQ ID NO: 74 (G3383), SEQ ID NO: 76 (G3515), and SEQ ID NO: 164 (G3737); Zea mays), SEQ ID NO: 78 (G3516), SEQ ID NO: 80 (G3517), SEQ ID NO: 200 (G3794), SEQ ID NO: 166 (G3739) and Triticum aestivum SEQ ID NO: 162 (G3736).

G2053 (SEQ ID NO: 9 and 10)

Published Information

G2053 was identified in the sequence of BAC T27C4, GenBank accession number AC022287, released by the Arabidopsis Genome Initiative.

Experimental Observations

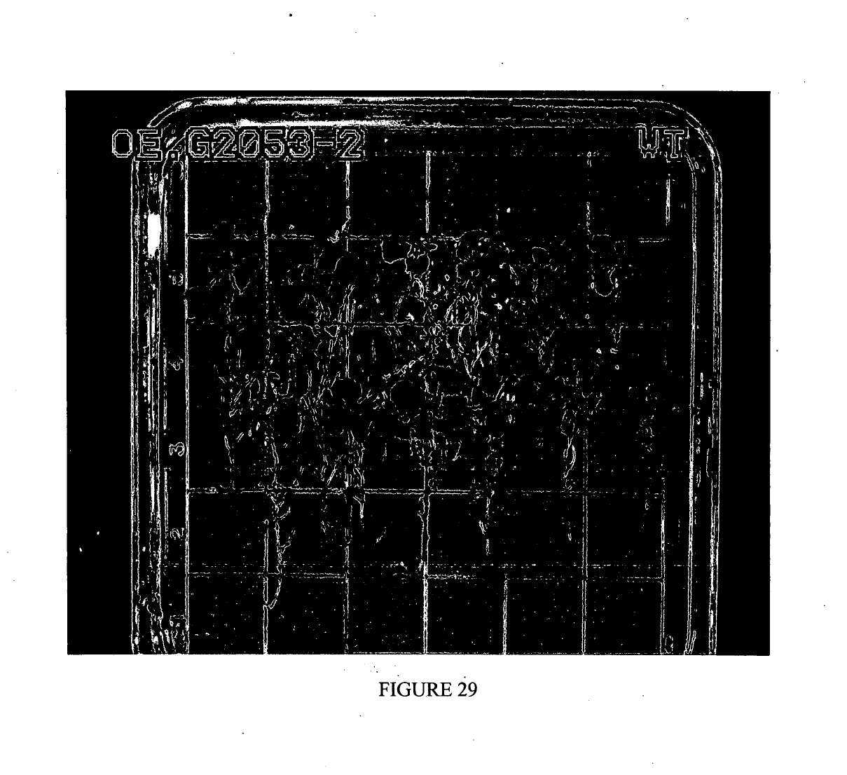

The function of G2053 was analyzed using transgenic plants in which the gene was expressed under the control of the 35S promoter. Overexpression of G2053 in Arabidopsis resulted in plants with altered osmotic stress tolerance. In a root growth assay on media containing high concentrations of PEG, G2053 overexpressors showed more root growth compared to wild-type controls (FIG. 29).

The osmotic stress tolerance assays suggested that this gene may confer drought tolerance, a supposition confirmed in soil-based assays in which G2053 overexpressors were significantly more drought tolerant than wild-type control plants (Tables 9 and 10).

Utilities

Based on the altered stress tolerance induced by G2053 overexpression, this transcription factor or its equivalogs could be used to alter a plant's response water deficit conditions and, therefore, could be used to engineer plants with enhanced tolerance to drought, salt stress, and freezing.

G2053 equivalogs include, for example, Arabidopsis thaliana SEQ ID NO: 20 (G515), SEQ ID NO: 22 (G516), and SEQ ID NO: 24 (G517)

G975 (SEQ ID NO: 237 and 238)

Published Information

After its discovery by us, G975 has appeared in the sequences released by the Arabidopsis Genome Initiative (BAC F9L1, GenBank accession number AC007591).

Closely Related Genes from Other Species

The non-Arabidopsis gene most highly related to G975 (as detected in BLAST searches, 11-5-99) is represented by L46408 BNAF1258 Mustard flower buds Brassica rapa cDNA clone F1258. The similarity between G975 and the Brassica rapa gene represented by EST L46408 extends beyond the conserved AP2 domain that characterizes the AP2/EREBP family. In fact, this Brassica rapa gene appears to be more closely related to G975 than Arabidopsis G1387, indicating that EST L46408 may represent a true G975 ortholog. The similarity between G975 and Arabidopsis G1387 also extends beyond the conserved AP2 domain.

Experimental Observations

G975 was discovered by us and is a new member of the AP2/EREBP family (EREBP subfamily) of transcription factors. G975 is expressed in flowers and, at lower levels, in shoots, leaves, and siliques. GC-FID and GC-MS analyses of leaves from G975 overexpressing plants have shown that the levels of C29, C31, and C33 alkanes were substantially increased (up to 10-fold) compared to control plants. A number of additional compounds of similar molecular weight, presumably also wax components, also accumulated to significantly higher levels in G975 overexpressing plants. Although total amounts of wax in G975 overexpressing plants have not yet been measured, C29 alkanes constitute close to 50% of the wax content in wild-type plants (Millar et al. (1998) Plant Cell 11: 1889-1902), indicating that a major increase in total wax content occurs in these transgenic plants. However, the transgenic plants had an almost normal phenotype (small morphological differences are detected in leaf appearance), indicating that overexpression of G975 is not deleterious to the plant. It is noteworthy that overexpression of G975 did not cause the dramatic alterations in plant morphology that have been reported for Arabidopsis plants in which the FATTY ACID ELONGATION1 gene was overexpressed (Millar et al. (1998) supra). G975 could specifically regulate the expression of some of the genes involved in wax metabolism. One Arabidopsis AP2 gene was found that is significantly more closely related to G975 than the rest of the members of the AP2/EREBP family. This other gene, G1387, may have a function, and therefore a utility, related to that of G975.

Plants overexpressing G975 were significantly larger and greener than wild-type control plants in a soil-based drought assay (Tables 9 and 10).

Utilities

G975 or its equivalogs could be used to improve a plant's tolerance to drought or low water conditions.

G975 or its equivalogs could be used to manipulate wax composition, amount, or distribution, which in turn could modify plant tolerance to drought and/or low humidity or resistance to insects, as well as plant appearance (shiny leaves). A possible application for this gene or its equivalogs might be in reducing the wax coating on sunflower seeds (the wax fouls the oil extraction system during sunflower seed processing for oil). For this purpose, antisense or co-suppression of the gene in a tissue specific manner might be useful.

G975 could also be used to specifically alter wax composition, amount, or distribution in those plants and crops from which wax is a valuable product.

G1069 (SEQ ID NO: 239 and 240)

Published Information

The sequence of G1069 was obtained from EU Arabidopsis sequencing project, GenBank accession number Z97336, based on its sequence similarity within the conserved domain to other AT-Hook related proteins in Arabidopsis.

Closely Related Genes from Other Species

G1069 protein shares a significant homology to a cDNA isolated from Lotus japonicus nodule library. Similarity between G1069 and the Lotus cDNA extends beyond the signature motif of the family to a level that would suggest the genes are orthologous. Therefore the gene represented by EST AW720668 may have a function and/or utility similar to that of G1069.

Experimental Observations

The sequence of G1069 was experimentally determined and the function of G1069 was analyzed using transgenic plants in which G1069 was expressed under the control of the 35S promoter.

Plants overexpressing G1069 showed changes in leaf architecture, reduced overall plant size, and retarded progression through the life cycle. This is a common phenomenon for most transgenic plants in which AT-HOOK proteins are overexpressed if the gene is predominantly expressed in root in the wild-type background. G1069 was predominantly expressed in roots, based on analysis of RT-PCR results. To minimize these detrimental effects, G1069 may be overexpressed under a tissue specific promoter such as root- or leaf-specific promoter or under inducible promoter.

One of G1069 overexpressing lines showed more tolerance to osmotic stress when they were germinated in high sucrose plates. This line also showed insensitivity to ABA in a germination assay.

The high sucrose and ABA assay results suggested that this gene may confer increased tolerance to other abiotic stresses when G1069 is overexpressed. This was subsequently confirmed in soil-based drought assays in which 35S::G1069 plants were more drought tolerant than wild-type control plants (Tables 9 and 10).

Utilities

The drought and osmotic stress results indicate that G1069 could be used to alter a plant's response to water deficit conditions and, therefore, the gene or its equivalogs could be used to engineer plants with enhanced tolerance to drought, salt stress, and freezing.

G1069 affects ABA sensitivity, and thus when transformed into a plant the gene or its equivalogs may diminish cold, drought, oxidative and other stress sensitivities, and also be used to alter plant architecture, and yield.

G916 (SEQ ED NO: 235 and 236)

Published Information

G916 corresponds to gene At4g04450, and it has also been described as WRKY42. No information is available about the function(s) of G916.

Experimental Observations

The complete cDNA sequence of G916 was experimentally determined. G916 appears to be expressed at low levels in a range of tissues, and was not significantly induced by any of the conditions tested.

A T-DNA insertion mutant for G916, displayed wild-type morphology. Overexpression of G916 produced a wide spectrum of developmental abnormalities in Arabidopsis. Many of the 35S::G916 seedlings were extremely tiny and showed an apparent lack of shoot organization. Such plants arrested growth and died at very early stages. Other individuals were small and displayed disproportionately long hypocotyls and narrow cotyledons. At later stages, the majority of surviving lines were markedly smaller than wild type, and formed rather weedy inflorescence stems that yielded very few flowers. Additionally, flowers often had poorly developed organs.

In addition, G916 overexpressing lines were larger than control wild-type seedlings in several germination assays. Larger seedlings were observed under conditions of high sucrose. In addition, 35S::G916 seedlings were larger and appeared to have less anthocyanin on high sucrose plates that were nitrogen deficient, with or without glutamine supplementation. The assays monitor the effect of C on N signaling through anthocyanin production. That 35S::G916 seedlings performed better under conditions of high sucrose alone makes it more difficult to interpret the better seedling performance under conditions of low nitrogen. Tissue specific or inducible expression of this gene could aid in sorting out the complex phenotypes caused by the constitutive overexpression of this gene.

The results of the high sucrose assays indicated that G916-overexpressing plants might be significantly more drought tolerant than control plants, which was subsequently confirmed in soil-based drought assays (Tables 9 and 10).

Utilities

The results of physiological assays indicate that G916 could be used to alter the sugar signaling in plants. The soil-based drought and sugar sensing assays indicate that G916 and its equivalogs may also be used to enhance a plant's drought or other osmotic stress tolerance.

The enhanced performance of G916 overexpression lines under low nitrogen conditions indicate that the gene could be used to engineer crops that could thrive under conditions of reduced nitrogen availability.

That 35S::G916 lines make less anthocyanin on high sucrose plus glutamine, indicates G916 might be used to modify carbon and nitrogen status, and hence assimilate partitioning.

Additionally, the morphological phenotypes shown by 35S::G916 seedlings indicate that the gene might be used to manipulate light responses such as shade avoidance.

G1820 (SEQ ID NO: 243 and 244)

Published Information

G1 820 is a member of the Hap5 subfamily of CCAAT-box-binding transcription factors. G1820 was identified as part of the BAC clone MBA10, accession number AB025619 released by the Arabidopsis Genome sequencing project.

Closely Related Genes from Other Species

G1820 is closely related to a soybean gene represented by EST335784 isolated from leaves infected with Colletotrichum trifolii. Similarity between G1820 and the soybean gene extends beyond the signature motif of the family to a level that would suggest the genes are orthologous. Therefore the gene represented by EST335784 may have a function and/or utility similar to that of G1820.

Experimental Observations

The complete sequence of G1820 was determined. The function of this gene was analyzed using transgenic plants in which G1820 was expressed under the control of the 35S promoter. G1820 overexpressing lines showed more tolerance to salt stress in a germination assay. They also showed insensitivity to ABA, with the three lines analyzed showing the phenotype. The salt and ABA phenotypes could be related to the plants' increased tolerance to osmotic stress, which was subsequently confirmed in soil-based drought assays in which 35S::G1820 plants were significantly more drought-tolerant than wild-type control plants (Tables 9 and 10).

Interestingly, overexpression of G1820 also consistently reduced the time to flowering. Under continuous light conditions at 20-25 C, the 35S::G1820 transformants displayed visible flower buds several days earlier than control plants. The primary shoots of these plants typically started flower initiation 1-4 leaf plastochrons sooner than those of wild type. Such effects were observed in all three T2 populations and in a substantial number of primary transformants.

When biochemical assays were performed, some changes in leaf fames were detected. In one line, an increase in the percentage of 18:3 and a decrease in 16:1 were observed. Otherwise, G1820 overexpressors behaved similarly to wild-type controls in all biochemical assays performed. As determined by RT-PCR, G1820 was highly expressed in embryos and siliques. No expression of G1820 was detected in the other tissues tested. G1820 expression appeared to be induced in rosette leaves by cold and drought stress treatments, and overexpressing lines showed tolerance to water deficit and high salt conditions.

One possible explanation for the complexity of the G1820 overexpression phenotype is that the gene is somehow involved in the cross talk between ABA and GA signal transduction pathways. It is well known that seed dormancy and germination are regulated by the plant hormones abscisic acid (ABA) and gibberellin (GA). These two hormones act antagonistically with each other. ABA induces seed dormancy in maturing embryos and inhibits germination of seeds. GA breaks seed dormancy and promotes germination. It is conceivable that the flowering time and ABA insensitive phenotypes observed in the G1820 overexpressors are related to an enhanced sensitivity to GA, or an increase in the level of GA, and that the phenotype of the overexpressors is unrelated to ABA. In Arabidopsis, GA is thought to be required to promote flowering in non-inductive photoperiods. However, the drought and salt tolerant phenotypes would indicate that ABA signal transduction is also perturbed in these plants. It seems counterintuitive for a plant with salt and drought tolerance to be ABA insensitive since ABA seems to activate signal transduction pathways involved in tolerance to salt and dehydration stresses. One explanation is that ABA levels in the G1820 overexpressors are also high but that the plant is unable to perceive or transduce the signal.

G1820 overexpressors also had decreased seed oil content and increased seed protein content compared to wild-type plants

Utilities

G1820 and its equivalogs may be used to enhance a plant's tolerance to drought conditions. The osmotic stress results indicated that G1820 or its equivalogs could be used to alter a plant's response to additional water deficit conditions and can be used to engineer plants with enhanced tolerance to salt stress, and freezing. Evaporation from the soil surface causes upward water movement and salt accumulation in the upper soil layer where the seeds are placed. Thus, germination normally takes place at a salt concentration much higher than the mean salt concentration of in the whole soil profile. Increased salt tolerance during the germination stage of a crop plant would impact survivability and yield.

G1820 affects ABA sensitivity, and thus when transformed into a plant this transcription factor or its equivalogs may diminish cold, drought, oxidative and other stress sensitivities, and also be used to alter plant architecture, and yield.

G1820 or its equivalogs could also be used to accelerate flowering time.

G1820 or its equivalogs may be used to modify levels of saturation in oils.

G1820 or its equivalogs may be used to seed protein content.

The promoter of G1820 could be used to drive seed-specific gene expression.

G1820 or equivalog overexpression may be used to alter seed protein content, which may be very important for the nutritional value and production of various food products

G2701 (SEQ ID NO: 245 and 246)

Published Information

G2701 was identified in the sequence of BAC F11B9, GenBank accession number AC073395, released by the Arabidopsis Genome Initiative.

Experimental Observations

The function of G2701 was analyzed using transgenic plants in which the gene was expressed under the control of the 35S promoter. Overexpression of G2701 is Arabidopsis resulted in plants that were wild-type in morphology and in the biochemical analyses performed. However, 35S::G2701 transgenic plants were more tolerant to osmotic stress in a germination assay, the seedlings were greener with expanded cotyledons and longer roots than wild-type controls when germinated on plates containing either high salt or high sucrose. The phenotype was repeated in all three lines.

The results of the high sucrose and salt assays suggested that this gene would confer increased tolerance to other abiotic stresses when G2701 is overexpressed, which was subsequently confirmed in soil-based drought assays, in which 35S::G2701 plants were significantly more drought tolerant than wild-type control plants (Tables 9 and 10).

G2701 was expressed ubiquitously in Arabidopsis according to RT-PCR, and the level of G2701 expression in leaf tissue was essentially unchanged in response to environmental stress related conditions.

Potential Applications

G2701 or its equivalogs could be used to alter a plant's response to water deficit conditions and therefore, could be used to engineer plants with enhanced tolerance to drought, salt stress, and freezing.

G2854 (SEQ ID NO: 251 and 256)

Published Information

The sequence of G2854 was obtained from the Arabidopsis genome sequencing project, GenBank accession number AL161566, nid=7269538, based on its sequence similarity within the conserved domain to other ACBF-like related proteins in Arabidopsis. To date, there is no published information regarding the functions of this gene.

Experimental Observations The 5′ and 3′ ends of G2854 were determined by RACE. The function of G2854 was analyzed using transgenic plants in which G2854 was expressed under the control of the 35S promoter. 35S::G2854 transformants showed increased germination efficiency on sucrose plates compared to wild-type controls. These results suggested a possible role for G2854 in conferring drought tolerance in plants. This supposition was confirmed in soil-based drought assays, in which plants overexpressing G2854 performed significantly better than wild-type plants (Tables 9 and 10).

Utilities

G2854 and its equivalogs may be used to confer improved drought tolerance in plants.

G2854 and its equivalogs might also be used to generate crop plants with altered sugar sensing. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships). Glucose-specific hexose-sensing has been described in plants and implicated in cell division and repression of ‘famine’ genes (photosynthetic or glyoxylate cycles). The potential utilities of a gene involved in glucose-specific sugar sensing are to alter energy balance, photosynthetic rate, carbohydrate accumulation, biomass production, source-sink relationships, and senescence.

G2789 (SEQ ID NO: 247 and 248)

Published Information

The sequence of G2789 was obtained from Arabidopsis genomic sequencing project, GenBank accession number AL162295, based on its sequence similarity within the conserved domain to other AT-hook related proteins in Arabidopsis. G2789 corresponds to gene T4C21_{—}280 (CAB82691). To date, there is no published information regarding the functions of this gene.

Closely Related Genes from Other Species

G2789 protein shows extensive sequence similarity with Medicago truncatula cDNA clones (AL366947 and BG647144), an Oryza sativa chromosome 6 clone (AP003526) and a tomato crown gall Lycopersicon esculentum cDNA clone (BG13445 1).

Exiperimental Observations

The complete sequence of G2789 was determined. G2789 is expressed at moderate levels in roots, flowers, embryos, siliques, and germinating seeds. It was not detectable in rosette leaves or shoots. No significant induction of G2789 was observed in rosette leaves by any condition tested.

The function of this gene was analyzed using transgenic plants in which G2789 was expressed under the control of the 35S promoter. Overexpression of G2789 in Arabidopsis resulted in seedlings that are ABA insensitive and osmotic stress tolerant. In a germination assay on ABA containing media, G2789 transgenic seedlings showed enhanced seedling vigor. In a similar germination assay on media containing high concentrations of sucrose, the G2789 overexpressors also showed enhanced seedling vigor. In a repeat experiment on individual lines, all three lines show the phenotype. The combination of ABA insensitivity and better germination under osmotic stress was also observed for G1820. It is possible that ABA insensitivity at the germination stage promotes germination despite unfavorable conditions.

The osmotic stress tolerance and enhanced seedling vigor on ABA phenotypes suggested that G2789 overexpressors would be more tolerant to drought conditions This supposition was confirmed by soil-based drought assays, in which plants overexpressing G2789 performed significantly better in conditions of water deprivation than wild-type plants (Tables 9 and 10).

Utilities

G2789 could be used to alter a plant's response to water deficit conditions and therefore, could be used to engineer plants with enhanced tolerance to drought, salt stress, and freezing.

G634 (SEQ ED NO: 231 and 232)

Published Information

G634 was initially identified as public partial cDNAs sequences for GTL1 and GTL2 which are splice variants of the same gene (Small et al (1998) Proc. Natl. Acad. Sci. U S A. 95:3318-3322). The published expression pattern of GTL1 shows that G634 is highly expressed in siliques and not expressed in leaves, stems, flowers or roots. There is no published information on the function of G634.

Closely Related Genes from Other Species

The closest non-Arabidopsis relative of G634 is the O. sativa gt-2 gene (EMBO J. (1992) 11:4131-4144), which is proposed to bind and regulate the phyA promoter. In addition, the pea DNA-binding protein DF1 (13786451) shows strong homology to G634. The homology of these proteins to G634 extends to outside of the conserved domains and thus these genes are likely to be orthologs of G634.

Experimental Observations

The boundaries of G634 in were experimentally determined and the function of G634 was investigated by constitutively expressing G634 using the CaMV 35S promoter.

Three constructs were made for G634: P324, P1374 and P1717. P324 was found to encode a truncated protein. P1374 and P1717 represent full length splice variants of G634; P1374, the shorter of the two splice variants was used for the experiments described here. The longest available cDNA (P1717), confirmed by RACE, has the same ATG and stop codons as the genomic sequence.

Plants overexpressing G634 from construct P1374 showed a dramatic increase the density of trichomes, which additionally appear larger in size. The increase in trichome density was most noticeable on later arising rosette leaves, cauline leaves, inflorescence stems and sepals with the stem trichomes being more highly branched than controls. Approximately half of the primary transformants and two of three T2 lines showed the phenotype. Apart from slight smallness, there did not appear to be any other clear phenotype associated with the overexpression of G634. However, a reduction in germination was observed in T2 seeds grown in culture. It is not clear whether this defect was due to the quality of the seed lot tested or whether this characteristic is related to the transgene overexpression.

RT PCR data showed that G634 is potentially preferentially expressed in flowers and germinating seedlings, and induced by auxin. The role of auxin in trichome initiation and development has not been established in the published literature.

The increase in trichome density observed in G634 overexpressors suggested a possible role for this gene in drought-stress tolerance, a presumption subsequently confirmed in soil-based drought assays (Tables 9 and 10).

Utilities

Trichome glands on the surface of many higher plants produce and secrete exudates that give protection from the elements and pests such as insects, microbes and herbivores. These exudates may physically immobilize insects and spores, may be insecticidal or ant-microbial or they may allergens or irritants to protect against herbivores. Trichomes have also been suggested to decrease transpiration by decreasing leaf surface air flow, and by exuding chemicals that protect the leaf from the sun.

Depending on the plant species, varying amounts of diverse secondary biochemicals (often lipophilic terpenes) are produced and exuded or volatilized by trichomes. These exotic secondary biochemicals, which are relatively easy to extract because they are on the surface of the leaf, have been widely used in such products as flavors and aromas, drugs, pesticides and cosmetics. One class of secondary metabolites, the diterpenes, can effect several biological systems such as tumor progression, prostaglandin synthesis and tissue inflammation. In addition, diterpenes can act as insect pheromones, termite allomones, and can exhibit neurotoxic, cytotoxic and antimitotic activities. As a result of this functional diversity, diterpenes have been the target of research several pharmaceutical ventures. In most cases where the metabolic pathways are impossible to engineer, increasing trichome density or size on leaves may be the only way to increase plant productivity.

Thus, the use of G634 and its equivalogs to increase trichome density, size or type may therefore have profound utilities in so called molecular farming practices (i.e. the use of trichomes as a manufacturing system for complex secondary metabolites), and in producing resistant insect and herbivore resistant plants.

G634 and its equivalogs may also be used to increase the drought tolerance of plants.

G175 (SEQ ID NO: 223 and 224)

Published Information

G175 was identified in the sequence of P1 clone M3E9 (Gene AT4g26440/M3E9.130; GenBank accession number CAB79499). No information is available about the function(s) of G175.

Closely Related Genes from Other Species

The non-Arabidopsis most highly related gene to G175 is Nicotiana tabacum NtWRKY4 (as identified by BLAST searches; GenBank accession number BAA8603 1). Similarly between G175 and the tobacco gene extends beyond the signature motif of the family to a level that would suggest that the genes might be orthologs. Therefore NtWRKY4 may have a function and/or utility similar to that of G175. No further information is available about NtWRKY4.

Experimental Observations

The complete cDNA sequence of G175 was determined by us. The function of this gene was studied using transgenic plants in which G175 was expressed under the control of the 35S promoter. 35S::G175 plants are more tolerant to osmotic stress conditions (better germination in NaCl and sucrose containing media). The plants were otherwise wild-type in morphology and development.

G175 appears to be specifically expressed in floral tissues, and also appears to be induced elsewhere by heat and salt stress.

The results of the osmotic stress assays and heat and salt stress expression analyses suggested that G175 could be used to confer drought tolerance in plants, a supposition that was confirmed in soil-based assays in which G175-overexpressing plants were shown to be more tolerant to water deprivation than wild-type control plants (Tables 9 and 10).

Utilities

G175 and its equivalogs can be used to improve drought tolerance and increase germination under adverse osmotic stress conditions, which could impact survivability and yield. The promoter of G175 could also be used to drive flower specific expression.

G2839 (SEQ ID NO: 249 and 250)

Published Information

G2839 (At3g46080) was identified in the sequence of BAC F12M12 (GenBank accession number AL355775) based on its sequence similarity within the conserved domain to other C2H2 related proteins in Arabidopsis. There is no published or public information about the function of G2839.

Experimental Observations

The function of G2839 was studied using transgenic plants in which the gene was expressed under the control of the 35 S promoter. Few primary transformants were generated, suggesting that G2839 overexpression can be lethal. T1 lines displayed stunted growth and development, and yielded very few or zero seeds. Inflorescences were poorly developed. In one line, flower pedicels were very short and flowers and siliques were oriented downwards. G2839 overexpressors showed a phenotype in a germination assay on media containing high sucrose: seedlings were green and had high germination rates. Thus, the gene appeared to influence sugar sensing and/or osmotic stress responses.

G2839 is similar to two other Arabidopsis sequences, G354 and G353. Flower phenotypes in which pedicels were very short and flowers and siliques were oriented downwards have been described for G353 and G354 and are also similar to the brevipedicellus mutant (Koomneef et al. (1983) J. Hered. 74: 265-272; Venglat et al. (2002) Proc. Natl. Acad. Sci. USA. 99:4730-4735; Douglas et al. (2002) Plant Cell. 14:547-558. Interestingly 35S::G353 lines also showed increased resistance to osmotic stress.

Supplementing the results of the high sucrose germination assay, G2839 was shown to be more tolerant to water deprivation than wild-type control plants in soil-based drought assays (Tables 9 and 10).

Utilities

The phenotypes observed in physiology assays indicate that G2839 might be used to generate crop plants with altered sugar sensing. Since the gene appears to be associated with the response to osmotic stress, the gene could be used to engineer cold and dehydration tolerance. The latter was confirmed by the soil-based drought assay.

The morphological phenotype shown by 35S::G2839 lines indicate that the gene might be used to alter inflorescence architecture. In particular, a reduction in pedicel length and a change in the position at which flowers and fruits are held, might influence harvesting or pollination efficiency. Additionally, such changes might produce attractive novel forms for the ornamental markets.

G1452 (SEQ ID NO: 241 and 242)

Published Information

G1452 was identified in the sequence of clones T22013, F12K2 with accession number AC006233 released by the Arabidopsis Genome Initiative. No information is available about the function(s) of G1452.

Closely Related Genes from Other Species

G1452 does not show extensive sequence similarity with known genes from other plant species outside of the conserved NAC domain.

Experimental Observations

The function of G1452 was analyzed using transgenic plants in which the gene was expressed under the control of the 35S promoter.

Overexpression of G1452 produced changes in leaf development and markedly delayed the onset of flowering. 35S::G1452 plants produced dark green, flat, rounded leaves, and typically formed flower buds between 2 and 14 days later than controls. Additionally, some of the transformants were noted to have rather low trichome density on leaves and stems. At later stages of life cycle, 35S::G1452 appeared to develop slowly and senesced considerably later than wild-type controls.

G1452 overexpressors were more tolerant to high sucrose-induced osmotic stress than wild-type control plants, were more tolerant to high salt than controls, and were insensitive to ABA in separate germination assays. These results suggested that G1452 may be used to confer improved survival in drought, which was confirmed in soil-based drought assays where G1452-overexpressors fared significantly better than wild-type control plants (Tables 9 and 10).

Utilities

G1452 could be used to alter a plant's response to water deficit conditions and therefore, could be used to engineer plants with enhanced tolerance to drought and salt stress.

On the basis of the analyses performed to date, G1452 could be use to alter plant growth and development.

G3083 (SEQ ID NO: 253 and 254)

Published Information

G3083 (At3g14880) was identified as part of the BAC clone K15M2, GenBank accession number AP000370 (nid=5541653). No published information is available on the function of G3083.

Experimental Observations

The 5′- and 3′-ends of G3083 were determined by RACE and the function of the gene was assessed by analysis of transgenic Arabidopsis lines in which a genomic clone was constitutively expressed from a 35S promoter. 35S::G3083 plants were indistinguishable from wild-type controls in the morphological analysis.

In the physiological analysis, two out of the three 35S::G3083 lines tested, displayed an enhanced ability to germinate on plates containing high levels of sodium chloride. This suggested that G3083 might function as part of a response pathway to abiotic stress, which was further indicated in soil-based drought assays in which one line of a G3083 overexpressor was shown to be significantly more tolerant to water deprivation than wild-type control plants.

Utilities

Based on the increased salt tolerance exhibited by the 35S::G3083 lines in physiology assays, this gene might be used to engineer salt tolerant crops and trees that can flourish in drought or in salinified soils. The latter condition is of particular importance early in the lifecycle, since evaporation from the soil surface causes upward water movement, and salt accumulates in the upper soil layer where the seeds are placed. Thus, germination normally takes place at a salt concentration much higher than the mean salt level in the whole soil profile. Increased salt tolerance during the germination stage of a crop plant would therefore enhance survivability and yield.

G489 (SEQ ED NO: 229 and 230)

Published Information

G489 was identified from a BAC sequence that showed high sequence homology to AtHAP5-like transcription factors in Arabidopsis. No published information is available regarding the function of this gene.

Closely Related Genes from Other Species

G489 has no significant homology to any other non-Arabidopsis plant protein in the database outside the conserved domain.

Experimental Observations

The function of G489 was analyzed through its ectopic overexpression in plants.

RT-PCR analysis of endogenous levels of G489 transcripts indicates that this gene is expressed constitutively in all tissues tested. A cDNA array experiment confirms the RT-PCR derived tissue distribution data. G489 was not induced above basal levels in response to the stress treatments tested.

G489 overexpressors were more tolerant to high NaCl stress, showing more root growth and leaf expansion compared to the controls in culture. Two well characterized ways in which NaCl toxicity is manifested in the plant is through general osmotic stress and potassium deficiency due to the inhibition of its transport. These lines were more tolerant to osmotic stress, showing more root growth on mannitol containing media; however, they were not more tolerant to potassium deficiency.

The involvement of G489 in a response pathway to abiotic stress was further confirmed in soil-based drought assays, where the overexpressors were observed to be more tolerant to water deprivation conditions than wild-type control plants (Table 10).

Utilities

The potential utilities of this gene include the ability to confer drought and salt tolerance during the growth and developmental stages of a crop plant. This would most likely impact yield and or biomass.

G303 (SEQ ED NO: 225 and 226)

Published Information

G303 corresponds to gene MNA5.5 (BAB11554.1). There is no published information regarding the functions of this gene.

Closely Related Genes from Other Species

G303 does not show extensive sequence similarity with known genes from other plant species outside of the conserved basic HLH domain.

Experimental Observations

The complete sequence of G303 was determined. G303 was detected at very low levels in roots and rosette leaves. It did not appear to be induced by any condition tested. No altered morphological or biochemical phenotypes were detected in G303 overexpressing plants.

The function of this gene was analyzed using transgenic plants in which G303 was expressed under the control of the 35S promoter. G303 overexpressing plants showed more tolerance to osmotic stress vigor than wild-type controls in a germination assay in three separate experiments on high salt and high sucrose.

The involvement of G303 in a response pathway to abiotic stress was further confirmed in soil-based drought assays, in which the plants overexpressing G303 were found to be more tolerant to drought than the wild-type controls in the experiment (Table 10).

Utilities

G303 may be useful for enhancing drought tolerance and seed germination under high salt conditions or other conditions of osmotic stress (e.g., freezing).

G2992 (SEQ ED NO: 49 and 50)

Published Information

G2992 corresponds to gene F24J1.29 within BAC clone F24J1 (GenBank accession AC021046) derived from chromosome 1. We identified this locus as a novel member of the ZF-HB family and no data regarding its function are currently in the public domain (as of 8/5/02).

Experimental Observations

The boundaries of G2992 were determined by RACE, and a clone was PCR-amplified from cDNA derived from mixed tissue samples. The function of G2992 was then assessed by analysis of transgenic Arabidopsis lines in which the cDNA was constitutively expressed from a 35S CaMV promoter.

Morphological studies revealed that overexpression of G2992 can accelerate the onset of reproductive development, reduce plant size, and produce changes in leaf shape.

35S::G2992 T2 populations displayed an enhanced ability to germinate on plates containing high levels of sodium chloride. The role of G2992 in a response pathway to abiotic stress was affirmed by a soil-based drought assay, in which it was shown that G2992 overexpressors were, on average, more tolerant to water deprivation conditions in soil-based drought assays than wild-type plants (Table 10), and one of the lines tested was significantly more drought tolerant than the wild-type controls.

Utilities

Based on the phenotypes observed in morphological and physiological assays, G2992 might be have a number of applications.

Given the drought and salt tolerance exhibited by 35S::G2992 transformants, the gene and its equivalogs might be used to engineer drought and salt tolerant crops and trees that can flourish in drought conditions and salinified soils.

The early flowering exhibited by 35S::G2992 lines, indicates that the gene might be used to manipulate flowering time in commercial species. In particular, G2992 could be applied to accelerate flowering or eliminate any requirements for vernalization. In some instances, a faster cycling time might allow additional harvests of a crop to be made within a given growing season. Shortening generation times could also help speed-up breeding programs, particularly in species such as trees, which typically grow for many years before flowering. Conversely, it might be possible to modify the activity of G2992 (or its equivalogs) to delay flowering in order to achieve an increase in biomass and yield.

Finally, the effects of G2992 overexpression on leaf shape suggest that the gene might be used to modify plant architecture.

G682 (SEQ ID NO: 233 and 234)

Published Information

G682 was identified from the Arabidopsis BAC, AF007269, based on sequence similarity to other members of the Myb family within the conserved domain. To date, no functional data is available for this gene.

Closely Related Genes from Other Species

G682 has no significant homology to any other non-Arabidopsis plant protein in the database outside the conserved Myb domain.

Experimental Observations

The function of G682 was analyzed through its ectopic overexpression in plants.

RT-PCR analysis of the endogenous levels of G682 transcripts indicated that this gene is expressed in all tissues tested, however, a very low level of transcript is detected in roots and shoots. Array tissue print data suggests that G682 is expressed primarily, but not exclusively, in flower tissue.

An array experiment was performed on G682 overexpressing line 5. The data from this one experiment indicates that this gene could be a negative regulator of chloroplast development and/or light dependent development because the gene Albino3 and many chloroplast genes are repressed. Albino3 functions to regulate chloroplast development (Plant Cell (1997) 9: 717-730). The gene G682 is itself is induced 20-fold. Other than a few additional transcription factors, very few genes are induced as a result of the ectopic expression of G682. These plants are not pale in color, making it uncertain how to relate the morphological and physiological data with the gene profiling data. The array experiment needs to be repeated with additional lines.

G682 overexpressors are glabrous, have tufts of more root hairs and germinated better under heat stress conditions. Older plants were not more tolerant to heat stress compared to wild-type controls. At the time these experiments were performed, it was suggested that further experiments were needed to address whether or not the heat germination phenotype of the G682 overexpressors was related to water deficit stress tolerance in the germinating seedling, and correlated with a possible drought tolerance phenotype. More recent experiments have shown that G682 overexpressors were, on average, more tolerant to water deprivation conditions in soil-based drought assays than wild-type plants (Table 10), and two of three lines were significantly more drought tolerant than the wild-type controls.

Utilities

The utility of this gene and its equivalogs would be to confer heat tolerance to germinating seeds and drought tolerance in plants.

G1073 (SEQ ID NO: 301 and 302), AtHRC1

Published Information

G1073 has been identified in the sequence of a BAC clone from chromosome 4 (BAC clone F23E12, gene F23E12.50, GenBank accession number AL022604), released by EU Arabidopsis Sequencing Project.

Closely Related Genes from Other Species

G1073 has similarity to Medicago truncatula cDNA clones (GenBank accession number AW574000 and AW560824) and Glycine max cDNA clones (AW349284 and AM736668 ) in the database.

Experimental Observations, Increased Biomass and Size and Other Observations

The function of G1073 was analyzed using transgenic plants in which G1073 was expressed under the control of the cauliflower mosaic virus 35S promoter (these transgenic plants are referred to as “35S::G1073”). Transgenic plants overexpressing G1073 were substantially larger than wild-type controls, with at least a 60% increase in biomass (Table 8) . The increased mass of 35S::G1073 transgenic plants was attributed to enlargement of multiple organ types including stems, roots and floral organs; other than the size differences, these organs were not affected in their overall morphology. 35S::G1073 plants exhibited an increase of the width (but not length) of mature leaf organs, produced 2-3 more rosette leaves, and had enlarged cauline leaves in comparison to corresponding wild-type leaves. Overexpression of G1073 resulted in an increase in both leaf mass and leaf area per plant, and leaf morphology (G1073 overexpressors tended to produce more serrated leaves). We also found that root mass was increased in the transgenic plants, and that floral organs were also enlarged. An increase of approximately 40% in stem diameter was observed in the transgenic plants. Images from the stem cross-sections of 35S::G1073 plants revealed that cortical cells are large and that vascular bundles contained more cells in the phloem and xylem relative to wild type. Petal size in the 35S::G1073 lines was increased by 40-50% compared to wild type controls. Petal epidermal cells in those same lines were approximately 25-30% larger than those of the control plants. Furthermore, 15-20% more epidermal cells per petal were produced compared to wild type. Thus, in petals and stems, the increase in size was associated with an increase in cell size as well as in cell number.

Seed yield was also increased compared to control plants. 5S::G1073 lines showed an increase of at least 70% in seed yield (Table 8). This increased seed production was associated with an increased number of siliques per plant, rather than seeds per silique.

TABLE 8
Comparison of biomass and seed yield production in Arabidopsis
wild-type and two 35S::G1073 overexpressing lines

| Line | Fresh Weight (g) | Dry Weight (g) | Seed (g) |
|---|---|---|---|
| Wild-type | 3.43 ± 0.70 | 0.73 ± 0.20 | 0.17 ± 0.07 |
| 35S::G1073-3 | 5.74 ± 1.74 | 1.17 ± 0.30 | 0.31 ± 0.08 |
| 35S::G1073-4 | 6.54 ± 2.19 | 1.38 ± 0.44 | 0.35 ± 0.12 |

All 35S::G1073 lines tested (10/10) exhibited significantly improved salt tolerance. Most of these lines also showed a sugar sensing phenotype, exhibiting improved germination on high sucrose media. One line showed increased heat germination tolerance. Flowering of G1073 overexpressing plants was delayed. Leaves of G1073 overexpressing plants were generally more serrated than those of wild-type plants. Improved drought tolerance was observed in 35S::G1073 transgenic lines.

A number of the CUT1::G1073 lines tested exhibited significantly improved salt tolerance and sugar sensing on high sucrose. One line showed improved germination on high mannitol.

Half of the ARSK::G1073 lines tested (5/10) showed improved germination on high salt, and two lines showed improved germination in cold relative to controls.

Utilities of G1073

Large size and late flowering produced as a result of G1073 or equivalog overexpression would be extremely useful in crops where the vegetative portion of the plant is the marketable portion (often vegetative growth stops when plants make the transition to flowering). In this case, it would be advantageous to prevent or delay flowering with the use of this gene or its equivalogs in order to increase yield (biomass). Prevention of flowering by this gene or its equivalogs would be useful in these same crops in order to prevent the spread of transgenic pollen and/or to prevent seed set. This gene or its equivalogs could also be used to manipulate leaf shape, abiotic stress tolerance, including drought and salt tolerance, and seed yield.

Rice Sequences G3399 and G3407 (SEQ ID NOs: 341, 342, 355 and 356), OsHRC2 and OsHRC7

Published Information

The sequences of G3399 and G3407 were discovered based on their similarity to G1073 as determined by BLAST analysis of a proprietary database, To date, there is no published information regarding the functions of either gene or polypeptide.

Experimental Observations

A number of Arabidopsis lines overexpressing G3399 and G3407 under the control of the 35S promoter were found be larger, with broader leaves and larger rosettes than wild-type control plants.

Utilities of G3399 and G3407

G3399 and G3407 could be used to increase a plant's biomass.

G3399 and G3407 may be also used to alter a plant's response to water deficit conditions and, therefore, could be used to engineer plants with enhanced tolerance to drought, salt stress, and freezing.

Soybean Sequences G3456,G3459 and G3460 (SEQ ID NOs: 385, 386, 389, 390, 391 and 392), GmHRC2, GmHRC7 and GmHRC8

Published Information

The sequences of G3456,G3459 and G3460 were discovered based on their similarity to G1073 as determined by BLAST analysis of a proprietary database, To date, there is no published information regarding the functions of either gene or polypeptide.

Experimental Observations

A significant number of Arabidopsis lines overexpressing G3456,G3459 and G3460 under the control of the 35S promoter were found be larger, with broader leaves and larger rosettes than wild-type control plants.

Utilities of G3456, G3459 and G3460

G3456, G3459 and G3460 can be used to increase a plant's biomass.

G3456, G3459 and G3460 may be also used to alter a plant's response to water deficit conditions and, therefore, could be used to engineer plants with enhanced tolerance to drought, salt stress, and freezing.

G481 (Polynucleotide SEQ ID NO: 289 and 290)

Published Information

G481 is equivalent to AtHAP3a which was identified by Edwards et al., ((1998) Plant Physiol. 117: 1015-1022) as an EST with extensive sequence homology to the yeast HAP3. Northern blot data from five different tissue samples indicates that G481 is primarily expressed in flower and/or silique, and root tissue. No other functional data is available for G481 in Arabidopsis.

Closely Related Genes from Other Species

There are several genes in the database from higher plants that show significant homology to G481 including, X59714 from corn, and two ESTs from tomato, AI486503 and AI782351.

Experimental Observations

The function of G481 was analyzed through its ectopic overexpression in plants. Except for darker color in one line (noted below), plants overexpressing G481 had a wild-type morphology. G481 overexpressors were found to be more tolerant to high sucrose and high salt, having better germination, longer radicles, and more cotyledon expansion. There was a consistent difference in the hypocotyl and root elongation in the overexpressor compared to wild-type controls. These results indicated that G481 is involved in sucrose-specific sugar sensing. Sucrose-sensing has been implicated in the regulation of source-sink relationships in plants.

In the T2 generation, one overexpressing line was darker green than wild-type plants, which may indicate a higher photosynthetic rate that would be consistent with the role of G481 in sugar sensing.

35S::G481 plants were also significantly larger and greener in a soil-based drought assay than wild-type controls plants After eight days of drought treatment overexpressing lines had a darker green and less withered appearance than those in the control group. The differences in appearance between the control and G481-overexpressing plants after they were rewatered was even more striking. Eleven of twelve plants of this set of control plants died after rewatering, indicating the inability to recover following severe water deprivation, whereas all nine of the overexpressor plants of the line shown recovered from this drought treatment. These results were typical of a number of control and 35S::G481-overexpressing lines.

One line of plants in which G481 was overexpressed under the control of the ARSKl root-specific promoter was found to germinate better under cold conditions than wild-type plants.

Interestingly, in one Arabidopsis line in which G481 was knocked out, the plants were found to be more sensitive to high salt in a plate-based assay than wild-type plants, which indicates the importance of the role played by G481 in regulating osmotic stress tolerance, and demonstrates that the gene is both necessary and sufficient to fulfill that function.

A number of the 35S::G481 plants evaluated had a late flowering phenotype.

Utilities

The potential utility of G481 includes altering photosynthetic rate, which could also impact yield in vegetative tissues as well as seed. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships).

Since G481 overexpressing plants performed better than controls in drought experiments, this gene or its equivalogs may be used to improve seedling vigor, plant survival, as well as yield, quality, and range.

G482 (Polynucleotide SEQ ID NO: 291 and 292)

Published Information

G482, a paralog of G481, is equivalent to AtHAP3b which was identified by Edwards et al. (1998) Plant Physiol. 117: 1015-1022) as an EST with homology to the yeast gene HAP3b. Their northern blot data suggests that AtHAP3b is expressed primarily in roots. No other functional information regarding G482 is publicly available.

Closely Related Genes from Other Species

The closest homology in the non-Arabidopsis plant database is within the B domain of G482, and therefore no potentially orthologous genes are available in the public domain.

Experimental Observations

RT-PCR analysis of endogenous levels of G482 transcripts indicated that this gene is expressed constitutively in all tissues tested. A cDNA array experiment supports the RT-PCR derived tissue distribution data. G482 is not induced above basal levels in response to any environmental stress treatments tested.

A T-DNA insertion mutant for G482 was analyzed and was found to flower slightly later than control plants.

The function of G482 was also analyzed through its ectopic overexpression in plants. Plants overexpressing G482 had a wild-type morphology. Germination assays to measure salt tolerance demonstrated increased seedling growth when germinated on the high salt medium.

35S::G482 transgenic plants also displayed an osmotic stress response phenotype similar to 35S::G481 transgenic lines. Five of ten overexpressing lines had increased seedling growth on medium containing 80% MS plus vitamins with 300 mM mannitol.

Three of ten 35S::G482 lines also demonstrated enhanced germination relative to controls after a 6 hour exposure to 32° C.

The majority of these 35S::G482 lines also demonstrated a slightly early flowering phenotype.

Utilities

The potential utilities of this gene include the ability to confer osmotic stress tolerance, as measured by salt, heat tolerance and improved germination in mannitol-containing media, during the germination stage of a crop plant. This would most likely impact survivability and yield. Evaporation of water from the soil surface causes upward water movement and salt accumulation in the upper soil layer, where the seeds are placed. Thus, germination normally takes place at a salt concentration much higher than the mean salt concentration in the whole soil profile.

Improved osmotic stress tolerance is also likely to result in enhanced seedling vigor, plant survival, improved yield, quality, and range. Osmotic stress assays, including subjecting plants to aqueous dissolved sugars, are often used as surrogate assays for improved water-stress (e.g., drought) response. Thus, G482 may also be used to improve plant performance under conditions of water deprivation, including increased seedling vigor, plant survival, yield, quality, and range.

Rice G3395 and Soy G3470 (Polynucleotide SEQ ID NOs: 333 and 395, Respectively, and Polypeptide SEQ ID NOs: 334 and 396, Respectively)

Published Information

G3395 (rice) and G3470 (soybean) are orthologs of G481 and G482, and are members of the HAP3-like subfamily of CCAAT-box binding transcription factors. G3395 corresponds to polypeptide BAC76331 (“NF-YB subunit of rice”).

Experimental Observations

The functions of G3395 and G3470 were analyzed through their ectopic overexpression in plants. One of the lines of 35S::G3395 overexpressors tested was found to be more tolerant to high salt levels, producing larger and greener seedlings in a high salt germination assay.

Seven of ten lines of 35S::G3470 overexpressors were found to be significantly more tolerant to high salt in a plate-based germination assay.

Utilities

The potential utilities of these two genes, G3395 and G3470, and their equivalogs, include the ability to confer tolerance to drought and other osmotic stresses, including during the germination stage of a crop plant. Equivalogs of G3395 and G3470 include, for example, Arabidopsis sequences G481 (SEQ ID NO: 290), G482 (SEQ ID NO: 292), G485 (SEQ ID NO: 294), G486 (SEQ ID NO: 296), G1248 (SEQ ID NO: 308), G1364 (SEQ ID NO: 310), G1781 (SEQ ID NO: 312), G2345 (SEQ ID NO: 322), G2718 (SEQ ID NO: 326), rice sequences G3394 (SEQ ID NO: 332), G3396 (SEQ ID NO: 336), G3397 (SEQ ID NO: 338), G3398 (SEQ ID NO: 340), G3429 (SEQ ID NO: 360), G3835 (SEQ ID NO: 416), G3836 (SEQ ID NO: 418), corn sequences G3434 (SEQ ID NO: 364), G3435 (SEQ ID NO: 366), G3436 (SEQ ID NO: 368), G3437 (SEQ ID NO: 370), and soy sequences G3470 (SEQ ID NO: 396), G3471 (SEQ ID NO: 398), G3472 (SEQ ID NO: 400), G3473 (SEQ ID NO: 402), G3474 (SEQ ID NO: 404), G3475 (SEQ ID NO: 406), G3476 (SEQ ID NO: 408), G3477 (SEQ ID NO: 410), G3478 (SEQ ID NO: 412), and G3837 (SEQ ID NO: 420).

Table 9 presents the results obtained in an assay in which Arabidopsis plants were subjected to water deprivation for seven to eight days. At the end of this dry-down period, each pot was assigned a numeric score depending on the health of its plants. A score of 0 to 6 was assigned based on a plant's color and general appearance, with plants that were all brown receiving a “0” and, at the other end of the spectrum, plants that had an excellent appearance (all green) receiving a “6“. The mean of the recorded numeric score of all pots of a given genotype per line of all flats tested is presented in order of decreasing health.

TABLE 9
Comparison of recorded numeric score
plants subjected to drought treatment.

| GID | Mean score |
|---|---|
| G2133 | 5.875 |
| G634 | 4.778 |
| G922 | 4.667 |
| G916 | 4.6 |
| G1274 | 4.273 |
| G864 | 3.733 |
| G2999 | 3.7 |
| G2992 | 3.7 |
| G353 | 3.6 |
| G47 | 3.459 |
| G2053 | 3.404 |
| G975 | 3.393 |
| G489 | 3.364 |
| G1792 | 3.281 |
| G1820 | 3.2 |
| G2453 | 3.2 |
| G2140 | 3.139 |
| G2701 | 3.108 |
| G3086 | 3.056 |
| G611 | 3.048 |
| G1452 | 3.042 |
| G481 | 3.041 |
| G624 | 3.000 |
| G2854 | 2.829 |
| G303 | 2.812 |
| G2839 | 2.783 |
| G2789 | 2.708 |
| G188 | 2.692 |
| G325 | 2.556 |
| G2776 | 2.513 |
| G175 | 2.467 |
| G2110 | 2.432 |
| G1206 | 2.412 |
| G682 | 2.381 |
| G1730 | 2.341 |
| G2969 | 2.333 |
| G2998 | 2.333 |
| G1069 | 2.316 |
| Wild-type | 2.284 |

Table 10 compares the survival ratings of Arabidopsis plants overexpressing various polypeptides, evaluated after seven to eight days of drought treatment, rewatering, and two to three days of a recovery period Values indicate the median odds of survival within a given flat (the 50th percentile of survival within each pot of a given genotype per line divided by the average wild-type survival in the flat).

TABLE 10
Survival ratings of Arabidopsis plants after
drought and rewatering treatment

| | Median per |
|---|---|
| GID | flat |
| G2133 | 3.365 |
| G1274 | 2.059 |
| G922 | 1.406 |
| G2999 | 1.255 |
| G3086 | 1.179 |
| G354 | 1.167 |
| G1792 | 1.161 |
| G2053 | 1.091 |
| G975 | 1.090 |
| G1069 | 1.037 |
| G916 | 1.023 |
| G2701 | 1.000 |
| G1820 | 1.000 |
| G47 | 0.921 |
| G2854 | 0.889 |
| G2789 | 0.845 |
| G481 | 0.843 |
| G634 | 0.834 |
| G175 | 0.814 |
| G2839 | 0.805 |
| G1452 | 0.803 |
| Wild-type | 0.800 |

### Example X

### Identification of Homologous Sequences

This example describes identification of genes that are orthologous to Arabidopsis thaliana transcription factors from a computer homology search.

Homologous sequences, including those of paralogs and orthologs from Arabidopsis and other plant species, were identified using database sequence search tools, such as the Basic Local Alignment Search Tool (BLAST) (Altschul et al. (1990) J. Mol. Biol. 215: 403-410; and Altschul et al. (1997) Nucleic Acid Res. 25: 3389-3402). The tblastx sequence analysis programs were employed using the BLOSUM-62 scoring matrix (Henikoff and Henikoff (1992) Proc. Natl. Acad. Sci. 89: 10915-10919). The entire NCBI GenBank database was filtered for sequences from all plants except Arabidopsis thaliana by selecting all entries in the NCBI GenBank database associated with NCBI taxonomic ID 33090 (Viridiplantae; all plants) and excluding entries associated with taxonomic ID 3701 (Arabidopsis thaliana).

These sequences are compared to sequences representing genes of the invention, for example, SEQ ID NO: 1, 11, 87, 89, 91, 93, 95, 97, 99, using the Washington University TBLASTX algorithm (version 2.0a19MP) at the default settings using gapped alignments with the filter “off”. For each gene of the invention, for example, SEQ ID NO: 1, 11, 87, 89, 91, 93, 95, 97, 99, individual comparisons were ordered by probability score (P-value), where the score reflects the probability that a particular alignment occurred by chance. For example, a score of 3.6E40 is 3.6×10-40. In addition to P-values, comparisons were also scored by percentage identity. Percentage identity reflects the degree to which two segments of DNA or protein are identical over a particular length. Examples of sequences so identified are presented in Table 6. The percent sequence identity among these sequences can be as low as 47%, or even lower sequence identity.

Candidate paralogous sequences were identified among Arabidopsis transcription factors through alignment, identity, and phylogenic relationships. Candidate orthologous sequences were identified from proprietary unigene sets of plant gene sequences in Zea mays, Glycine max and Oryza sativa based on significant homology to Arabidopsis transcription factors. These candidates were reciprocally compared to the set of Arabidopsis transcription factors. If the candidate showed maximal similarity in the protein domain to the eliciting transcription factor or to a paralog of the eliciting transcription factor, then it was considered to be an ortholog. Identified non-Arabidopsis sequences that were shown in this manner to be orthologous to the Arabidopsis sequences are provided in Table 6.

### Example XI

### Screen of Plant cDNA Library for Sequence Encoding a Transcription Factor DNA Binding Domain That Binds To a Transcription Factor Binding Promoter Element and Demonstration of Protein Transcription Regulation Activity

The “one-hybrid” strategy (Li and Herskowitz (1993) Science 262: 1870-1874) is used to screen for plant cDNA clones encoding a polypeptide comprising a transcription factor DNA binding domain, a conserved domain. In brief, yeast strains are constructed that contain a lacZ reporter gene with either wild-type or mutant transcription factor binding promoter element sequences in place of the normal UAS (upstream activator sequence) of the GALL promoter. Yeast reporter strains are constructed that carry transcription factor binding promoter element sequences as UAS elements are operably linked upstream (5′) of a lacZ reporter gene with a minimal GAL1 promoter. The strains are transformed with a plant expression library that contains random cDNA inserts fused to the GAL4 activation domain (GAL4-ACT) and screened for blue colony formation on X-gal-treated filters (X-gal: 5-bromo-4-chloro-3-indolyl-β-D-galactoside; Invitrogen Corporation, Carlsbad Calif.). Alternatively, the strains are transformed with a cDNA polynucleotide encoding a known transcription factor DNA binding domain polypeptide sequence.

Yeast strains carrying these reporter constructs produce low levels of beta-galactosidase and form white colonies on filters containing X-gal. The reporter strains carrying wild-type transcription factor binding promoter element sequences are transformed with a polynucleotide that encodes a polypeptide comprising a plant transcription factor DNA binding domain operably linked to the acidic activator domain of the yeast GAL4 transcription factor, “GAL4-ACT”. The clones that contain a polynucleotide encoding a transcription factor DNA binding domain operably linked to GLA4-ACT can bind upstream of the lacZ reporter genes carrying the wild-type transcription factor binding promoter element sequence, activate transcription of the lacZ gene and result in yeast forming blue colonies on X-gal-treated filters.

Upon screening about 2×10⁶ yeast transformants, positive cDNA clones are isolated; i.e., clones that cause yeast strains carrying lacZ reporters operably linked to wild-type transcription factor binding promoter elements to form blue colonies on X-gal-treated filters. The cDNA clones do not cause a yeast strain carrying a mutant type transcription factor binding promoter elements fused to LacZ to turn blue. Thus, a polynucleotide encoding transcription factor DNA binding domain, a conserved domain, is shown to activate transcription of a gene.

### Example XII

### Gel Shift Assays

The presence of a transcription factor comprising a DNA binding domain which binds to a DNA transcription factor binding element is evaluated using the following gel shift assay. The transcription factor is recombinantly expressed and isolated from E. coli or isolated from plant material. Total soluble protein, including transcription factor, (40 ng) is incubated at room temperature in 10 μl of 1× binding buffer (15 mM HEPES (pH 7.9), 1 mM EDTA, 30 mM KCl, 5% glycerol, 5% bovine serum albumin, 1 mM DTT) plus 50 ng poly(dl-dC):poly(dl-dC) (Pharmacia, Piscataway N.J.) with or without 100 ng competitor DNA. After 10 minutes incubation, probe DNA comprising a DNA transcription factor binding element (1 ng) that has been ³²P-labeled by end-filling (Sambrook et al. (1989) supra) is added and the mixture incubated for an additional 10 minutes. Samples are loaded onto polyacrylamide gels (4% w/v) and fractionated by electrophoresis at 150V for 2 h (Sambrook et al. supra). The degree of transcription factor-probe DNA binding is visualized using autoradiography. Probes and competitor DNAs are prepared from oligonucleotide inserts ligated into the BamHI site of pUC118 (Vieira et al. (1987) Methods Enzymol. 153: 3-11). Orientation and concatenation number of the inserts are determined by dideoxy DNA sequence analysis (Sambrook et al. supra). Inserts are recovered after restriction digestion with EcoRI and HindIII and fractionation on polyacrylamide gels (12% w/v) (Sambrook et al. supra).

### Example XIII

### Introduction of Polynucleotides into Dicotyledonous Plants

Any of the sequences of the invention may be recombined into an expression vector for the purpose of transforming plants for the purpose of modifying plant traits, including increasing the tolerance of plants to abiotic stress, also including drought stress.

The transcription factor sequences used to generate transgenic plants may include, for example, any of the polynucleotide sequences found in the sequence listing, which incorporates SEQ ID NO: 2N-1, where N=1-210. Also included in the invention are related sequences that confer abiotic stress tolerance and are homologous with respect to SEQ ID NO: 2N-1, where N=1-210 by virtue of being substantially identical to those sequences, or that hybridizes to the complement of any of SEQ ID NO: 2N-1, where N=1-2 10 under stringent conditions (for example, conditions that include two wash steps of 6×SSC and 65° C., each step being 10-30 minutes in duration). All of the sequences of the invention encode polypeptides that have the property of regulating abiotic stress tolerance in a plant when the polypeptides are overexpressed. For example, the paralogs and orthologs of G2133, which include SEQ ID NO: 1, 11, 87, 89, 91, 93, 95, 97, 99, or polynucleotide sequences encoding SEQ ID NO: 2, 12, 88, 90, 92, 94, 96, 98, 100, paralogous, and orthologous sequences, and nucleotide sequences that hybridize over their full length to the complement of these polynucleotide sequences under stringent conditions. are specifically included in the invention. Examples of an expression vectors that may be used includes, for example, pMEN20 or pMEN65. The expression vector is then transformed into a plant, often by using the technique of transforming a plant cell. If a plant cell is the subject of the transformation procedure, it is then regenerated into a plant and allowed to overexpress the polypeptide encoded by aforementioned nucleic acid sequences.

The cloning vector may also be introduced into a variety of cereal plants by means well known in the art such as, for example, direct DNA transfer or ]Agrobacterium tumefaciens-mediated transformation or other methods (see below). It is now routine to produce transgenic plants using most dicot plants (see Weissbach and Weissbach, (1989) supra; Gelvin et al. (1990) supra; Herrera-Estrella et al. (1983) supra; Bevan (1984) supra; and Klee (1985) supra).

After abiotic-stress tolerant plants are produced, the transgenic plants may be crossed with another plant or selfed or to produce seed; which may be used to generate progeny plants having increased tolerance to abiotic stress. Generally, the progeny plants will express mRNA that encodes a DNA-binding protein having a conserved domain (e.g., an AP2 domain) that binds to a DNA molecule, regulates its expression, and induces the expression of genes and polypeptides that confer to the plant the desirable trait (e.g., abiotic stress tolerance). In these progeny plants, the mRNA may be expressed at a level greater than in a non-transformed plant that does not overexpress the DNA-binding protein.

Methods for analysis of traits are routine in the art and examples are disclosed above. Analysis includes identification and selection of plants that exhibit improved abiotic stress tolerance. The goal of the identification and selection steps is to find plants that show improved tolerance to, for example, drought, chilling, heat, germination in cold conditions, or low nutrient (e.g., nitrogen) conditions.

### Example XIV

### Transformation of Cereal Plants With an Expression Vector

Cereal plants such as, but not limited to, corn, wheat, rice, sorghum, or barley, may also be transformed with the present polynucleotide sequences in pMEN20 or pMEN65 expression vectors for the purpose of modifying plant traits. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art such as, for example, direct DNA transfer or Agrobacterium tumefaciens-mediated transformation. It is now routine to produce transgenic plants of most cereal crops (Vasil (1994) Plant Mol. Biol. 25: 925-937) such as corn, wheat, rice, sorghum (Cassas et al. (1993) Proc. Natl. Acad. Sci. 90: 11212-11216, and barley (Wan and Lemeaux (1994) Plant Physiol. 104:37-48. DNA transfer methods such as the microprojectile can be used for corn (Fromm et al. (1990) Bio/Technol. 8: 833-839); Gordon-Kamm et al. (1990) Plant Cell 2: 603-618; Ishida (1990) Nature Biotechnol. 14:745-750), wheat (Vasil et al. (1992) Bio/Technol. 10:667-674; Vasil et al. (1993) Bio/Technol. 11:1553-1558; Weeks et al. (1993) Plant Physiol. 102:1077-1084), rice (Christou (1991) Bio/Technol. 9:957-962; Hiei et al. (1994) Plant J. 6:271-282; Aldemita and Hodges (1996) Planta 199:612-617; and Hiei et al. (1997) Plant Mol. Biol. 35:205-218). For most cereal plants, embryogenic cells derived from immature scutellum tissues are the preferred cellular targets for transformation (Hiei et al. (1997) Plant Mol. Biol. 35:205-218; Vasil (1994) Plant Mol. Biol. 25: 925-937).

Vectors according to the present invention may be transformed into corn embryogenic cells derived from immature scutellar tissue by using microprojectile bombardment, with the A188XB73 genotype as the preferred genotype (Fromm et al. (1990) Bio/Technol. 8: 833-839; Gordon-Kamm et al. (1990) Plant Cell 2: 603-618). After microprojectile bombardment the tissues are selected on phosphinothricin to identify the transgenic embryogenic cells (Gordon-Kamm et al. (1990) Plant Cell 2: 603-618). Transgenic plants are regenerated by standard corn regeneration techniques (Fromm et al. (1990) Bio/Technol. 8: 833-839; Gordon-Kamm et al. (1990) Plant Cell 2: 603-618).

The plasmids prepared as described above can also be used to produce transgenic wheat and rice plants (Christou (1991) Bio/Technol. 9:957-962; Hiei et al. (1994) Plant J. 6:271-282; Aldemita and Hodges (1996) Planta 199:612-617; and Hiei et al. (1997) Plant Mol. Biol. 35:205-218) that coordinately express genes of interest by following standard transformation protocols known to those skilled in the art for rice and wheat (Vasil et al. (1992) Bio/Technol. 10:667-674; Vasil et al. (1993) Bio/Technol. 11: 1553-1558; and Weeks et al. (1993) Plant Physiol. 102:1077-1084), where the bar gene is used as the selectable marker.

### Example XV

### Transformation of Tomato and Soy Plants

Numerous protocols for the transformation of tomato and soy plants have been previously described, and are well known in the art. Gruber et al. ((1993) in Methods in Plant Molecular Biology and Biotechnology, p. 89-119, Glick and Thompson, eds., CRC Press, Inc., Boca Raton) describe several expression vectors and culture methods that may be used for cell or tissue transformation and subsequent regeneration. For soybean transformation, methods are described by Miki et al. (1993) in Methods in Plant Molecular Biology and Biotechnology, p. 67-88, Glick and Thompson, eds., CRC Press, Inc., Boca Raton; and U.S. Pat. No. 5,563,055, (Townsend and Thomas), issued Oct. 8, 1996.

There are a substantial number of alternatives to Agrobacterium-mediated transformation protocols, other methods for the purpose of transferring exogenous genes into soybeans or tomatoes. One such method is microprojectile-mediated transformation, in which DNA on the surface of microprojectile particles is driven into plant tissues with a biolistic device (see, for example, Sanford et al., (1987) Part. Sci. Technol. 5:27-37; Christou et al. (1992) Plant. J. 2: 275-281; Sanford (1993) Methods Enzymol. 217: 483-509; Klein et al. (1987) Nature 327: 70-73; U.S. Pat. No.5,015,580 (Christou et al), issued May 14, 1991; and U.S. Pat. No. 5,322,783 (Tomes et al.), issued Jun. 21, 1994.

Alternatively, sonication methods (see, for example, Zhang et al. (1991) Bio/Technology 9: 996-997); direct uptake of DNA into protoplasts using CaCl2 precipitation, polyvinyl alcohol or poly-L-ornithine (see, for example, Hain et al. (1985) Mol. Gen. Genet. 199: 161-168; Draper et al., Plant Cell Physiol. 23: 451-458 (1982)); liposome or spheroplast fusion (see, for example, Deshayes et al. (1985) EMBO J., 4: 2731-2737; Christou et al. (1987) Proc. Natl. Acad. Sci. U.S.A. 84: 3962-3966); and electroporation of protoplasts and whole cells and tissues (see, for example, Donn et al.(1990) in Abstracts of VIIth International Congress on Plant Cell and Tissue Culture IAPTC, A2-38: 53; D'Halluin et al. (1992) Plant Cell 4: 1495-1505;and Spencer et al. (1994) Plant Mol. Biol. 24: 51-61) have been used to introduce foreign DNA and expression vectors into plants.

After plants or plant cells are transformed (and the latter regenerated into plants) the transgenic plant thus generated may be crossed with itself or a plant from the same line, a non-transformed or wild-type plant, or another transformed plant from a different transgenic line of plants. Crossing provides the advantages of being able to produce new and perhaps stable transgenic varieties. Genes and the traits they confer that have been introduced into a tomato or soybean line may be moved into distinct line of plants using traditional backcrossing techniques well known in the art. Transformation of tomato plants may be conducted using the protocols of Koornneef et al (1986) In Tomato Biotechnology: Alan R. Liss, Inc., 169-178,and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 liM a-naphthalene acetic acid and 4.4 μM 6-benzylaminopurine. The explants are then infected with a diluted overnight culture of Agrobacterium tumefaciens containing an expression vector comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD₆₀₀ of 0.8.

Following the cocultivation, the cotyledon explants are transferred to Petri dishes with selective medium consisting of MS medium supplemented with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a medium containing kanamycin sulfate is regarded as a positive indication of a successful transformation.

Transformation of soybean plants may be conducted using the methods found in, for example, U.S. Pat. No. 5,563,055 (Townsend et al., issued Oct. 8, 1996), described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide of the invention are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium which has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

### Example XVI

### Genes that Confer Significant Improvements to Non-Arabidopsis Species

The function of specific orthologs of the sequences in the Sequence Listing may be analyzed through their ectopic overexpression in plants, using the CaMV 35S or other appropriate promoter, identified above. These genes include polynucleotide sequences found in the Sequence Listing such as those found in Arabidopsis thaliana SEQ ID NO: 2 (G47) and SEQ ID NO: 12 (G2133); Oryza sativa (japonica cultivar-group) SEQ ID NO: 98 (G3649), SEQ ID NO: 100 (G3651), and SEQ ID NO: 90 (G3644); Glycine max SEQ ID NO: 88 (G3643); Zinnia elegans SEQ ID NO: 96 (G3647); Brassica rapa subsp. Pekinensis SEQ ID NO: 92 (G3645); and Brassica oleracea SEQ ID NO: 94 (G3646). The polynucleotide and polypeptide sequences derived from monocots may be used to transform both monocot and dicot plants, and those derived from dicots may be used to transform either group, although some of these sequences will function best if the gene is transformed into a plant from the same group as that from which the sequence is derived.

Seeds of these transgenic plants are subjected to germination assays to measure sucrose sensing. Sterile monocot seeds, including, but not limited to, corn, rice, wheat, rye and sorghum, as well as dicots including, but not limited to soybean and alfalfa, are sown on 80% MS medium plus vitamins with 9.4% sucrose; control media lack sucrose. All assay plates are then incubated at 22° C. under 24-hour light, 120-130 μEin/m²/s, in a growth chamber. Evaluation of germination and seedling vigor is then conducted three days after planting. Overexpressors of these genes may be found to be more tolerant to high sucrose by having better germination, longer radicles, and more cotyledon expansion. These results would indicate that overexpressors of the orthologs in the Sequence Listing are involved in sucrose-specific sugar sensing.

Plants overexpressing these orthologs may also be subjected to soil-based drought assays to identify those lines that are more tolerant to water deprivation than wild-type control plants. Generally, ortholog overexpressing plants will appear significantly larger and greener, with less wilting or desiccation, than wild-type controls plants, particularly after a period of water deprivation is followed by rewatering and a subsequent incubation period.

### Example XVII

### Identification of Orthologous and Paralogous Sequences

Orthologs to Arabidopsis genes may identified by several methods, including hybridization, amplification, or bioinformatically. This example describes how one may identify homologs to the Arabidopsis AP2 family transcription factor CBF1 (polynucleotide SEQ ID NO: 421, encoded polypeptide SEQ ID NO: 422), which confers tolerance to abiotic stresses (Thomashow et al. (2002) U.S. Pat. No. 6,417,428), and an example to confirm the function of homologous sequences. In this example, orthologs to CBF1 were found in canola (Brassica napus) using polymerase chain reaction (PCR).

Degenerate primers were designed for regions of AP2 binding domain and outside of the AP2 (carboxyl terminal domain):

Mol 368 (reverse)

(SEQ ID NO:429)

5′-CAY CCN ATH TAY MGN GGN GT -3′
Mol 378 (forward)

(SEQ ID NO:430)

5′-GGN ARN ARC ATN CCY TCN GCC -3′
(Y: C/T, N: A/C/G/T, H: A/C/T, M: A/C, R: A/G)

Primer Mol 368 is in the AP2 binding domain of CBF1 (amino acid sequence: His-Pro-Ile-Tyr-Arg-Gly-Val) while primer Mol 378 is outside the AP2 domain (carboxyl terminal domain) (amino acid sequence: Met-Ala-Glu-Gly-Met-Leu-Leu-Pro).

The genomic DNA isolated from B. napus was PCR-amplified by using these primers following these conditions: an initial denaturation step of 2 min at 93° C.; 35 cycles of 93° C. for 1 min, 55° C. for 1 min, and 72° C. for 1 min ; and a final incubation of 7 min at 72° C. at the end of

The PCR products were separated by electrophoresis on a 1.2% agarose gel and transferred to nylon membrane and hybridized with the AT CBF1 probe prepared from Arabidopsis genomic DNA by PCR amplification. The hybridized products were visualized by colorimetric detection system (Boehringer Mannheim) and the corresponding bands from a similar agarose gel were isolated using the Qiagen Extraction Kit (Qiagen). The DNA fragments were ligated into the TA clone vector from TOPO TA Cloning Kit (Invitrogen) and transformed into E. coli strain TOP 10 (Invitrogen).

Seven colonies were picked and the inserts were sequenced on an ABI 377 machine from both strands of sense and antisense after plasmid DNA isolation. The DNA sequence was edited by sequencer and aligned with the AtCBF1 by GCG software and NCBI blast searching.

The nucleic acid sequence and amino acid sequence of one canola ortholog found in this manner (bnCBF1; polynucleotide SEQ ID NO: 427 and polypeptide SEQ ID NO: 428) identified by this process is shown in the Sequence Listing.

The aligned amino acid sequences show that the bnCBF1 gene has 88% identity with the Arabidopsis sequence in the AP2 domain region and 85% identity with the Arabidopsis sequence outside the AP2 domain when aligned for two insertion sequences that are outside the AP2 domain.

Similarly, paralogous sequences to Arabidopsis genes, such as CBF1, may also be identified.

Two paralogs of CBF1 from Arabidopsis thaliana: CBF2 and CBF3. CBF2 and CBF3 have been cloned and sequenced as described below. The sequences of the DNA SEQ ID NO: 421, 423 and 425 and encoded proteins SEQ ID NO: 422, 424 and 426 are set forth in the Sequence Listing.

A lambda cDNA library prepared from RNA isolated from Arabidopsis thaliana ecotype Columbia (Lin and Thomashow (1992) Plant Physiol. 99: 519-525) was screened for recombinant clones that carried inserts related to the CBF1 gene (Stockinger et al. (1997) Proc. Natl. Acad. Sci. 94:1035-1040). CBF1 was ³²P-radiolabeled by random priming (Sambrook et al. supra) and used to screen the library by the plaque-lift technique using standard stringent hybridization and wash conditions (Hajela et al. (1990) Plant Physiol. 93:1246-1252; Sambrook et al. supra) 6×SSPE buffer, 60° C. for hybridization and 0.1×SSPE buffer and 60° C. for washes). Twelve positively hybridizing clones were obtained and the DNA sequences of the cDNA inserts were determined. The results indicated that the clones fell into three classes. One class carried inserts corresponding to CBF1. The two other classes carried sequences corresponding to two different homologs of CBF1, designated CBF2 and CBF3. The nucleic acid sequences and predicted protein coding sequences for Arabidopsis CBF1, CBF2 and CBF3 are listed in the Sequence Listing (SEQ ID NOs:421, 423, 425 and SEQ ID NOs: 422, 424, and 426, respectively). The nucleic acid sequences and predicted protein coding sequence for Brassica napus CBF ortholog is listed in the Sequence Listing (SEQ ID NOs: 427 and 428, respectively).

A comparison of the nucleic acid sequences of Arabidopsis CBF1, CBF2 and CBF3 indicate that they are 83 to 85% identical as shown in Table 11.

TABLE 11

Percent identity^{a}

| | DNA^{b} | Polypeptide |
|---|---|---|
| cbf1/cbf2 | 85 | 86 |
| cbf1/cbf3 | 83 | 84 |
| cbf2/cbf3 | 84 | 85 |

^{a}Percent identity was determined using the Clustal algorithm from the Megalign program (DNASTAR, Inc.).
^{b}Comparisons of the nucleic acid sequences of the open reading frames are shown.

Similarly, the amino acid sequences of the three CBF polypeptides range from 84 to 86% identity. An alignment of the three amino acidic sequences reveals that most of the differences in amino acid sequence occur in the acidic C-terminal half of the polypeptide. This region of CBF1 serves as an activation domain in both yeast and Arabidopsis (not shown).

Residues 47 to 106 of CBF1 correspond to the AP2 domain of the protein, a DNA binding motif that to date, has only been found in plant proteins. A comparison of the AP2 domains of CBF1, CBF2 and CBF3 indicates that there are a few differences in amino acid sequence. These differences in amino acid sequence might have an effect on DNA binding specificity.

### Example XVIII

### Transformation of Canola With a Plasmid Containing CBF1, CBF2, or CBF3

After identifying homologous genes to CBF1, canola was transformed with a plasmid containing the Arabidopsis CBF1, CBF2, or CBF3 genes cloned into the vector pGA643 (An (1987) Methods Enzymol. 253: 292). In these constructs the CBF genes were expressed constitutively under the CaMV 35S promoter. In addition, the CBF1 gene was cloned under the control of the Arabidopsis COR15 promoter in the same vector pGA643. Each construct was transformed into Agrobacterium strain GV3101. Transformed Agrobacteria were grown for 2 days in minimal AB medium containing appropriate antibiotics.

Spring canola (B. napus cv. Westar) was transformed using the protocol of Moloney et al. ((1989) Plant Cell Reports 8: 238) with some modifications as described. Briefly, seeds were sterilized and plated on half strength MS medium, containing 1% sucrose. Plates were incubated at 24° C. under 60-80 μE/m²s light using a 16 hour light/8 hour dark photoperiod. Cotyledons from 4-5 day old seedlings were collected, the petioles cut and dipped into the Agrobacterium solution. The dipped cotyledons were placed on co-cultivation medium at a density of 20 cotyledons/plate and incubated as described above for 3 days. Explants were transferred to the same media, but containing 300 mg/l timentin (SmithKline Beecham, Pa.) and thinned to 10 cotyledons/plate. After 7 days explants were transferred to Selection/Regeneration medium. Transfers were continued every 2-3 weeks (2 or 3 times) until shoots had developed. Shoots were transferred to Shoot-Elongation medium every 2-3 weeks. Healthy looking shoots were transferred to rooting medium. Once good roots had developed, the plants were placed into moist potting soil.

The transformed plants were then analyzed for the presence of the NPTII gene/kanamycin resistance by ELISA, using the ELISA NPTII kit from 5Prime-3Prime Inc. (Boulder, Colo.). Approximately 70% of the screened plants were NPTII positive. Only those plants were further analyzed.

From Northern blot analysis of the plants that were transformed with the constitutively expressing constructs, showed expression of the CBF genes and all CBF genes were capable of inducing the Brassica napus cold-regulated gene BN115 (homolog of the Arabidopsis COR15 gene). Most of the transgenic plants appear to exhibit a normal growth phenotype. As expected, the transgenic plants are more freezing tolerant than the wild-type plants. Using the electrolyte leakage of leaves test, the control showed a 50% leakage at −2 to −3° C. Spring canola transformed with either CBF1 or CBF2 showed a 50% leakage at −6 to −7° C. Spring canola transformed with CBF3 shows a 50% leakage at about −10 to −15° C. Winter canola transformed with CBF3 may show a 50% leakage at about −16 to −20° C. Furthermore, if the spring or winter canola are cold acclimated the transformed plants may exhibit a further increase in freezing tolerance of at least −2° C.

To test salinity tolerance of the transformed plants, plants were watered with 150 mM NaCl. Plants overexpressing CBF1, CBF2 or CBF3 grew better compared with plants that had not been transformed with CBF1, CBF2 or CBF3.

These results demonstrate that homologs of Arabidopsis transcription factors can be identified and shown to confer similar functions in non-Arabidopsis plant species.

### Example XIX

### Cloning of Transcription Factor Promoters

Promoters are isolated from transcription factor genes that have gene expression patterns useful for a range of applications, as determined by methods well known in the art (including transcript profile analysis with cDNA or oligonucleotide microarrays, Northern blot analysis, semi-quantitative or quantitative RT-PCR). Interesting gene expression profiles are revealed by determining transcript abundance for a selected transcription factor gene after exposure of plants to a range of different experimental conditions, and in a range of different tissue or organ types, or developmental stages. Experimental conditions to which plants are exposed for this purpose includes cold, heat, drought, osmotic challenge, varied hormone concentrations (ABA, GA, auxin, cytokinin, salicylic acid, brassinosteroid), pathogen and pest challenge. The tissue types and developmental stages include stem, root, flower, rosette leaves, cauline leaves, siliques, germinating seed, and meristematic tissue. The set of expression levels provides a pattern that is determined by the regulatory elements of the gene promoter.

Transcription factor promoters for the genes disclosed herein are obtained by cloning 1.5 kb to 2.0 kb of genomic sequence immediately upstream of the translation start codon for the coding sequence of the encoded transcription factor protein. This region includes the 5′-UTR of the transcription factor gene, which can comprise regulatory elements. The 1.5 kb to 2.0 kb region is cloned through PCR methods, using primers that include one in the 3′ direction located at the translation start codon (including appropriate adaptor sequence), and one in the 5′ direction located from 1.5 kb to 2.0 kb upstream of the translation start codon (including appropriate adaptor sequence). The desired fragments are PCR-amplified from Arabidopsis Col-0 genomic DNA using high-fidelity Taq DNA polymerase to minimize the incorporation of point mutation(s). The cloning primers incorporate two rare restriction sites, such as Not1 and Sfi1, found at low frequency throughout the Arabidopsis genome. Additional restriction sites are used in the instances where a Not1 or Sfi1 restriction site is present within the promoter.

The 1.5-2.0 kb fragment upstream from the translation start codon, including the 5′-untranslated region of the transcription factor, is cloned in a binary transformation vector immediately upstream of a suitable reporter gene, or a transactivator gene that is capable of programming expression of a reporter gene in a second gene construct. Reporter genes used include green fluorescent protein (and related fluorescent protein color variants), beta-glucuronidase, and luciferase. Suitable transactivator genes include LexA-GAL4, along with a transactivatable reporter in a second binary plasmid (as disclosed in U.S. patent application Ser. No. 09/958,131, incorporated herein by reference). The binary plasmid(s) is transferred into Agrobacterium and the structure of the plasmid confirmed by PCR. These strains are introduced into Arabidopsis plants as described in other examples, and gene expression patterns determined according to standard methods know to one skilled in the art for monitoring GFP fluorescence, beta-glucuronidase activity, or luminescence.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A recombinant polynucleotide comprising a nucleotide sequence that hybridizes to SEQ ID NO: 11 or the complement of SEQ ID NO: 11 under stringent conditions that include two wash steps of 6×SSC and 65° C. for 10-30 minutes per step.

2. The recombinant polynucleotide of claim 1, wherein the recombinant polynucleotide is operably linked to at least one regulatory element being effective in controlling expression of the recombinant polynucleotide when the recombinant polynucleotide is transformed into a plant.

3. The recombinant polynucleotide of claim 1, wherein the recombinant polynucleotide is incorporated into an expression vector.

4. The recombinant polynucleotide of claim 3, wherein the recombinant polynucleotide is incorporated into a cultured host cell.

5. The recombinant polynucleotide of claim 1, wherein the recombinant polynucleotide encodes a polypeptide comprising the AP2 domain of SEQ ID NO: 12.

6. The recombinant polynucleotide of claim 1, wherein the recombinant polynucleotide encodes a polypeptide comprising SEQ ID NO: 12.

7. The recombinant polynucleotide of claim 1, wherein said recombinant polynucleotide comprises SEQ ID NO: 11.

8. A transgenic plant comprising the recombinant polynucleotide of claim 1.

9. Seed produced from the transgenic plant claim 8.

10. A recombinant polynucleotide comprising a nucleotide sequence that hybridizes to the nucleotide bases 53 to 256 of SEQ ID NO: 11 or the complement of nucleotide bases 53 to 256 of SEQ ID NO: 11 under stringent conditions that include two wash steps of 6×SSC and 65° C. for 10-30 minutes per step.

11. A transgenic plant comprising the recombinant polynucleotide of claim 10.

12. Seed produced from the transgenic plant claim 11.

13. A transgenic plant comprising a recombinant polynucleotide encoding a polypeptide;
wherein the polypeptide has the property of regulating abiotic stress tolerance in a plant when the polypeptide is overexpressed wherein the recombinant polynucleotide a nucleotide sequence selected from the group consisting of:
(a) SEQ ID NO: 2N-1, where N=1-210;
(b) a nucleic acid sequence that hybridizes to the nucleotide sequence or the complement of the nucleotide sequence of (a) under stringent conditions that include two wash steps of 6×SSC and 65° C., for 10-30 minutes per step; and
(c) a nucleic acid sequence that is substantially identical to the nucleotide sequence of (a).

14. A transgenic plant comprising a recombinant polynucleotide encoding a polypeptide having an AP2 domain, wherein the polypeptide has the property of SEQ ID NO: 12 of regulating abiotic stress tolerance in a plant when the polypeptide is overexpressed, wherein:
the AP2 domain is sufficiently homologous to the AP2 domain of SEQ ID NO: 12 that the polypeptide binds to a transcription-regulating region of DNA.

15. The transgenic plant of claim 14, wherein the recombinant polynucleotide has a nucleotide sequence that hybridizes to SEQ ID NO: 11 or to the complement of SEQ ID NO 11 under stringent conditions that include two wash steps of 6×SSC and 65° C. of 10-30 minutes per step.

16. The transgenic plant of claim 14, wherein the recombinant polynucleotide has a nucleotide sequence that hybridizes to the complement of nucleotide bases 53-256 of SEQ ID NO 11 under stringent conditions that include two wash steps of 6×SSC and 65° C. of 10-30 minutes per step.

17. The transgenic plant of claim 14, wherein the recombinant polynucleotide comprises SEQ ID NO: 11.

18. The transgenic plant of claim 14, wherein the polypeptide comprises SEQ ID NO: 12.

19. The transgenic plant of claim 14, wherein the transgenic plant is selected from the group consisting of: soybean, wheat, corn, potato, cotton, rice, oilseed rape, sunflower, alfalfa, clover, sugarcane, turf, banana, blackberry, blueberry, strawberry, raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, watermelon, mint and other labiates, citrus, fruit trees, rosaceous fruits, and brassicas.

20. The transgenic plant of claim 14, wherein the recombinant polynucleotide comprises a constitutive, inducible, or tissue-specific promoter that is operably linked to a region of the recombinant polynucleotide that encodes the polypeptide.

21. The transgenic plant of claim 14, wherein the polypeptide is selected from the group consisting of SEQ ID NOs: 2, 12, 88, 90, 92, 94, 96, 98, and 100.

22. Seed produced from the transgenic plant of claim 14.

23. A transgenic plant that overexpresses a recombinant polynucleotide comprising a nucleotide sequence that hybridizes to SEQ ID NO: 11 or the complement of SEQ ID NO 11 under stringent conditions including two wash steps of 6×SSC and 65° C. for 10-30 minutes per step;
wherein said transgenic plant has increased abiotic stress tolerance as compared to a non-transformed plant that does not overexpress a polypeptide encoded by the recombinant polynucleotide.

24. The transgenic plant of claim 23, wherein said abiotic stress tolerance is selected from the group consisting of tolerance to drought, tolerance to chilling, germination in cold conditions, and tolerance to low nitrogen.

25. A method for producing a transgenic plant having increased tolerance to abiotic stress, the method steps comprising:
(a) providing an expression vector comprising a nucleotide sequence that hybridizes to the complement of SEQ ID NO 11 under stringent conditions that include two wash steps of 6×SSC and 65° C., each step being 10-30 minutes in duration;
(b) introducing the expression vector into a plant cell;
(c) growing the plant cell into a plant, and allowing the plant to overexpress a polypeptide encoded by the nucleotide sequence, said polypeptide having the property of increasing abiotic stress tolerance in the transgenic plant as compared to a non-transformed plant that does not overexpress the polypeptide;
(d) identifying an abiotic stress tolerant plant so produced with increased abiotic stress tolerance by comparing the transgenic plant with one or more non-transformed plants that do not overexpress the polypeptide; and
(e) selecting said abiotic stress tolerant plant with increased abiotic stress tolerance.

26. The method of claim 25, the method steps further comprising:
(e) selfing or crossing said abiotic stress tolerant plant with itself or another plant, respectively, to produce seed; and
(f) growing a progeny plant from the seed, wherein said progeny plant has increased tolerance to the abiotic stress.

27. The method of claim 26, wherein:
said progeny plant expresses mRNA that encodes a DNA-binding protein having an AP2 domain that binds to a DNA molecule, regulates expression of said DNA molecule, which induces the overexpression of the polypeptide; and
said mRNA is expressed in the progeny plant at a level greater than a non-transformed plant
that does not overexpress said DNA-binding protein.

28. The method of claim 25, wherein the abiotic stress tolerance is selected from the group consisting of tolerance to drought, tolerance to chilling, germination in cold conditions, and tolerance to low nitrogen.

29. The method of claim 25, wherein the transgenic plant is produced from a plant selected from the group consisting of: soybean, wheat, corn, potato, cotton, rice, oilseed rape, sunflower, alfalfa, clover, sugarcane, turf, banana, blackberry, blueberry, strawberry, raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, watermelon, mint and other labiates, citrus, fruit trees, rosaceous fruits, and brassicas.

30. A method for increasing a plant's tolerance to abiotic stress, said method comprising:
(a) providing a vector comprising:
(i) a polynucleotide sequence, wherein the polynucleotide sequence encodes a polypeptide having an AP2 domain, and the polypeptide has the property of SEQ ID NO:12 of regulating abiotic stress tolerance in a plant; and
(ii) regulatory elements flanking the polynucleotide sequence, said regulatory elements being effective to control expression of said polynucleotide sequence; and
(b) transforming a target plant with said vector to generate a transformed plant with increased tolerance to abiotic stress as compared to a control plant that does not overexpress the polypeptide.

31. The method of claim 30, wherein the polynucleotide comprises a nucleotide sequence that hybridizes to SEQ ID NO: 11 or the complement of SEQ ID NO: 11 under stringent conditions of two wash steps of 6×SSC and 65° C. per step.

32. The method of claim 30, wherein said polynucleotide comprises SEQ ID NO: 11; or a nucleotide sequence that encodes SEQ ID NO: 12.

33. The method of claim 30, wherein said abiotic stress tolerance is selected from the group consisting of tolerance to drought, tolerance to chilling, germination in cold conditions, and tolerance to low nitrogen.

34. A method for increasing a plant's tolerance to drought stress, the method comprising:
(a) providing a vector comprising:
(i) a polynucleotide sequence, wherein the polynucleotide sequence encodes SEQ ID NO 12; and
(ii) regulatory elements flanking the polynucleotide sequence, said regulatory elements being effective to control expression of said polynucleotide sequence; and
(b) transforming a target plant with the vector to generate a transformed plant with increased tolerance to drought stress as compared to a control plant that does not overexpress SEQ ID NO 12.
